SEQUENCE LISTING (37 sequences)

SEQ ID NO: 1 - DNA, 25 bp, Anas sp.
gggcaacatccagcaaaatcaatgg

SEQ ID NO: 2 - DNA, 30 bp, Anas sp.
gctgcggaatggctaaaagggcccccgacc

SEQ ID NO: 3 - DNA, 34 bp, Anas sp.
ccaacactagatcacgaaacccacgtcaccgggg

SEQ ID NO: 4 - DNA, 34 bp, Anas sp.
ggttgtgatctagtgctttgggtgcagtggcccc

SEQ ID NO: 5 - DNA, 3021 bp, Anas sp.
catgctcatttgaaagcttatgcaaaaattaacgaggaatcactggatagggctaggagattgctttggtggcattacaactgtttactgtggggagaagctcaagttactaactatatttctcgtttgcgtacttggttgtcaactcctgagaaatatagaggtagagatgccccgaccattgaagcaatcactagaccaatccaggtggctcagggaggcagaaaaacaactacgggtactagaaaacctcgtggactcgaacctagaagaagaaaagttaaaaccacagttgtctatgggagaagacgttcaaagtcccgggaaaggagagcccctacaccccaacgtgcgggctcccctctcccacgtagttcgagcagccaccatagatctccctcgcctaggaaataaattacctgctaggcatcacttaggtaaattgtcaggactatatcaaatgaagggctgtacttttaacccagaatggaaagtaccagatatttcggatactcattttaatttagatgtagttaatgagtgcccttcccgaaattggaaatatttgactccagccaaattctggcccaagagcatttcctactttcctgtccaggtaggggttaaaccaaagtatcctgacaatgtgatgcaacatgaatcaatagtaggtaaatatttaaccaggctctatgaagcaggaatcctttataagcggatatctaaacatttggtcacatttaaaggtcagccttataattgggaacagcaacaccttgtcaatcaacatcacatttatgatggggcaacatccagcaaaatcaatggacgtcagacggatagaaggaggagaaatactgttaaaccaacttgccggaaggatgatcccaaaagggactttgacatggtcaggcaagtttccaacactagatcacgtgttagaccatgtgcaaacaatggaggagataaacaccctccagaatcagggagcttggcctgctggggcgggaaggagagtaggattatcaaatccgactcctcaagagattcctcagccccagtggactcccgaggaagaccaaaaagcacgcgaagcttttcgccgttatcaagaagaaagaccaccggaaaccaccaccattcctccgtcttcccctcctcagtggaagctacaacccggggacgatccactcctgggaaatcagtctctcctcgagactcatccgctataccagtcagaaccagcggtgccagtgataaaaactccccccttgaagaagaaaatgtctggtaccttcgggggaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttaggaataagaataaactttgacaaaaccacgccttctccggtgaatgaaataagattcctcggttaccagattgatgaaaatttcatgaagattgaagaaagcagatggaaagaattaaggactgtaatcaagaaaataaaagtaggagaatggtatgactggaaatgtattcaaagatttgtggggcatttgaattttgttttgccttttactaaaggtaatattgaaatgttaaaaccaatgtatgctgctattactaaccaagtaaactttagcttctcttcatcctataggactttgttatataaactaacaatgggtgtgtgtaaattaagaataaagccaaagtcctctgtacctttgccacgtgtagctacagatgctaccccaacacatggcgcaatatcccatatcaccggcgggagcgcagtgtttgctttttcaaaggtcagagatatacatgttcaggaactattgatgtcttgtttagccaagataatgattaaaccacgttgtctcttatctgattcaacttttgtttgccataagcgttatcagacgttaccatggcattttgctatgttggccaaacaattgctcaaaccgatacaattgtactttgtcccgagcaaatataatcctgctgacggcccatccaggcacaaacctcctgattggacggcttttccatacacccctctctcgaaagcaatatatattccacataggctatgtggaacttaagaattacacccctctccttcggagctgcttgccaaggtatctttacgtctacattgctgttgtcgtgtgtgactgtacctttggtatgtaccattgtttatgattcttgcttatatatggatatcaatgcttctagagccttagccaatgtgtatgatctaccagatgatttctttccaaaaatagatgatcttgttagagatgctaaagacgctttagagccttattggaaatcagattcaataaagaaacatgttttgattgcaactcactttgtggatctcattgaagacttctggcagactacacagggcatgcatgaaatagccgaatcattaagagctgttatacctcccactactactcctgttccaccgggttatcttattcagcacgaggaagctgaagagatacctttgggagatttatttaaacaccaagaagaaaggatagtaagtttccaacccgactatccgattacggctagaatt

SEQ ID NO: 6 - DNA, 984 bp, Anas sp.
  CDS (1)..(984)
atggggcaacatccagcaaaatcaatggacgtcagacggatagaaggaggagaaatactgttaaaccaacttgccggaaggatgatcccaaaagggactttgacatggtcaggcaagtttccaacactagatcacgtgttagaccatgtgcaaacaatggaggagataaacaccctccagaatcagggagcttggcctgctggggcgggaaggagagtaggattatcaaatccgactcctcaagagattcctcagccccagtggactcccgaggaagaccaaaaagcacgcgaagcttttcgccgttatcaagaagaaagaccaccggaaaccaccaccattcctccgtcttcccctcctcagtggaagctacaacccggggacgatccactcctgggaaatcagtctctcctcgagactcatccgctataccagtcagaaccagcggtgccagtgataaaaactccccccttgaagaagaaaatgtctggtaccttcgggggaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagt

SEQ ID NO: 7 - protein, 328 aa, Anas sp.
MGQHPAKSMDVRRIEGGEILLNQLAGRMIPKGTLTWSGKFPTLDHVLDHVQTMEEINTLQNQGAWPAGAGRRVGLSNPTPQEIPQPQWTPEEDQKAREAFRRYQEERPPETTTIPPSSPPQWKLQPGDDPLLGNQSLLETHPLYQSEPAVPVIKTPPLKKKMSGTFGGILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKS

SEQ ID NO: 8 - DNA, 501 bp, Anas sp.
  CDS (1)..(501)
atgtctggtaccttcgggggaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagt

SEQ ID NO: 9 - protein, 167 aa, Anas sp.
MSGTFGGILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKS

SEQ ID NO: 10 - DNA, 483 bp, Anas sp.
  CDS (1)..(483)
atggggcaacatccagcaaaatcaatggacgtcagacggatagaaggaggagaaatactgttaaaccaacttgccggaaggatgatcccaaaagggactttgacatggtcaggcaagtttccaacactagatcacgtgttagaccatgtgcaaacaatggaggagataaacaccctccagaatcagggagcttggcctgctggggcgggaaggagagtaggattatcaaatccgactcctcaagagattcctcagccccagtggactcccgaggaagaccaaaaagcacgcgaagcttttcgccgttatcaagaagaaagaccaccggaaaccaccaccattcctccgtcttcccctcctcagtggaagctacaacccggggacgatccactcctgggaaatcagtctctcctcgagactcatccgctataccagtcagaaccagcggtgccagtgataaaaactccccccttgaagaagaaa

SEQ ID NO: 11 - protein, 161 aa, Anas sp.
MGQHPAKSMDVRRIEGGEILLNQLAGRMIPKGTLTWSGKFPTLDHVLDHVQTMEEINTLQNQGAWPAGAGRRVGLSNPTPQEIPQPQWTPEEDQKAREAFRRYQEERPPETTTIPPSSPPQWKLQPGDDPLLGNQSLLETHPLYQSEPAVPVIKTPPLKKK

SEQ ID NO: 12 - DNA, 501 bp, Anas sp.; identical to SEQ ID NO: 8
  CDS (1)..(501)

SEQ ID NO: 13 - protein, 167 aa, Anas sp.; identical to SEQ ID NO: 9

SEQ ID NO: 14 - DNA, 9599 bp, Hepatitis C virus
gccagccccctgatgggggcgacactccaccatgaatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgactgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggggaaatgccggccgcaccacggctgggcttgttggtctccttacaccaggcgccaagcagaacatccaactgatcaacaccaacggcagttggcacatcaatagcacggccttgaattgcaatgaaagccttaacaccggctggttagcagggctcttctatcaacacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgcttccgcaaacatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccgttgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttccttctgcttgcagacgcgcgcgtctgctcctgcttgtggatgatgttactcatatcccaagcggaggcggctttggagaacctcgtaatactcaatgcagcatccctggccgggacgcacggtcttgtgtccttcctcgtgttcttctgctttgcgtggtatctgaagggtaggtgggtgcccggagcggtctacgccctctacgggatgtggcctctcctcctgctcctgctggcgttgcctcagcgggcatacgcactggacacggaggtggccgcgtcgtgtggcggcgttgttcttgtcgggttaatggcgctgactctgtcgccatattacaagcgctatatcagctggtgcatgtggtggcttcagtattttctgaccagagtagaagcgcaactgcacgtgtgggttccccccctcaacgtccggggggggcgcgatgccgtcatcttactcatgtgtgtagtacacccgaccctggtatttgacatcaccaaactactcctggccatcttcggacccctttggattcttcaagccagtttgcttaaagtcccctacttcgtgcgcgttcaaggccttctccggatctgcgcgctagcgcggaagatagccggaggtcattacgtgcaaatggccatcatcaagttaggggcgcttactggcacctatgtgtataaccatctcacccctcttcgagactgggcgcacaacggcctgcgagatctggccgtggctgtggaaccagtcgtcttctcccgaatggagaccaagctcatcacgtggggggcagataccgccgcgtgcggtgacatcatcaacggcttgcccgtctctgcccgtaggggccaggagatactgcttgggccagccgacggaatggtctccaaggggtggaggttgctggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgagcacctgggtgctcgttggcggcgtcctggctgctctggccgcgtattgcctgtcaacaggctgcgtggtcatagtgggcaggatcgtcttgtccgggaagccggcaattatacctgacagggaggttctctaccaggagttcgatgagatggaagagtgctctcagcacttaccgtacatcgagcaagggatgatgctcgctgagcagttcaagcagaaggccctcggcctcctgcagaccgcgtcccgccatgcagaggttatcacccctgctgtccagaccaactggcagaaactcgaggtcttttgggcgaagcacatgtggaatttcatcagtgggatacaatacttggcgggcctgtcaacgctgcctggtaaccccgccattgcttcattgatggcttttacagctgccgtcaccagcccactaaccactggccaaaccctcctcttcaacatattgggggggtgggtggctgcccagctcgccgcccccggtgccgctactgcctttgtgggtgctggcctagctggcgccgccatcggcagcgttggactggggaaggtcctcgtggacattcttgcagggtatggcgcgggcgtggcgggagctcttgtagcattcaagatcatgagcggtgaggtcccctccacggaggacctggtcaatctgctgcccgccatcctctcgcctggagcccttgtagtcggtgtggtctgcgcagcaatactgcgccggcacgttggcccgggcgagggggcagtgcaatggatgaaccggctaatagccttcgcctcccgggggaaccatgtttcccccacgcactacgtgccggagagcgatgcagccgcccgcgtcactgccatactcagcagcctcactgtaacccagctcctgaggcgactgcatcagtggataagctcggagtgtaccactccatgctccggttcctggctaagggacatctgggactggatatgcgaggtgctgagcgactttaagacctggctgaaagccaagctcatgccacaactgcctgggattccctttgtgtcctgccagcgcgggtataggggggtctggcgaggagacggcattatgcacactcgctgccactgtggagctgagatcactggacatgtcaaaaacgggacgatgaggatcgtcggtcctaggacctgcaggaacatgtggagtgggacgttccccattaacgcctacaccacgggcccctgtactccccttcctgcgccgaactataagttcgcgctgtggagggtgtctgcagaggaatacgtggagataaggcgggtgggggacttccactacgtatcgggtatgactactgacaatcttaaatgcccgtgccagatcccatcgcccgaatttttcacagaattggacggggtgcgcctacacaggtttgcgcccccttgcaagcccttgctgcgggaggaggtatcattcagagtaggactccacgagtacccggtggggtcgcaattaccttgcgagcccgaaccggacgtagccgtgttgacgtccatgctcactgatccctcccatataacagcagaggcggccgggagaaggttggcgagagggtcacccccttctatggccagctcctcggctagccagctgtccgctccatctctcaaggcaacttgcaccgccaaccatgactcccctgacgccgagctcatagaggctaacctcctgtggaggcaggagatgggcggcaacatcaccagggttgagtcagagaacaaagtggtgattctggactccttcgatccgcttgtggcagaggaggatgagcgggaggtctccgtacctgcagaaattctgcggaagtctcggagattcgcccgggccctgcccgtctgggcgcggccggactacaaccccccgctagtagagacgtggaaaaagcctgactacgaaccacctgtggtccatggctgcccgctaccacctccacggtcccctcctgtgcctccgcctcggaaaaagcgtacggtggtcctcaccgaatcaaccctatctactgccttggccgagcttgccaccaaaagttttggcagctcctcaacttccggcattacgggcgacaatacgacaacatcctctgagcccgccccttctggctgcccccccgactccgacgttgagtcctattcttccatgccccccctggagggggagcctggggatccggatctcagcgacgggtcatggtcgacggtcagtagtggggccgacacggaagatgtcgtgtgctgctcaatgtcttattcctggacaggcgcactcgtcaccccgtgcgctgcggaagaacaaaaactgcccatcaacgcactgagcaactcgttgctacgccatcacaatctggtgtattccaccacttcacgcagtgcttgccaaaggcagaagaaagtcacatttgacagactgcaagttctggacagccattaccaggacgtgctcaaggaggtcaaagcagcggcgtcaaaagtgaaggctaacttgctatccgtagaggaagcttgcagcctgacgcccccacattcagccaaatccaagtttggctatggggcaaaagacgtccgttgccatgccagaaaggccgtagcccacatcaactccgtgtggaaagaccttctggaagacagtgtaacaccaatagacactaccatcatggccaagaacgaggttttctgcgttcagcctgagaaggggggtcgtaagccagctcgtctcatcgtgttccccgacctgggcgtgcgcgtgtgcgagaagatggccctgtacgacgtggttagcaagctccccctggccgtgatgggaagctcctacggattccaatactcaccaggacagcgggttgaattcctcgtgcaagcgtggaagtccaagaagaccccgatggggttctcgtatgatacccgctgttttgactccacagtcactgagagcgacatccgtacggaggaggcaatttaccaatgttgtgacctggacccccaagcccgcgtggccatcaagtccctcactgagaggctttatgttgggggccctcttaccaattcaaggggggaaaactgcggctaccgcaggtgccgcgcgagcggcgtactgacaactagctgtggtaacaccctcacttgctacatcaaggcccgggcagcctgtcgagccgcagggctccaggactgcaccatgctcgtgtgtggcgacgacttagtcgttatctgtgaaagtgcgggggtccaggaggacgcggcgagcctgagagccttcacggaggctatgaccaggtactccgccccccccggggaccccccacaaccagaatacgacttggagcttataacatcatgctcctccaacgtgtcagtcgcccacgacggcgctggaaagagggtctactaccttacccgtgaccctacaacccccctcgcgagagccgcgtgggagacagcaagacacactccagtcaattcctggctaggcaacataatcatgtttgcccccacactgtgggcgaggatgatactgatgacccatttctttagcgtcctcatagccagggatcagcttgaacaggctcttaactgtgagatctacggagcctgctactccatagaaccactggatctacctccaatcattcaaagactccatggcctcagcgcattttcactccacagttactctccaggtgaaatcaatagggtggccgcatgcctcagaaaacttggggtcccgcccttgcgagcttggagacaccgggcccggagcgtccgcgctaggcttctgtccagaggaggcagggctgccatatgtggcaagtacctcttcaactgggcagtaagaacaaagctcaaactcactccaatagcggccgctggccggctggacttgtccggttggttcacggctggctacagcgggggagacatttatcacagcgtgtctcatgcccggccccgctggttctggttttgcctactcctgctcgctgcaggggtaggcatctacctcctccccaaccgatgaaggttggggtaaacactccggcctcttaagccatttcctgttttttttttttttttttttttttttttctttttttttttctttcctttccttctttttttcctttctttttcccttctttaatggtggctccatcttagccctagtcacggctagctgtgaaaggtccgtgagccgcatgactgcagagagtgctgatactggcctctctgcagatcatgt

SEQ ID NO: 15 - protein, 3011 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYALYGMWPLLLLLLALPQRAYALDTEVAASCGGVVLVGLMALTLSPYYKRYISWCMWWLQYFLTRVEAQLHVWVPPLNVRGGRDAVILLMCVVHPTLVFDITKLLLAIFGPLWILQASLLKVPYFVRVQGLLRICALARKIAGGHYVQMAIIKLGALTGTYVYNHLTPLRDWAHNGLRDLAVAVEPVVFSRMETKLITWGADTAACGDIINGLPVSARRGQEILLGPADGMVSKGWRLLAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTSTWVLVGGVLAALAAYCLSTGCVVIVGRIVLSGKPAIIPDREVLYQEFDEMEECSQHLPYIEQGMMLAEQFKQKALGLLQTASRHAEVITPAVQTNWQKLEVFWAKHMWNFISGIQYLAGLSTLPGNPAIASLMAFTAAVTSPLTTGQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPRTCRNMWSGTFPINAYTTGPCTPLPAPNYKFALWRVSAEEYVEIRRVGDFHYVSGMTTDNLKCPCQIPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREVSVPAEILRKSRRFARALPVWARPDYNPPLVETWKKPDYEPPVVHGCPLPPPRSPPVPPPRKKRTVVLTESTLSTALAELATKSFGSSSTSGITGDNTTTSSEPAPSGCPPDSDVESYSSMPPLEGEPGDPDLSDGSWSTVSSGADTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVAHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALNCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 16 - DNA, 2076 bp, influenza virus
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaagagagagaagaggattatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgattattgacaaaatgaacactcagtttgaggccgttggaagggaatttaacaacttagaaaggagaatagagaatttaaacaagaagatggaagacgggttcctagatgtctggacttataatgctgaacttctagttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaacggaacgtatgactacccgcagtattcagaagaagcaagactaaaaagagaggaaataagtggagtaaaattggaatcaataggaatttacggtaccttcgggggaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 17 - DNA, 2082 bp, influenza virus
atggagaaaatagtgcttctttttgcaatagtcagtcttgttaaaagtgatcagatttgcattggttaccatgcaaacaactcgacagagcaggttgacacaataatggaaaagaacgttactgttacacatgcccaagacatactggaaaagacacacaatgggaagctctgcgatctagatggagtgaagcctctaattttgagagattgtagtgtagctggatggctcctcggaaacccaatgtgtgacgaattcatcaatgtgccggaatggtcttacatagtggagaaggccaatccagtcaatgacctctgttacccaggggatttcaatgactatgaagaattgaaacacctattgagcagaataaaccattttgagaaaattcagatcatccccaaaagttcttggtccagtcatgaagcctcattgggggtgagctcagcatgtccataccagggaaagtcctcctttttcagaaatgtggtatggcttatcaaaaagaacagtacatacccaacaataaagaggagctacaataataccaaccaagaagatcttttggtactgtgggggattcaccatcctaatgatgcggcagagcagacaaagctctatcaaaacccaaccacctatatttccgttgggacatcaacactaaaccagagattggtaccaagaatagctactagatccaaagtaaacgggcaaagtggaaggatggagttcttctggacaattttaaaaccgaatgatgcaatcaacttcgagagtaatggaaatttcattgctccagaatatgcatacaaaattgtcaagaaaggggactcaacaattatgaaaagtgaattggaatatggtaactgcaacaccaagtgtcaaactccaatgggggcgataaactctagcatgccattccacaatatacaccctctcaccatcggggaatgccccaaatatgtgaaatcaaacagattagtccttgcgactgggctcagaaatacccctcaaagagagagaagaggattatttggagctatagcaggttttatagagggaggatggcagggaatggtagatggttggtatgggtaccaccatagcaacgagcaggggagtgggtacgctgcagacaaagaatccactcaaaaggcaatagatggagtcaccaataaggtcaactcgattattgacaaaatgaacactcagtttgaggccgttggaagggaatttaacaacttagaaaggagaatagagaatttaaacaagaagatggaagacgggttcctagatgtctggacttataatgctgaacttctagttctcatggaaaatgagagaactctagactttcatgactcaaatgtcaagaacctttacgacaaggtccgactacagcttagggataatgcaaaggagctgggtaacggttgtttcgagttctatcataaatgtgataatgaatgtatggaaagtgtaagaaacggaacgtatgactacccgcagtattcagaagaagcaagactaaaaagagaggaaataagtggagtaaaattggaatcaataggaatttaccaaatattgtcaatttattctacagtggcgagctccctagcactggcaatcatggtagctggtctatccttatggatgtgctccctgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 18 - DNA, 2550 bp, HIV virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagataagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagacataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaacatgtggcaagaagtaggaaaagcaatgtatgcccctcccatcagaggacaaattagatgttcatcaaatattacagggctgatattaacaagagatggtggaaataaccacaataatgataccgagacctttagacctggaggaggagatatgagggacaattggagaagtgaattatataaatataaagtagtaaaaattgaaccattaggagtagcacccaccaaggcaaagagaagagtggtgcagagagaaaccggtgcagtgggaacaataggagctatgttccttgggttcttgggagcagcaggaagcactatgggcgcagcgtcaataacgctgacggtacaggccagactattattgtctggtatagtgcaacagcagaacaacttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaaacgtctggtaccttcgggggaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 19 - DNA, 2550 bp, HIV virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagataagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagacataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaacatgtggcaagaagtaggaaaagcaatgtatgcccctcccatcagaggacaaattagatgttcatcaaatattacagggctgatattaacaagagatggtggaaataaccacaataatgataccgagacctttagacctggaggaggagatatgagggacaattggagaagtgaattatataaatataaagtagtaaaaattgaaccattaggagtagcacccaccaaggcaaagagaagagtggtgcagagagaaaaaagagcagtgggaacaataggagctatgttccttgggttcttgggagcagcaggaagcactatgggcgcagcgtcaataacgctgacggtacaggccagactattattgtctggtatagtgcaacagcagaacaacttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaaacgtctggtaccttcgggggaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 20 - DNA, 2165 bp, Hepatitis C virus
acgcgtatgggttgcagtttttctatatttcttctggcgttgctgagttgtctgaccgtccccgcctcagcgtatcaagtacggaatagttctggcctttaccatgtcactaatgactgcccaaattctagcattgtatatgaagccgccgatgccatcctgcacaccccggggtgcgtgccctgcgtcagggaaggcaacgcctctagatgttgggtcgccgtgactcctaccgtggctacgcgcgacgggaagttgccaacaacacagctgcggcggcacatagatctgctggtcgggtccgcaaccctctgctcagctctctatgtaggagacttgtgtgggtccgtttttctggtgggtcaactgttcacattctccccaaggcgacattggaccactcaggattgtaattgttctatctatccgggacacattaccggacaccggatggcctgggacatgatgatgaattggtcccccactgctgcgctggtggttgcccagctgctccgcatccctcaagctattatggacatgatcgctggcgcacattggggggtgttggcaggaattgcctacttctctatggttggcaactgggctaaggtgctcgttgtcctcctgttgttcgcaggtgtagatgcggaaacacatgtcactggcggcaatgctggccgcaccacagccggcctggtggggctgctcacccccggagcaaaacaaaacattcagctgattaataccaacggttcctggcacatcaacagcacagcactgaactgcaacgaaagtctgaataccgggtggctggctggactcttttaccagcataagttcaattcaagcggatgcccagagcgcctggctagctgtaggcgactcaccgactttgcccaggggtggggacccatctcatatgccaatggctctggtttggatgagcgcccctactgttggcactacccacctagaccttgcggcatcgtgcctgccaagagtgtctgtggacccgtttattgtttcacaccttctccagtagtggttggaacgactgacaggagcggtgccccgacatacagttggggagcaaatgataccgacgtatttgtcttgaacaatacaaggcccccgctcggaaactggttcggttgcacatggatgaactccaccggcttcacaaaagtgtgtggcgccccaccttgcgttatcgggggcgtgggtaacaacaccctcctctgccctacggattgctttcggaaacatcccgaggcgacttattcaaggtgcgggtcaggcccgtggattacgccccggtgtatggtggattacccctacagactgtggcactacccttgcactattaactacaccatcttcaaggttcgaatgtatgtgggcggggtcgagcatcgactggaggcagcttgcaattggacgagaggcgagcgctgtgacctcgaagacagagatcgaagtgagcttagccccttgcttttgtcaaccacccagtggcaggtgctgccatgctccttcaccacccttcccgcactgtctaccggcctgatacacctgcaccagaacatcgtcgacgtgcagtacctgtatggggtggggtcaagcatagctagctgggccattaaatgggaatacgtcgtgctgctcttccttttgctggccgacgccagagtgtgtagttgcctttggatgatgcttctgatctcacaagctgagcggctgagacggcttgactggtggtggatctctctgagctctccaaagggcaaaatgcagtgtgcattccaggatactggggcccagatcagcccacactatgtgggaagctgtccctggggatgccctggctttctgtggacatacctgaggctctttatcatttttcttttgatcctgctcgtggctgccggtcttctgtacctcactgacaacggctccaccattcttgggaagttgcagtgggcatcagtgagcgccctgtttagttccatctcctcccttctgccttccgatccaaaaagtctggttgcgctcactttcggactgagcctgatatggatgactagcagttccgccacacagactttggtgacactgacacagctggctacgttgagcgcactcttctataagtcttaggcggccgc

SEQ ID NO: 21 - protein, 722 aa, Hepatitis C virus
  misc_feature (719)..(719): Xaa can be any naturally occurring amino acid
TRMGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAERLRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSXAAA

SEQ ID NO: 22 - DNA, 2076 bp, influenza virus; identical to SEQ ID NO: 16

SEQ ID NO: 23 - protein, 692 aa, influenza virus
  misc_feature (692)..(692): Xaa can be any naturally occurring amino acid
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYGTFGGILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 24 - DNA, 2082 bp, influenza virus; identical to SEQ ID NO: 17

SEQ ID NO: 25 - protein, 694 aa, influenza virus
  misc_feature (694)..(694): Xaa can be any naturally occurring amino acid
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNTPQRERRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSLRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 26 - DNA, 2550 bp, HIV virus; identical to SEQ ID NO: 18

SEQ ID NO: 27 - protein, 850 aa, HIV virus
  misc_feature (850)..(850): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQRETGAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKSLEQIWNNMTWMEWDREINNYTSLIHSLIEESQNQQEKNEQELLELDKWASLWNWFNITNWLWYIKTSGTFGGILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 28 - DNA, 2529 bp, HIV virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagataagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagacataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaacatgtggcaagaagtaggaaaagcaatgtatgcccctcccatcagaggacaaattagatgttcatcaaatattacagggctgatattaacaagagatggtggaaataaccacaataatgataccgagacctttagacctggaggaggagatatgagggacaattggagaagtgaattatataaatataaagtagtaaaaattgaaccattaggagtagcacccaccaaggcaaagagaagagtggtgcagagagaaaccggtgcagtgggaacaataggagctatgttccttgggttcttgggagcagcaggaagcactatgggcgcagcgtcaataacgctgacggtacaggccagactattattgtctggtatagtgcaacagcagaacaacttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 29 - protein, 843 aa, HIV virus
  misc_feature (843)..(843): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQRETGAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKSLEQIWNNMTWMEWDREINNYTSLIHSLIEESQNQQEKNEQELLELDKWASLWNWFNITNWLWYIKILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 30 - DNA, 2550 bp, HIV virus; identical to SEQ ID NO: 19

SEQ ID NO: 31 - protein, 850 aa, HIV virus
  misc_feature (850)..(850): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKSLEQIWNNMTWMEWDREINNYTSLIHSLIEESQNQQEKNEQELLELDKWASLWNWFNITNWLWYIKTSGTFGGILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 32 - DNA, 2579 bp, HIV virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagataagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagacataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaacatgtggcaagaagtaggaaaagcaatgtatgcccctcccatcagaggacaaattagatgttcatcaaatattacagggctgatattaacaagagatggtggaaataaccacaataatgataccgagacctttagacctggaggaggagatatgagggacaattggagaagtgaattatataaatataaagtagtaaaaattgaaccattaggagtagcacccaccaaggcaaagagaagagtggtgcagagagaaaaaagagcagtgggaacaataggagctatgttccttgggttcttgggagcagcaggaagcactatgggcgcagcgtcaataacgctgacggtacaggccagactattattgtctggtatagtgcaacagcagaacaacttgctgagggctattgaggcgcaacagcatctgttgcaactcacagtctggggcatcaagcagctccaggcaagagtcctggctgtggaaagatacctaagggatcaacagctcctagggatttggggttgctctggaaaactatggattccctagttgtcgaggatccctaaaccccaacgagaccttttctcatctgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaaatactagctggcctaatcggattactggtaagctttttcttgttgataaaaattctagaaatactgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 33 - protein, 827 aa, HIV virus
  misc_feature (827)..(827): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKSINNYTSLIHSLIEESQNQQEKNEQELLELDKWASLWNWFNITNWLWYIKILAGLIGLLVSFFLLIKILEILRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 34 - DNA, 2532 bp, HIV virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagataagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagacataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaacatgtggcaagaagtaggaaaagcaatgtatgcccctcccatcagaggacaaattagatgttcatcaaatattacagggctgatattaacaagagatggtggaaataaccacaataatgataccgagacctttagacctggaggaggagatatgagggacaattggagaagtgaattatataaatataaagtagtaaaaattgaaccattaggagtagcacccaccaaggcaaagagaagagtggtgcagagagaaaccggtgcagtgggaacaataggagctatgttccttgggttcttgggagcagcaggaagcactatgggcgcagcgtcaataacgctgacggtacaggccagactattattgtctggtatagtgcaacagcagaacaacttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgctgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 35 - protein, 844 aa, HIV virus
  misc_feature (844)..(844): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQRETGAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVLRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

SEQ ID NO: 36 - DNA, 2532 bp, HIV virus
atgagagtgaaggagaagtatcagcacttgtggagatgggggtggaaatggggcaccatgctccttgggatattgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagataagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagacataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaacatgtggcaagaagtaggaaaagcaatgtatgcccctcccatcagaggacaaattagatgttcatcaaatattacagggctgatattaacaagagatggtggaaataaccacaataatgataccgagacctttagacctggaggaggagatatgagggacaattggagaagtgaattatataaatataaagtagtaaaaattgaaccattaggagtagcacccaccaaggcaaagagaagagtggtgcagagagaaaaaagagcagtgggaacaataggagctatgttccttgggttcttgggagcagcaggaagcactatgggcgcagcgtcaataacgctgacggtacaggccagactattattgtctggtatagtgcaacagcagaacaacttgctgagggctattgaggcgcaacagcatctgttgcaactcacagtctggggcatcaagcagctccaggcaagagtcctggctgtggaaagatacctaagggatcaacagctcctagggatttggggttgctctggaaaactcatctgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgctgaggaggctagattggtggtggatttctctcagttctccaaagggaaaaatgcaatgcgctttccaagatactggagcccaaatctctccacattacgtaggatcttgcccgtggggatgcccaggatttctttggacctatctcaggctttttatcatcttcctcttaatcctgctagtagcagcaggcttgctgtatctgacggacaacgggtctactattttaggaaagctccaatgggcgtcggtctcagcccttttctcctccatctcttcactactgccctcggatccgaaatctctcgtcgctttaacgtttggactttcacttatatggatgacttcctcctctgccacccaaacgctcgtcaccttaacgcaattagccacgctgtctgctcttttttacaagagttag

SEQ ID NO: 37 - protein, 844 aa, HIV virus
  misc_feature (844)..(844): Xaa can be any naturally occurring amino acid
MRVKEKYQHLWRWGWKWGTMLLGILMICSATEKLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGDIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGNNHNNDTETFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLAVERYLRDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVLRRLDWWWISLSSPKGKMQCAFQDTGAQISPHYVGSCPWGCPGFLWTYLRLFIIFLLILLVAAGLLYLTDNGSTILGKLQWASVSALFSSISSLLPSDPKSLVALTFGLSLIWMTSSSATQTLVTLTQLATLSALFYKSX

(19) United States
(12) Patent Application Publication
(10) Pub. No.: US 2010/0120092 A1
(43) Pub. Date: May 13, 2010

(54) RECOMBINANT PROTEINS AND VIRUS LIKE PARTICLES COMPRISING L AND S POLYPEPTIDES OF AVIAN HEPADNAVIRIDAE AND METHODS, NUCLEIC ACID CONSTRUCTS, VECTORS AND HOST CELLS FOR PRODUCING SAME

(75) Inventors: Elizabeth Vera Ludmilla Grgacic, Victoria (AU); David Andrew Anderson, Victoria (AU); Paxton Loke, Victoria (AU); Robin Fredric Anders, Victoria (AU)

Correspondence Address: SCULLY SCOTT MURPHY & PRESSER, PC, 400 GARDEN CITY PLAZA, SUITE 300, GARDEN CITY, NY, 11530, US

(73) Assignee: HEPGENICS PTY LTD., Malvern, Victoria (AU)

(21) Appl. No.: 12/439,268

(22) PCT Filed: Aug. 29, 2007
(86) PCT No.: PCT/AU2007/001241

§ 371 Date: Jan. 13, 2010

(30) Foreign Application Priority Data
Aug. 30, 2006 (AU) 2006904731
Aug. 31, 2006 (AU) 2006904757

(51) Int. Cl. C12P 21/04; C12N 7/00; C12N 5/07; C12N 5/10; C12N 1/19; C12N 15/63; C07H 21/04

(52) U.S. Cl. 435/69.7; 435/235.1; 435/348; 435/349; 435/366; 435/255.1; 435/320.1; 536/23.4

(57) ABSTRACT

The specification discloses chimeric or recombinant virus-like particles comprising (i) S polypeptide of an avian hepadnavirus and (ii) a chimeric fusion protein comprising a polypeptide of interest covalently attached to a particle-associating portion of L polypeptide of an avian hepadnavirus, wherein the polypeptide of interest comprises a transmembrane domain or a protein binding domain or motif and wherein the chimeric fusion protein further comprises a second or further polypeptide of interest comprising a transmembrane domain and/or a protein binding domain or motif, wherein the second or further polypeptide is associated with the virus-like particle via non-peptide bonds. It is proposed that such VLPs more closely resemble the naturally occurring configuration of antigenic complexes or pathogens. The chimeric virus-like particles are illustrated using viral envelope proteins from measles, hepatitis C virus, influenza A and HIV and by polyproteins from Plasmodium surface proteins. Nucleic acid constructs, vectors, host cells comprising same and methods of producing virus-like particles and nucleic acid constructs are also described.

### FIELD

The specification relates generally to immunogenic recombinant virus-like particles (VLPs) comprising heterologous polypeptides and to methods of making same.

### BACKGROUND

Bibliographic details of the publications referred to by author in this specification are collected at the end of the description.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The hepadnaviruses are a family of enveloped DNA viruses. Assembly of mammalian hepadnaviruses, such as hepatitis B virus, is complex and mature virions are formed by the interaction of preformed cytoplasmic core particles with pre-assembled surface proteins on the host endoplasmic reticulum (ER) membrane. Following interaction with appropriate portions of envelope proteins, the nucleocapsids bud into the lumen of the ER along with a 1000-fold excess of empty, subviral particles (SVPs) and assembly is completed in an intermediate, pre-Golgi compartment (as reviewed by Nassal, Curr. Top, Microbiol. Immunol., 214:297-337, 1996).

In many studies, virus-like particles (VLPs) have proven to be promising candidate vaccines since they: (i) do not comprise a nucleocapsid and are non-infectious and therefore safe to produce and use, (ii) are more immunogenic than subunit vaccines because they provide the necessary spatial structure for display of epitopes, and (iii) elicit humoral, cell-mediated and importantly, mucosal immunity (Krueger et al., Biol. Chem., 380:275-276, 1998).

A recent example of a successful VLP vaccine, approved for use in many countries, is the recombinant papillomavirus major capsid protein (L1) VLP, which prevents infection by inducing a strong neutralizing antibody response (Frazer, Virus Research, 89:271-274, 2002).

The hepatitis B virus (HBV) subviral particle (HBsAg-S) has been viewed as a candidate to produce recombinant VLPs. Several studies have examined which domains are suitable for insertion of foreign epitopes (Bruss et al., EMBO J., 13:2273-2279, 1994; Delpeyroux et al., J. Mol. Biol., 195:343-350, 1987), including N terminal fusion of the preS domain (Prange et al., J. Gen. Virol., 76:2131-2140, 1995a). Most recently, particles carrying small, 35 amino acid insertions of the hepatitis C virus (HCV) hypervariable region 1 of the E2 envelope protein into the exposed ‘a’ determinant in the second hydrophilic loop have successfully elicited antibody responses (Netter et al., J. Virol., 75:2130-2141, 2001). Notably, there have been limitations to the size of the inserts tolerated for particle stability and a loss of immune reactivity to the ‘a’ determinant of HBsAg when particles were produced in a mammalian cell system (Prange et al, 1995a, supra; Bruss J. Virol., 65:3813-3820, 1991).

Particle instability with large fusions has recently been overcome with a Dengue virus/HBsAg fusion by expression in yeast (Bisht et al., J. Biotechnology, 99:97-110, 2002).

However, in all these cases, in order to assemble chimeric particles, the recombinant S protein must assemble with wild type S subunits. These extended S chains present a difficulty for inclusion in the tight envelope lattice formed by the HBsAg (which excludes L) and so their number is limited, and consequently the immune response generated to the foreign epitopes is low.

International Application No. PCT/AU2004/000511, published as International Publication No. WO 2004/092387 discloses the production of recombinant VLPs derived from avian hepadnaviruses. The envelope protein of duck hepatitis B virus (DHBV) and other avian hepadnaviruses consists of two proteins, the large envelope protein (L) and the small envelope protein (S), which are produced by differential in-frame translation initiation from a single preS/S open reading frame. L and S polypeptides have a common C terminal membrane spanning or S domain, while L has an approximately 160 amino acid N-terminal extension (or preS domain) encompassing a receptor binding region. The S polypeptide is the major viral envelope constituent, which determines envelope curvature and can drive particle secretion even in the absence of the nucleocapsid. In contrast L polypeptide can only be exported when co-assembled with S.

The assembly of DHBV envelope proteins and their involvement in host cell entry are closely linked to a unique topological switch adopted by hepadnaviruses, in which a large N-terminal preS domain of the L protein is post-translationally translocated across the ER membrane. This process is regulated so that generally only approximately 50% of molecules have translocated N-termini and the mature particle contains mixed internal/external topologies, including a partially translocated or intermediate form.

As disclosed in WO 2004/092387, substantial regions of L polypeptide of DHBV are dispensable for L translocation and particle assembly, including regions in the S domain which have the same amino acid sequence as S polypeptide regions essential for particle assembly. Accordingly, L polypeptides are more flexible in their particle-association than S polypeptides and thus open to more extensive manipulation. Recombinant chimeric avian hepadnaviral virus-like particles (VLPs) have been generated comprising a small envelope (S) polypeptide and a fusion polypeptide comprising a polypeptide of interest (POI) and at least a particle-associating portion of a large envelope (L) polypeptide of an avian hepadnavirus. Because the L polypeptide is not excluded during VLP assembly and because it can be extensively manipulated to vector a heterologous polypeptide without significantly affecting particle stability, the invention disclosed in WO 2004/092387 provides an improved method for the presentation of recombinant antigens in the context of a VLP.

There is a need in the art for methods of further optimising the presentation of recombinant antigens in the context of a VLP in order to improve their ability to engender an effective immune response.

### SUMMARY

Throughout this specification and the claims which follow, unless the context requires otherwise, the word “comprise”, and variations such as “comprises” and “comprising”, will be understood to denote the inclusion of a stated integer or step or group of integers or steps but not the exclusion of any other integer or step or group of integers or steps. To avoid prolixity, it is also understood that any composition or method described as “comprising” (or “comprises”) one or more named integers or steps also describes the corresponding, more limited, composition or method “consisting essentially of” (or “consists essentially of”) the same named integers or steps, meaning that the composition or method includes the named essential integers or steps and may also include additional integers or steps that do not materially affect the basic and novel characteristic(s) of the composition or method. It is also understood that any composition or method described herein as “comprising” or “consisting essentially of” one or more named integers or steps also describes the corresponding, more limited, and closed-ended composition or method “consisting of” (or “consists of”) the named integers or steps to the exclusion of any other unnamed integer or step. In any composition or method disclosed herein, known or disclosed equivalents of any named essential integers or step may be substituted for that integers or step.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided after the claims.

Each embodiment in this specification is to be applied mutatis mutandis to every other embodiment unless expressly stated otherwise.

Viral envelope proteins of many viruses are produced by the virus as precursor polyproteins that are subsequently cleaved by viral or host proteinases and viral biogenesis takes place with one or each of the individual polypeptides. In the case of hepatitis C virus, although relatively little is known about the structure and biogenesis of HCV particles, envelope proteins E1 and E2 are produced as a precursor polyprotein translated from genomic RNA and subsequently cleaved. In intact viral particles, E1 and E2 are both present, anchored in the viral membrane by transmembrane domains. In the case of human immunodeficiency virus (HIV) envelope proteins, the ectodomain polyprotein, gp 160 is initially produced and subsequently cleaved to form gp120 and gp41. In this case, gp 41 is retained by the virus particle but a proportion of gp120 is lost from the virion surface due to weak interactions with gp41. Other examples include influenza hemagglutinin HAO which is cleaved to HA1 and HA2.

The inventors reasoned that a viral-like particle would have improved immunogenicity or antigenicity if the VLP contained antigens in a form that mimics as closely as possible their natural configuration in the virus. In the case of HCV envelope proteins, E1 and E2 heterodimerize via non-covalent bonds/interactions between their transmembrane domain regions. These antigens engender neutralising antibody responses to epitopes determined by E1, E2 and E1/E2. In accordance with the present invention, the inventors have unexpectedly found that a fusion protein comprising E1 and E2 as well as a particle-associating portion of L polypeptide of DHBV will assemble along with S polypeptide into VLPs, however, E1 and E2 are cleaved and associate in the VLP by means of non-peptide bond interactions (See FIGS. 16 and 17). The presence of conformational epitopes in the DHBV VLPs comprising HCV E1 and E2 was confirmed by ELISA (see FIG. 18). Here VLPs were recognised by Monoclonal Antibody H53 that is known to recognise conformational epitopes of HCV E2. Although the present invention is exemplified and described using particular sequences, the invention is not so limited and other sequences that satisfy the requirements of the herein described invention or functional variants of the herein described sequences are described and contemplated.

HCVE1E2-VLPs and E2-VLPs bind to the HCV receptor, CD81 (Example 24) and immunised animals show strong humoral and cellular immune responses (see Examples 25 and 26). As described in Example 29, chimeric recombinant VLPs were prepared from transfected cells, purified over sucrose density gradients and analysed using antibodies to E1, E2 or S. The results demonstrate assembly of E1 into VLPs via non-peptide bond interactions with the E2-S.

In another illustrative embodiment, assembly of influenza A HA VLPs is provided. A chimeric fusion protein comprising HAO of influenza A virus and the S domain of L polypeptide of DHBV is produced in eukaryotic cells. As shown in Examples 34 and 35, HAO is broken down into HA1 and 1-HA2-S elements and HA1 remains associated with the VLP by non-peptide linkage with HA2-S.

In another illustrative embodiment, assembly of HIV gp140 and gp160 VLPs is provided. Chimeric fusion proteins represented schematically in FIG. 42 were produced. As shown in Example 44, both cleaved and uncleaved forms, gp140 and gp160 forms, and TMD or no HIV TMD forms of HIV envelope protein are able to assemble into VLPs. As shown in Example 45, using a cleavable gp140, the polyprotein is cleaved but gp120 remains associated with the VLP and gp41-S by virtue of non-peptide linkage.

L and S polypeptides from other avian hepadnaviruses are contemplated such as, but not limited to heron (HHBV), snow goose (SGHBV) and hepadnaviruses which exhibit similar subviral particle morphology to DHBV, i.e., with L and S envelope proteins. The S domains of L and S polypeptides are highly conserved within all avian hepadnaviruses, exhibiting for example up to 70% amino acid similarity in the region between TM1 and TM2.

Accordingly and in a broad embodiment, the present invention provides a recombinant nucleic acid construct encoding a chimeric fusion protein, wherein the sequence encoding the fusion protein comprises i) a contiguous sequence encoding a precursor or polyprotein of two or more polypeptides of interest (POI) each comprising a transmembrane domain and/or a protein binding motif or domain, and ii) a sequence encoding a particle-associating portion of an L polypeptide of an avian hepadnavirus.

In one embodiment, the polypeptide is a viral envelope polypeptide. In some embodiments, the construct and/or the sequence encoding a fusion protein further comprises sequences encoding one or further polypeptides of interest (POI). In some embodiments, the fusion polypeptide is translated and the precursor polypeptide is cleaved within a cell to yield two or more polypeptides which associate with each other through non-peptide bond interactions.

In other embodiments, therefore, the recombinant nucleic acid construct is in an expression vector and the recombinant construct is expressed in cells together with S polypeptide of an avian hepadnavirus. According to this embodiment, the chimeric fusion protein and S polypeptide co-assemble into a recombinant VLP and the precursor polypeptide is cleaved in the cell to yield two or more polypeptides which are retained in the VLP through non-peptide bond interactions. In some embodiments, the transmembrane domain anchors the non-peptide bond bound polypeptide to the VLP. In some embodiments, anchoring is facilitated by transmembrane domain:transmembrane domain binding. In some embodiments, the protein binding motif or domain anchors the non-peptide bond bound polypeptide to the VLP. In some embodiments, anchoring is facilitated by non-peptide binding between protein binding motifs. In some embodiments, the viral envelope polypeptide is derived from Flavivirus, Coronavirus, Herpesvirus, Hepadnavirus, Retrovirus, Orthomyxovirus or Paramyxovirus family viruses.

In another aspect, the present invention provides an isolated host cell comprising a recombinant nucleic acid construct encoding a particle-associating protein of L polypeptide as described above. For the avoidance of doubt, it should be noted that the particle-associating portion of L polypeptide comprises all or part of the S polypeptide (see FIG. 5A) and thus sequences may be derived from S polypeptide or L polypeptide. In some embodiments the cell is an isolated mammalian including a human or avian cell. In other embodiments, the cell is a non-mammalian cell such as a yeast or insect cell. In some embodiments, the nucleic acid sequence is modified for optimal expression in the cell by methods understood in the art.

In another aspect, the present invention provides a recombinant hepadnavirus VLP comprising S polypeptide of an avian hepadnavirus or a functional variant thereof and i) a fusion protein comprising at least one polypeptide of interest covalently attached to a particle-associating portion of avian hepadnavirus L polypeptide or a functional variant thereof, wherein the polypeptide of interest comprises a transmembrane domain or a protein binding domain or motif and ii) at least a second or further polypeptide of interest comprising a transmembrane domain and/or a binding domain or motif, wherein the second or further protein is associated with the VLP via non-peptide bonds.

In another aspect, the present invention provides a recombinant hepadnavirus VLP comprising S polypeptide of an avian hepadnavirus or a functional variant thereof and i) a fusion protein comprising at least one viral envelope protein or a functional variant thereof covalently attached to a particle-associating portion of avian hepadnavirus L polypeptide or a functional variant thereof, wherein the viral envelope protein comprises a transmembrane domain or a protein binding domain or motif and ii) at least a second or further viral envelope protein or a functional variant thereof comprising a transmembrane domain and/or a binding domain or motif, wherein the second or further protein is associated with the VLP via non-peptide bonds. In some embodiments, the viral envelope protein forms conformational epitopes capable of inducing neutralising antibodies against naturally occurring enveloped viral particles. In other embodiments, the subject recombinant chimeric avian hepadnaviral VLP comprises heterologous proteins comprising at least up to about 500 amino acid residues. In some embodiments, the virus-like particles of the present invention are useful in vaccine compositions to promote an effective immune response.

In another aspect, the present invention provides methods of producing the recombinant hepadnaviral VLPs described above. In particular, as will be understood now by the skilled addressee, in some embodiments, the fusion protein is encoded by the above described recombinant nucleic acid constructs. That is, in some embodiments, the fusion protein comprises a precursor polyprotein that uses the same translocon for insertion of its constituent polypeptide and which is cleaved intracellularly such that the respective protein still covalently attached to the particle-associating portion of L polypeptide is retained in the VLP via a peptide bond, and the wherein the cleaved off respective protein is retained in the VLP via non-peptide bonds. In some embodiments, the methods comprise providing conditions for a recombinant nucleic acid construct as described above to direct expression of the fusion protein. In some embodiments, the method comprises transfecting or transducing a cell with an expression vector comprising the above recombinant constructs. In some embodiments, the fusion protein comprising the envelope precursor is co-expressed with S polypeptide in the same cell. That is, in some embodiments, cells are co-transfected with different constructs to provide the subject fusion protein and S polypeptide in the same cell. In other embodiments, dual expression constructs provide both the subject fusion protein and S polypeptide in the same cell.

In another embodiment, the subject VLPs are expressed in vivo. Such constructs and methods are useful for example in the context of DNA-VLP prime-boost strategies incorporating the administration of VLPs to a subject by administration sequentially of the VLP in nucleic acid and proteinaceous form, in either order. In some embodiment, the nucleic acid sequence is modified for optimum expression and stability in the subject.

The present invention further contemplates a method of treating or preventing an infection with an enveloped viral particle, said method comprising administering an effective amount of the herein described recombinant avian hepadnavirus VLPs in nucleic acid or proteinaceous form. In another embodiment the present invention provides a vaccine comprising the herein described VLPs. In particular, the vaccine comprises the subject VLPs that comprise conformational epitopes capable of inducing an effective immune response against corresponding naturally occurring enveloped viral particles. In other embodiments, the subject nucleic acid constructs, expression vectors, cells and VLPs are used in the manufacture of a medicament for the treatment or prevention of an infection by an enveloped viral particle. In some embodiments, the enveloped viral particle is a member of the Flavivirus family or other families such as but not restricted to Coronavirus, Herpesvirus, Hepadnavirus, Retrovirus, Orthomyxovirus or Paramyxovirus families where the mature viral envelope proteins are formed by proteolytic cleavage from a precursor polyprotein. In some embodiments, the viral protein is derived from HCV, HIV, influenza or measles virus. In other embodiments, the polypeptide of interest is an intracellular parasite surface protein, such as without limitation a Plasmodium or other Apicomplexa surface protein. In other embodiments, the polypeptide of interest is an immunogenic protein.

In another embodiment, the present invention includes a diagnostic kit comprising agents that specifically recognise the subject VLPs. In some embodiments, the kit comprises antibodies or antigen binding fragments thereof that specifically recognise the herein described VLPs.

The above summary is not and should not be seen in any way as an exhaustive recitation of all embodiments of the present invention.

### BRIEF DESCRIPTION OF THE FIGURES

Some figures contain colour representations or entities. Coloured versions of the figures are available from the Patentee upon request or from an appropriate Patent Office, A fee may be imposed if obtained from a Patent Office.

FIG. 1 is a schematic representation of a cloning strategy for generating ₚCDL-E2.465.

FIG. 2A is a schematic representation of the large (L) and small (S) envelope proteins of DHBV. L and S are produced by differential translation from a single open reading frame such that L protein consists of a preS domain of 161 amino acids and a C-terminal S domain of 167 amino acids, which comprises the S protein. The three transmembrane domains (TM) are indicated by the boxes. FIG. 2B is a schematic representation of L showing where the 82 amino acid portion of the HCV E2 ectodomain (from aa 384 to 465) was inserted into the preS domain, generating the E2.465/L chimeric envelope protein. FIG. 2C provides the results of a Western Blot showing that the E2.465/L chimera is translocated across the ER. Protease protection analysis of ER microsomes prepared from LMH cells transfected with pCDL-E2.465 and pCI-S (an S protein expression plasmid). Microsomes samples were subjected to digestion with trypsin in the absence or presence of the detergent, NP-40, or left untreated, as denoted above each lane. Protease protection of E2.465/L chains was analysed by SDS-PAGE and Western blotting with a monoclonal anti-S antibody, which detects both E2.465/L and S proteins. Protection of E2.465/L from trypsin digestion (middle lane) is an indication of translocation to the ER lumen. FIG. 2D provides the results of a Western Blot showing that the E2.465/L chimera is assembled into particles. Intracellular particles were isolated from avian hepatoma (LMH) cells transfected with pCDL-E2.465 and pCI-S by freeze-thawing cells 3 times, centrifugation to obtain the cytosolic fraction for sedimentation of particles through 20% sucrose on to a 70% sucrose cushion at 38,000 r.p.m. (SW41 rotor Beckman). The particle fraction at the 20-70% sucrose interface was methanol precipitated prior to SDS-PAGE and analysis of envelope proteins by Western blotting. FIG. 2E is a schematic representation modelling the membrane orientations of L protein on the ER (depicted as microsome vesicles), showing the process of post-translational translocation to the microsome lumen, which confers protection from trypsin digestion of the E2.465/L hybrid chains. During particle assembly assembled envelope proteins bud from the ER into the ER lumen taking the inner leaflet of the ER membrane. Particles are exported from the cell via the cellular vesicular export pathway enabling isolation of particles both from the cytosolic (as shown in D) and extracellular compartments. Envelope protein domains translocated to the ER lumen are thus ultimately exposed to the outside of the assembled particle, as indicated by the schematic diagram of a particle.

FIG. 3 is a representation showing the genomic nucleotide sequence of DHBV.

FIG. 4 is a representation showing the amino acid sequences of L and S polypeptides of DHBV. Start sites are underlined and stop sites are starred (*).

FIG. 5A is a schematic representation of DHBV L and DHBV S protein.

FIG. 5B to 511 is a schematic representation of DHBV L chimeras. Boxes labelled TM1 to 3 represent the transmembrane domains. Numbers along the length of the DHBV L represent amino acid positions relative to the DHBV L sequence. The V indicates a deletion in TM1.

FIGS. 6A and 6B is a schematic representation showing results of Western blots of a membrane fraction of LMH cells transfected with pSigLΔTM1-E2.661 (A) or pCDLΔTM1-MSP2 (B). MW markers (40-120 kDa) are included to indicate the size of the chimeric L proteins.

FIGS. 7A and 7B is a schematic representation showing a Western blot of fractions from a sucrose step gradient showing that DL/S (A) and chimeric L VLPs, DLΔTM1-E2.465/S (B) and DLΔTM1-HpreS (C) produced in yeast have the same particle density, VLPs produced in yeast sedimented through 20% sucrose on to a 70% cushion were further sedimented on a 20-70% sucrose step gradient for 5 hours at 38,000 rpm. Fractions collected from the gradient (Nos: 2-11) were run on an SDS-PAGE and Western blotted with a monoclonal against the DHBV S domain.

FIG. 8 is a schematic representation showing a Western blot of DHBV L protein probed with the sequential bleeds of one rat immunised with DL/S VLPs produced in yeast. Pre refers to the bleed taken before immunisation and Nos: 1-5 represent rat sera obtained at 3, 6, 9 and 13 weeks. See Example 14.

FIG. 9 is a graphical representation showing the strong immune response to DHBV VLPs comprising the ectodomain of E2 (amino acids 384 to 661) of HCV sequence H771a genotype (NCBI Accession No. AF011751.3). Antibody responses were measured by measuring the concentration of anti-E2 antibody (OD450-620) over a time covering 9 weeks with different concentrations of VLP (0.2 μg, 1 μg, 5 μg and 25 μg) see Example 15.

FIG. 10 is a schematic representation of the dosage response over time against the log10 anti-E2 titre in individual animals from the experiment referred to in FIG. 9.

FIG. 11 is a graphical representation showing the T-cell response in animals administered various doses (0.2 μg, 1 μg, 5 μg and 25 μg) of DHBV-VLP comprising the ectodomain of E2 of HCV. T-cell response were measured in vitro by IFN-γ ELISPOT assay after E2-VLP stimulation.

FIG. 12 is a photographic representation of dendritic cells analysed by immunofluorescence microscopy to detect DHBV-VLPs (B) relative to chimeric HCV E2-VLPs (A).

FIG. 13 is a graphical representation of FACs analysis of dendritic cells over time after uptake of chimeric HCV E2-VLPs. Expression of cell surface markers associated with dendritic cell maturation is observed (see Example 16).

FIG. 14 is a schematic representation of the DNA construct used to express E1 and E2 in tandem to allow their incorporation into VLPs, and the proposed final topology of the mature E1 and E2-DS proteins within the DHBV VLP. (A) is a schematic representation of E1E2-DS tandem construct for expression of full length HCV E1 and full length HCV E2-DHBV S fusion protein as described in more detain in Example 17. (B) is a schematic representation of the synthesis, translocation across the endoplasmic reticulum (ER) and cleavage events of the E, E2-DS polyproteins as described in Example 17.

FIG. 15 is a schematic representation of the strategy used for production of the plasmid pCI E1E2-DS, which encodes hepatitis C virus E1 and E2 fused to DHBV S protein. Processing of this polypeptide in the cell yields E1 non-covalently associated with the fusion protein of E2-S, as shown schematically in FIG. 14, which in turn forms VLPs in association with S as previously shown for E2-VLPs.

FIG. 16 is a photographic representation showing expression of E1 and E2 proteins from pCI E1E2-DS as described in Example 18.

FIG. 17 is a representation of data showing assembly of VLPs containing both E1 and E2 proteins as described in Example 19.

FIG. 18 is a graphical representation showing the formation of conformational HCV epitopes on VLPs containing both E1 and E2 as described in Example 20.

FIG. 19 is a graphical representation showing that VLPs incorporating the MSP2 surface protein of Plasmodium falciparum (malaria, strain 3D7) induce strong antibody responses in Balb/C mice (H-2d), without the use of adjuvants. This is in contrast to the lack of immunogenicity of MSP2 from this strain of P. falciparum in H-2d Balb/C mice without adjuvant (Pye et al, Vaccine, 15:1017-1023, 1997), and demonstrates that VLPs are especially suited to the presentation of antigens such as MSP2 (which was previously known as MSA-2).

FIG. 20 is a schematic representation of various embodiments described herein.

FIG. 21 is a graphical representation showing the strong immunogenicity of MSP2-VLPs. The strong immunogenicity of the MSP2-VLPs is further demonstrated in the individual endpoint titres of sera from mice immunised with MSP2-VLPs

FIG. 22 is a graphical representation of data showing that MSP2-VLPs are highly immunogenic in rabbits. A group of 6 rabbits were immunised with 10 μg MSP2-VLPs without adjuvant, and all animals developed high levels of anti-MSP2 antibody after a single dose or after two doses.

FIG. 23 is a graphical representation of data showing that HCV E1E2-VLPs and E2-VLPs bind to the HCV receptor, CD81 as described in Example 24.

FIG. 24 is a graphical representation of data showing strong antibody responses to E1E2-VLPs and E2-VLPs produced in cell culture. Groups of six mice were immunised and antibody responses were measured 3 weeks after each dose by ELISA using E2 antigen. Both forms of VLPs were highly immunogenic in mice (see Example 25).

FIG. 25 is a graphical representation of data showing significant cellular immune responses to E1E2-VLPs and E2-VLPs produced in cell culture (see Example 26).

FIG. 26 is a photographic representation showing increased expression of E1E2-S using a codon-optimised gene (see Example 27).

FIG. 27 is a photographic representation of western blots showing increased expression of E1E2-S using codon-optimised gene. Increased expression of the E1 protein is detected by Western immunoblotting with the E1-specific monoclonal antibody A4 (A, compare lanes 5, 6 and 7 [codon-optimised E1 expression] to lanes 2, 3 and 4 [non codon-optimised E1 expression]). Similar amounts of E2 were produced in both codon-optimised and non codon-optimised constructs as detected by Western immunoblotting with goat antibodies to E2 (B, compare lanes as in A) (see Example 28).

FIG. 28 is a photographic representation of western blots showing increased incorporation of E1E2-S in VLPs using a codon-optimised gene, and assembly of E1 into VLPs via non-peptide bond interactions with the E2-S in chimeric VLPs (see Example 29).

FIG. 29 is a schematic representation of two different constructs for expression and assembly of influenza HA-VLPs. The equivalent constructs were made for expression in mammalian cells and in yeast (Saccharomyces cerevisiae) using appropriate plasmid vectors. Analysis of both constructs in mammalian cells, and of the TMD construct only in yeast, is shown in the following Figures.

FIG. 30 is a nucleotide sequence of the gene encoding fusion protein of H5 HA (ectodomain construct; H5ecto) and S, Nucleotides encoding the HA part of the gene are shown in lower case, and nucleotides encoding the S part of the gene are boxed and in upper case.

FIG. 31 is a nucleotide sequence of the gene encoding fusion protein of H5 HA (TMD construct; H5TMD) and S. Nucleotides encoding the HA part of the gene are shown in lower case, and nucleotides encoding the S part of the gene are boxed and in upper case.

FIG. 32 is a schematic representation of H5ecto and H5TMD constructs, and indirect immunofluorescence detection of HA-S expression in transfected 293T cells, stained with H5 HA-specific monoclonal antibody 149 (green), nuclei stained red. Both the H5ecto and H5TMD constructs express significant amounts of HA-reactive antigen in cell culture.

FIG. 33 is a photographic representation of western blots showing the assembly of influenza A HA H5ecto-VLPs in cell culture (see Example 31).

FIG. 34 is a graphical representation showing assembly of influenza A H5TMD-VLPs and H5ecto-VLPs in cell culture detected by ELISA of sucrose density gradient fractions with HA-specific monoclonal antibody 149 and S-specific monoclonal antibody 7C12 (see Example 32).

FIG. 35 is a photographic representation of a western blot showing that H5 VLPs exhibit the correct conformation of HA1 and HA2-S after trypsin digestion (see Example 33).

FIG. 36 is a photographic representation of western blots showing the H5ecto-VLPs and H5TMD-VLPs digested with trypsin. The HA1 part of the HA molecule remains associated with the VLP by virtue of its non-peptide linkage with the HA2-S part of the protein. Following trypsin digestion (B) or control (mock) digestion (A), VLPs were sedimented over sucrose gradients as shown in the schematic, and the fraction 3 interface containing VLPs, as well as fractions 5 and 6 containing soluble proteins, were analysed by SDS-PAGE and Western immunoblotting with rabbit H5 HA-specific antibody (see Example 34).

FIG. 37 is a photographic representation showing assembly of influenza A H5TMD-VLPs in yeast (Saccharomyces cerevisiae), detected by western immunoblotting of sucrose density gradient fractions with H5 HA-specific rabbit antibody and S-specific monoclonal antibody 7C12 (see Example 35).

FIG. 38 is a graphical representation showing proper assembly and folding of influenza A H5TMD-VLPs produced in yeast, detected by ELISA of sucrose density gradient fractions with HA-specific monoclonal antibody 149 (see Example 36).

FIG. 39 is a graphical representation showing trypsin digestion of influenza A H5TMD-VLPs detected by ELISA of sucrose density gradient fractions with HA-specific monoclonal antibody 149. Complete digestion of the HA0-S to yield HA1 and HA2-S (as shown in western blots) results in a moderate decrease in ELISA reactivity with a range of HA-specific monoclonal (149, 11A8, 8D2) and a polyclonal antibody (H5R3), and corresponding decrease in the amount of S protein reactivity (MAb 7C12).

FIG. 40 is a photographic representation showing correct conformation of HA1 and HA2-S and lack of complex glycans in trypsin or Endo H digestion of influenza A H5TMD-VLPs detected by Western blotting of VLPs from sucrose density gradient fractions with HA-specific rabbit antibody (see Example 38).

FIG. 41 is a photographic representation showing glycan sensitivity of E1E2-VLPs and E2-VLPs. Glycoproteins present on chimeric HCV VLPs show limited amounts of complex glycosylation, with mostly mannose residues present (sensitive to endoglycosidase H as well as N-glycosidase F) (see Example 39).

FIG. 42 is a schematical representation of expression constructs for various forms of the human immunodeficiency virus (HIV) envelope glycoproteins to allow incorporation into VLPs. All constructs contain the signal peptide and ectodomain of HIV gp 140, which is fused either directly to the N-terminus of the S protein (A, C); directly to the N-terminus of transmembrane domain 1 of the S protein (B, D), or includes the native transmembrane domain 1 (therefore gp160 rather than gp140) which is fused to the N-terminus of the first cytosolic loop of S, thus replacing the S TM1 (E, F). Wild-type gp140/gp160 contains a furin cleavage site that results in proteolytic processing to give gp120 and gp41 fragments, or in this case gp120 and gp41-S fragments. Mutants which abolish this furin cleavage (gp140unc or gp160unc) are shown in A, B and E; wild-type cleavage sites (gp140c or gp160c) are shown in C, D and F.

FIG. 43 is a representation of the nucleotide sequence of the gene encoding fusion protein of HIV gp140 (uncleaved—mutation of furin cleavage site, codons shown in lowercase and boxed) and S. Nucleotides encoding the HIV part of the gene are shown in lowercase (mutated furin cleavage site boxed), and nucleotides encoding the S part of the gene are shown in uppercase and boxed. This corresponds to Construct A in FIG. 42.

FIG. 44 is a representation of the nucleotide sequence of the gene encoding fusion protein of HIV gp140 (cleaved—wild-type furin cleavage site) and S. Nucleotides encoding the HIV part of the gene are shown in lowercase (furin cleavage site shown in box), and nucleotides encoding the S part of the gene are shown in uppercase and boxed. This corresponds to Construct C in FIG. 42.

FIG. 45 is a photographic representation of immunofluorescence results showing expression of HIV gp140-S detected by indirect immunofluorescence with HIV envelope-specific monoclonal antibody 2G12.

FIG. 46 is a photographic representation of western blots showing assembly of HIV gp140-S and S into VLPs detected with a combination of HIV envelope-specific patient serum and monoclonal antibody 7C12 (A), and cosedimentation with wild-type DHBV VLPs (containing DHBV L protein and S protein) detected by Western immunoblotting with monoclonal antibody 7C12 alone (B).

FIG. 47 is a graphical representation showing assembly of HIV 140-S and S into VLPs detected by ELISA with a combination of HIV envelope-specific monoclonal antibody 2G12 and monoclonal antibody 7C12 (A), and cosedimentation with wild-type DHBV VLPs (containing DHBV L protein and S protein) detected by ELISA with monoclonal antibody 7C12 and showing no reactivity with 2G12 (B).

FIG. 48 is a graphical representation showing assembly of various forms of HIV gp140-S or gp160-S together with S into VLPs, detected by ELISA with a combination of HIV envelope-specific monoclonal antibody 2G12 and monoclonal antibody 7C12 (see Example 44).

FIG. 49 is a photographic representation showing the gp140cDS construct (Construct C in FIG. 42) in which the furin cleavage site is wild-type. The gp120 part of the HIV envelope protein is cleaved by furin protease during synthesis and assembly, but remains associated with the VLP by virtue of its non-peptide linkage with the gp41-DS part of the protein, which is assembled into the VLPs. Envelope polypeptide gp120 sediments in association with the VLPs and is detected by Western immunoblotting with patient anti-HIV serum (outlined with a box for clarity).

FIGS. 50 to 58 provide nucleotide and amino acid sequences of HCV E1E2-S, Influenza HA-S and HIV gp160-S constructs.

### DETAILED DESCRIPTION OF THE PREFERRED EMBODIMENTS

Before describing the present invention in detail, it is to be understood that unless otherwise indicated, the subject invention is not limited to specific formulations of components, manufacturing methods, dosage regimens, or the like, as such may vary. It is also to be understood that the terminology used herein is for the purpose of describing particular embodiments only and is not intended to be limiting.

As used in this specification, the singular forms “a”, “an” and “the” include plural aspects unless the context clearly dictates otherwise. Thus, for example, reference to a “viral protein” includes a single viral protein, as well as two or more viral proteins; and so forth.

The term “virus-like particle” is used in its broadest sense to mean a particle or three dimensional proteinaceous structure which, like sub-viral particles of enveloped viruses, form particles by self-assembly or folding of envelope polypeptides within a lipid bilayer. The virus-like particles of the present invention may be recombinant or synthetic or may comprise a combination of synthetic and recombinant components.

Reference herein to the term “protein”, “polypeptide” or “proteinaceous” means a polymer of amino acids and should not be limited to any particular length. Therefore, the term includes proteins, oligopeptides, peptides and epitopes. The term does not exclude modifications of the polypeptide, for example myristylation, glycosylation, phosphorylation, addition of N-terminal signal sequences and the like. Analogs of polypeptides are encompassed and include the use of strategies to impose conformational constraints on the proteinaceous molecule.

The terms “fusion polypeptide” or “chimeric polypeptide” or “hybrid polypeptide” are interchangeably used to mean a polypeptide, protein or peptide comprising two or more associated polypeptides which are expressed as part of the same expression product, or which are generated by synthetic means. The components of the fusion protein are thus covalently linked by peptide bonds. The terms “chimeric” and “hybrid” indicates that the fusion partners do not exist together in nature and that they are derived from different species. Fusion polypeptides may comprise two or more L and POI polypeptides and intervening regions such as, for example, linker or spacer regions. In particular, regions which permit or directly or indirectly facilitate a surface topology or increase protease resistance for the polypeptide of interest in the particle are contemplated, for example, N-terminal signal sequences. An example of a signal sequence is preprolactin however there are many other suitable signal sequences, as will be understood by one of skill in the art. An example of a spacer region is a transmembrane domain. Alternatively, or in addition, regions which promote a cytosolic topology may be included. Polypeptide topology in a viral particle may be assessed for example by protease protection assay or by determining interactivity with antibodies determined by the L polypeptide, S polypeptide, the polypeptide of interest or epitopes generated through fusion of these polypeptides.

The term “polypeptide of interest” means any polypeptide which is contemplated for delivery to a human subject or animal as part of a virus-like particle. In some embodiments, the polypeptide of interest is produced as a precursor or polypeptide and comprises a cleavage site such that the respective polypeptides or polypeptides of interest are generated after cleavage. In some embodiments, the polypeptide of interest further comprises a transmembrane domain or a protein binding domain or motif. For example, two or more, or a matrix, of polypeptides involved in promoting and/or mediating a particular biochemical or physiological reaction may be delivered to a subject in viral particle form. A illustrative reaction contemplated is an immune response to an antigen. Accordingly the term includes any antigenic polypeptide of interest. Antigenic polypeptides may be co-expressed with immunopotentiating polypeptides such as cytokines as is well known in the art. The polypeptides and peptides of the present invention may furthermore be expressed or synthesised in L with molecules which serve as targeting and/or marker molecules such as, without limitation, molecules which assist in targeting and/or marking particular cells, such as dendritic or other antigen presenting cells. In an illustrative embodiment, the POI is a viral envelope polypeptide such as without limitation E1 and/or E2 of HCV. In some embodiments, the polypeptide of interest is a heterologous polypeptide that does not naturally occur in avian or other hepadnaviruses. In other embodiments, the heterologous polypeptide is modified to remove one or more cleavage sites.

“Operably connected” and the like refer to a linkage of polypeptide elements in a functional relationship. A polypeptide sequence is “operably connected” when it is placed into a functional relationship with another polypeptide sequence. For instance, a polypeptide is operably connected to a transmembrane domain or protein binding domain if the transmembrane domain or protein binding domain affects the position or binding of the polypeptide in the VLP. In some embodiments, the viral envelope polyprotein or polypeptide employs a transmembrane domain derived from avian hepadnavirus. In other embodiments the viral envelope polypeptide or polyprotein employs its own transmembrane domain. In other embodiments, transmembrane domains may be from a heterologous source such as from a different virus species or strain. In relation to polynucleotide sequences “operably connected” and the like refer to a linkage of polynucleotide elements in a functional relationship. A nucleic acid sequence is “operably connected” when it is placed into a functional relationship with another nucleic acid sequence. For instance, a promoter or enhancer is operably connected to a coding sequence if it affects the transcription of the coding sequence. Operably connected means that the nucleic acid sequences being linked are typically contiguous and, where necessary to join two protein coding regions, contiguous and in reading frame. A coding sequence is “operably connected to” another coding sequence when RNA polymerase will transcribe the two coding sequences into a single mRNA, which is then translated into a single polypeptide having amino acids derived from both coding sequences. The coding sequences need not be contiguous to one another so long as the expressed sequences are ultimately processed to produce the desired protein.

“Subject” as used herein refers to an animal, preferably a mammal and more preferably human who can benefit from administration of the viral particles of the present invention. There is no limitation on the type of animal that could benefit from the presently described molecules. A patient regardless of whether a human or non-human animal may be referred to as an individual, subject, animal, host or recipient. The molecules and methods of the present invention have applications in human medicine, veterinary medicine as well as in general, domestic or wild animal husbandry. For convenience, an “animal” includes an avian species such as a poultry bird, an aviary bird or game bird. The preferred animals are humans or other primates, livestock animals, laboratory test animals, companion animals or captive wild animals. Examples of laboratory test animals include ducks, snow geese, mice, rats, rabbits, guinea pigs and hamsters. Rabbits and rodent animals, such as rats and mice, provide a convenient test system or animal model. Livestock animals include sheep, cows, pigs, goats, horses and donkeys. Non-mammalian animals such as avian species, fish and amphibians are also contemplated.

The terms “antigen” or “antigenic polypeptide” are used in their broadest sense to include polypeptides which are capable of inducing an immune response in a subject. The antigenic polypeptide may comprise single epitope regions through to multiple epitope regions including repeated epitope regions. The antigenic polypeptide may derive from a single or multiple sources although antigens from infectious agents, such as, for example, viruses, bacteria, fungi, protozoa, trematodes, nematodes, prions and the like are contemplated, as are tumour-related antigens. Antigenic regions of many agents and tumour-related proteins are well known in the art. Antigens are, for example, those from parasites, bacteria, viruses, cancers and those described herein include antigens from E1 and E2 polypeptide of Hepatitis C virus, MSP2 polypeptide from P. falciparum, HA polypeptide from influenza A virus, gp140 polypeptide from human immunodeficiency virus, and H protein from measles virus (see Example 22). As is well know to those skilled in the art, effective immune responses for prophylactic or therapeutic vaccines generally elicit strong CTL and/or T-helper cell responses as well as strong humoral responses.

The antigenic polypeptide of interest may comprise epitope regions from two or more polypeptides from different organisms, species or subspecies. For example, viral and bacterial or multiple viral or multiple bacterial infectious may be vaccinated for simultaneously.

The phrase “particle-associating portion” means for all L polypeptides, that or those portions of the L polypeptide which is/are required for L polypeptide incorporation into virus-like particles. For example, the TM1 region of the S domain of L is not required for L association with the particle and may be omitted from the L-polypeptide used herein. Indeed, as contemplated herein, the sequences downstream of TM1 (or downstream of TM2 and the 5′ cysteine loop) of L polypeptide are sufficient for particle-association. Similarly, the preS domain of L is not required for assembly of L in the particles. The S domain of L absent TM1 is an example of a particle-associating portion of L. Many different particle-association portions are clearly available pursuant to the present invention. The nature of this portion is flexible and functional fragments and variants of L may be determined empirically using methods known in the art and referred to herein.

Although a minimum functional portion of L may be advantageous in some applications, the present invention extends full length L polypeptides interspersed with a POI or wherein the POI is terminally appended. Preferably the POI is introduced into surface exposed portions of L.

Exemplary portions of L are: the C-terminal portion of L comprising TM2 and TM3, or functional variants capable of assembling with S-polypeptides into DHBV-VLP; amino acids 24 to 167 of DHBV S domain or aa 190 to 328 of L domain of functional variants that comprise the loop, signal, anchor sequence (TM2) and downstream transmembrane domain (TM3); more preferably at least TM2 (including the 5′ cysteine loop between TM1 and TM2) and downstream sequences of L polypeptide of DHBV. The TM1 sequence may be substituted with the TM domain sequence of the POI. In one particular embodiment of the present invention, the polypeptide or polyprotein is located at the amino terminal side of the S domain amino acid sequence of the L polypeptide or the S domain minus the TM1 domain. In another embodiment, the POI is located in the pre-S domain of the L polypeptide or N terminally to the L polypeptide.

In some embodiments, by introducing one or more viral envelope polypeptide or polyprotein into the pre-S domain of L or N terminally to the S domain of L or N-terminally to the S domain absent TM1, the viral envelope polypeptide or polyprotein is translocated along with L into a particle structure made up primarily of S polypeptide. This facilitates a high copy number of viral envelope polypeptide or polyprotein per VLP.

The term “derived from” means that a particular element or group of elements has originated from the source described, but has not necessarily been obtained directly from the specified source.

The term “isolated” includes reference to VLPs having undergone at least one purification step, conveniently described in terms of the percentage of pure or homogeneous material in a sample. Preferred forms include material which is at least 50% pure, more preferably at least 60%, more preferably at least 70%, more preferably at least about 80%, still more preferably at least about 90% pure VLP material in a sample.

In some embodiments the specification provides a recombinant nucleic acid construct encoding a chimeric fusion protein wherein the nucleic acid comprises i) a contiguous sequence of nucleotides encoding a polyprotein of two or more polypeptides and ii) a sequence of nucleotides encoding a virus-like particle-associating portion of an L polypeptide of an avian hepadnavirus. In some embodiments, the chimeric fusion protein comprises a polyprotein of two or more polypeptides of interest and comprises a particle-associating portion of L polypeptide, and wherein each of said polypeptides is operably connected to a transmembrane domain and/or a protein binding domain. In other embodiments, the polyprotein is a precursor of two or more polypeptides of interest each comprising a transmembrane domain and/or a protein binding domain. In further embodiments, the transmembrane domain is derived from the polyprotein or from an avian hepadnavirus L or S polypeptide. In some embodiments, the transmembrane domain or protein binding domain mediates binding of at least one polyprotein derived polypeptide to the VLP via non-peptide bonds. In one illustrative embodiment, the polyprotein is Plasmodium MSP2 polypeptide.

In one aspect, of the present invention provides a recombinant nucleic acid construct encoding a chimeric fusion protein, wherein the sequence encoding the fusion protein comprises i) a contiguous sequence encoding a precursor or polyprotein of two or more POIs or viral envelope polypeptides each comprising a transmembrane domain and/or a protein binding motif or domain, and ii) a sequence encoding a particle-associating portion of an L polypeptide of an avian hepadnavirus. In some embodiments the chimeric fusion protein is immunogenic.

In other embodiments, a recombinant nucleic acid construct is provided which encodes a chimeric fusion protein wherein the nucleic acid comprises i) a contiguous sequence of nucleotides encoding a polyprotein of two or more virus envelope polypeptides and ii) a sequence of nucleotides encoding a virus-like particle-associating portion of an L polypeptide of an avian hepadnavirus. In some embodiments, the chimeric fusion protein comprises a polyprotein of two or more virus envelope polypeptide and comprises a particle-associating portion of L polypeptide, and wherein each of said polypeptides is operably connected to a transmembrane domain and/or a protein binding domain. In other embodiments, the polyprotein is a precursor of two or more virus envelope polypeptides each comprising a transmembrane domain and/or a protein binding domain. In other embodiments, the transmembrane domain is derived from the viral envelope polyprotein or from an avian hepadnavirus L or S polypeptide. In still further embodiments, the transmembrane domain or protein binding domain mediates binding of at least one viral envelope protein to the VLP via non-peptide bonds. In a different embodiments, the protein binding domain contains residues for the formation of a disulphide bond between said envelope polypeptides or between an envelope polypeptide and L or S polypeptide.

In relation to embodiments concerning virus envelope polyproteins the virus envelope polypeptide is selected from the group comprising a Flavivirus, Coronavirus, Herpesvirus, Hepadnavirus, Retrovirus, Orthomyxovirus or Paramyxovirus envelope polypeptide or a functional variant thereof. In some embodiments, the virus envelope protein is a Flaviviridae (eg hepatitis C virus), Orthomyxoviridae (eg influenza), Paramyxovirus (eg measles virus) or Retroviridae (eg human immunodeficiency virus (HIV)) virus envelope polypeptide or a functional variant thereof.

In some embodiments, the particle-associating portion of L polypeptide comprises all or part of the S domain of L polypeptide of avian hepadnavirus, the S domain minus the TM1 domain, or the S domain minus the TM1, TM2 and N′ cysteine loop. In other embodiments, the sequence of nucleotides encoding a particle-associating portion of L polypeptide is selected from SEQ ID NO: 8, nucleotides 1581 to 2076 of SEQ ID NO: 16, nucleotides 1663 to 2082 of SEQ ID NO: 17 or nucleotides 2047 to 2550 of SEQ ID NO: 18, or a functional variant of one of these having at least 95% sequence identity thereto or a functional variant of one of these which hybridises to its complement under at least medium stringency hybridisation conditions.

In one illustrative embodiment the polyprotein is E1E1 of hepatitis C virus. As described herein in one example, the nucleotide sequence encoding the polyprotein-S construct is as set forth in SEQ ID NO: 20 or a functional variant thereof having at least 95% sequence identity thereto or a sequence that hybridises to SEQ ID NO:20 or to a complementary sequence thereof under at least medium stringency hybridisation conditions.

In another illustrative example, the polyprotein is HAO of influenza A virus. As described in the Examples, one nucleotide sequence encoding the chimeric fusion protein comprises the nucleotide sequence as set forth in SEQ ID NO: 22 or 24 or a functional variant thereof having at least 95% sequence identity thereto or a sequence that hybridises to SEQ ID NO: 22 or 24 or a complementary sequence of either of these under at least medium stringency hybridisation conditions.

In another illustrative embodiment, the polyprotein is gp160 or gp140 of HIV. In some embodiments, the polyprotein includes an endogenous transmembrane domain. In other embodiments, the polyprotein does not comprise an endogenous transmembrane domain. In other embodiments, the sequence encodes an endogenous cleavage site in the polyprotein. In another embodiment, the sequence encoding an endogenous cleavage site is mutated to prevent cleavage. In some embodiments the nucleotide sequence encoding the chimeric fusion protein comprises the nucleotide sequence as set forth in SEQ ID NO: 18, 19, 26, 28, 30, 32, 34, or 36 or a functional variant thereof having at least 95% sequence identity thereto or a sequence that hybridises to a complementary sequence thereof under at least medium stringency hybridisation conditions.

In another embodiment the fusion protein comprising E1E2 comprises a sequence of amino acids as set forth in SEQ ID NO: 21 or a functional portion thereof or a functional variant thereof having at least 95% sequence identity.

In another illustrative embodiment, the fusion protein comprising HA comprises a sequence of amino acids as set forth in SEQ ID NO: 23 or 25 or a functional portion thereof or a functional variant thereof having at least 95% sequence identity.

In another illustrative embodiment, the fusion protein comprising HIV gp 140 or gp160 comprises a sequence of amino acids as set forth in SEQ ID NO: 27, 29, 31, 33, 35, or 37 or a functional portion thereof or a functional variant thereof having at least 95% sequence identity.

In some embodiments, the avian hepadnavirus is duck hepatitis B virus (DHBV). In some other embodiments the construct further encodes a small envelope (S) polypeptide of an avian hepadnavirus or this polypeptide may be provided by a separate construct or source.

The recombinant construct is capable of forming a virus-like particle (VLP) in conjunction with S polypeptide comprising i) a fusion polypeptide comprising a POI or viral envelope polypeptide or a functional variant thereof and at least a portion of the S domain of a large envelope (L) polypeptide of an avian hepadnavirus such as DHBV or a functional variant thereof; and ii) a small envelope (5) polypeptide of an avian hepadnavirus such as DHBV or a functional variant thereof; and wherein a second or further POI or viral envelope polypeptide is attached to the VLP or the first and/or further polypeptide through non-peptide bond interactions.

In one embodiment, the viral envelope polypeptide is derived from HCV and is an E1 or E2 polypeptide. E1 and/or E2 may be produced as a fusion with L, and E1 and/or E2 may be associated with the VLP/fusion polypeptide by non-peptide bonds.

In other embodiments, the interaction between transmembrane domains of the respective polypeptides anchors the non-peptide bond bound peptide to the VLP. In other embodiments, a binding domain or motif is encoded by the recombinant nucleic acid construct to allow the respective polypeptides comprising binding domains to interact to attach the non-peptide bond bound polypeptide to the VLP. In some embodiments, a combination of transmembrane domains or motifs and protein binding motif or domain is employed. In some embodiments, the binding motif or domain contain residues suitable for the formation of disulphide bonds between respective polypeptides. In further embodiments, the binding domain sequence is derived from the amyloid-like amino acid sequences derived from the merozoite surface protein (MSP) surface protein of Plasmodium. In some embodiments, the particle-associating portion of L polypeptide comprises the loop, signal anchor sequence (TM2) and the downstream transmembrane (TM3). In other embodiments, the nucleotide sequence encoding the viral envelope polypeptide sequence is modified by removal of cleavage sites to ensure that at least one of the respective polypeptides is retained covalently attached to the particle-associating portion of avian hepadnavirus L polypeptide. In further embodiments, a dual expression construct permits co-expression of the subject fusion protein and S polypeptide in cells.

The non-peptide bond interactions are mediated by transmembrane domains or protein-binding domains or motifs. As the skilled person will appreciate, non-peptide bonds include hydrogen bonds, Van der Waals forces, electrostatic interactions, hydrophobic interactions and disulphide bonds. At least a part of the viral envelope polypeptides is exposed on the surface of the virus-like particle.

The recombinant construct is conveniently employed in an expression vector in order to effect expression of polypeptides in single, dual or multiple expression systems. Accordingly, expression vectors are contemplated comprising the nucleic acid constructs as herein described operably connected to an expression control sequence. In some embodiments, the nucleic acid construct and expression vector further comprise sequences encoding S polypeptide of avian hepadnavirus.

In one embodiment, the virus-like particles of the present invention are useful in vaccine compositions to promote an effective immune response. The present invention provides an immunogenic composition comprising the recombinant virus-like particles as described herein and a pharmacologically acceptable carrier.

In particular, the virus-like particles are advantageously a suitable size to be taken up by antigen presenting cells, such as dendritic cells. Specifically, in relation to mammalian hepadnavirus particles, these are typically approximately 20 nanometers, while those of avian hepadnaviruses are pleomorphic and are typically between 35 and 60 nanometers in diameter. An effective immune response is typically one which is capable of reducing the number of target antigens in a subject and may prevent infections or development of disease conditions (prophylactic vaccine) or may treat current infections or conditions (therapeutic vaccination).

In some embodiments and without being bound to any particular theory, the VLPs of the present invention are capable of stimulating humoral and/or cell mediated immune responses. In some embodiments, heterologous antigens are targeted to appropriate pathways of MHC class I and class II antigen processing and presentation, and are targeted for dendritic cells which initiate, in particular T-cell responses.

In some embodiments, the L polypeptide comprises or consists of an amino acid sequence substantially set forth in all or part of SEQ ID NO: 7, SEQ ID NO: 9, or an amino acid sequence having at least 50% identity to SEQ ID NO: 7 or SEQ ID NO: 9 or a functional variant or fragment of either of these sequences. Even more preferably, the percentage similarity exceeds 60% identity, more preferably 70% identity, still more preferably at least about 80%, still more preferably about 90-95% identity. Preferred L polypeptides are derived from an avian hepadnavirus such as but not limited to DHBV. Importantly, the hepadnavirus or the envelope polypeptides employed in the present invention do not exclude L from VLP assembly. Functional variants of the instant L polypeptide include derivatives, fragments, parts or portions of a reference or parent molecule which retain the ability of the L polypeptide to associate with the particle formed by S polypeptide, or at least where such ability is not substantially lost.

Functional variants of the instant S polypeptide retain the ability of the S polypeptide to form virus-like particles, or at least where such ability is not substantially lost. Substantial loss would mean that the L particle is assembled with S in particles at a ratio of less than about 1:4 or more preferably less than about 1:8, even more preferably less than about 1:12, still even more preferably less than about 1:16. A preferred S polypeptide is derived from an avian hepadnavirus such as but not limited to DHBV or comprises or consists of an amino acid sequence substantially set forth in SEQ ID NO: 13.

The term “functional variant” also extends to polypeptides having one or more amino acid mutations or modifications and retaining the functional activity of the reference molecule. In the case of L or S, functional refers to VLP formation. In relation to POIs, functional refers to antigenicity or immunogenicity. Mutations may be derived from additions, insertions, deletions or substitutions of amino acids. Substitutions are preferably conservative amino acid substitutions within the following groups: glycine and alanine; valine, isoleucine and leucine; aspartic acid and glutamic acid; asparagine and glutamine; serine and threonine; lysine and arginine; and phenyl alanine and tyrosine. Modifications may include the addition of flanking sequences which enhance viral particle assembly or stability in a host cell. Functional variants of the heterologous polypeptides include polypeptides modified to enhance binding to the recombinant VLP and the conformational structure of the recombinant VLP.

In some embodiments, variants have at least 60% amino acid similarity, or more preferably at least 80%, or most preferably 90% or greater similarity to all or a functional part of the parent (or reference) molecules.

In another aspect the present specification described a recombinant virus-like particle comprising S polypeptide of avian hepadnavirus and i) a chimeric fusion protein comprising a viral envelope polypeptide produced from a polyprotein, covalently attached to a particle-associating portion of L polypeptide of avian hepadnavirus and ii) a second or further viral envelope polypeptide also produced from said polyprotein, associated with the virus-like particle by a non-peptide bond. In some embodiments, the chimeric fusion protein comprises a polyprotein of two or more virus envelope polypeptide and comprises a particle-associating portion of L polypeptide, and wherein each of said polypeptides is operably connected to a transmembrane domain and/or a protein binding domain. In other embodiments, the polyprotein is a precursor of two or more virus envelope polypeptides each comprising a transmembrane domain and/or a protein binding domain. In other embodiments, the transmembrane domain is derived from the viral envelope polyprotein or from an avian hepadnavirus L or S polypeptide. In another embodiment, the transmembrane domain or protein binding domain mediates binding of at least one viral envelope protein to the VLP via non-peptide bonds. In a still further embodiment, the protein binding domain contains residues for the formation of a disulphide bond between said envelope polypeptides or between an envelope polypeptide and L or S polypeptide.

In accordance with this aspect of the invention, the recombinant virus-like particle virus envelope polypeptide is a Flavivirus, Coronavirus, Herpesvirus, Hepadnavirus, Retrovirus, Orthomyxovirus or Paramyxovirus envelope polypeptide or a functional variant thereof.

In another embodiment, the virus envelope protein is a Flaviviridae (eg hepatitis C virus), Orthomyxoviridae (eg influenza), Paramyxovirus (eg measles virus) or Retroviridae (eg human immunodeficiency virus (HIV)) virus envelope polypeptide or a functional variant thereof.

As stated in relation the nucleic acid constructs, the particle-associating portion of L polypeptide comprises, in some embodiments, all or part of the S domain of L polypeptide of avian hepadnavirus, the S domain minus the TM1 domain, or the S domain minus the TM1, TM2 and N cysteine loop. In an illustrative embodiment, the particle-associating portion of L polypeptide is encoded by a Sequence of nucleotides selected from SEQ ID NO: 8, nucleotides 1581 to 2076 of SEQ ID NO: 16, nucleotides 1663 to 2082 of SEQ ID NO: 17 or nucleotides 2047 to 2550 of SEQ ID NO: 18, or a functional variant of one of these having at least 95% sequence identity thereto or a functional variant of one of these which hybridises to its complement under at least medium stringency hybridisation conditions.

In some embodiments, the polyprotein is E1E1 of hepatitis C virus. Here, in one illustrative example, the chimeric fusion protein is encoded by the nucleotide sequence as set forth in SEQ ID NO: 20 or a functional variant thereof having at least 95% sequence identity thereto or a sequence that hybridises to SEQ ID NO:20 or to a complementary sequence thereof under at least medium stringency hybridisation conditions.

In another example the recombinant virus-like particle polyprotein is HAO of influenza A virus. Here, in one embodiment, the chimeric fusion protein is encoded by the nucleotide sequence set forth in SEQ ID NO: 22 or 24 or a functional variant thereof having at least 95% sequence identity thereto or a sequence that hybridises to SEQ ID NO: 22 or 24 or a complementary sequence of either of these under at least medium stringency hybridisation conditions.

In another example wherein the polyprotein is gp 160 or gp 140 of HIV, the polyprotein may include an endogenous transmembrane domain or this may be absent. similarly, cleavage sites may be present or absent. In an illustrative example, the chimeric fusion protein is encoded by the nucleotide sequence as set forth in SEQ ID NO: 18, 19, 26, 28, 30, 32, 34, or 36 or a functional variant thereof having at least 95% sequence identity thereto or a sequence that hybridises to a complementary sequence thereof under at least medium stringency hybridisation conditions.

In another aspect, the present invention provides a recombinant hepadnavirus VLP comprising S polypeptide and i) a fusion protein comprising at least one POT covalently attached to a particle-associating portion of avian hepadnavirus L polypeptide wherein the POI comprises a transmembrane domain or a binding domain or motif and ii) at least a second or further POI comprising a transmembrane domain or a binding domain or motif, wherein the second or further protein is associated with the VLP via non-peptide bonds.

In another aspect, the present invention provides a recombinant hepadnavirus VLP comprising S polypeptide and i) a fusion protein comprising at least one viral envelope protein covalently attached to a particle-associating portion of avian hepadnavirus L polypeptide wherein the viral envelope protein comprises a transmembrane domain or a binding domain or motif and ii) at least a second or further viral envelope protein comprising a transmembrane domain or a binding domain or motif, wherein the second or further protein is associated with the VLP via non-peptide bonds.

In some embodiment, the fusion polypeptide comprises a POI or viral envelope protein and a particle-associating portion of a hepadnaviral L polypeptide wherein said L polypeptide comprises a sequence of amino acids substantially as set forth in SEQ ID NO: 7 or SEQ ID NO: 9 or an amino acid sequence having at least about 50% similarity thereto, or a functional derivative or homolog thereof. In another embodiment, the fusion polypeptide comprises a viral envelope protein and a particle-associating portion of a hepadnaviral L polypeptide wherein said L polypeptide is encoded by a sequence of nucleotides substantially as set forth in SEQ ID NO: 6 or SEQ ID NO: 8 or a sequence of nucleotides capable of hybridizing to SEQ ID NO: 6 or SEQ ID NO: 8, or a complementary form thereof under medium stringency conditions.

The VLPs of the present invention are assembled in vitro or in vivo using techniques which are well known to those of ordinary skill in the art such as those described or referred to herein or summarised in Sambrook et al. Specifically, expression plasmids are designed to express one or more recombinant envelope proteins.

In another aspect, the present invention provides an isolated or recombinant polypeptide for use in the assembly of a VLP comprising a viral envelope polypeptide of interest (POI) and at least a particle-associating portion of a large envelope polypeptide (L) of an avian hepadnavirus such as DHBV or a functional derivative or homolog thereof.

In a related aspect, the present invention provides a recombinant polypeptide capable of assembling into a VLP when expressed in a cell, said polypeptide comprising a polypeptide of interest (POI) and at least a particle-associating portion of a large envelope polypeptide (L) of an avian hepadnavirus such as DHBV or a functional derivative of homolog thereof. Preferably, the particle-associating portion of L comprises at least the S domain of L or the S domain of L minus the TM1 domain or a functional derivative thereof. Still more preferably, the POI or viral envelope polypeptide is located in the pre-S domain of L or at the amino terminal side of the S domain of L, or the S domain minus the TM1 domain of L.

Avian hepadnaviruses exhibit considerable sequence identity and sequences having greater than 70%, 80%, 90%, 95% Or 99% identity to recited sequences are contemplated.

The present invention extends to the use in the manufacture of a VLP, of a hepadnaviral L polypeptide or particle-associating portion thereof encoded by a sequence of nucleotides substantially as set forth in SEQ ID NO: 6 or SEQ ID NO: 8 or having at least about 50% similarity to SEQ ID NO: 6 or SEQ ID NO: 8 or a contiguous sequence of nucleotides capable of hybridizing to a complementary form SEQ ID NO: 6 or SEQ ID NO: 8 under hybridisation conditions of medium stringency wherein the L polypeptide is fused to a viral envelope polypeptide as described herein comprising a transmembrane domain or a protein-binding domain or motif. In some embodiments, the L polypeptide comprises a signal sequence. Such sequences are particularly useful in enhancing surface expression of a POI in the VLP. Preferred L polypeptides are DHBV L polypeptide or functional derivative thereof.

In another embodiment, the specification provides a method of producing a protein, the method comprising culturing the cell of as herein described for a time and under conditions permitting expression under the control of the expression control sequence, and optionally purifying the polypeptide from the cell or medium of the cell. In another embodiment, the method comprising culturing cells comprising an expression vector or vectors encoding S and chimeric L polypeptide for a time and under conditions permitting expression under the control of the expression control sequence and formation of a virus-like particle, and optionally purifying the virus-like particle from the cell or medium of the cell. The invention extends to a recombinant virus-like particle produced by these methods using the herein described nucleic acids.

In yet another aspect, the present invention provides a nucleic acid molecule comprising a sequence of nucleotides encoding a fusion polypeptide suitable for use in making a recombinant VLP, wherein said nucleic acid molecule encodes a POI or viral envelope precursor or polyprotein and a particle-associating portion of an L polypeptide and wherein the sequence of nucleotides encoding the particle-associating portion of an L polypeptide comprises the sequence set forth in SEQ ID NO: 6 or SEQ ID NO: 8 or a functional part thereof or a contiguous sequence of nucleotides capable of hybridizing thereto or to a complementary form thereof under low stringency hybridisation conditions, or a functional variant thereof.

In yet still another aspect, the present invention provides a nucleic acid molecule comprising a sequence of nucleotides encoding a fusion polypeptide suitable for use in making a recombinant VLP, wherein said nucleic acid molecule encodes a POI or viral envelope precursor or polyprotein and a particle-associating portion of an L polypeptide and wherein the nucleic acid encoding the particle-associating portion of an L polypeptide encodes the amino acid sequence set forth in all or part of SEQ ID NO: 7 or SEQ ID NO: 9 or an amino acid sequence having at least about 50% similarity thereto, or a functional variant thereof.

In another aspect, the present invention provides a nucleic acid molecule comprising a sequence of nucleotides encoding a fusion polypeptide suitable for use in making a recombinant VLP, wherein said nucleic acid molecule encodes a viral envelope polypeptide or viral envelope polyprotein and a particle-associating portion of an L polypeptide and wherein the nucleic acid encoding the particle-associating portion of an L polypeptide encodes the amino acid sequence set forth in SEQ ID NO: 7 or SEQ ID NO: 9.

Complementary forms of all or part the nucleic acid molecules of the present invention are expressly contemplated.

The terms “nucleic acids”, “nucleotide” and “polynucleotide” include RNA, cDNA, genomic DNA, synthetic forms and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog (such as the morpholine ring), internucleotide modifications such as uncharged linkages (e.g. methyl phosphonates, phosphotriesters, phosphoamidates, carbamates, etc.), charged linkages (e.g. phosphorothioates, phosphorodithioates, etc.), pendent moieties (e.g. polypeptides), intercalators (e.g. acridine, psoralen, etc.), chelators, allylators and modified linkages (e.g. α-anomeric nucleic acids, etc.). Also included are synthetic molecules that mimic polynucleotides in their ability to bind to a designated sequence via hydrogen binding and other chemical interactions. Such molecules are known in the art and include, for example, those in which peptide linkages substitute for phosphate linkages in the backbone of the molecule.

The term “similarity” as used herein includes exact “identity” between compared sequences at the nucleotide or amino acid level. Where there is non-identity at the nucleotide level, “similarity” includes differences between sequences which result in different amino acids that are nevertheless related to each other at the structural, functional, biochemical and/or conformational levels.

Where there is non-identity at the amino acid level, “similarity” includes amino acids that are nevertheless related to each other at the structural, functional, biochemical and/or conformational levels. In a particularly preferred embodiment, nucleotide and sequence comparisons are made at the level of identity rather than similarity.

In some embodiments, the viral envelope protein or polypeptide (i.e. more than one, preferably 2 or 3 or 4 polypeptides) is derived from Flavivirus, Coronavirus, Herpesvirus, Hepadnavirus, Retrovirus, Orthomyxovirus or Paramyxovirus family viruses.

Terms used to describe sequence relationships between two or more polynucleotides or polypeptides include “reference sequence”, “comparison window”, “sequence similarity”, “sequence identity”, “percentage of sequence similarity”, “percentage of sequence identity”, “substantially similar” and “substantial identity”. A “reference sequence” is at least 9 to 12 but frequently 15 to 18 and often at least 21 to 25 or above, such as 30 monomer units, inclusive of nucleotides and amino acid residues, in length. Because two polynucleotides may each comprise (1) a sequence (i.e. only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of typically 12 contiguous residues that is compared to a reference sequence. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e. resulting in the highest percentage homology over the comparison window) generated by any of the various methods selected. Reference also may be made to the BLAST family of programs as, for example, disclosed by Altschul et al, Nucleic Acid Research, 25:3389-3402, 1997. A detailed discussion of sequence analysis can be found in Unit 19.3 of Ausubel et a, supra.

The terms “sequence similarity” and “sequence identity” as used herein refers to the extent that sequences are identical or functionally or structurally similar on a nucleotide-by-nucleotide basis or an amino acid-by-amino acid basis over a window of comparison. Thus, a “percentage of sequence identity”, for example, is calculated by comparing two optimally aligned sequences over the window of comparison, determining the number of positions at which the identical nucleic acid base (e.g. A, T, C, G, I) or the identical amino acid residue (e.g. Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software. Similar comments apply in relation to sequence similarity. Conservative amino acid changes may be considered to provide similar sequences but not identical sequences.

An illustrative nucleotide sequence (cDNA) encoding strain H77 (genotype 1a) of HCV is available publicly at NCBI Accession No. AF011751. In some embodiments, the full length HCV E1/E2 sequences inclusive of their own signal sequences consists of aa 172 to 746 of this sequence (SEQ ID NO: 15).

Functional variants of the instant nucleic acid molecules include derivatives or fragments thereof or sequences having one or second nucleotide mutations or modifications. In an illustrative embodiment, a single point mutation is introduced A746R at the junction of E2 and P7 to block cleavage of the signal sequence fused to DHBV S sequence (SignalP-NN prediction). Signal sequence prediction tools are routinely available in the art as described for example by Nielsen et al in Protein Engineering, 10:1-6, 1997. In an illustrative embodiment, the SignalP 3.0 server predicts the presence and location of signal peptide cleavage sites in amino acid sequences from different organisms: including eukaryotes. The method incorporates a prediction of cleavage sites and a signal peptide/non-signal peptide prediction based on a combination of several artificial neural networks and hidden Markov models.

Transmembrane domains are routinely identified and may be added to, deleted from and/or moved within the subject POIs. The transmembrane domains may be derived from the L or S polypeptide or it may be derived from the polypeptide of interest. There are a number of publications that described transmembrane domains and their prediction including TMHMM: prediction of transmembrane helices in proteins; Tmpred: prediction of transmembrane regions and orientation; HMMTOP: prediction of transmembrane helices and topology of proteins; SOSUI: Classification and secondary structure prediction of membrane proteins.

Mutations include one or several nucleotide deletions, insertions or substitutions. Alternatively or in addition, derivatives may be modified by the addition of sequences or moieties to enhance function such as enhanced stability or activity or to introduce new activity. For example, modifications may comprise the addition of fusogenic agents to enhance membrane permeability, modifications to affect pre or post-transcriptional modifications events, or to generate fusion proteins comprising labels, tags and other modifications for identification, purification and so forth.

Functional variants of the subject nucleic acid molecules retain the ability of the parent or reference molecule to encode a polypeptide comprising antigenic or immunogenic sequences capable of determining an immune response in subjects. Fragments of the nucleic acid molecules may include parts or one or more portions thereof, which have at least the function of the parent or enhanced function.

Functional homologs of the instant nucleic acid sequences include orthologous gene sequences from different species which are related by common phylogenic decent and also gene sequences from other species which are similar to the instant nucleic acid molecules as a result of convergent evolution, wherein the homologs are functionally and structurally related to the instant nucleic acid sequences and are consequently readily identified and/or isolated by hybridization based methods or by sequence comparison with published genome databases. For example, the nucleotide sequence of approximately 20 avian hepadnaviruses are publicly available (Triyatni et al, J. Gen. Virol, 82:373-378, 2001). Similarly, a large number of viral envelope genes have been sequenced and nucleotide and amino acid sequence of these molecules from a large range of species and strains are available.

Similarity at the nucleic acid level may be assessed in assays exploiting different stringency of hybridization conditions as is well known in the art and is, for example, described in Ausubel et al, supra.

Reference herein to stringent hybridization conditions preferably means conditions which permit selective hybridization or annealing between molecules which are substantially similar. The hybridization temperature composition and ionic strength of the hybridization solution which meet this criteria will vary depending upon a number of well characterized factors such as length, degree of complementarity and GC content. For longer sequences it is generally possible to calculate the expected melting point of duplex nucleic acid sequences under various conditions. Hybridization may be to all or part of the instant polynucleotides with the minimum length being sufficient to provide specificity and functionality of their encoded polypeptides.

Low stringency hybridization conditions includes and encompasses from at least about 0 to at least about 15% v/v formamide and from at least about 1 M to at least about 2 M salt for hybridization, and at least about 1 M to at least about 2 M salt for washing conditions. Generally, low stringency is at from about 25-30° C. to about 42° C. The temperature may be altered and higher temperatures used to replace formamide and/or to give alternative stringency conditions.

Medium stringency includes and encompasses from at least about 16% v/v to at least about 30% v/v formamide and from at least about 0.5 M to at least about 0.9 M salt for hybridization, and at least about 0.5 M to at least about 0.9 M salt for washing conditions. High stringency includes and encompasses from at least about 31% v/v to at least about 50% v/v formamide and from at least about 0.01 M to at least about 0.15 M salt for hybridization, and at least about 0.01 M to at least about 0.15 M salt for washing conditions. In general, washing is carried out Tₘ=69.3+0.41 (G+C %). However, the Tₘ of a duplex DNA decreases by 1° C. with every increase of 1% in the number of mismatch base pairs (Bonner et al, Cold Spring Harb. Symp. Quant. Biol., 38:308-10, 1974). Formamide is optional in these hybridization conditions. Accordingly, particularly preferred levels of stringency are defined as follows: low stringency is 6×SSC buffer, 0.1% w/v SDS at 25-42° C.; a moderate stringency is 2×SSC buffer, 0.1% w/v SDS at a temperature in the range 20° C. to 65° C.; high stringency is 0.1×SSC buffer, 0.1% w/v SDS at a temperature of at least 65° C.

Vectors, preferably contain cloning sites and are capable of autonomous replication in a defined host cell. Alternatively, the vector may integrate into the genome and replicate together with the chromosome into which it has been introduced. Vectors may also include selection markers, if required.

Examples of selectable markers include genes conferring resistance to compounds such as antibiotics, genes conferring the ability to grow on selected substrates, genes encoding proteins that produce detectable signals such as luminescence. A wide variety of such markers are known and available, including, for example, antibiotic resistance genes such as the neomycin resistance gene (neo) and the hygromycin resistance gene (hyg). Selectable markers also include genes conferring the ability to grow on certain media substrates such as the tk gene (thymidine kinase) or the hprt gene (hypoxanthine phosphoribosyltransferase) which confer the ability to grow on HAT medium (hypoxanthine, aminopterin and thymidine); and the bacterial gpt gene (guanine/xanthine phosphoribosyltransferase) which allows growth on MAX medium (mycophenolic acid, adenine and xanthine). Other selectable markers for use in mammalian cells and plasmids carrying a variety of selectable markers are described in Sambrook et al., Molecular Cloning—A Laboratory Manual, Cold Spring Harbour, New York, USA, 1990.

The selectable marker may depend on its own promoter for expression and the marker gene may not necessarily be derived from human genomes (e.g. prokaryotic marker genes may be used in human cells). However, it is preferable to replace the original promoter with transcriptional machinery known to function in the recipient cells. A large number of transcriptional initiation regions are available for such purposes including, for example, metallothionein promoters, thymidine kinase promoters, β-actin promoters, immunoglobulin promoters, SV40 promoters and human cytomegalovirus promoters. A widely used example is the pSV2-neo plasmid which has the bacterial neomycin phosphotransferase gene under control of the SV40 early promoter and confers in mammalian cells resistance to G418 (an antibiotic related to neomycin). A number of other variations may be employed to enhance expression of the selectable markers in animal cells, such as the addition of a poly(A) sequence and the addition of synthetic translation initiation sequences. Both constitutive and inducible promoters may be used.

As will be understood by those skilled in the art, the nucleic acid molecules of the present invention may be further modified to ensure their suitability for expression in a range of cells. Such techniques and strategies are well known to those skilled in the art and may be conveniently referred to in Ausubel et al, Eds short protocols in Molecular Biology, John Wiley and Sons, 5^{th} Edition, 2002 and/or Sambrook et al, supra.

Viral expression vectors are conveniently employed to deliver the recombinant construct to cells or cell lines with high efficiency. Retroviral vectors are preferred as they are capable of infecting a wide range of cells and of maintaining stable delivery. Lentiviral vectors are copied along with the chromosomal DNA when the cells divide so that unlike, for example some adenoviral vectors that are lost from dividing cells, the lentiviral vector is retained in the cell line.

In an illustrative embodiment, expression of VLPs in cell culture uses replication-defective retroviral vectors that deliver genes coded as RNA which are reverse transcribed in the cell and integrate stably into the host cell genome. In particular, the GPEx system (Catalent Pharma solutions, USA) is contemplated which uses replication defective retroviral vectors derived from Moloney murine leukemia virus (MLV) and pseudotyped with vesicular stomatitis virus G-protein to stably insert single copies of genes into dividing cells.

To ensure expression, the nucleotide sequences encoding the viral envelope POI and the L polypeptide components are operatively linked to one or more expression control sequences. Preferably the two or more such nucleotide sequences are in the same reading frame.

As mentioned, the invention contemplates an expression vector comprising the nucleic acid described herein operably connected to an expression control sequence.

The invention also extends to a cultured cell comprising the vector s and nucleic acids of the present invention. In some embodiments, cultured cells are provided transfected with the vector encoding chimeric fusion proteins, or the progeny of said cell, wherein the cell is also transfected with an expression vector comprising a sequence of nucleotides encoding a polypeptide having the function of an S polypeptide of avian hepadnavirus. As discussed herein eukaryotic cells are especially preferred.

In one embodiment, expression vectors are conveniently stably integrated into the genome of host cells and expression is driven by integrated promoters.

The present invention also extends to microorganisms or host cells transformed or transfected or otherwise comprising a recombinant nucleic acid construct encoding a chimeric fusion protein, wherein the sequence encoding the fusion protein comprises i) a contiguous sequence encoding a precursor of two or more POI or viral envelope polypeptides each comprising a transmembrane domain and/or a protein binding motif or domain, and ii) a sequence encoding a particle-associating portion of an L polypeptide of an avian hepadnavirus. Compositions suitable for treating a subject with HCV or at risk of infection with HCV, include VLPs comprising S polypeptide and i) a fusion protein comprising at least one POI or viral envelope protein covalently attached to a particle-associating portion of avian hepadnavirus L polypeptide wherein the POI or viral envelope protein comprises a transmembrane domain or a binding domain or motif and ii) at least a second or further POI or viral envelope protein comprising a transmembrane domain or a binding domain or motif, wherein the second or further protein is associated with the VLP via non-peptide bonds. In one embodiment, the fusion protein comprises E1 or E2 of HCV. In another embodiment, the second or further viral envelope protein is E1 or E2 of HCV.

Prokaryotic or eukaryotic host cells may be used to produce the subject VLPs. Typically, prokaryotic cells include E. coli and Bacillis sp. Eukaryotic cells include yeast, fungi, mammalian, avian and insect cells. In an illustrative embodiment, the host organism is a yeast cell such as Hansenula polymorpha (Artes Biotechnology, GmbH). Such cells are useful for providing controlled levels of expression for the two component protein in chimeric avian hepanavirus VLPs.

In some embodiments, the invention provides a method of producing the recombinant hepadnavirus VLPs as described above.

E1/E2 VLPs, for example, may be produced in yeast using a yeast expression system. In one example of this process, the DNA sequences corresponding to chimeric E1/E2 polyprotein are subcloned into pYES-DHBV L plasmid, replacing the L ectodomain sequence. The yeast strain, such as INVSc-1 yeast stain is co-transformed with the pYES-E1/E2 and a DHBV S expression plasmid for expression and assembly of chimeric E1/E2 VLPs. E1/E2 VLPs are extracted and purified by sedimentation through sucrose step gradients. The yeast system allows for production of large quantities of VLPs for further analysis, including visualization by transmission electron microscopy, and for assessment of vaccine potential in larger animals, including macaques.

In a further example of this method a dual expression construct is conveniently employed (such as pTandem-1 and pBudCE4.1) for expression of the E1/E2 chimeric L polyprotein and the DHBV S protein in mammalian cells and expression of genes in transfected cells tested by Western blotting and IF with anti-E2 and DHBV S monoclonal antibodies. Assembly of chimeric VLPs may be assessed by sedimentation through sucrose gradients and Western blotting and heterodimerisation of E1 and E2 assessed by co-immunoprecipitation and binding to conformation-specific Mabs 1153 and 9/27 in a VLP ELISA, as performed for HCV E2 VLPs.

The present invention also relates to a vaccine comprising the herein described VLPs, in admixture with a suitable pharmaceutically acceptable diluent or carrier. The vaccine may be lyophilized prior to use and may furthermore be admixed with suitable adjuvants. Accordingly the vaccine may be in kit form. In some embodiments, the present invention provides a vaccine having confirmed VLP production in cell culture, the DNA construct will be used to immunise Balb/c mice, such that the VLPs are expressed in vivo. Antibody to E2 are assessed using in house recombinant E2 ELISA and by immunofluorescence staining in E1/E2-vaccinia virus infected cells and CMI responses to recombinant protein and HCV peptide pools are assessed by IFN-gamma ELISPOT and proliferation assays. Neutralising antibody are assessed by the HCV pseudotyped HIV-1 particle entry assay (Drummer et al., FEBS Lett., 546:385, 2003). The use of this DNA vaccine construct to assess immune responses is two-fold: (1) it will enable a more rapid assessment of its immunogenicity before E1/E2 VLP expression is performed in yeast and (2) it will provide the potential to use it as part of a DNA-VLP prime-boost vaccine strategy.

By “pharmaceutically acceptable” carrier, or diluent is meant a pharmaceutical vehicle comprised of a material that is not biologically or otherwise undesirable, i.e. the material may be administered to a subject along with the selected active agent without causing any or a substantial adverse reaction, Carriers may include excipients and other additives such as diluents, detergents, coloring agents, wetting or emulsifying agents, pH buffering agents, preservatives, and the like.

The terms “composition” “compound”, “active agent”, “pharmacological agent” or “physiological agent”, “medicament”, “agent” and “drug” are used to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “active agent”, “pharmacologically active agent”, “medicament”, “active” and “drug” are used, then it is to be understood that this includes the active agent per se as well as pharmaceutically acceptable, pharmacologically active salts, esters, amides, prodrugs, or pro-forms, enantiomers, metabolites, analogs, etc. The term “agent” is not to be construed as a chemical compound only but extends to peptides, polypeptides and proteins as well as genetic molecules such as RNA, DNA and chemical analogs thereof.

An “effective amount” means an amount necessary to at least partially attain the desired immunological response. An effective amount for a human subject lies in the range of about 0.1 ng/kg body weight/dose to about 1 g/kg body weight/dose. In some embodiments, the range is about 1μ to 1 g, about 1 mg to 1 g, 1 mg to 500 mg, 1 mg to 250 mg, 1 mg to 50 mg, or 1μ to 1 mg/kg body weight/dose. Dosage regimes are adjusted to suit the exigencies of the situation and may be adjusted to produce the optimum therapeutic or prophylactic dose. For example, several doses may be provided daily, weekly, monthly or other appropriate time intervals.

The VLPs, and polypeptide nucleic acid molecules of the present invention can be formulated in pharmaceutical compositions which are prepared according to conventional pharmaceutical compounding techniques. See, for example, Remington's Pharmaceutical Sciences, 18^{th} Ed. (1990, Mack Publishing, Company, Easton, Pa., U.S.A.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g. topical, intravenous, oral, intrathecal, epineural or parenteral.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, International Patent Publication No. WO 96/11698.

For parenteral administration, the compound may be dissolved in a pharmaceutical carrier and administered as either a solution of a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered and the rate and time-course of administration will depend on the nature and severity of the condition being treated. Prescription of treatment, e.g. decisions on dosage, timing, etc. is within the responsibility of general practitioners or specialists and typically takes account of the disorder to be treated, the condition of the individual patient, the site of delivery, the method of administration and other factors known to practitioners. Examples of techniques and protocols can be found in Remington's Pharmaceutical Sciences, supra.

Alternatively, targeting therapies may be used to deliver the active agent more specifically to certain types of cell, by the use of targeting systems such as antibodies or cell specific ligands or specific nucleic acid molecules. Targeting may be desirable for a variety of reasons, e.g. if the agent is self-antigenic or if it would not otherwise be able to enter the target cells.

Instead of administering these agents directly, they could be produced in the target cell, e.g. in a viral vector such as described below or in a cell based delivery system such as described in U.S. Pat. No. 5,550,050 and International Patent Publication Nos. WO 92/19195, WO 94/25503, WO 95/01203, WO 95/05452, WO 96/02286, WO 96/02646, WO 96/40871, WO 96/40959 and WO 97/12635. The vector could be targeted to the target cells. The cell based delivery system is designed to be implanted in a patient's body at the desired target site and contains a coding sequence for the target agent. Alternatively, the agent could be administered in a precursor form for conversion to the active form by an activating agent produced in, or targeted to, the cells to be treated. See, for example, European Patent Application No. 0 425 731A and International Patent Publication No. WO 90/07936.

Vaccine composition may alternatively comprise nucleic acid molecules encoding the recombinant VLPs.

Gene transfer systems known in the art may be useful in the practice of genetic manipulation. These include viral and non-viral transfer methods. A number of viruses have been used as gene transfer vectors or as the basis for preparing gene transfer vectors, including papovaviruses (e.g. SV40, Madzak et al., J. Gen. Virol. 73:1533-1536, 1992), adenovirus (Berkner, Curr. Top. Microbiol. Immunol., 158:39-66, 1992; Berkner et al., BioTechniques, 6:616-629, 1988; Gorziglia et al., J. Virol., 66:4407-4412, 1992; Quantin et al., Proc. Natl. Acad. Sci. USA, 89:2581-2584, 1992; Rosenfeld et al., Cell, 68:143-155, 1992; Wilkinson et al., Nucleic Acids Res., 20:2233-2239, 1992; Stratford-Perricaudet et al., Hum. Gene Ther., 1:241-256, 1990; Schneider et al., Nature Genetics, 18:180-183, 1998), vaccinia virus (Moss, Curr. Top. Microbiol. Immunol., 158:25-38, 1992; Moss, Proc. Natl. Acad. Sci. USA, 93:11341-11348, 1996), adeno-associated virus (Muzyczka, Curr. Top. Microbiol. Immunol., 158:97-129, 1992; Ohi et al., Gene, 89:279-282, 1990; Russell et al., Nature Genetics 18:323-328, 1998), herpesviruses including HSV and EBV (Margolskee, Curr. Top., Microbiol. Immunol., 158:67-95, 1992; Johnson et al, J. Virol., 66:2952-2965, 1992; Fink et al., Hum. Gene Ther., 3:11-19, 1992; Breakefield et al., Mol. Neurobiol., 1:339-371, 1987; Freese et al., Biochem. Pharmacol., 40:2189-2199, 1990; Fink et al., Ann. Rev. Neurosci., 19:265-287, 1996), lentiviruses (Naldini et al., Science, 272:263-267, 1996), Sindbis and Semliki Forest virus (Berglund et al., Biotechnology, 11:916-920, 1993) and retroviruses of avian (Bandyopadhyay et al., Mol. Cell. Biol., 4:749-754, 1984; Petropoulos et al., J. Viol., 66:3391-3397, 1992), murine (Miller, Curr. Top. Microbiol. Immunol. 158:1-24, 1992; Miller et al., Mol. Cell. Biol., 5:431-437, 1985; Sorge et al., Mol. Cell. Biol., 4:1730-1737, 1984; Mann et al., J. Virol., 54:401-407, 1985; Miller et al., J. Virol., 62:4337-4345, 1988) and human (Shimada et al., 3. Clin. Invest., 88:1043-1047, 1991; Helseth et al., 3. Virol., 64:2416-2420, 1990; Page et al J. Virol., 64:5270-5276, 1990; Buchschacher et al., J. Virol., 66:2731-2739, 1982) origin.

Non-viral gene transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

In an approach which combines biological and physical gene transfer methods, plasmid DNA of any size is combined with a polylysine-conjugated antibody specific to the adenovirus hexon protein and the resulting complex is bound to an adenovirus vector. The trimolecular complex is then used to infect cells. The adenovirus vector permits efficient binding, internalization and degradation of the endosome before the coupled DNA is damaged. For other techniques for the delivery of adenovirus based vectors, see U.S. Pat. No. 5,691,198.

Liposome/DNA complexes have been shown to be capable of mediating direct in vivo gene transfer. While in standard liposome preparations the gene transfer process is non-specific, localized in vivo uptake and expression have been reported in tumor deposits, for example, following direct in situ administration.

General methods for generating the viral particles of the present invention are well known to skilled practitioners.

Another aspect of the present invention is directed to antibodies or their binding fragments to the fusion and/or associated polypeptides of the present invention. The present specification contemplates an antibody which specifically recognises a virus-like particle according to any of the embodiments disclosed herein. Antibodies may be monoclonal or polyclonal and techniques for their manufacture are very well known. The term “antibody” is used in the broadest sense and specifically covers single monoclonal antibodies and antibody compositions with polyepitopic specificity. The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The monoclonal antibodies herein include hybrid and recombinant antibodies produced by splicing a variable (including hypervariable) domain of an antibody, such as an anti-E1E2 or anti-HA2-S antibody or antibody fragment (e.g., Fab, F(ab′).sub.2, and Fv), so long as they exhibit the desired biological activity. See, e.g. U.S. Pat. No. 4,816,567 and Mage and Lamoyi, in Monoclonal Antibody Production Techniques and Applications, pp. 79-97 (Marcel Dekker, Inc.: New York, 1987). Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, Nature, 256:495 (1975), or may be made by recombinant DNA methods. U.S. Pat. No. 4,816,567. The “monoclonal antibodies” may also be isolated from phage libraries generated using the techniques described in McCafferty et al., Nature, 348:552-554 (1990), for example. Antibodies or their fragments which specifically recognise determinants of the fusion or associated polypeptide E1 and E2 of HCV of the present invention are particularly preferred.

The present invention provides a method of treating, ameliorating or prophylactically preventing an infection or condition in a subject, said method comprising administering to a subject or to a particular site in the subject an effective amount of a composition comprising a recombinant nucleic acid construct encoding a chimeric fusion protein, wherein the sequence encoding the fusion protein comprises i) a contiguous sequence encoding a precursor or polyprotein of two or more polypeptides of interest (POI) each comprising a transmembrane domain and/or a protein binding motif or domain, and ii) a sequence encoding a particle-associating portion of an L polypeptide of an avian hepadnavirus. The sequence encoding the fusion protein comprises a cleavage site between the components of the precursor or polyprotein so that the precursor is cleaved after expression in a cell.

In another embodiment, the compositions comprises a recombinant hepadnavirus VLP comprising S polypeptide of an avian hepadnavirus or a functional variant thereof and i) a fusion protein comprising at least one polypeptide of interest covalently attached to a particle-associating portion of avian hepadnavirus L polypeptide or a functional variant thereof, wherein the polypeptide of interest comprises a transmembrane domain or a protein binding domain or motif and ii) at least a second or further polypeptide of interest comprising a transmembrane domain and/or a binding domain or motif, wherein the second or further protein is associated with the VLP via non-peptide bonds.

In some embodiments, the subject VLPs, when present in the subject, are capable of inducing or enhancing an immune response to an infecting organism comprising a native form of the POI presented by the VLP.

The invention also provides expression vectors and host cells comprising a recombinant hepadnavirus VLP comprising S polypeptide of an avian hepadnavirus or a functional variant thereof and i) a fusion protein comprising at least one polypeptide of interest covalently attached to a particle-associating portion of avian hepadnavirus L polypeptide or a functional variant thereof, wherein the polypeptide of interest comprises a transmembrane domain or a protein binding domain or motif and ii) at least a second or further polypeptide of interest comprising a transmembrane domain and/or a binding domain or motif, wherein the second or further protein is associated with the VLP via non-peptide bonds.

In another embodiment, the present invention provides a diagnostic kit comprising the subject VLPs or specific binding molecules, antibodies or antibody-binding fragments thereof determined by the subject VLPs/POIs. In some embodiments, the VLP is capable of inducing neutralising antibodies and/or an effective cell mediated immune response. Where the POI is a viral envelope protein, preferred viruses are from Flavivirus, Coronavirus, Herpesvirus, Hepadnavirus, Retrovirus, Orthomyxovirus or Paramyxovirus family viruses.

The present invention is further described by the further non-limiting Examples.

### Example 1

### Substitution of a.a. 45-125 of DHBV preS with the N Terminal 82 Amino Acids of the Ectodomain of HCV E2 by Fusion PCR

Plasmid pCDL-E2.465 encodes a chimeric L protein consisting (from N to the C terminus) of DHBV L aa 1-4; HCV E2 aa 384 to 465; DHBV L aa 126 to 328. This represents an insertion of a protein of interest of 82 amino acids. Fusion PCR (overlap extension PCR) was used as described by Ho et al, Gene, 77:51, 1989. Overlapping primers were each paired with an outside primer complementary to the plus strand of pCDL-w.t. or the minus strand of HCV construct in 2 first round PCR reactions using pfu enzyme. The PCR products (153 by from pCDL as template and 272 by with the HCV template) from each reaction were purified using a Qiagen min elute kit and the two purified products used as the template for the fusion PCR reaction using the outside primers. The 578 by fusion PCR product was purified and digested with Xma1, cutting at nt 1743 of the HCV primer sequence and with Aat II, cutting at nt 831 of DHBV L. The plasmid, pMDL-w.t., carrying unique Xma1 and Aat II sites in the DHBV preS coding sequence was used as vector. The digested PCR fragment and the large fragment of the cut vector were excised from an agarose gel and purified using a Prep-a-gene kit (Bio-Rad). Competent cells (DH5a cells) were transformed with the ligated plasmid and transformants selected from ampicillin plates.

Positive clones were detected by restriction enzyme digestion of purified DNA using a restriction site which is also present in the HCV E2 ectodomain sequence (Bsa1).

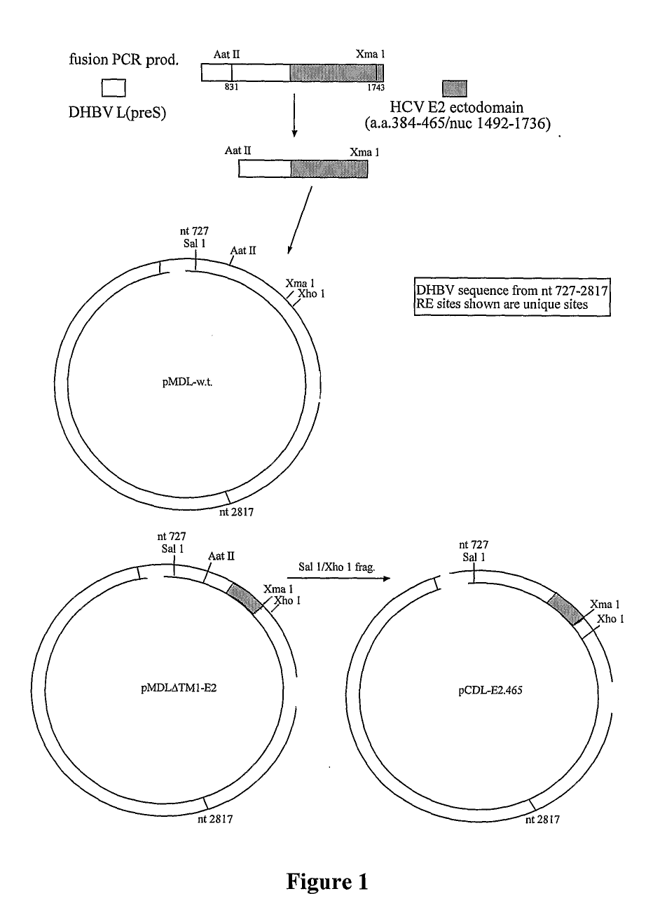

A Sal1/Xho 1 fragment of pMDL-E2 containing the E2 insert was subcloned into pCDL-w.t. (DHBV L expression plasmid with CMV promoter) using the same unique restriction sites (see FIG. 1). Bsa 1 digestion was used again to confirm the presence of the E2 insert in pCDL-E2.465. The CDL-E2.465 clone was also confirmed by sequencing, covering the region of preS-E2-preS and part of S to nucleotide 1581.

Outside Primers
P804
5′ GGGCAACATCCAGCAAAATCAATGG 3′
(SEQ ID NO: 1 DHBV nt 804-828)
P-1719
5′ GCTGCGGAATGGCTAAAAGGGCCCCCGACC 3′ (SEQ ID NO:
2 HCV nt 1719-1749 with an Xmal RE site inserted,
shown underlined)
Overlapping Chimeric Primers (plain type =
DHBVpreS; bold = HCV E2)
P1492 (refers to nt at start of E2 sequence)
CCAACACTAGATCACGAAACCCACGTCACCGGGG
(SEQ ID NO: 3)
P-1492
GGTTGTGATCTAGTGCTTTGGGTGCAGTGGCCCC
(SEQ ID NO: 4)
Templates:
pCDL-wt (DHBV L expression plasmid); p90/HCV
FL-longpU

### Example 2

### Expression and Analysis of CDL-E2 in Avian Hepatoma (LMH) Cells

The avian hepatoma cell line, LMH was co-transfected with 5 μg each of pCDL-E2.465 and pCI-S (Gazina et al, Virology, 242:266, 1998) using the dextran sulphate method (Grgacic et al, J. Gen. Virol., 79:2743, 1998). Day 3 post-transfection media were collected for assessment of exported particles and cells either processed for cytosolic fractionation and assessment of intracellular particle formation or isolation of microsomes for protease protection analysis or isolation of the membrane fraction for assessment of protein expression (Grgacic, J. Gen. Virol., 83:1635, 2002).

### Example 3

### Isolation of Intracellular and Extracellular Particles

(i) Extracellular particles: Media from transfected LMH cells was harvested day 3 post-transfection and clarified of non-adherent cells by centrifugation for 5 min at 2,000 rpm.
(ii) Intracellular particles: Cell monolayers were washed twice with PBS and harvested by scraping cells into 1 ml PBS. Harvested cells were freeze/thawed three times with vigorous vortexing upon thawing. The cytosol fraction (supernatant) was obtained by centrifugation for 1 min at 10,000 rpm in an Eppendorf centrifuge. This procedure has been used in this laboratory to release DHBV particles from transfected cells capable of infecting primary duck hepatocytes.

Particles in the clarified media or cytosol fraction were diluted to 6 ml with PBS and pelleted for 3 h at 38,000 rpm in an SW40 rotor (Beckman) through 3 ml of 20% sucrose onto a 2 ml 70% sucrose cushion. The fraction at the 20-70% interface was collected from the bottom, methanol precipitated for 16 h at −20° C. followed by separation on 13% SDS-PAGE and Western blotting.

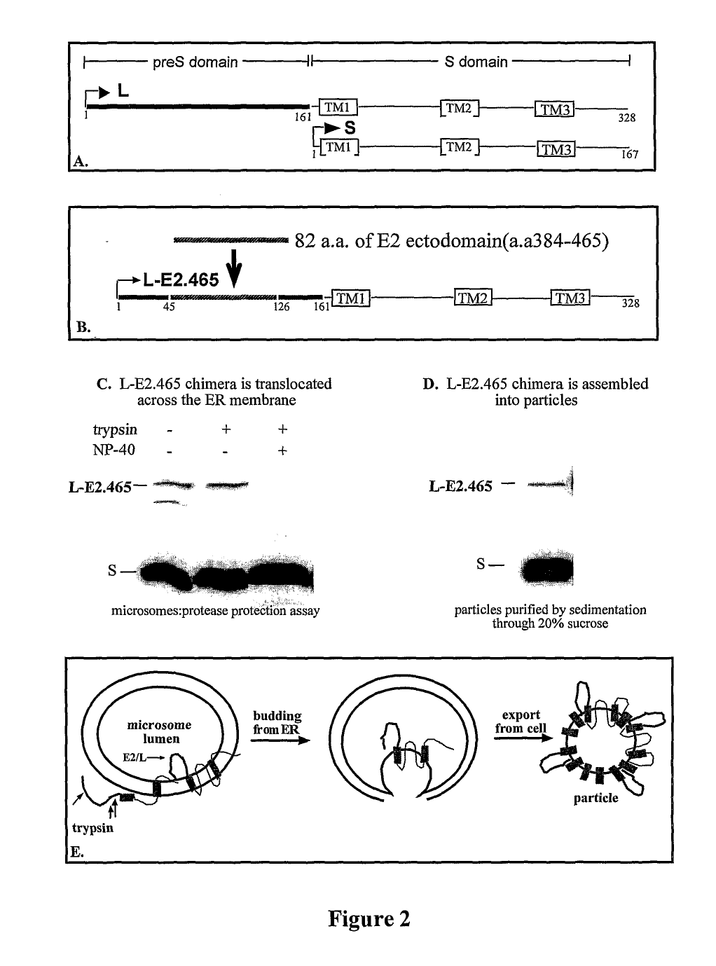

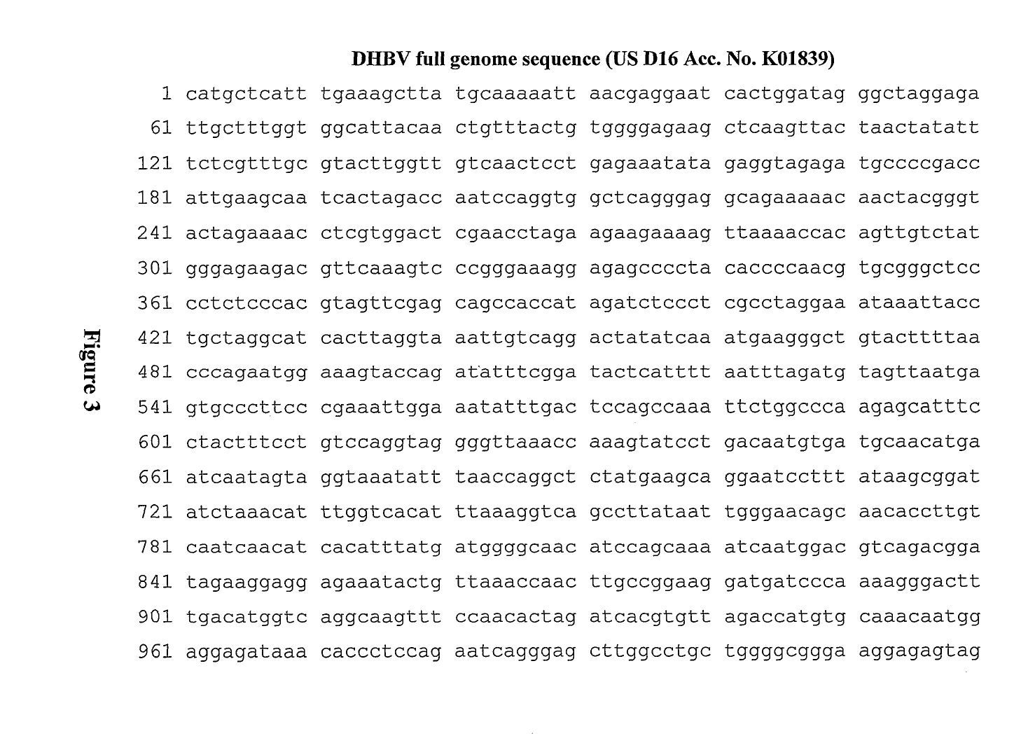

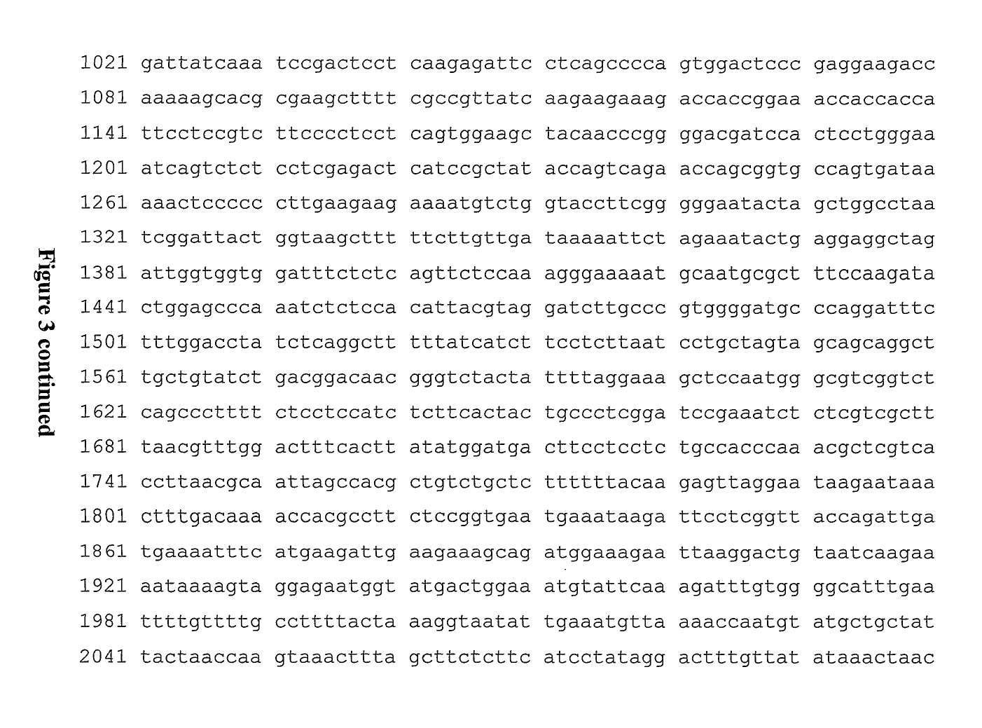

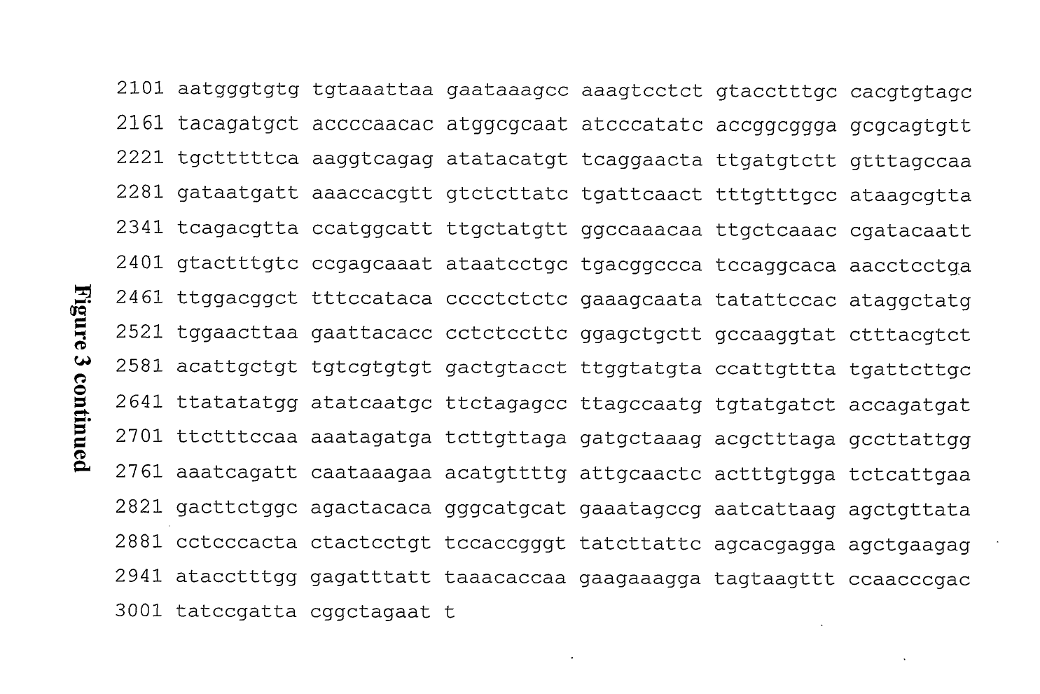

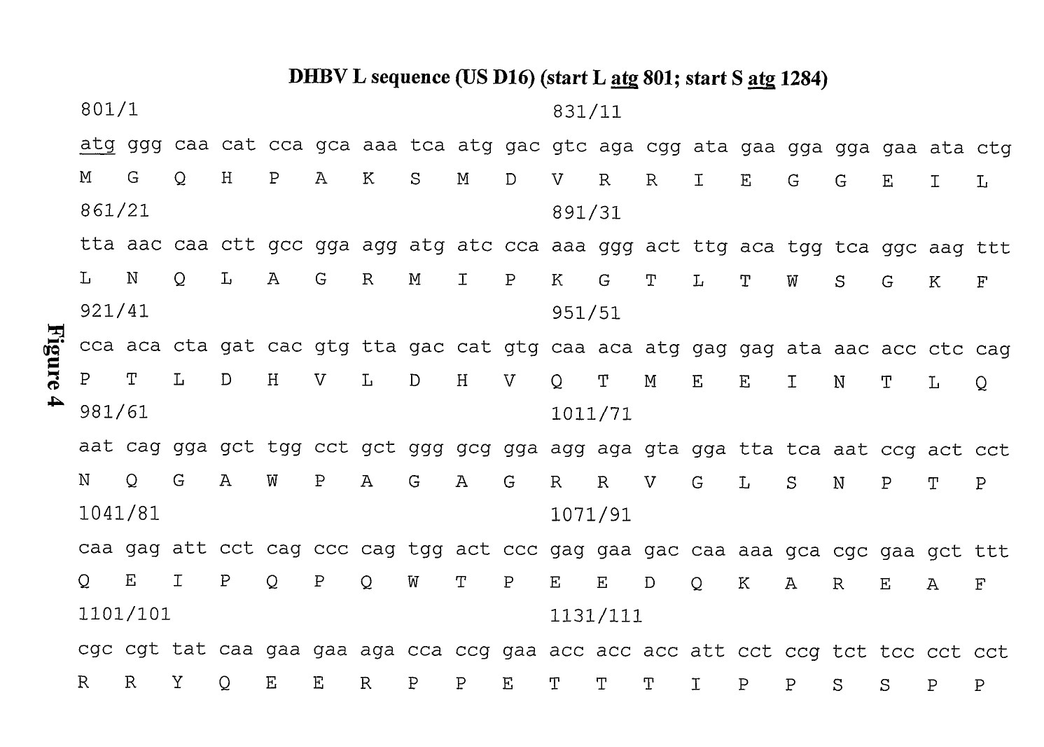

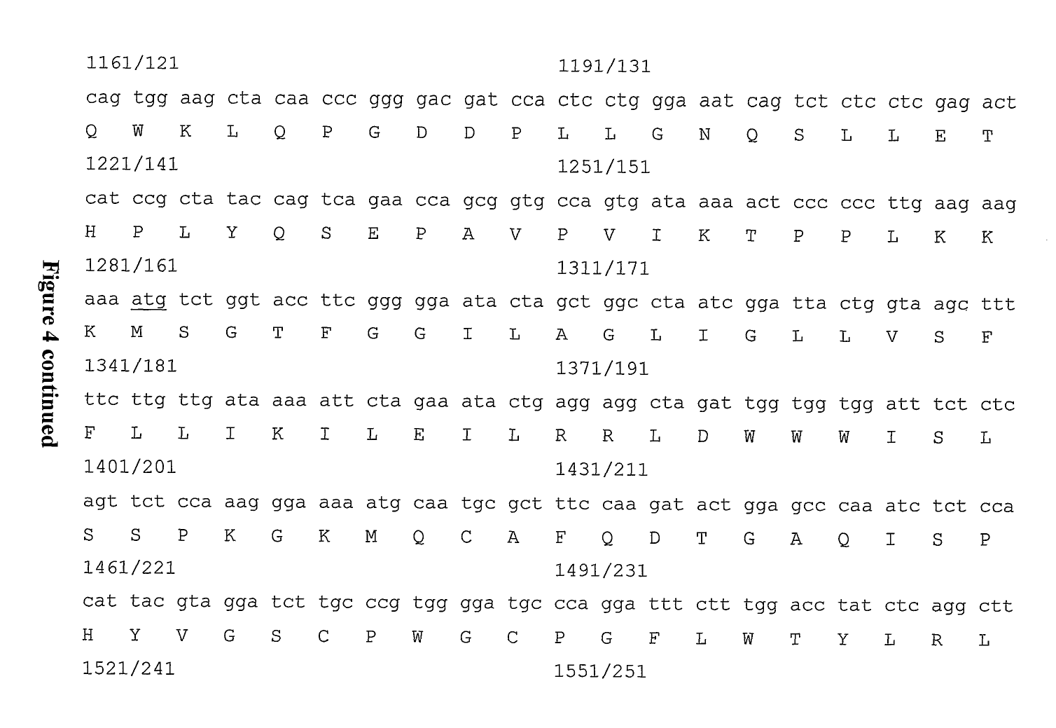

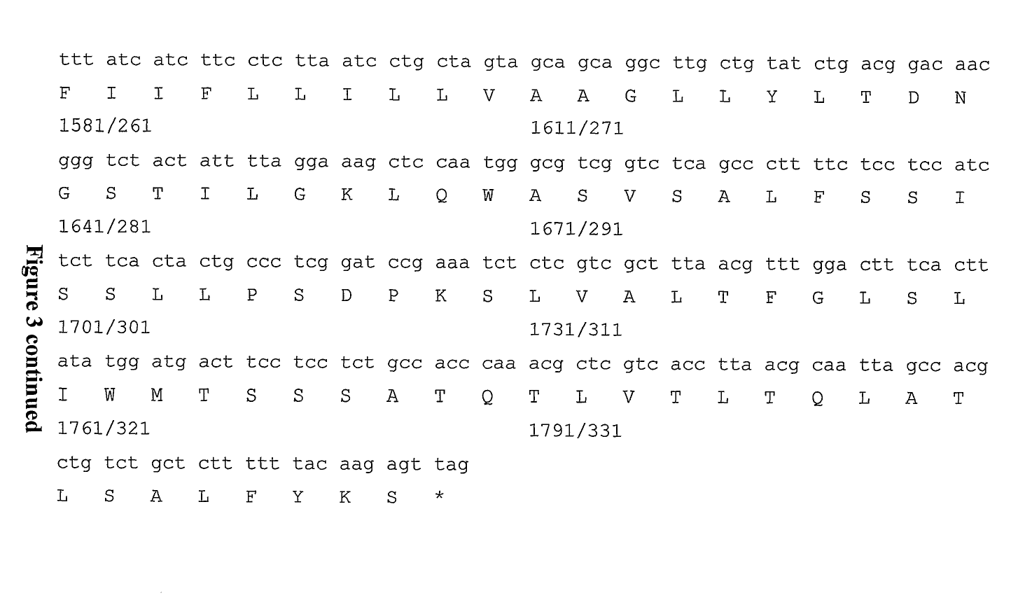

As shown in FIG. 2D, Western Blot show that the E2.465/L chimera is assembled into particles. Intracellular particles were isolated from avian hepatoma (LMH) cells transfected with pCDL-E2.465 and pCI-S by freeze-thawing cells 3 times, centrifugation to obtain the cytosolic fraction for sedimentation of particles through 20% sucrose on to a 70% sucrose cushion at 38,000 r.p.m. (SW41 rotor Beckman). The particle fraction at the 20-70% sucrose interface was methanol precipitated prior to SDS-PAGE and analysis of envelope proteins by Western blotting.

### Example 4

### Protease Protection Analysis

Microsomes were prepared according to the method of Prange et al., EMBO J., 14:247, 1995b with modifications. Transfected LMH cells (two 30 mm diameter wells) were washed in cold Tris-buffered saline (TBS: 50 mM Tris-HCl, pH 7.5; 150 mM NaCl).

The monolayers in each well were incubated on ice with 0.4 ml 0.1×TBS for 10 minutes and then harvested by scraping, pooled and dispersed by drawing 5 times through a 26 G needle. The homogenate was adjusted to 1×TBS with 5×TBS and centrifuged for 20 min at 2,500 rpm at 4° C. to remove unbroken cells and nuclei. The supernatant was removed and set aside while the pellet was again dispersed in 300 μl TBS and centrifuged as before.

Supernatants were pooled and layered onto 2.7 ml 250 mM sucrose in TBS and centrifuged for 30 min. at 38,000 rpm at 4° C. in an SW-60 rotor (Beckman). The microsomal pellets were washed once with TBS and resuspended in 65 μl TBS.

For trypsin protection analysis the microsomal preparation was divided into three 20 μl aliquots. One sample was left untreated while the remaining two were treated with 25 μg/ml of trypsin (TPCK treated; Worthington Biochem. Corp. NJ. USA) with or without 0.5% NP-40 for 1 h. on ice.

### Example 5

### Western Blot Analysis

Proteins were separated by SDS-PAGE (13% acrylamide) and transferred to nitrocellulose membrane (Schleicher and Schüll) using a Trans-Blot SD semi-dry transfer cell (Biorad). Membranes were blocked for 1 h with 3% skim milk in PBS plus 0.3% Tween 20 (PBST). Membranes were probed with monoclonal anti-S (7C.12) (Pugh et al, J. Virol., 69:4814, 1995) for 1 h in 1% skim milk; PBST, then washed with PBST and probed with goat anti-mouse Ig:horse radish peroxidase (Amersham) in 1% skim milk PBST. After a final wash in PBST (3×10 min.) proteins bands were visualised by enhanced chemiluminescence (ECL) (Amersham).

As shown in FIG. 2C, the E2.465/L chimera is translocated across the ER. Protease protection analysis of ER microsomes prepared from LMH cells transfected with pCDL-E2.465 and pCI-S (an S protein expression plasmid). Microsomes samples were subjected to digestion with trypsin in the absence or presence of the detergent, NP-40, or left untreated, as denoted above each lane. Protease protection of E2.465/L chains was analysed by SDS-PAGE and Western blotting with a monoclonal anti-S antibody, which detects both E2.465/L and S proteins. Protection of E2.465/L from trypsin digestion (middle lane) is an indication of translocation to the ER lumen.

### Example 6

### Construction of Strategically Selected Chimeric DHBV VLPs to Define their Carrying Capacity as a Potential Vaccine Delivery Vehicle

The receptor binding region as well as the C terminus of preS is exposed to the DHBV subviral particle surface. These exposed regions, flanked by the membrane spanning S domain, are believed to be further stabilised through anchorage at the N terminus by the myristylation signal. The HCV E2 ectodomain inserted into this region of preS was similarly exposed and stabilised. PreS sequences are substituted by equivalent or larger sized foreign sequences or alternatively fused in frame to the N terminus of the S domain of L by fusion PCR.

To aid translocation of the chimeric L polypeptides, an L construct with a signal sequence such as the preprolactin signal sequence fused to the N terminus, which causes co-translational translocation of L, is also used. These SigL chains can assemble with S subunits and be exported as particles. Translocation of the chimeric preS domains is monitored by the protease protection assay and antibody mapping of the topology on the assembled particle by immunoprecipitation. Particles are purified by sucrose gradient sedimentation and analysed by EM/immunogold labelling for VLP formation. Pulse-chase metabolic labelling are performed to assess that the proportion of recombinant chains relative to S (approximately 1:4 for wild type DHBV) is maintained in the assembled particle.

### Example 7

### Construction and Analysis of the E2.465/L Chimera with TM1 Deleted in L

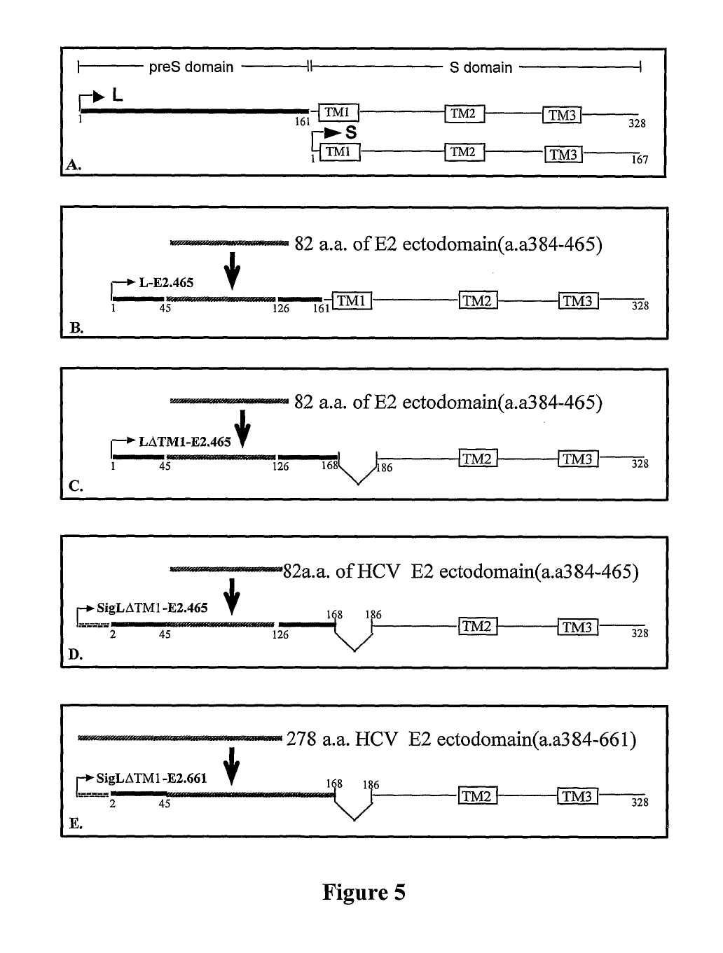

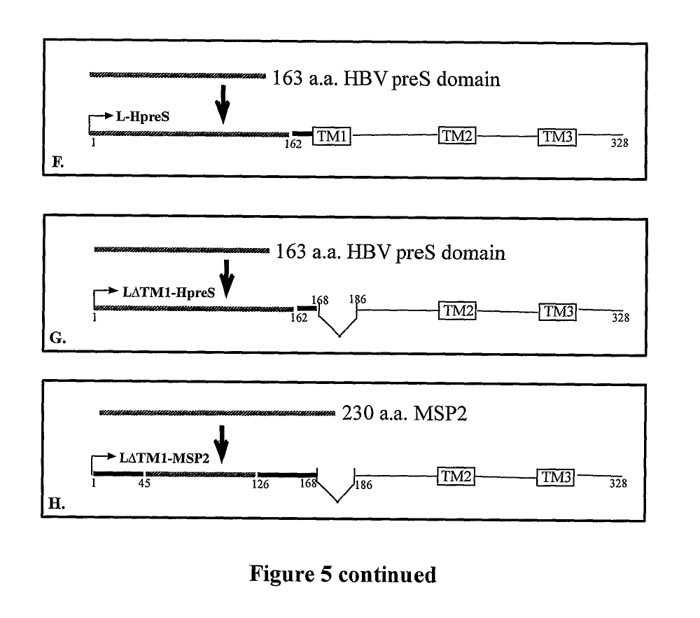

pCDLΔTM1-E2.465 encodes a chimeric L protein consisting (from the N to the C terminus) of DHBV L a.a. 1-45; HCV E2 a.a. 384-465; DHBV L a.a. 126-328 with a deletion of 18 a.a. of transmembrane domain 1 at a.a. 168-186 (see FIG. 5B). This represents an insertion of a protein of interest of 82 a.a.

pCDLΔTM1-E2.465 was constructed by subcloning a Sal 1/BstEII fragment (encompassing preS/E2 and S domain sequences) of pMDLΔTM1-E2.465 into pCDL-w.t. Transfer of the insert was confirmed by restriction enzyme digestion with Bsa 1. Expression and analysis of pCDLΔTM1-E2.465 was done in LMH cells as described in Examples 2, 3 and 4. Expression of the E2.465/LΔTM1 chimera both as protein and as assembled particles, was greater than that observed with the pCDL-E2.465 plasmid. Constructs with the TM1 deletion can be used for all chimeric DHBV VLPs, if required.

### Example 8

### Construction of an E2.465/LΔTM1 Chimera with an N Terminal Signal Sequence (Preprolactin)

pSigLΔTM1-E2.465 encodes a chimeric L protein consisting (from the N to the C terminus) Preprolactin signal sequence a.a. 1-26; DHBV L a.a. 2-45; HCV E2 a.a. 384-465; DHBV L a.a. 126-328 with a deletion of 18 a.a. of transmembrane domain 1 at a.a. 168-186 (see FIG. 5D). This represents an insertion of a protein of interest of 82 amino acids.

Signal sequences fused to the N terminus of DHBV L cause the L protein to be co-translationally translocated across the ER membrane which in turn results in glycosylation of the L protein (Swaymeye et al., J. Virol., 71:9434, 1997; Gazina et al, 1998 (supra)).

Firstly, pCDSigLΔTM1-E2.465 was constructed by sequential subcloning, first of an AatII/Kpn 1 fragment of pMDLΔTM1-E2.465 into pMDSigLΔTM1-E2.465 and then a Sal 1/BstEII fragment of the latter into pCDSig\tilde{L}A PpuMul/BstEII fragment from pCDSigLΔTM1-E2.465 containing the E2.465 sequences was subcloned into the same sites in PPL-L, encoding the preprolactin signal sequence at the N terminus of DHBV L (Gazina et al, 1998 (supra)). The resulting plasmid was assigned the name, pSigLΔTM1-E2.465. pSigLΔTM1-E2.465 expressed the chimeric L-E2.465 protein at similar levels to the PPL-L protein when examined as in Example 2.

### Example 9

### Construction and Analysis of the E2.661/L Chimera Comprising the Entire Ectodomain of HCV E2

pSigLΔTM1-E2.661 encodes a chimeric L protein consisting (from the N to the C terminus) Preprolactin signal sequence a.a. 1-26; DHBV L a.a. 2-45; HCV E2 a.a. 384-661; DHBV L a.a. 168-328 (see FIG. 5E). This represents an insertion of a protein of interest of 278 amino acids.

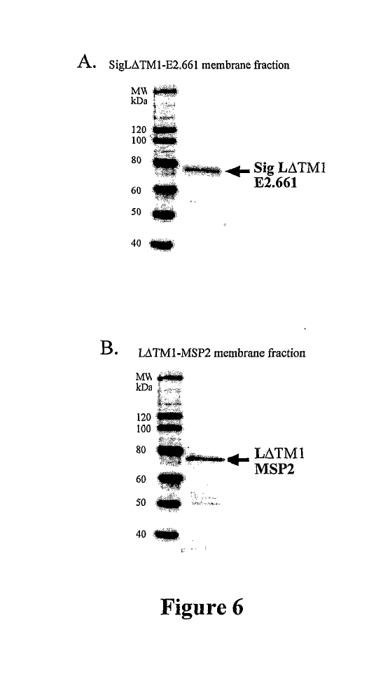

The E2.661/L chimera incorporates a.a. 384-661 of HCV E2, i.e., the 278 amino acid ectodomain of E2 into the preS domain of DHBV L. The E2.661/L was constructed by PCR using a primer to the sequence at the start site if HCV E2 (nt 1490) and reverse primer covering nt 2321 at the end of the ectodomain of E2 and incorporating a Kpn 1 restriction enzyme site. The PCR product was digested with Nae 1 (nt 1517 of E2) and Kpn 1 and inserted into the same sites in pCDLΔTM1-E2.465 to create pCDLΔTM1-E2.661. Incorporation of the PPL signal sequence to this construct was done by a three-way ligation of the following fragments: a Nar1/BstE II fragment of pCDLΔTM1-E2.661 encompassing the E2 sequence; a Bgl II/Nar 1 fragment of the pSigLΔTM1-E2.465 encompassing the preprolactin sequence and part of E2 and a Bgl II/BstE II fragment of PPL-L providing the remaining vector sequences. Expression of the resulting construct pSigLΔTM1-E2.661 was shown in LMH cells as described in Example 2 (see FIG. 6A).

### Example 10

### Construction and Analysis of HBVpreS/L and HBVpreS/LΔ Chimeras

pCDL-HBVpreS encodes a chimeric L protein consisting (from the N to the C terminus) of HBV preS a.a. 1-163; DHBV L a.a. 162-328. pCDLΔ-HBVpreS encodes a chimeric L protein consisting (from the N to the C terminus) of HBV preS a.a. 1-163; DHBV L a.a. 162-328 with a deletion of 18 a.a. of transmembrane domain 1 at a.a. 168-186 (see FIG. 5F to 5G). These constructs represents an insertion of a protein of interest of 163 amino acids.

Genome sequences encoding the preS domain (a.a. 1-163) of HBV (strain ayw) and the S domain of DHBV were amplified by PCR from plasmids encoding the respective L proteins, then joined by fusion PCR. The HBV preS primer introduced a Sal 1 restriction site upstream of the initiation of HBV preS. The fusion PCR product was digested using Sal 1 and a BstEII restriction site in the DHBV sequence upstream of the DHBV primer and ligated into the same sites in pCDL-w.t. to create the pCDL-HBVpreS plasmid. The HBV preS sequence, which has no sequence homology with the DHBV preS (Sprengel et al, J. Med. Virol., 15:323, 1985), is thus directly fused to the S domain of DHBV L in these constructs.

pCDLΔ-HBVpreS was constructed by a three-way ligation of the following fragments: a Sal1/Kpn1 fragment of pCDL-HBVpreS encompassing the HBV preS sequence, a Kpn1/BstEII fragment of pCDLΔTM1 encompassing the DTM1 region and a Sal 1/BstEII fragment of pCDL-w.t. providing the remaining vector sequences.

Expression and analysis of pCDL-HBVpreS and pCDLΔ-HBVpreS was done in LMH cells as described in Examples 2, 3 and 4.

### Example 11

### Construction and Analysis of the P. falciparum MSP2/LΔTM1 Chimera

pCDLΔTM1-MSP2 encodes a chimeric L protein consisting (from the N to the C terminus) of DHBV L a.a. 1-30; P. falciparum MSP2 a.a. 20-249; DHBV L a.a. 126-328 with a deletion of 18 a.a. of transmembrane domain 1 at a.a. 168-186 (see FIG. 5H). This represents an insertion of a protein of interest of 230 amino acids.

The malaria pathogen, Plasmodium falciparum (isolate NF54 clone 3D7) MSP2 gene was cloned by fusion PCR. Two sets of primers were used for generating separate templates for fusion. The first set encompassed a forward primer incorporating the SalI restriction enzyme site of pCDLΔTM1 and a reverse primer overlapping the first 27 bp of MSP2 and the second set consisted of a forward primer overlapping DHBV L and a reverse primer incorporating a XmaI restriction enzyme site into MSP2. The separate templates were then joined by fusion PCR, restricted by Sail and XmaI and ligated into the vector. The ligated insert-vector was transformed into E. coli DH5 alpha, creating the chimeric vector pCDL ΔTM1-MSP2. Expression of the resulting construct was shown in LMH cells as described in Example 2 (see FIG. 6B).

### Example 12

### Production of DHBV VLPs in Saccharomyces cerevisiae

For the purposes of scaling-up production of recombinant DHBV VLPs and chimeric DHBV VLPs for immunisation studies, a yeast inducible expression system was used. DHBV DNA encoding the large envelope protein was cloned into the pYES2 vector (Invitrogen) by PCR using a primer to the sequence upstream (starting at nt 762) of the start site of DHBV L (nt 801) and incorporating a Sac I restriction enzyme site and a reverse primer covering nucleotide 1910 and incorporating an Eco R1 restriction site. The PCR product was digested and inserted into the Sac1 and EcoR1 sites in the multicloning site of the vector. The pYES2 vector carries an ampicillin resistance gene for selection of clones in E. coli. Clones containing the DHBV L gene (pYES-DL) were confirmed by restriction enzyme digestion and one was selected for transformation of the yeast strain, INVSc1.

A yeast expression plasmid for DHBV S gene expression, pMB-DS (Klingmuller et al., J. Virol., 67:7414-7422, 1993), was used for co-transformation of the INVSc1 strain with pYES-DL. The pYES2 vector carries a URA3 gene for selection of transformants in yeast and pMB-DS carries a LEU2 selection marker. The INVSC-1 stain used will not grow in media deficient in leucine, uracil, histidine and tryptophan. Co-transformants were therefore selected for growth on media lacking both leucine and uracil. Transformation of competent INVSc-1 cells was done according to the manufacturer's (INVITROGEN) instructions. Both plasmids have a GAL1 promoter for high level inducible protein expression in yeast by galactose and repression by glucose. Transformants were grown in yeast synthetic drop-out media without uracil and leucine (SC-UL) with 2% glucose for 2 days and then induced for protein expression by the substitution of glucose with 2% galactose and grown in YEP (1% yeast extract; 2% peptone) media for a further 24-48 hours.

DHBV L and S protein expression was examined by Western blotting following extraction of protein in yeast cells with acid-washed glass beads and vigorous vortexing followed by centrifugation. Supernatants were analysed by Western blotting with anti-S monoclonal as described in Example 5. Transformants (DL/S), which expressed the greatest amount of L and S protein were selected and stored as glycerol stocks.

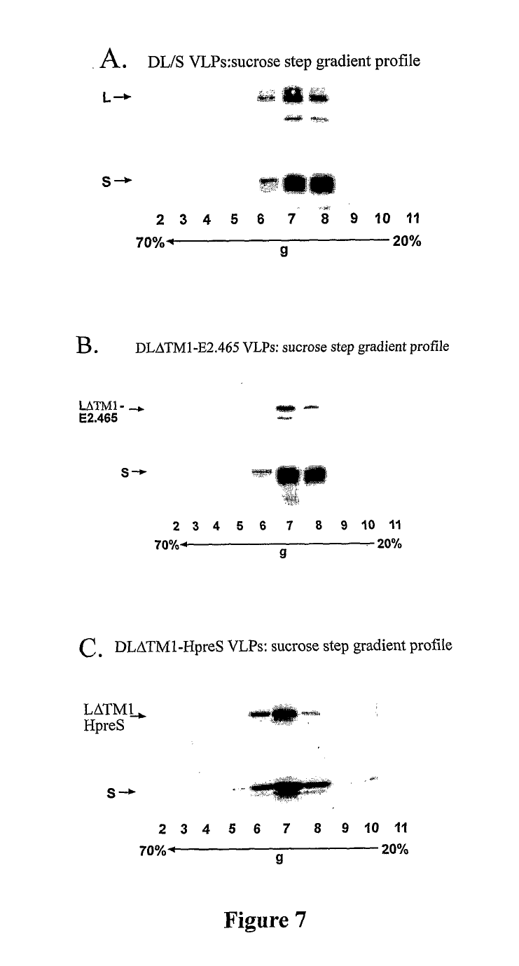

For analysis of DHBV VLP production: A 50 ml yeast culture of DL/S was extracted and the supernatant loaded onto 20% sucrose above a 70% sucrose cushion and centrifuged at 38,000 rpm for 3 hours in a SW41 rotor (Beckman). The fraction at the 20-70% interface was then loaded onto a 20-70% sucrose step gradient and centrifuge for 5 hours at 38,000 rpm (Grgacic et al., J. Viral., 74:5116, 2000). Fractions were collected from the bottom of the gradient and analysed by Western blotting. Serum-derived DHBV subviral particles sediment at approximately 30% sucrose (peak fractions 7 and 8). The yeast-derived DHBV VLPs were similarly shown to sediment largely at 30% sucrose (see FIG. 7A).

Transmission Electron Microscopy (TEM) of yeast-derived particles was conducted. Particles for TEM were sucrose gradient purified and further buffer exchanged with phosphate buffered saline using a Vivaspin 20 desalting and concentration device (Vivascience) prior to negative staining with uranyl acetate. A comparison of TEM of serum-derived DHBV subviral particles and yeast-derived DL/S particles showed similar particle morphology and size (approx 40-60 nm).

Variations on the pYES-DL construct to include the deletion in transmembrane domain 1 and the preprolactin signal sequence with and without the deletion in transmembrane domain I were made. pYES-DLΔTM1 was constructed by subcloning an Aat II/Bst EII fragment of pMDLΔTM1 containing the region of the TM1 deletion into pYES-DL using the same restriction enzyme sites. pYES-SigL was constructed by subcloning a Sac 1/Bst EII fragment of PPL-L containing the signal sequence into pYES-DL using the same restriction sites. pYESSigLΔTM1 was constructed by subcloning an Aat II/BST Eii fragment of pMDLLTM1 containing the TM1 deleted region into pYES-SigL using the same restriction sites. VLP production and analysis in yeast was done as described above. DLΔTM1/S, SigL/S and SigLΔTM1/S VLPs were shown to have the same sedimentation profile as DL/S particles in a sucrose step gradient.

### Example 13

### Production of Chimeric DHBV VLPs in Saccharomyces cerevisiae

pYES-DL-E2.465 and pYES-DLΔTM1-E2.465 were constructed by subcloning E2 encompassing sequences from pMDLΔTM1-E2.465 in an Aat II/Xma1 fragment for the former and an Aat II/Bst EII fragment for the latter into the same sites in pYES-DL. INVSc-1 cell were co-transformed with either pYES-DL-E2.465 and pMB-DS or pYES-DLΔTM1-E2.465 and pMB-DS for chimeric particle production and analysis in yeast as described in Example 11. The DLΔTM1-E2.465 VLPs were shown to have the same sedimentation profile as DL/S particles in a sucrose step gradient (see FIG. 7B) and have a similar morphology to DL/S particles by TEM.

pYES-DLΔTM1-HpreS was constructed by PCR using a primer to the sequence upstream (starting at nt 4091) of the start site of HBV preS (nt 4138) and incorporating a Sac 1 restriction enzyme site and a reverse primer covering nucleotide 1910 of the DHBV sequence and incorporating an Eco R1 restriction site. The PCR product was digested and inserted into the Sac1 and EcoR1 sites in the multicloning site of the PYES vector. Chimeric particle production and analysis in yeast was done as described in Example 11. DLΔTM1-HpreS VLPs were shown to have the same sedimentation profile as DL/S particles in a sucrose step gradient (see FIG. 7C) and have a similar morphology to DL/S particles by TEM.

### Example 14

### Analysis of Immunogenicity of DHBV VLPs Produced in Yeast

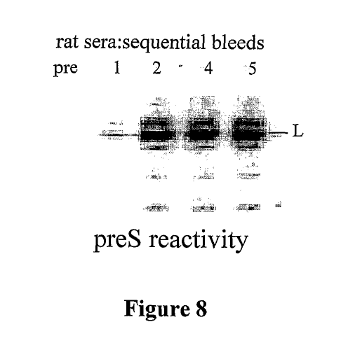

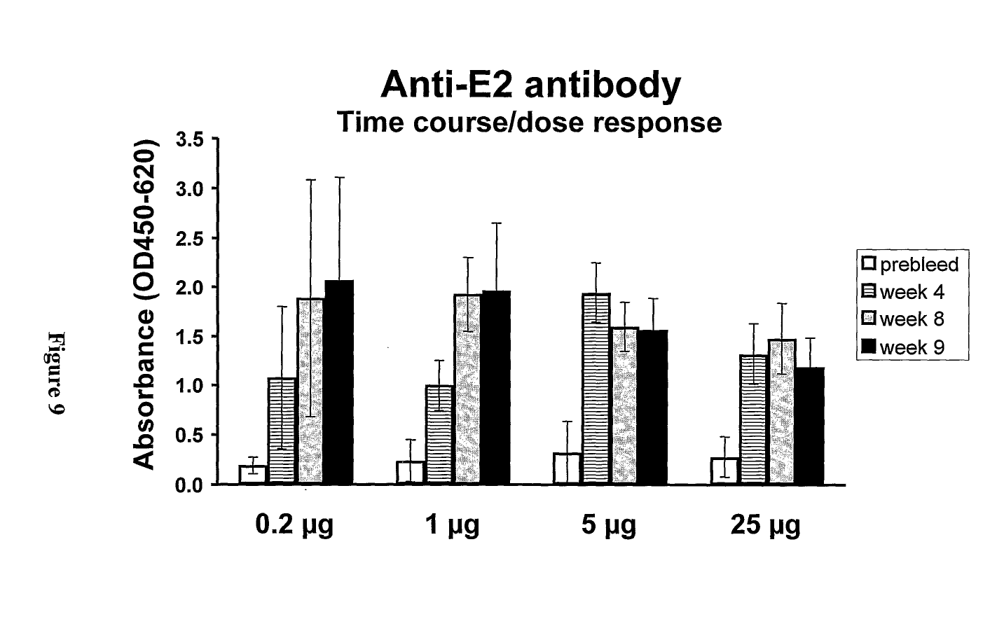

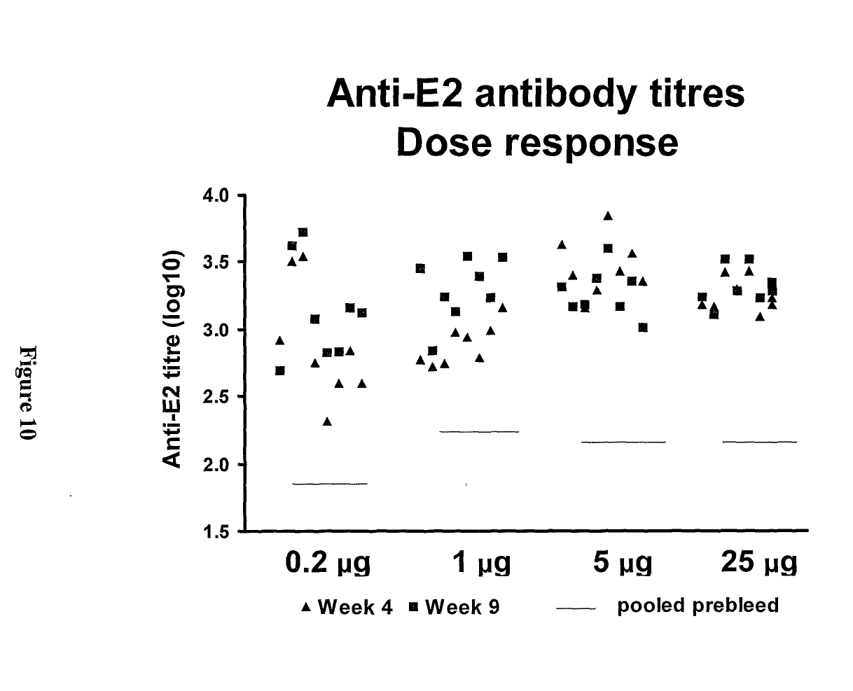

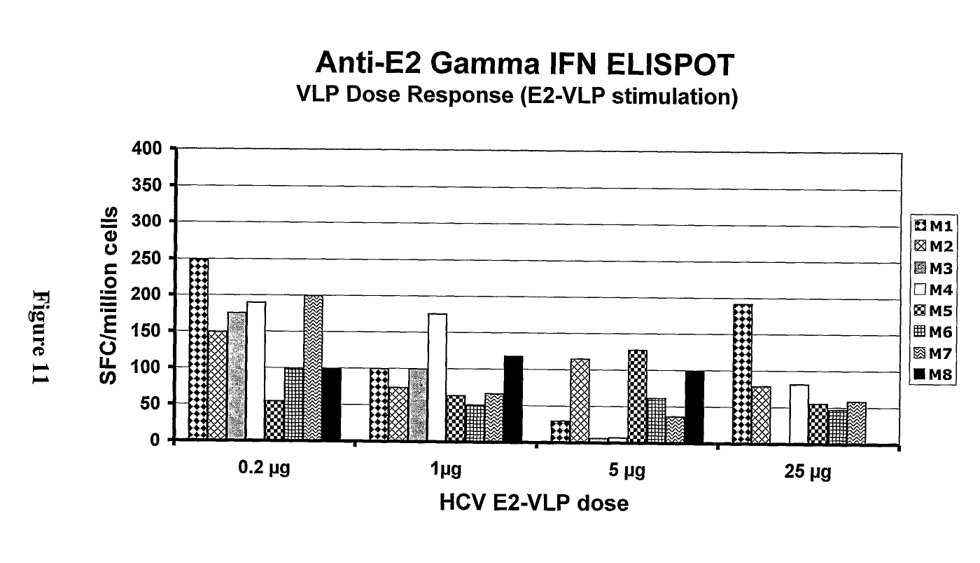

DL/S VLPs were used to immunize rats. DL/S VLP production: A 100 ml yeast culture of DL/S was extracted and the supernatant loaded onto two 20% sucrose above a 70% sucrose cushion and centrifuged at 38,000 rpm for 3 hours in a SW41 rotor (Beckman). The portion of the pelleted VLPs was examined by SDS-PAGE and Coomassie Brilliant Blue protein staining against a standard protein to estimate the amount of VLP protein. Approximately 10 μg doses of DL/S in a total of 200 μl were injected in rats i.m. Rats were put into three groups of six rats with each group receiving DL/S VLPs either without the addition of an adjuvant or with the addition of alum or Titremax. Rats were bled 3 weeks following immunisation and subsequent boosts. Analysis of rat sera by Western blotting of DHBV L/S protein showed a strong and rapid immunoreactivity without the presence of adjuvant to the DHBV L protein with little or no response to DHBV S protein (see FIG. 8).

### Example 15

### DHBV VLPs Comprising E2 Glycoprotein of HCV Induced Strong Antibody and T-Cell Mediated Response

Strong antibody responses were detected to DHBV VLPs comprising the ectodomain of E2 (at 384 to 661) of HCV sequence H771a genotype (NCBI Accession No. AF011751.3; SEQ ID NO:15). As shown in FIG. 9, antibody responses were measured by measuring the concentration of anti-E2 antibody (OD450-620) over a time course of 9 weeks with different concentration of VLP (0.2 μg, 5 μg and 25 μg). FIG. 10 provides a schematic representation of the dosage response over time against the log10 anti-E2 titre in individual animals from the experiment referred to in FIG. 9.

T-cell responses were detected in animals administered various doses (0.2 μg, 1 μg, 5 μg and 25 μg) of DHBV-VLP comprising the ectodomain of E2 of HCV (see FIG. 11). T-cell response were measured in vitro after E2-VLP stimulation in an IFN-γ ELISPOT assay.

### Example 16

### DHBV VLPs are Taken up by Dendritic Cells

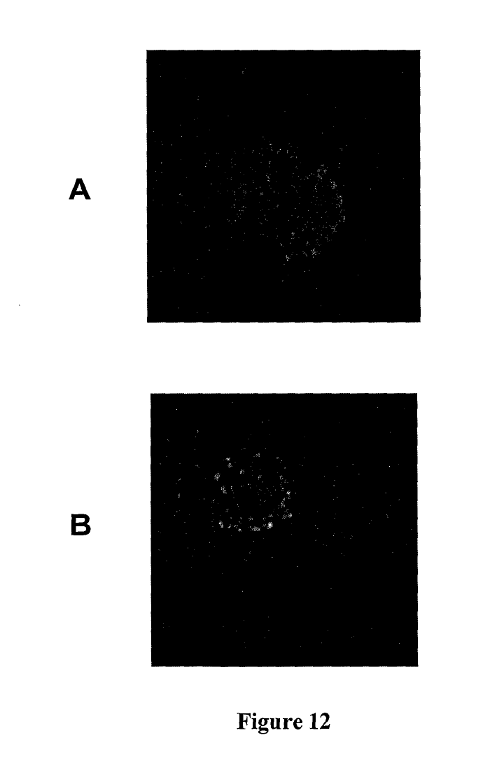

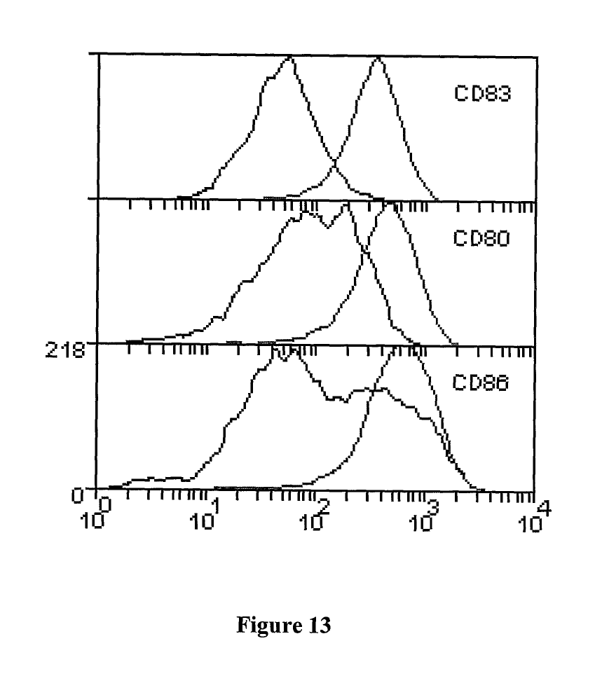

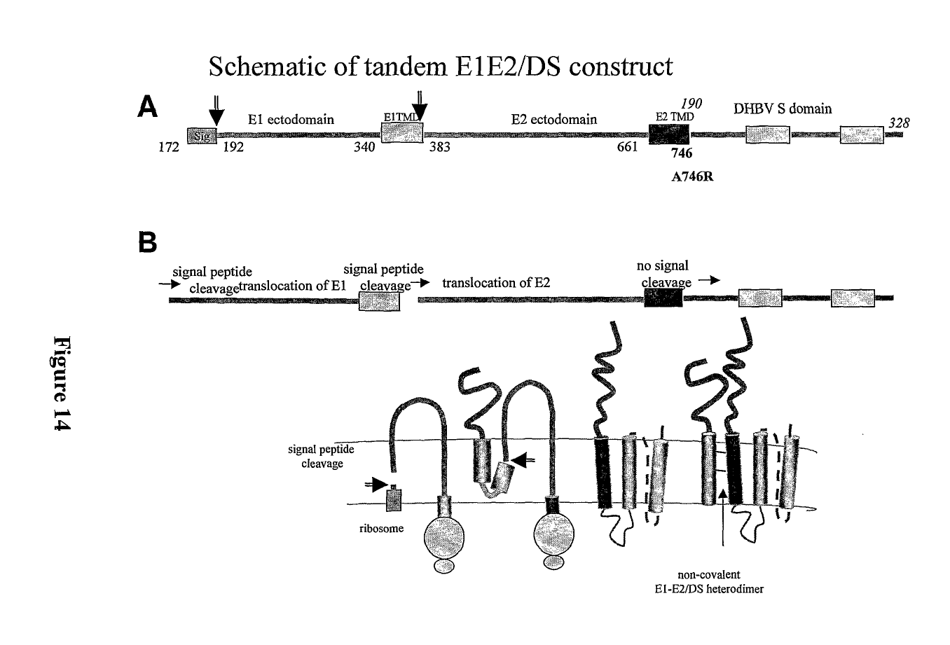

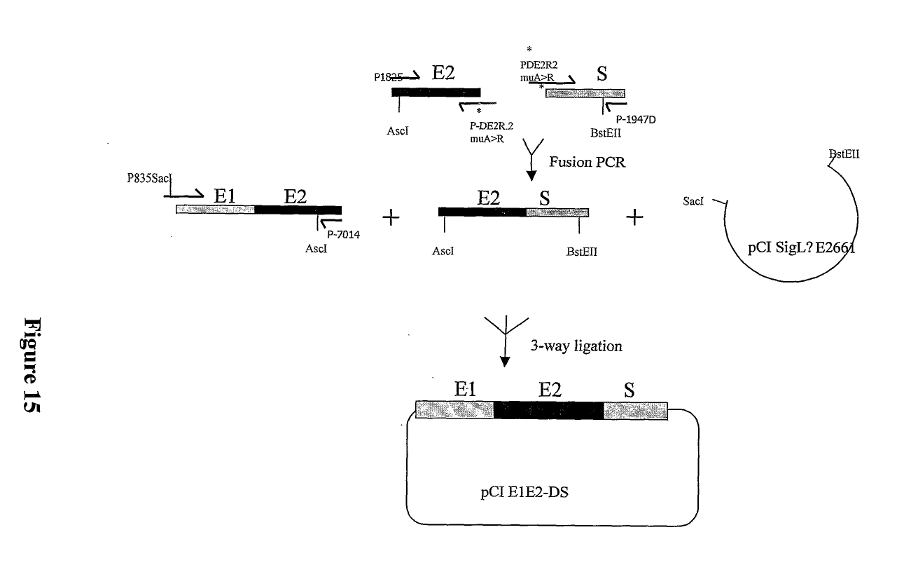

Uptake of wild type DHBV-VLPs and of DHBV-VLPs comprising the ectodomain of E2 of HCV was demonstrated in culture of human dendritic cells (see FIG. 12). Immature human dendritic cells that have taken up DHBV-VLPs comprising the ectodomain of E2 of HCV show evidence of functional maturation which is likely to further promote the immune response to antigens in the VLP (see FIG. 13 showing expression of dendritic cell markers associated with maturation).

### Example 17

### Recombinant VLPs Comprising Two or More Viral Envelope Protein that Occur Naturally Bound to Each Other or to the Viral Envelope by Non-Covalent Bonds

In the present invention, a VLP is provided in which a further part or parts of the POI is incorporated into the VLP by virtue of non-peptide bond interactions. The present invention provides a recombinant hepadnavirus VLP comprising i) a fusion protein comprising at least one viral envelope protein covalently attached to a particle-associating portion of avian hepadnavirus L polypeptide wherein the viral envelope protein comprises a transmembrane domain or a binding domain and ii) at least a second or further viral envelope protein comprising a transmembrane domain or a binding domain, wherein the second or further protein is associated with the VLP via non-peptide bonds. In some embodiments, the viral envelope protein forms conformational epitopes capable of inducing neutralising antibodies against naturally occurring enveloped viral particles.

In some embodiments, a VLP is provided in which the POI is comprised of two polyproteins each with its own transmembrane domain, wherein only one of the two polyproteins is incorporated as a fusion polypeptide with the small envelope polypeptide. The POI of the fusion polypeptide is incorporated in such a way that the native transmembrane domain of the POI replaces the TM1 of the S protein (particle-associating portion of the L protein). The second part of the POI is incorporated by virtue of interactions between the native transmembrane domains of the first and second parts of the POI. It will be clear that further examples can be anticipated in which the different portions of the POI may be associated via interactions between parts of the polypeptide other than transmembrane domains. These may include (without restriction) leucine zippers, amyloid domains, disulphide bonds or antibody-antigen interactions.

The E2 glycoprotein of HCV is normally synthesised as part of a precursor polypeptide together with the E1 glycoprotein, with the subsequent cleavage of the polypeptide to yield E1 and E2, and the E1 and E2 glycoproteins remain associated in the HCV virus particle by non-covalent interactions between their respective transmembrane domains (see Op de Beek et al., J. Gen. Virology, 82:2589-2595, 2001 and Penin et al., Structural Biology of Hepatitis C Virus Hepatology, 39:5-19, 2004.

As such, the function of E2 in the VLP, such as antigenic or immunogenic function are enhanced by its interactions with E1. In addition, E1 is also an important target of neutralising antibodies, and its inclusion in hepatitis C vaccines along with E2 is likely to enhance the overall immune response. Equally, the function of E1 will also be enhanced by its interactions with E2.

Because E1 is proteolytically cleaved from E2 during HCV biogenesis, it appeared unlikely that E1 could be incorporated into VLPs. However, a method is provided whereby, in one embodiment, E1 can be incorporated into VLPs via interactions with E2, resulting in the formation of VLPs containing E1 and E2 with enhanced function of E2 compared to VLPs containing E2 alone. In some embodiments, the present VLPs have the further advantage of containing E1 for the induction of additional E1-specific immune responses, with the E1 also having enhanced function compared to VLPs containing E1 alone.

A schematic representation of the DNA construct used to express E1 and E2 in tandem to allow their incorporation into VLPs and the proposed final topology of the mature E1 and E2-DS proteins within the VLP is provided in FIG. 14.

In one particular embodiment, an E1E2-DS tandem construct for expression of full length HCV E1 and full length HCV E2-DHBV S fusion protein is produced. pE1E2-DS encodes, as shown schematically in FIG. 14A, a chimeric HCV E1E2-DS envelope protein consisting (from the N to the C terminus) N terminal signal sequence for E1a.a. 172-192 of the HCV sequence H771a genotype (Accession No. AF011751-3); E1 ectodomain a.a. 190-340; E1 transmembrane domain (TMD) inclusive of the E2 signal sequence a.a. 340-383; the E2 ectodomain a.a. 384-661; the E2 transmembrane domain with a point mutation at Ala 746 to Arg to inhibit cleavage of the signal sequence within the TMD a.a. 661-746; the DHBV S domain a.a. 190-328 of the DHBV L sequence. The two pronged arrows indicate signal peptide cleavage sites which are utilised during synthesis of the polyproteins. This represents insertion of a POI of 554 amino acids (exclusive of the a.a. N terminal signal sequence of E1 which is likely to be cleaved during synthesis of the polypeptide).

As shown schematically in FIG. 14B, translocation occurs across the endoplasmic reticulum (ER) and cleavage occurs between the E1 and E2-DS polyproteins. Below in FIG. 14, the same events are depicted showing the topology of the polyproteins in the ER of the cell. The E1 TMD consists of a hydrophobic sequence followed by a polar region and another hydrophobic sequence and this is shown as a segmented cylinder which forms a hairpin during translocation allowing cleavage of the signal sequence and release of the E2 ectodomain from E1. In some embodiments, following synthesis, E2-DS interacts with E1 via their TMDs to form non-covalent heterodimers within the VLP structure. In another embodiment, E1 is incorporated in the VLP without direct association with the TMD of E2-DS.

To describe one embodiment, FIG. 15 shows a schematic of the strategy used for production of the plasmid pCI E1E2-DS, which encodes hepatitis C virus E1 and E2 fused to DHBV S protein. Processing of this polypeptide in the cell' yields E1 non-covalently associated with the fusion protein of E2-S, as shown schematically in FIG. 14, which in turn forms VLPs in association with S as described herein.

### Example 18

### pCI E1E2-DS Expresses E1 and E2 in Cell Culture

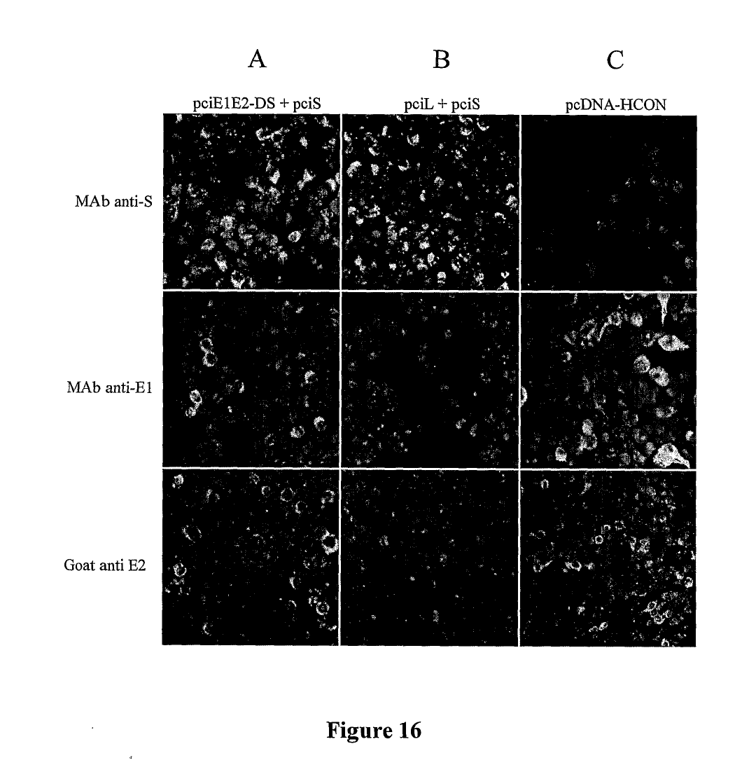

HEK 293T cells were transfected with plasmids expressing (A) pCI E1E2-DS+pCI-S, (B) pCI-S and pCI-L, (C) HCV envelope expression plasmid; HCV E1E2 (See FIG. 16). Slides were probed with either (i) MAb anti DHBV-S (7C12), (ii) MAb anti HCV E1 (A4) or (iii) goat anti-HCV E2. Antibodies detected with Alexa 488—anti IgG (green) then nuclei stained with propidium iodide (red). The results show that that cells transfected with pCI E1E2-DS (A) produce both E1 and E2 proteins. The presence of E1E2 complex is determined by immunoprecipitation as described in Dubuisson et al., J. of Virol. 68(16):6147-6160, 1994.

### Example 19

### Assembly of VLPs Containing Both E1 and E2 Proteins

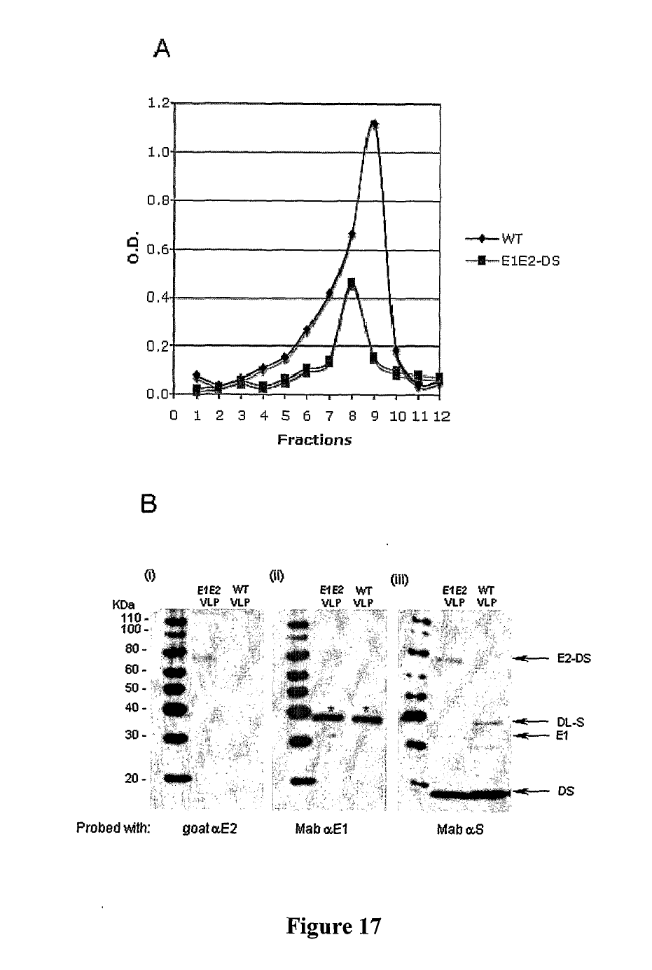

HEK 293T cells were co-transfected with plasmids to generate E1E2-DS VLPs (pCI E1E2-DS and pCI-S) or WT VLPs (pCI-L and pCI-S). Fractions 1 to 12 from ultracentrifugation through a 20-70% sucrose gradient were collected and analysed for VLP content by ELISA using monoclonal antibody against DHBV S protein (7C12) (See FIG. 17 (A). Peak fractions (8 and 9) were pooled, concentrated, and analysed by Western Blot (B). VLPs were probed sequentially with (i) goat anti HCV E2, then (ii) mouse anti HCV E1 (A4) or (iii) mouse anti DHBV S (7C12). The asterisk (*) denotes non-specific bands that are cross-reactive with E1 antibody. Note that VLPs of characteristic size and sedimentation behaviour contain both the E2 protein, as part of the fusion polypeptide with S protein, and the E1 protein that is present via non-covalent associations with the E2 protein.

### Example 20

### Formation of Conformational HCV Epitopes on VLPs Containing both E1 and E2

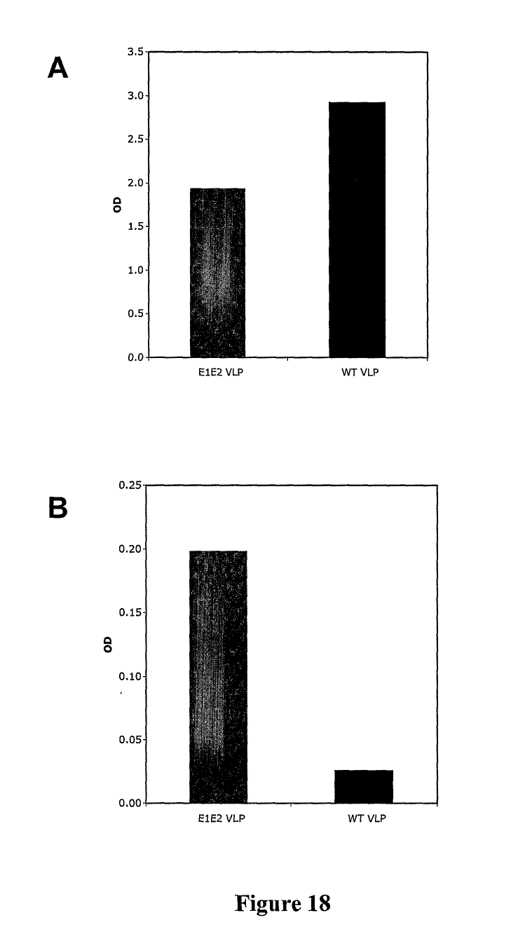

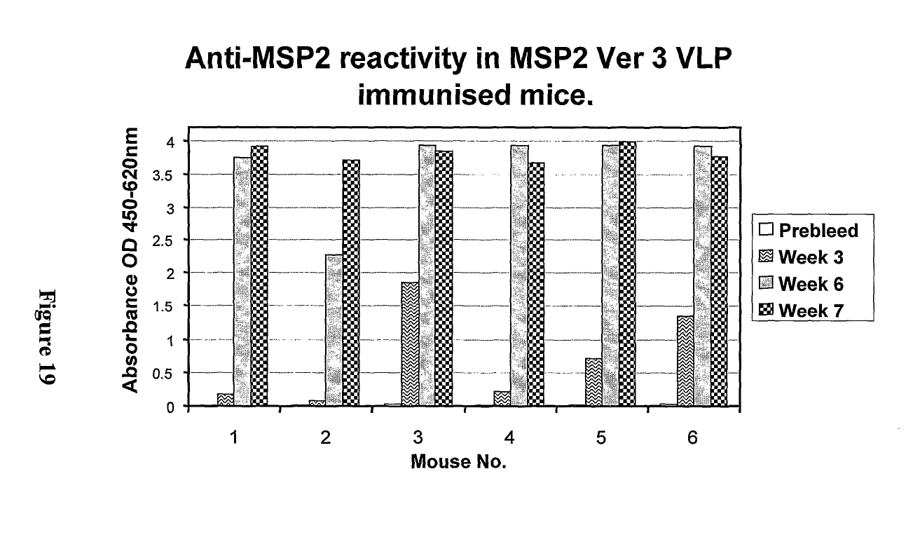

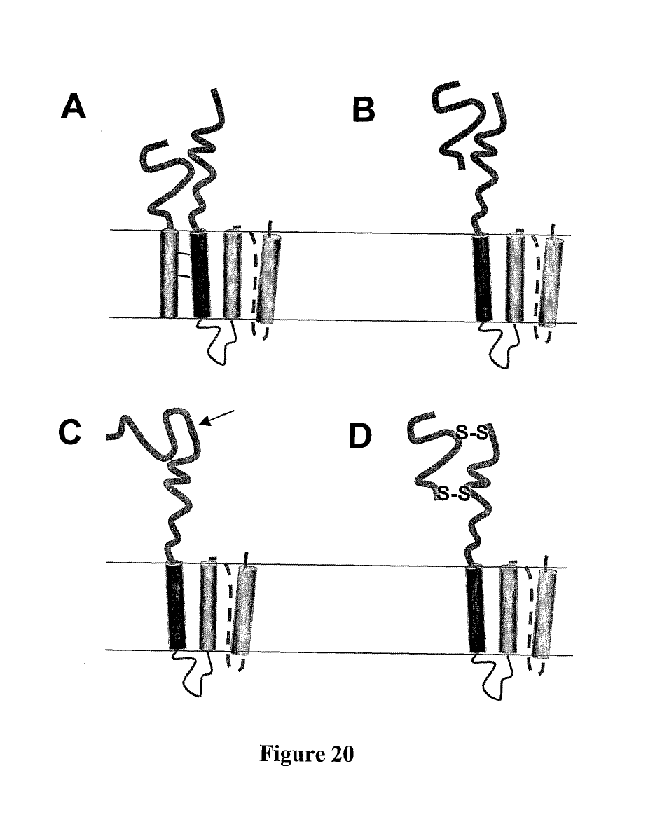

HEK 293T cells were co-transfected with pCI E1E2-DS and pCI-S (E1E2-VLPs) or pCI-L and pCI-S (wild-type [WT] VLPs), the cytosol fractions were collected and loaded on a linear sucrose gradient. The peak fractions of the sedimented VLPs (as in FIG. 17) were collected and analysed by VLP ELISA using the monoclonal antibodies (the results are presented in FIG. 18. (A) 7C12 (anti DHBV S) or (B) H53 (anti HCV E2, reacting to conformational E2 epitopes). E1E2-VLPs demonstrate significant reactivity with the H53 monoclonal antibody, whereas an excess amount of WT VLPs (shown by higher 7C12 reactivity) demonstrate no significant binding of H53.

It will be appreciated that the above examples are not limiting and VLPs could, in some embodiments, be constructed by one skilled in the art to include any POI that is preferentially assembled in a form that contains one part of the POI as a fusion protein and a second part of the POI as a non-covalently associated polypeptide. Preferred examples would include the envelope proteins of other members of the Flavivirus family and other virus families such as but not restricted to the Coronavirus, Herpesvirus, Hepadnavirus, Retrovirus, Orthomyxovirus or Paramyxovirus families where the mature viral envelope proteins are formed by proteolytic cleavage from a precursor polypeptide.

### Example 21

### VLPs Incorporating the MSP2 Surface Protein of Plasmodium falciparum

VLPs incorporating the MSP2 surface protein of Plasmodium falciparum (malaria, strain 3D7) induce strong antibody responses in Balb/C mice (H-2d), without the use of adjuvants (see FIG. 19). This is in contrast to the lack of immunogenicity of MSP2 from this strain of P. falciparum in H-2d Balb/C mice without adjuvant (Pye et al., 1997 (supra)), and demonstrates that VLPs are especially suited to the presentation of antigens such as MSP2 (which was previously known as MSA-2). In this example, the MSP2-VLPs were produced using the methods described in Example 11. Their strong immunogenicity is proposed to be related to the formation of non-covalent interactions between individual MSP2 chains on the VLPs. The formation of amyloid-like polymers of MSP2 has been recognised previously.

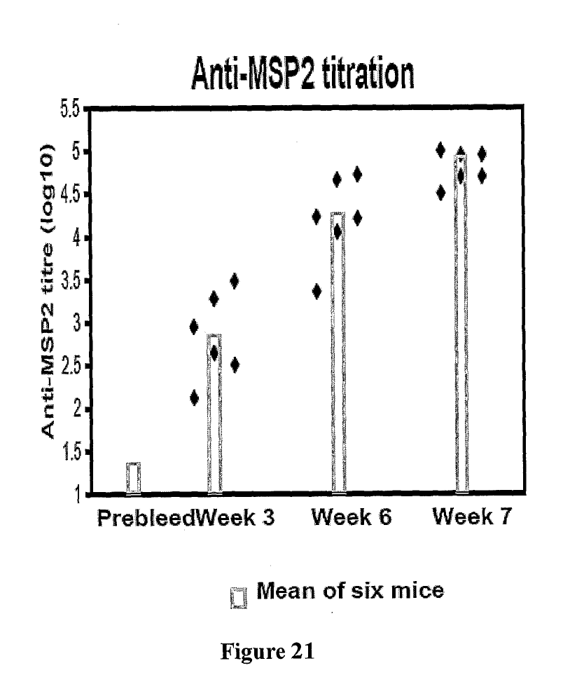

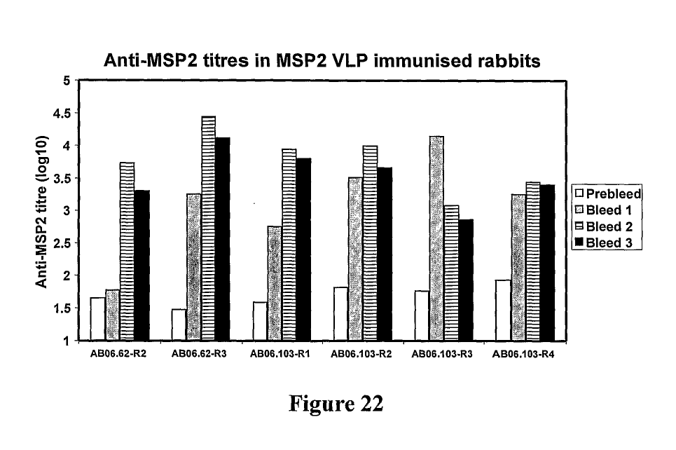

The strong immunogenicity of the MSP2-VLPs is further demonstrated in the individual endpoint titres of sera from mice immunised with MSP2-VLPs (see FIG. 21). MSP2-VLPs were also shown to be highly immunogenic in rabbits. A group of 6 rabbits were immunised with 10 μg MSP2-VLPs without adjuvant, and all animals developed high levels of anti-MSP2 antibody after a single dose (see FIG. 22).

### Example 22

### VLPs Incorporating a Range of Viral Envelope Proteins

Various examples of large and diverse POIs have been incorporated into chimeric DHBV VLPs. The size and diversity of such POIs demonstrates that any POI can most likely be incorporated into VLPs according to the present invention or as described in WO 2004/092387 incorporated herein. These POIs include: Hepatitis C virus N′ E2 (82 a.a.), Hepatitis C virus E2 ectodomain (278 a.a.), Hepatitis B virus preS (163 a.a), MSP2 of Plasmodium falciparum (230 a.a), Measles virus H protein (584 a.a.), EGFP (239 a.a), HIV-1 gp140 (684 a.a), Hepatitis C virus E1/E2 plus TMD (576 a.a); and Influenza A H1 or H5 HA (512 a.a).

### Example 23

### Further VLP Formats

Further modifications are contemplated in order to enhance the antigenic or immunogenic function of the subject VLPs. In one embodiment, the POT is linked by covalent disulphide linkages through cysteine or other amino acids to the fusion polypeptide, but is not linked by the alpha carbon backbone of the fusion polypeptide chain. In examples of this type, the disulphide linkages may be formed within a precursor polypeptide chain that is subsequently cleaved within the alpha carbon backbone such that the further part of the POI remains covalently associated via the disulphide linkages. The fusion (F) protein of Measles virus is one example of a viral envelope protein where the two fragments of the mature (cleaved) polyprotein remain covalently linked via disulphide bonds in the native viral particle. The hemagglutinin (HA) protein of influenza A virus is another example of a viral envelope protein where the two fragments of the mature (cleaved) polyprotein remain covalently linked via disulphide bonds in the native viral particle. Disulphide linkages may be formed by addition of any suitable peptide or polypeptide to the VLP under conditions that allow the formation of disulphide linkages between the fusion polypeptide and the added POI.

Schematic outlines of various aspects of the subject VLPs are shown in FIG. 20. (A) Non-covalent association of parts of the POI via interactions between transmembrane domains, as for hepatitis C virus E1E2. (B) Non-covalent association of parts of the POI via interactions between the same or other parts of the POI, as for MSP2. (C) Covalent association of parts of the POI via preventing cleavage of the precursor polypeptide

(shown by an arrow), as for HIV gp140. (D) Covalent association of parts of the POI by disulphide linkages between the parts of the POI, as for measles virus F protein or influenza HA protein.

In some embodiments, the incorporation of such further parts of a POI results in enhanced function, such as antigenic or immunogenic function, of the VLP as a whole. This may be achieved through alteration or modification of the parts of the POI that are incorporated as a fusion polypeptide, for example through enhancement of protein folding, protein binding and/or antigenic or immunogenic function of the further part of the POI that is incorporated into the VLP by virtue of its non-covalent or covalent association with the first part of the POI.

### Example 24

### Binding of HCV E1E2-VLPs and E2-VLPs to the HCV Receptor, CD81

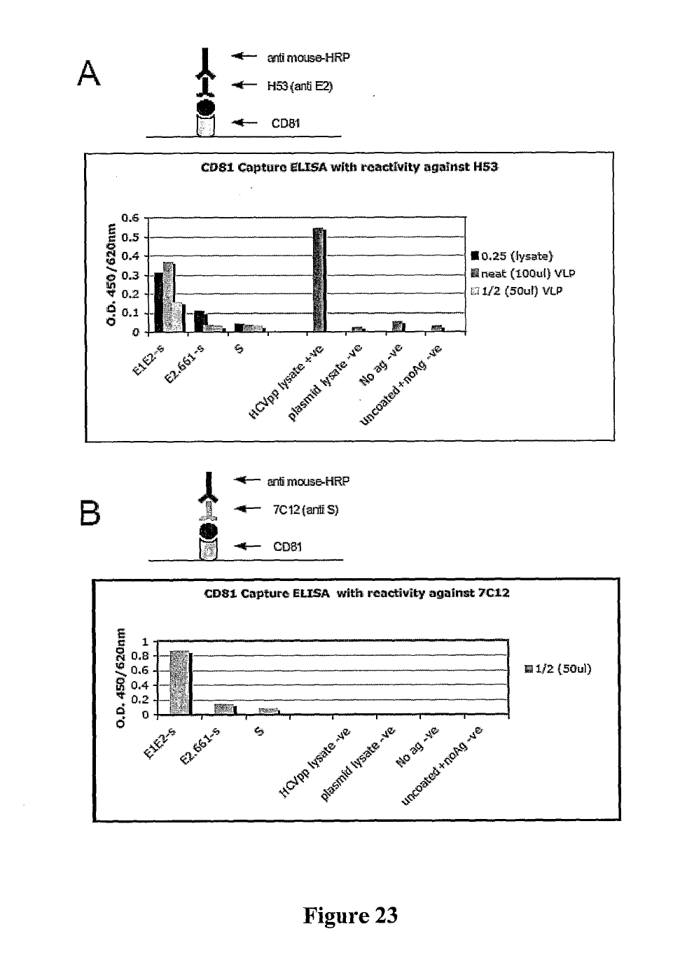

VLPs were prepared from transfected 293T cells, and purified by sucrose density gradient ultracentrifugation. Cell lysates (collected prior to sucrose gradient purification) were also tested. As shown in FIG. 23 binding of the E1E2-VLPs and E2-VLPs to recombinant CD81 immobilised on ELISA plates was detected using monoclonal antibody H53 (see FIG. 23(A)), and indicates that both E1E2-VLPs and E2-VLPs display the correct conformational folding of E2 to allow CD81 binding. As shown in FIG. 23(B), binding of the VLPs to CD81 is detected using monoclonal antibody 7C12 to the DHBV S protein, and indicates that the E1E2-VLPs are much more efficiently captured than the E2-VLPs, again showing good conformation of the E2 in the E1E2 complex on VLPs.

### Example 25

### Antibody Responses to E1E2-VLPs and E2-VLPs Produced in Cell Culture

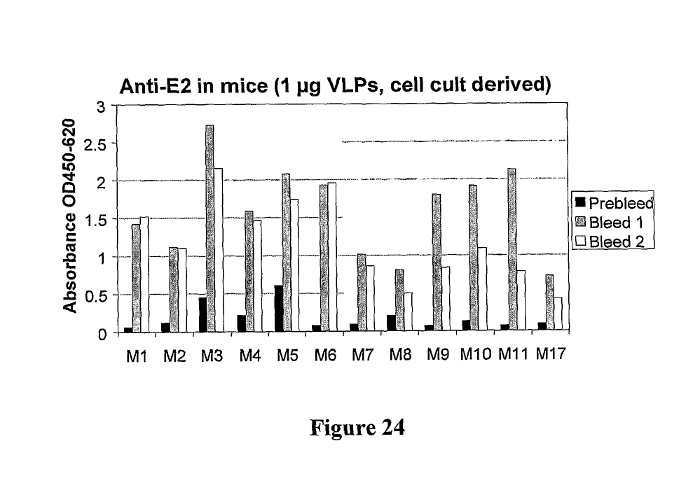

In order to measure the antibody responses to E1E2-VLPs and E2-VLPs groups of six mice were immunised at 3-week intervals with approximately 1 μg E1E2-VLPs (without adjuvant; mice M1-M6) or E2-VLPs (without adjuvant; M7-M17) prepared from 293T cells. Antibody responses were measured 3 weeks after each dose by ELISA using E2 antigen. As shown in FIG. 24 both forms of VLPs were highly immunogenic in mice although the anti-E2 response was greater in the case of E1E2-VLPs.

### Example 26

### Cellular Immune Responses to E1E2-VLPs and E2-VLPs Produced in Cell Culture

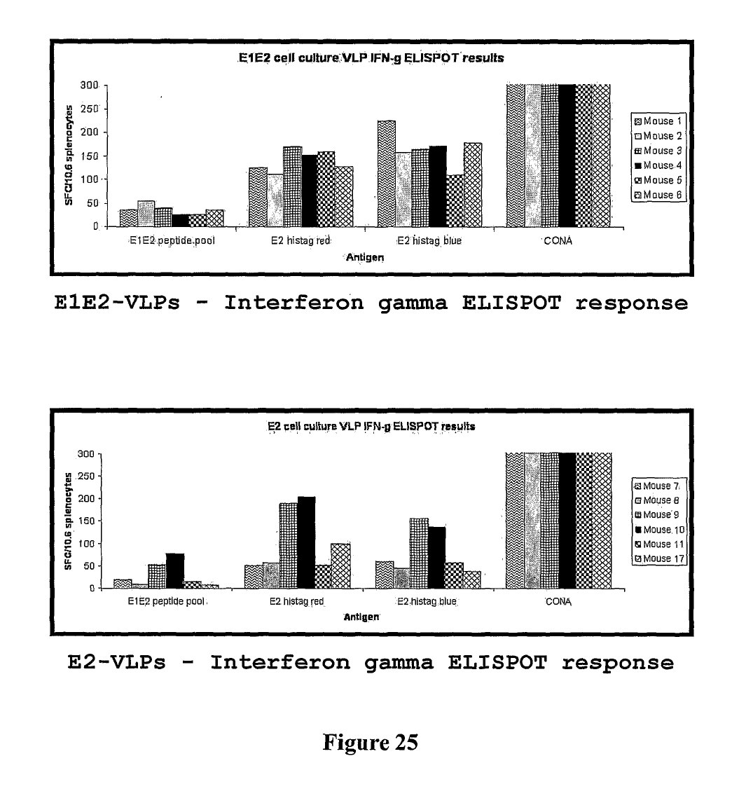

In order to measure the cellular immune response to E1E2-VLPs and E2-VLPs groups of six mice were immunised with approximately 1 μg VLPs (without adjuvant) at 3-week intervals. Cellular immune responses were measured 3 weeks after the final dose by gamma interferon ELISPOT using either a peptide pool derived from E1E2 region of the HCV genome, or two different recombinant HCV E2 proteins (E2 Histag Blue or E2 Histag Red), with ConA providing a positive control. As shown in FIG. 25 both forms of VLPs gave significant cellular immune responses in mice.

### Example 27

### Increased Expression of E1E2-S Using a Codon-Optimised Gene

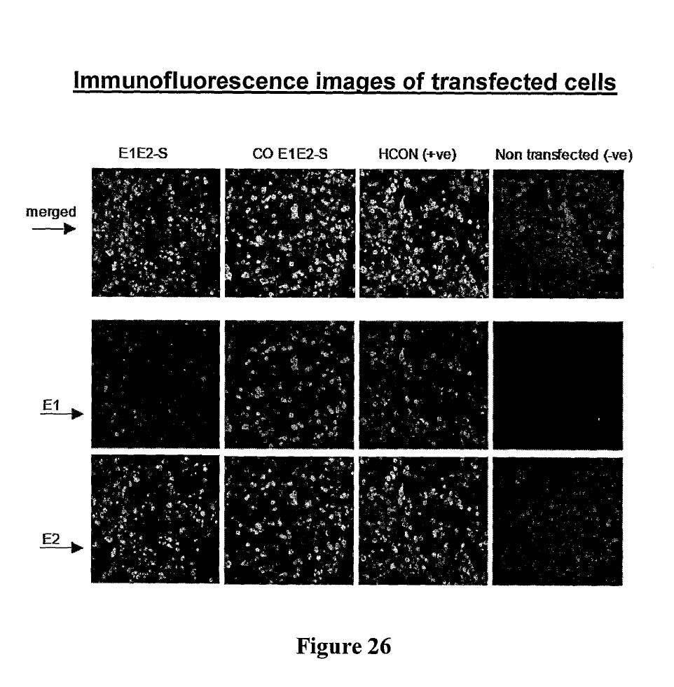

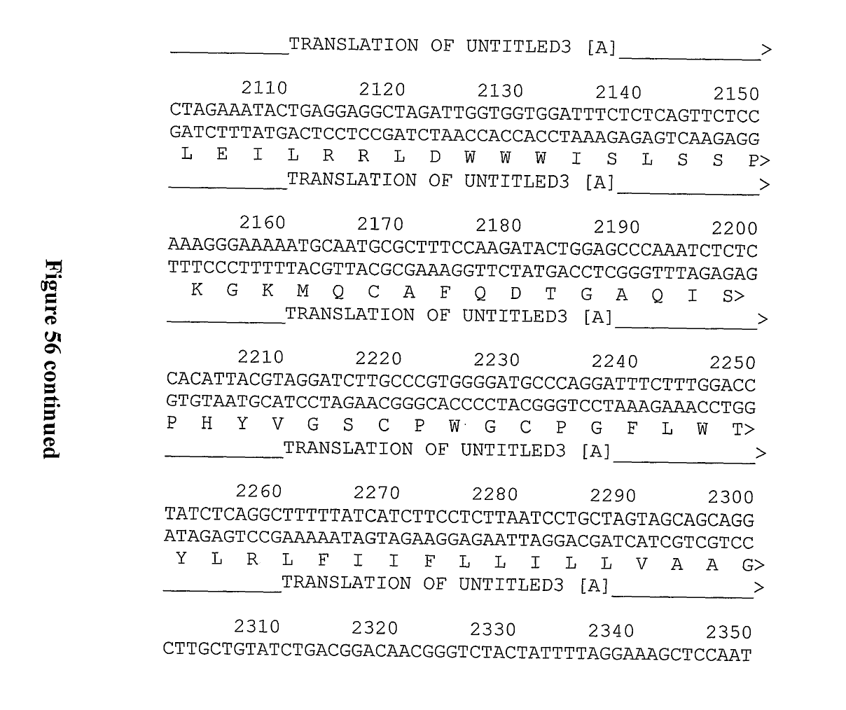

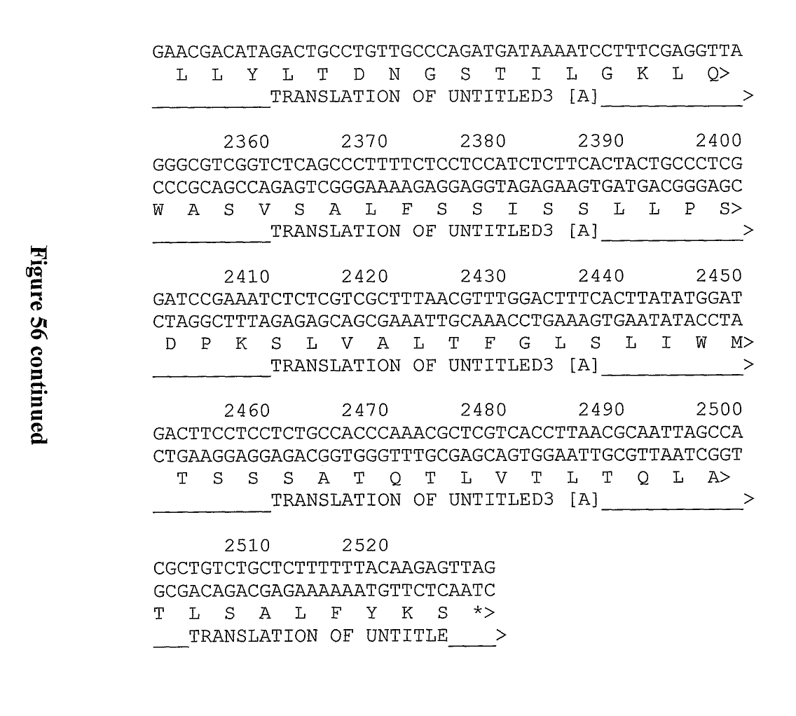

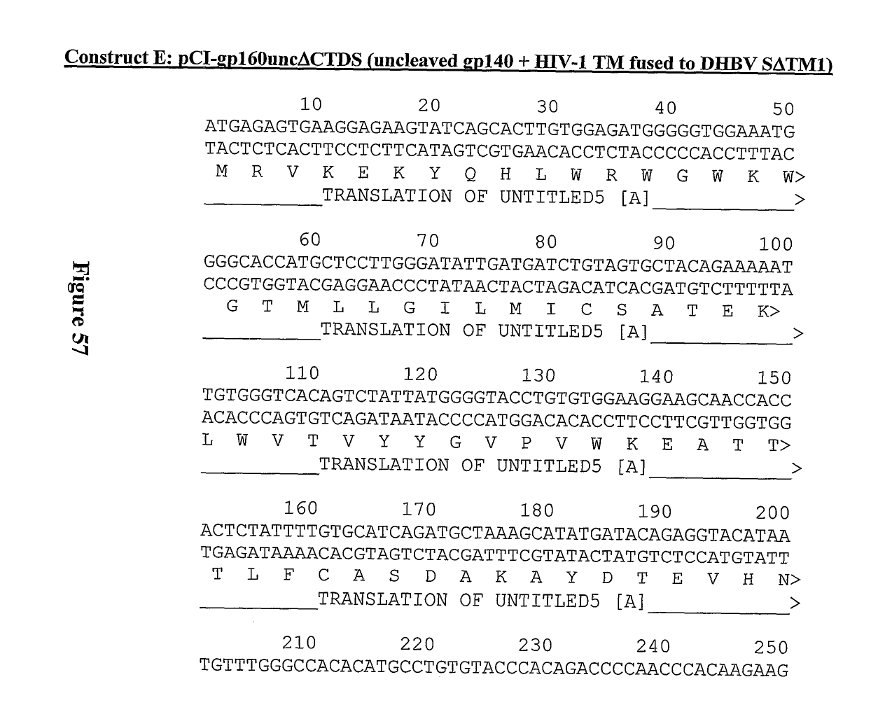

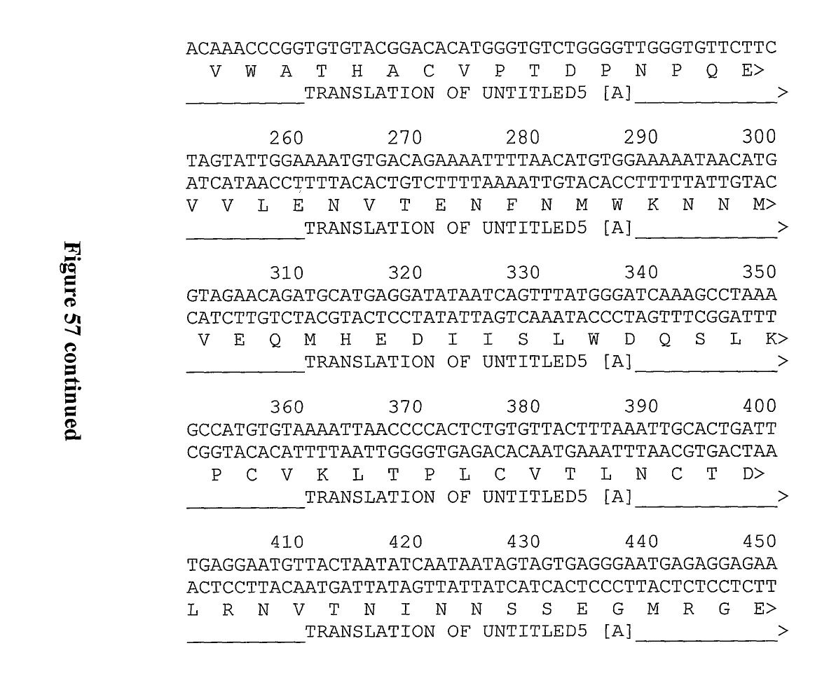

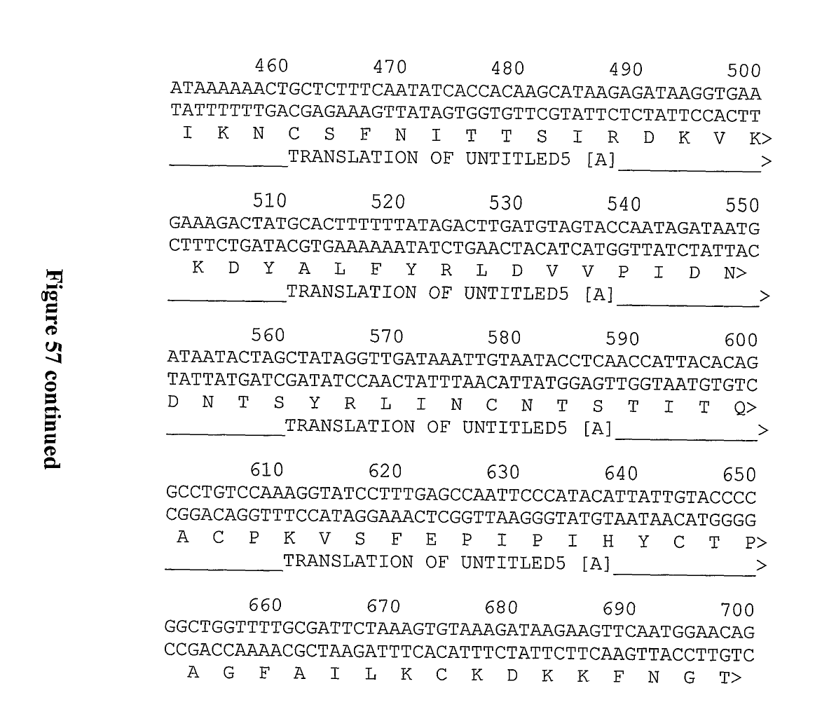

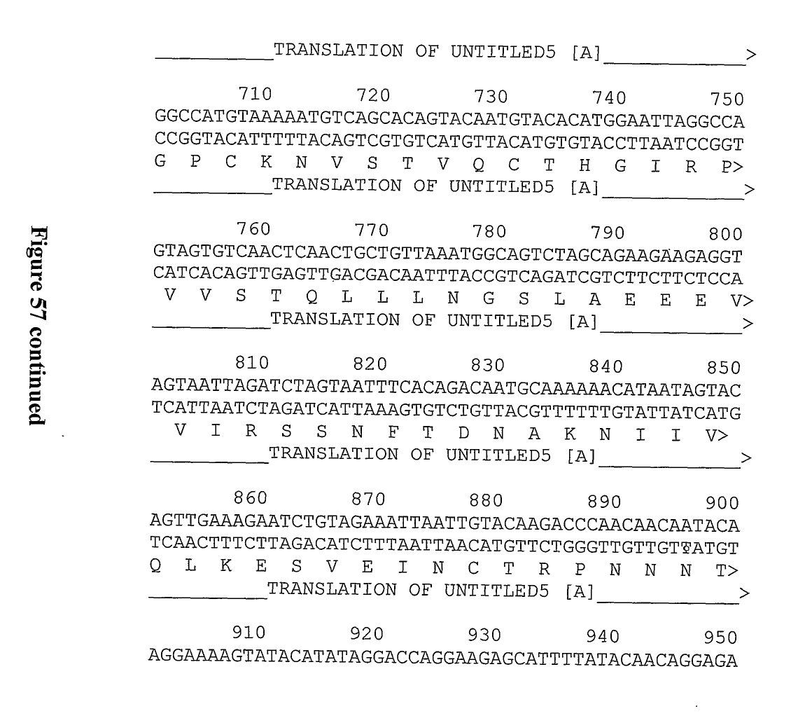

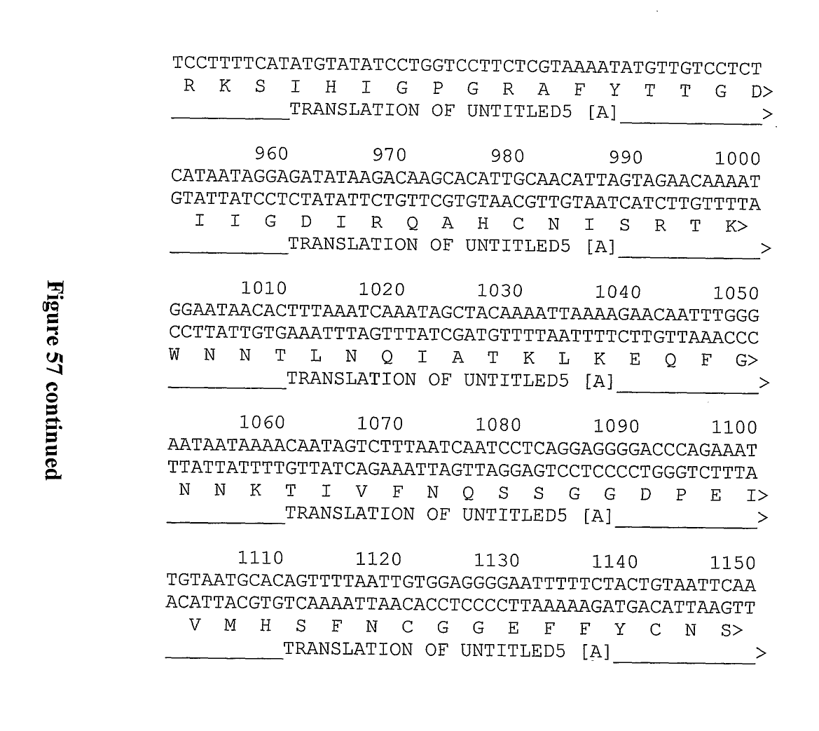

Increased amounts of E1E2-S were expressed in 293T cells following the use of a codon-optimised gene (CO E1E2-S) rather than the wild-type gene (E1E2-S), demonstrated by increased expression of the E1 protein detected by indirect immunofluorescence staining with the E1-specific monoclonal antibody A4 (red) see FIG. 26. Staining of E2 protein in the same cells was also enhanced in cells comprising the codon-optimised sequences (as also shown in FIG. 26 (goat anti-E2; green). Nuclei are stained blue (Toto 3 stain). The sequence of the codon optimised E1E2-S sequence is shown in FIG. 50 and in SEQ ID NO: 20 (nucleotide) and SEQ ID NO: 21 (amino acid).

### Example 28

### Increased Expression of E1E2-S Using Codon-Optimised Gene

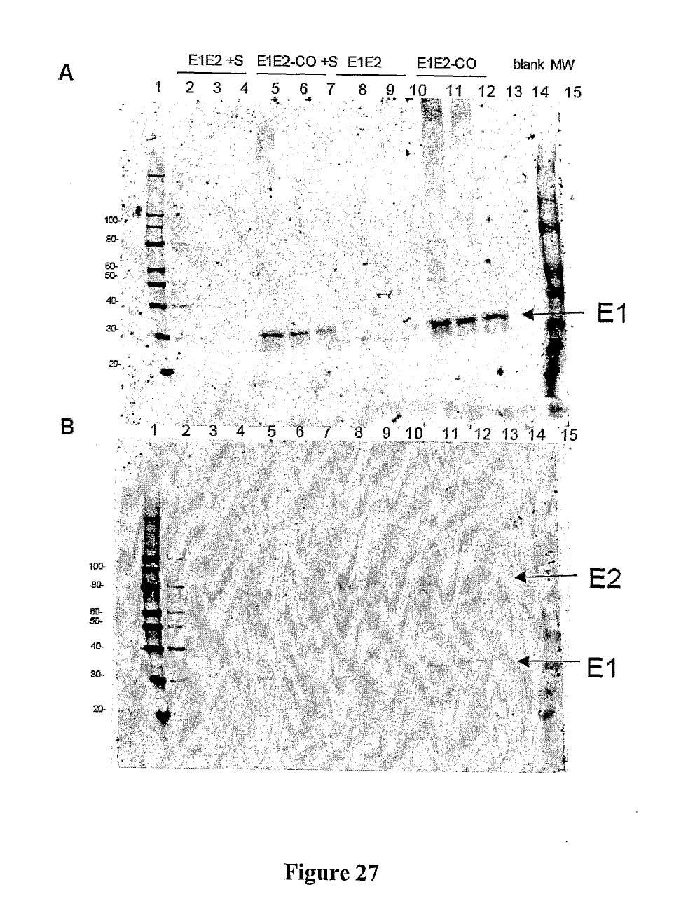

Increased amounts of E1E2-S were expressed in 293T cells following the use of the codon-optimised gene. As shown in FIG. 27, increased expression of the E1 protein was detected by Western immunoblotting with the E1-specific monoclonal antibody A4 (A, compare lanes 5, 6 and 7 [codon-optimised E1 expression] to lanes 2, 3 and 4 [non codo-optimised E1 expression]).

### Example 29

### Increased Incorporation of E1E2-S in VLPs Using Codon-Optimised Gene

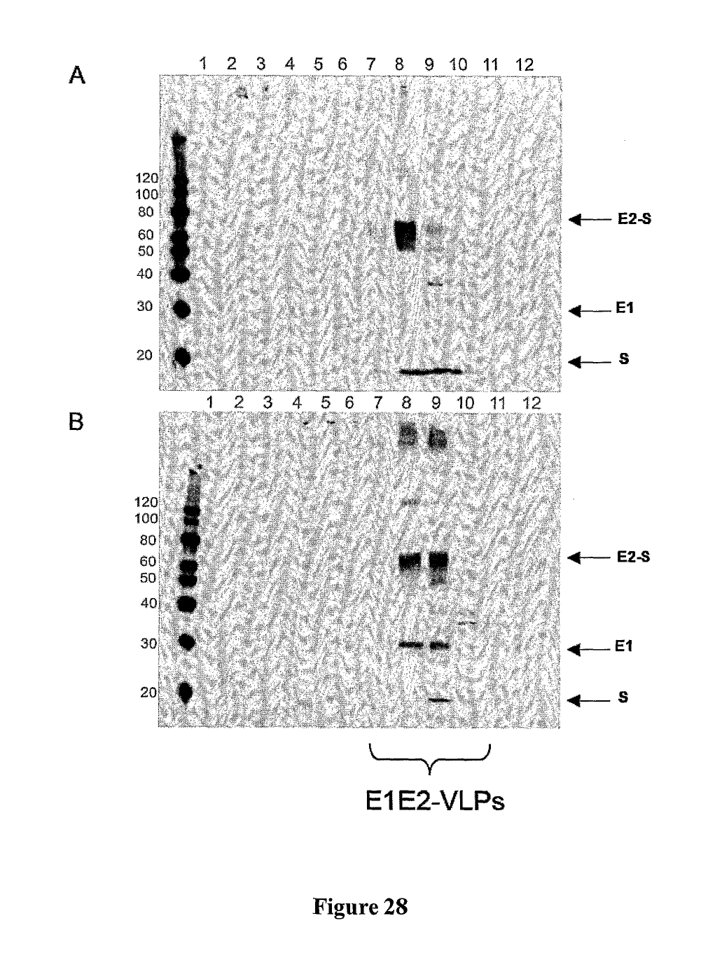

As shown in FIG. 28, increased amounts of E1 were incorporated into VLPs after expression of a codon-optimised gene (B), detected by Western blot staining of E1E2-VLPs with the E1-specific monoclonal antibody A4, goat antibody to E2, and monoclonal antibody 7C12 against the DHBV S protein. VLPs were prepared from transfected cells and purified over sucrose density gradients, with fractions (7-10) representing VLPs indicated. E1 incorporation into VLPs in non codon-optimised expression is reduced in this example (A). This example demonstrates the assembly of E1 into VLPs via non-peptide bond interactions with the E2-S in chimeric VLPs.

### Example 30

### The Two Different Constructs for Successful Expression and Assembly of Influenza HA-VLPs

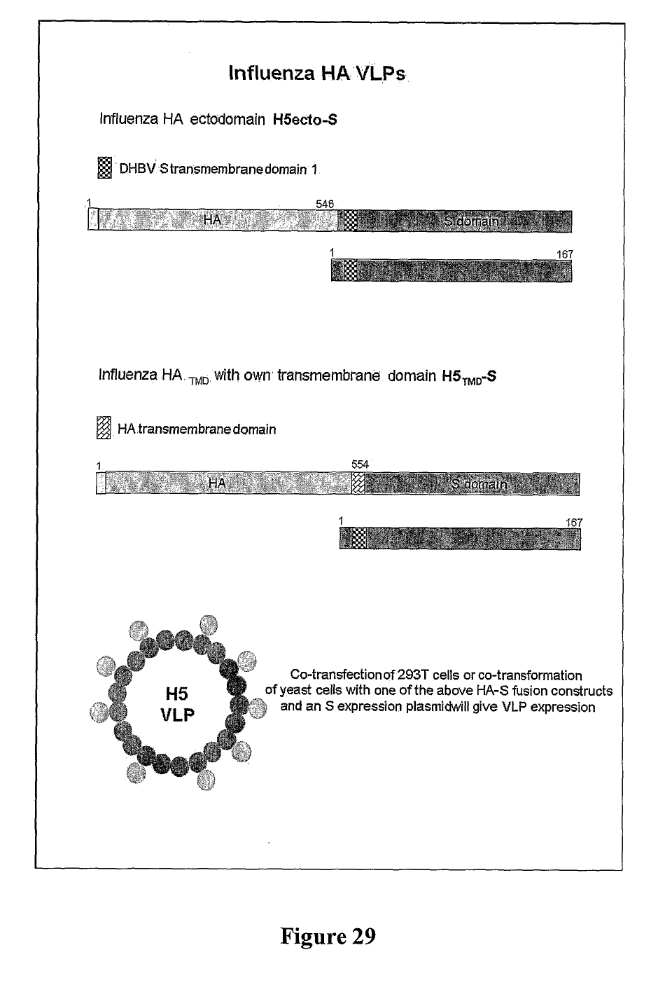

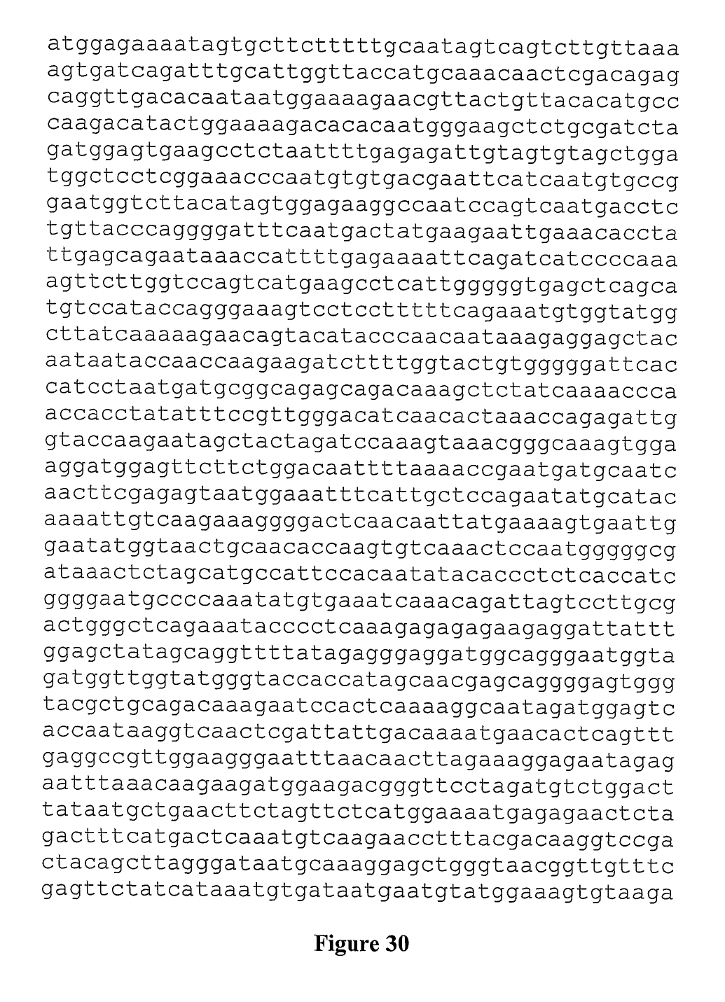

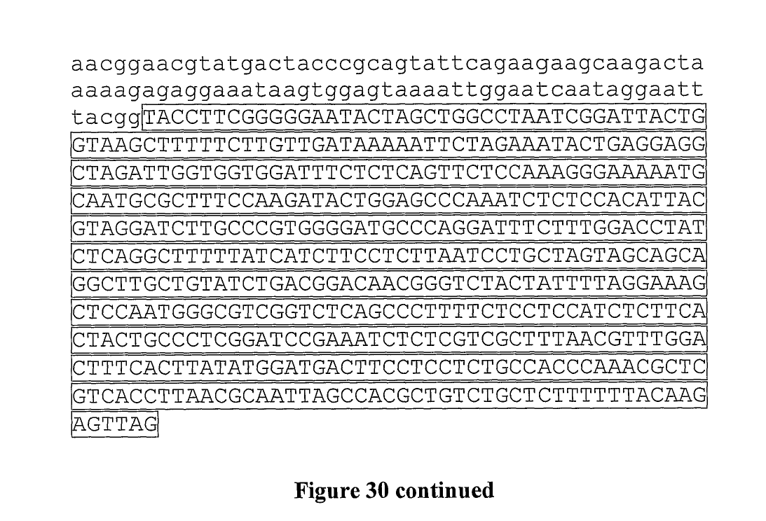

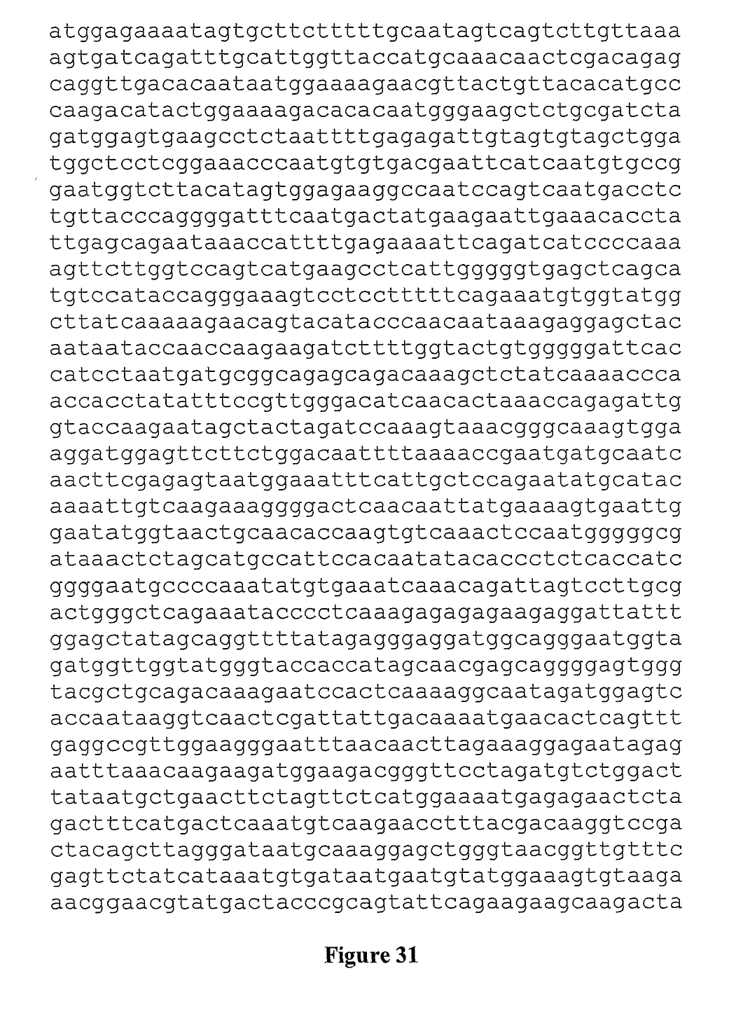

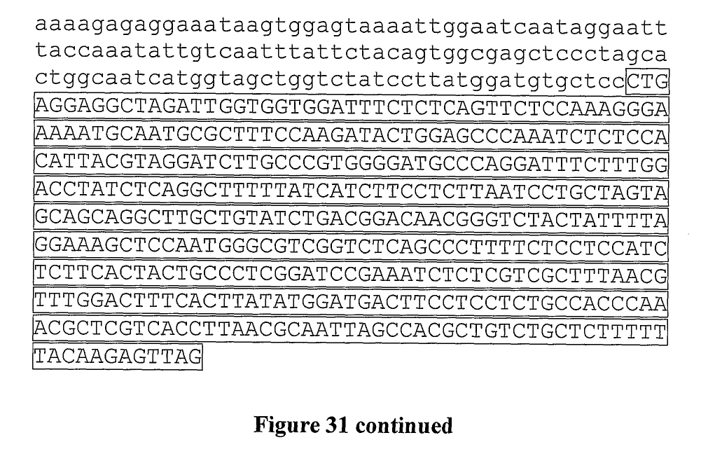

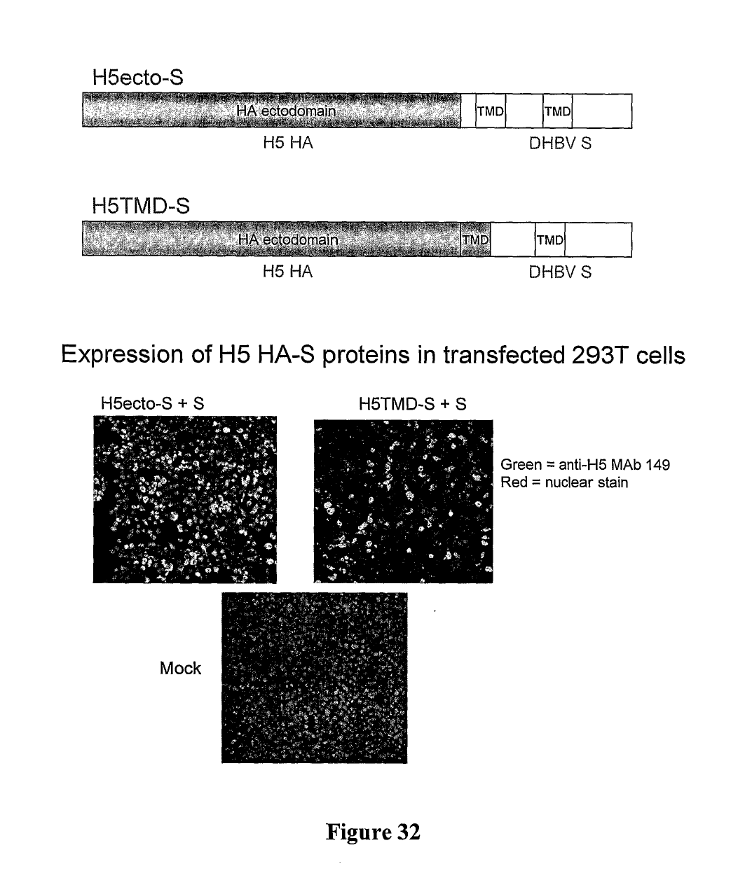

Constructs shown schematically in FIGS. 29 and 32 were made for expression in mammalian cells and in yeast (Saccharomyces cerevisiae) using appropriate plasmid vectors. Analysis of both constructs in mammalian cells, and of the TMD construct only in yeast, is shown in the following examples. For H5ecto-S, the ecto domain of influenza A HA is fused to the N-terminal end of the S-domain of L polypeptide and the TMD1 domain of L is present. In H5TMD-S, the ectodomain and the C-terminal transmembrane domain of HA are fused to the S-domain of L polypeptide and the TMD1 domain of L is absent. The nucleotide sequence of H5ecto-S is shown in FIG. 30 (the boxed nucleotides are the S-component) and SEQ ID NO: 16. The nucleotide sequence of H5TMD-S is shown in FIG. 31 (the boxed nucleotides are the S component) and SEQ ID NO: 17. HA-S expression was detected by immunofluorescence in transfected 293T cells stained with H5HA specific monoclonal antibody 149 (green, nuclei stained red). As shown in FIG. 32, both H5ecto-S and H5TMD-S express significant amounts of HA reactive antigen in cell culture.

### Example 31

### Assembly of Influenza A HA H5ecto and H5TMD VLPs

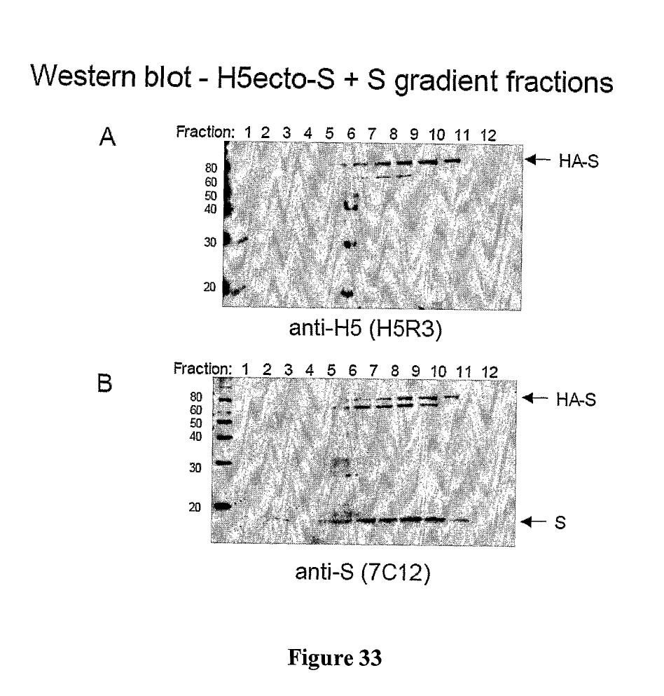

Assembly of influenza A HA H5ecto-VLPs in cell culture was detected by Western immunoblotting of sucrose density gradient fractions with H5 HA-specific rabbit antibody and S-specific monoclonal antibody 7C12 (see FIG. 33). 293T cells were cotransfected with the H5 HA ecto-S plasmids together with S expression plasmids, VLPs were harvested from the cell lysates and purified by sucrose density gradient ultracentrifugation. Fractions were concentrated by methanol precipitation, proteins separated by SDS-PAGE, and the antigens were detected with the relevant antibodies; FIG. 33(A) rabbit anti-H5 HA; FIG. 33(B) monoclonal anti-S plus rabbit anti-H5 HA (both antigens detected). H5ecto-S assembles into VLPs together with S, shown by its detection along with S in fractions typical for DHBV VLPs under these conditions. Similar results were seen for the H5TMD-VLPs (not shown).

### Example 32

### Assembly of Influenza A H5TMD-VLPs and H5ecto-VLPs in Cell Culture Detected by ELISA

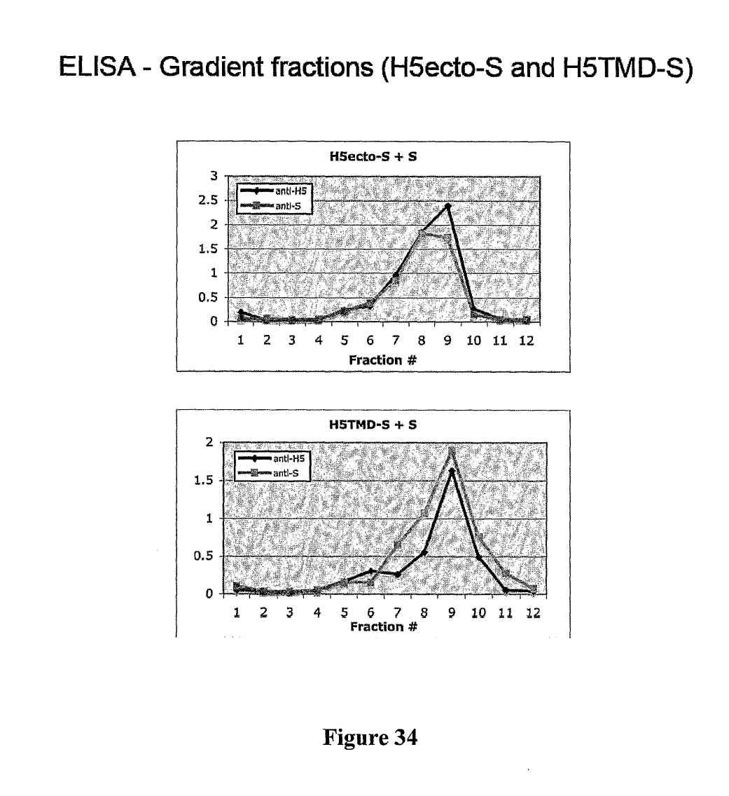

As shown in FIG. 34 assembly of influenza A H5TMD-VLPs and H5ecto-VLPs in cell culture was detected by ELISA of sucrose density gradient fractions with HA-specific monoclonal antibody 149 and S-specific monoclonal antibody 7C12. 293T cells were cotransfected with the indicated HA-S expression plasmids together with S expression plasmids, VLPs were harvested from the cell lysates and purified by sucrose density gradient ultracentrifugation. Fractions were applied to ELISA plates, and the antigens were detected with the relevant monoclonal antibodies. Note that both forms of HA-VLP contained high levels of HA-reactive antigen and sedimented in fractions typical for DHBV VLPs under these conditions.

### Example 33

### H5 VLPs Show Correct Conformation of HA1 and HA2-S

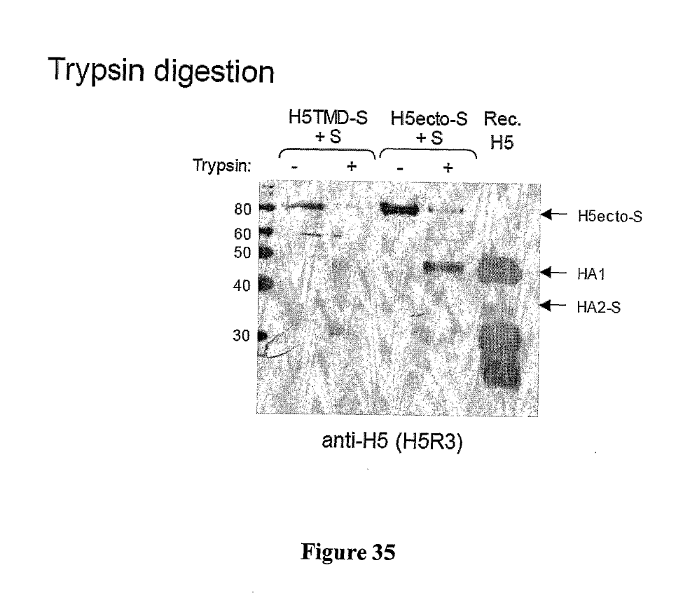

Trypsin digestion of influenza A H5TMD-VLPs and H5ecto-VLPs was detected by Western blotting of VLPs from sucrose density gradient fractions with H5 HA-specific rabbit antibody. As shown in FIG. 35, complete digestion of the HA0-S (H5ecto-S) to yield HA1 and HA2-S can be seen, showing that the chimeric protein has the correct (trypsin-resistant) conformation of HA1 and HA2-S.

### Example 34

### HA1 of HA Remains Associated with VLP by Non-Peptide Linkage with HA2-S

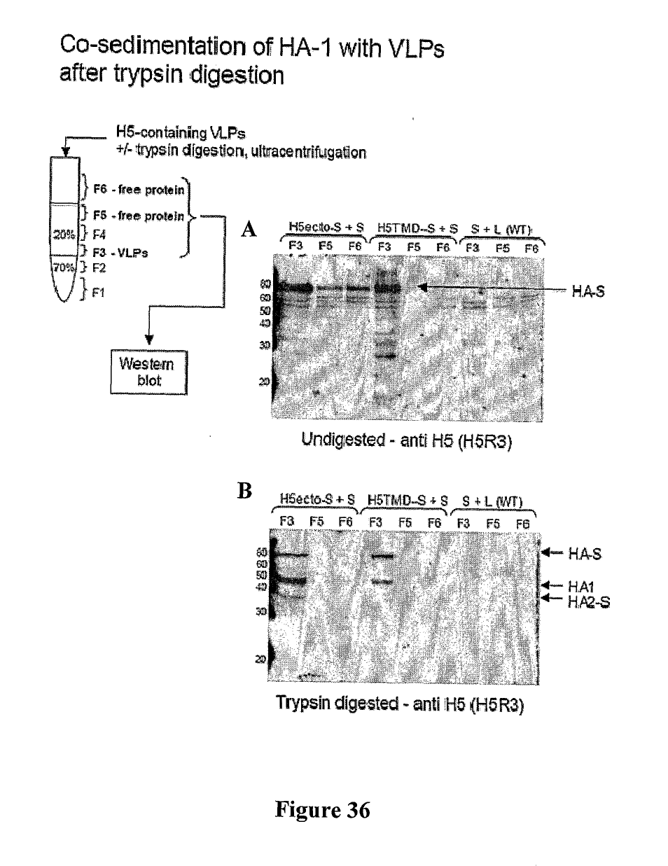

Using the H5ecto-VLPs and H5TMD-VLPs digested with trypsin as shown in the previous example, it can be shown that the HA1 part of the HA molecule remains associated with the VLP by virtue of its non-peptide linkage with the HA2-S part of the protein. As shown in FIG. 36, following trypsin digestion (B) or control (mock) digestion (A), VLPs were sedimented over sucrose gradients as shown in the schematic, and the fraction 3 interface containing VLPs, as well as fractions 5 and 6 containing soluble proteins, were analysed by SDS-PAGE and Western immunoblotting with rabbit H5 HA-specific antibody. All of the HA1 fragments remain associated with the HA2-S which remains incorporated into the VLPs. It can be assumed that the HA1 is associated with the HA2 subunit via the normal pattern of disulphide bonds.

### Example 35

### Assembly of Influenza A H5TMD-VLPs in Yeast Saccharomyces cerevisiae

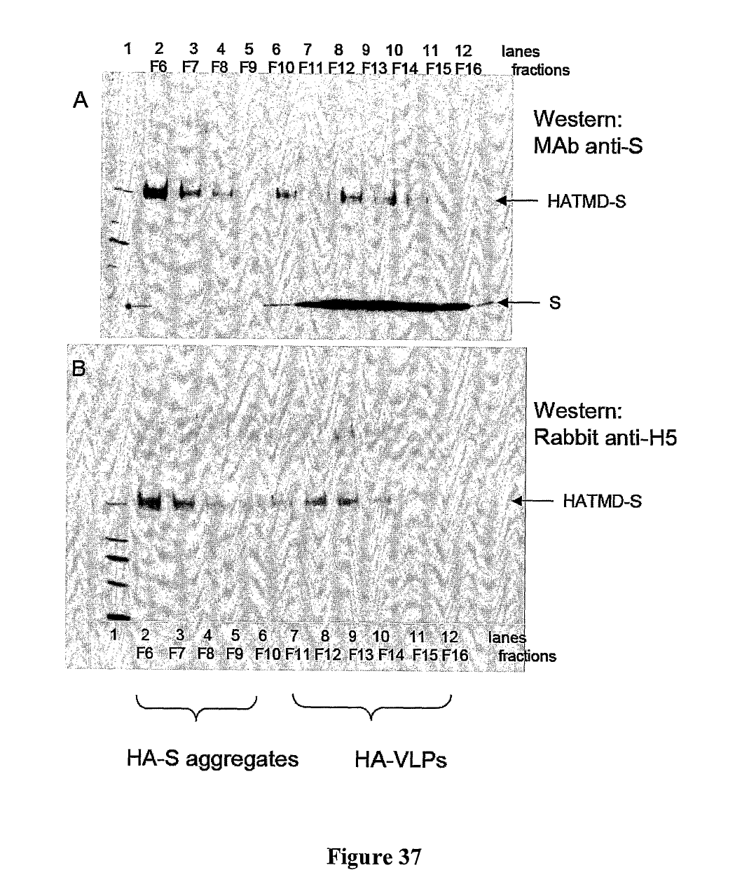

As shown in FIG. 37, assembly of influenza A H5TMD-VLPs in yeast (Saccharomyces cerevisiae) was detected by Western immunoblotting of sucrose density gradient fractions with H5 HA-specific rabbit antibody and S-specific monoclonal antibody 7C12.

Yeast cells were stably cotransformed with the H5 HA TMD-S plasmid together with S expression plasmid, VLPs were harvested from the cell lysates and purified by sucrose density gradient ultracentrifugation. Proteins in each fraction were separated by SDS-PAGE, and the antigens were detected with the relevant antibodies; FIG. 37(A) monoclonal anti-S; FIG. 37(B) rabbit anti-H5 HA. H5TMD-S assembles into VLPs together with S, shown by its detection along with S in fractions typical for DHBV VLPs under these conditions. In addition, a proportion of the H5TMD-S appears to form aggregates that do not contain S protein, and sediment towards the bottom of the gradient (HA-S aggregates), due to the relatively high level of expression of H5TMD-S to S protein in this transformed yeast cell line. It is likely that more balanced expression of the two proteins would result in higher levels of incorporation of HA-S into VLPs without the formation of aggregates.

### Example 36

### H5TMD-VLPs Show Correct Folding

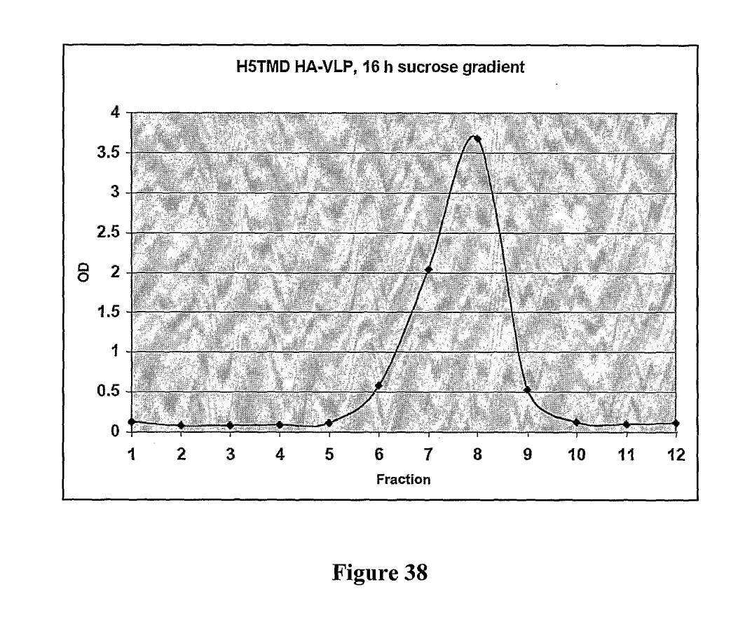

Assembly of influenza A H5TMD-VLPs produced in yeast was detected by ELISA of sucrose density gradient fractions with HA-specific monoclonal antibody 149. As shown in FIG. 38, the fractions containing H5TMD-S in VLPs are highly reactive in ELISA with this conformation-specific monoclonal antibody, whereas the fractions containing large amounts of H5TMD-S in aggregates rather than VLPs (corresponding to fractions 3-5 in this gradient) show no reactivity with this antibody. This indicates that proper folding of the H5TMD-S protein is dependent on co-assembly into VLPs.

### Example 37

### Trypsin Digestion of Influenza A H5TMD-VLPs Detected by ELISA of Sucrose Density Gradient Fractions with HA-Specific Monoclonal Antibody 149

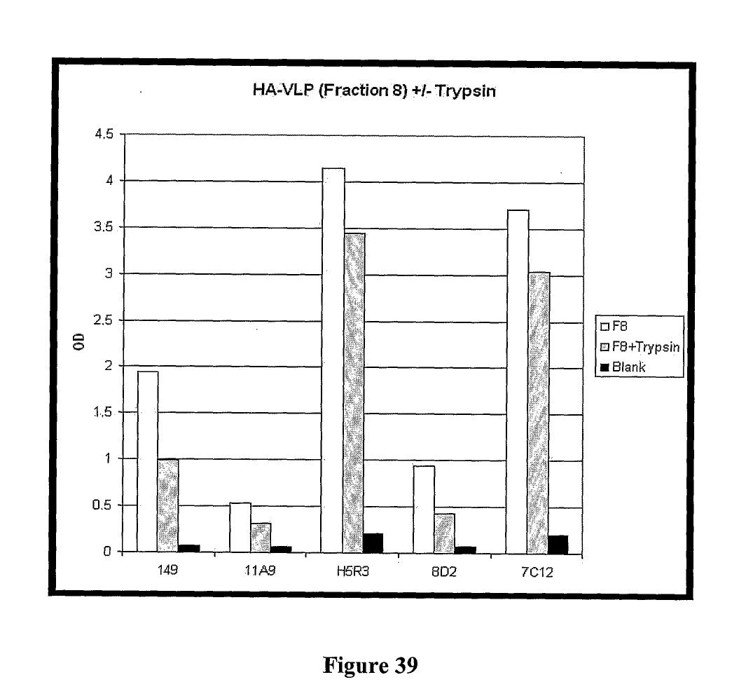

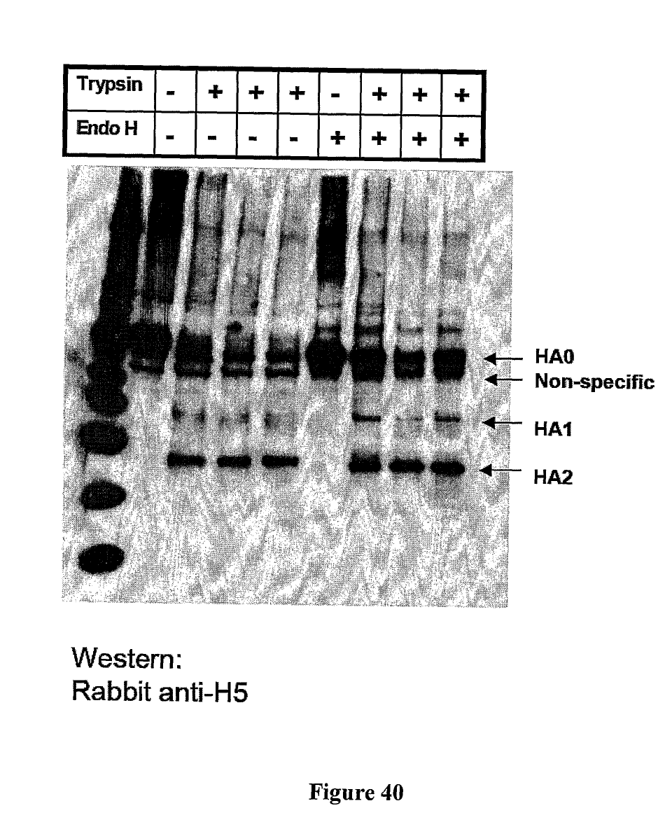

As shown in FIG. 39, complete digestion of the HA0-S to yield HA1 and HA2-S (as shown in western blots, see FIG. 35 and FIG. 40) results in a moderate decrease in ELISA reactivity with a range of HA-specific monoclonal (149, 11A8, 8D2) and a polyclonal antibody (H5R3), and corresponding decrease in the amount of S protein reactivity (MAb 7C12).

### Example 38

### Trypsin Digestion of Influenza A H5TMD-VLPs Detected by Western Blotting of VLPs from Sucrose Density Gradient Fractions with HA Specific Rabbit Antibody

As shown in FIG. 40, complete digestion of the HA0-S to yield HA1 and HA2-S occurs with the lowest concentration of trypsin, and the presence of increasing amounts of trypsin (from left to right) does not demonstrate any degradation of the protein, showing that it has the correct (trypsin-resistant) conformation of HA1 and HA2-S.

In addition, digestion of the VLPs with Endoglycosidase H (Endo H) shows that all of the glycans on the HA0-S, HA1 and HA2-S are completely sensitive to digestion with endoglycosidase H. This lack of complex glycans is consistent with the assembly pathway of duck hepatitis B virus and chimeric VLPs which bypasses the Golgi (the site of complex glycan processing), and can provide advantages for antigens on the subject chimeric VLPs because the lack of complex glycans should result in less masking of important neutralising epitopes. Masking of neutralizing epitopes by complex glycans is a well known problem which is particularly important for HIV and hepatitis C, but may also be important for other diseases (Helle et al., J. Virol., 81(15):8101-8111, 2007; Falkowska et al., J. Virol., 81(15):8072-8079, 2007; Losman et al., AIDS Res. Hum. Retroviruses., 17(11):1067-1076, 2001).

### Example 39

### Glycoproteins on Chimeric HCV YLPs Show Limited Complex Glycosylation

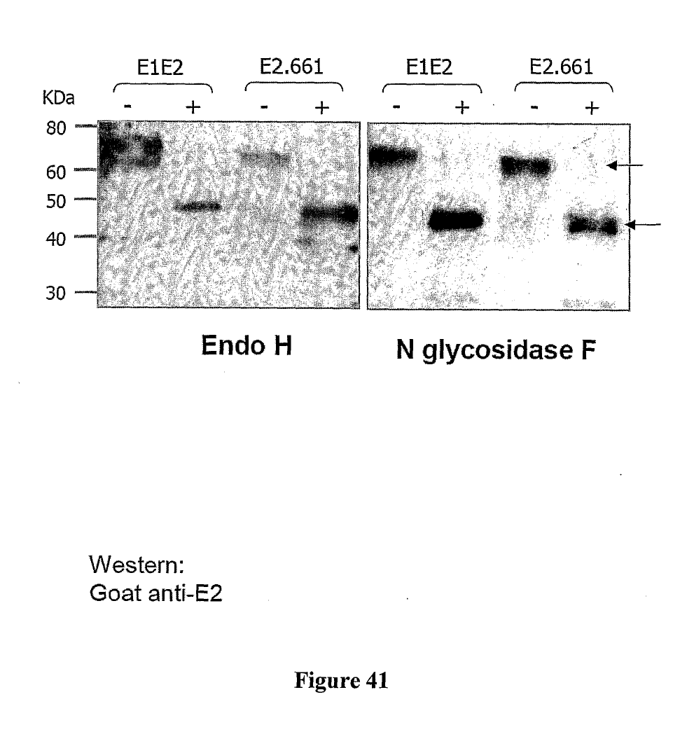

HCV E1E2-VLPs or E2-VLPs were prepared from transfected cells by sucrose density gradient ultracentrifugation, and portions were subjected to digestion with either endoglycosidase H (Endo H) or N-glycosidase F as shown. E2 in each sample was detected by Western immunoblotting with goat anti-E2 antibody following SDS-PAGE. The results shown in FIG. 41 indicate that all of the glycans on the E2 in both E1E2-VLPs and E2-VLPs is completely sensitive to digestion with endoglycosidase H, with no further reduction in molecular mass following N-glycosidase F treatment. This lack of complex glycans is consistent with the assembly pathway of duck hepatitis B virus and chimeric VLPs which bypasses the Golgi (the site of complex glycan processing), and can provide advantages for antigens on the subject chimeric VLPs because the lack of complex glycans should result in less masking of important neutralising epitopes. The sensitivity of influenza HA-VLPs to complete deglycosylation with endo H (previous figure) suggests that this is a common property of glycoproteins expressed on chimeric VLPs, consistent with the assembly pathway of duck hepatitis B virus and thus the chimeric VLPs. Glycoproteins present on chimeric HCV VLPs show limited amounts of complex glycosylation, with mostly mannose residues present (sensitive to endoglycosidase H as well as N-glycosidase F).

### Example 40

### Expression Constructs for Various Forms of the Human Immunodeficiency Virus (HIV) Envelope Glycoproteins to Allow Incorporation into VLPs

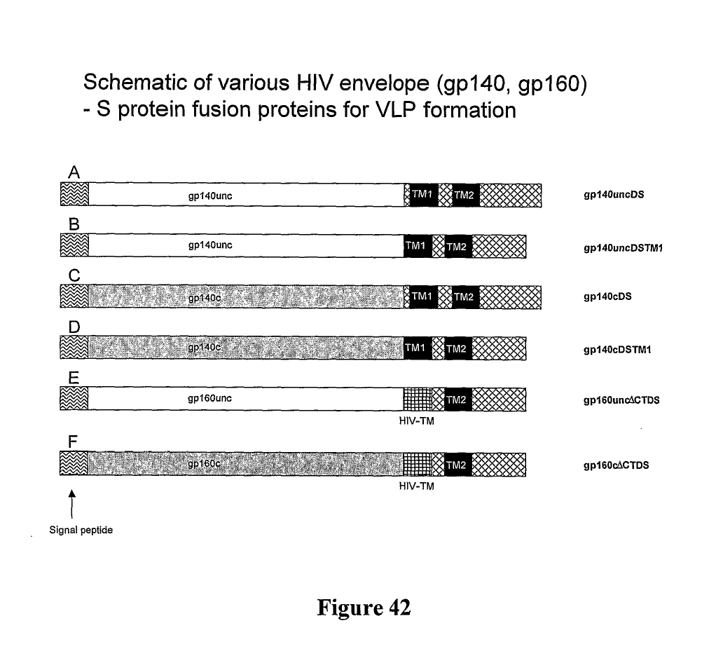

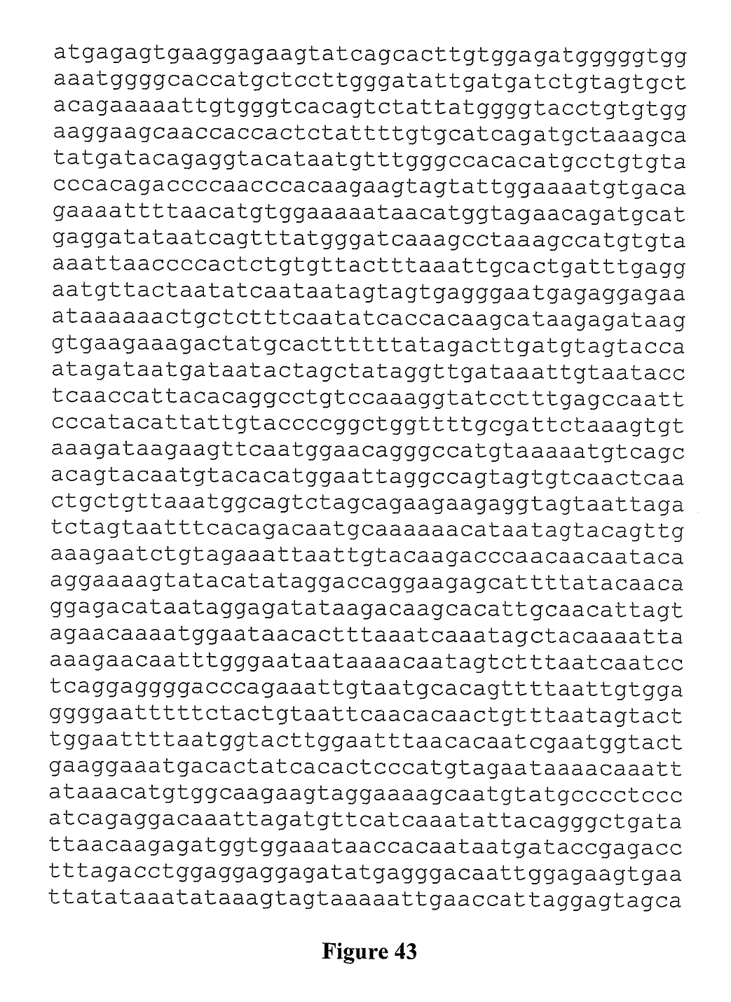

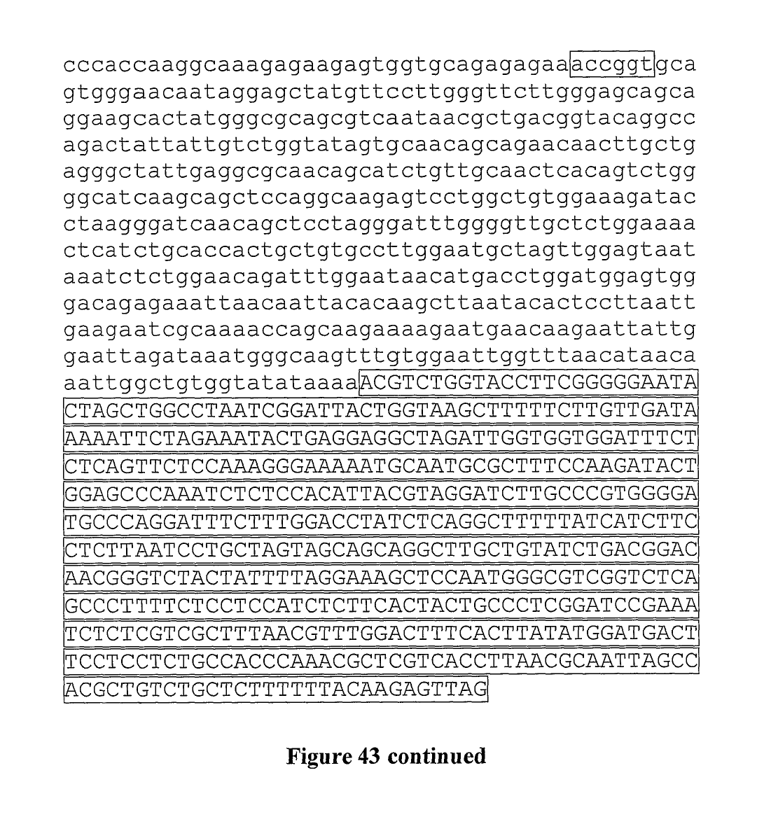

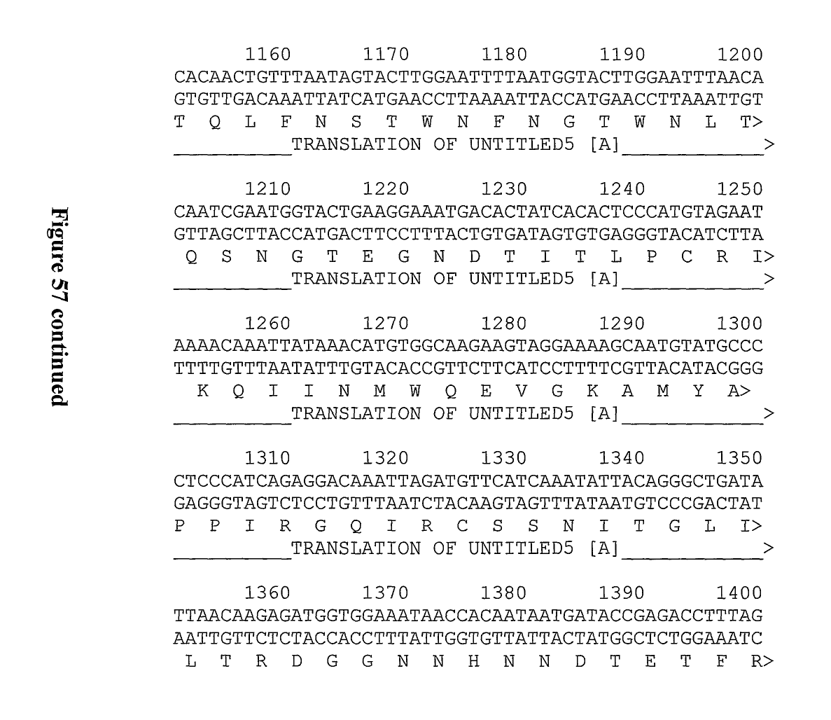

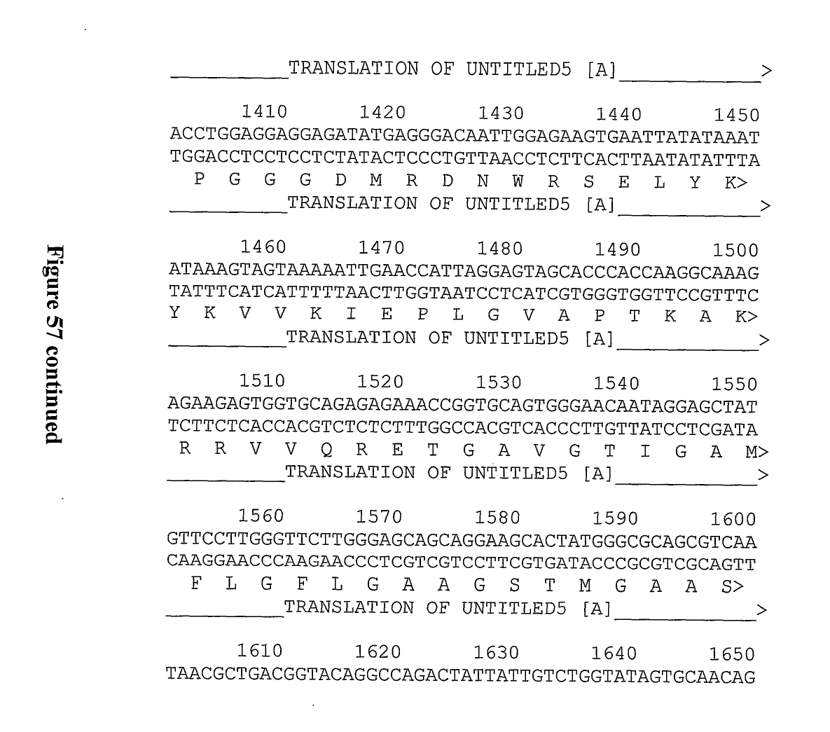

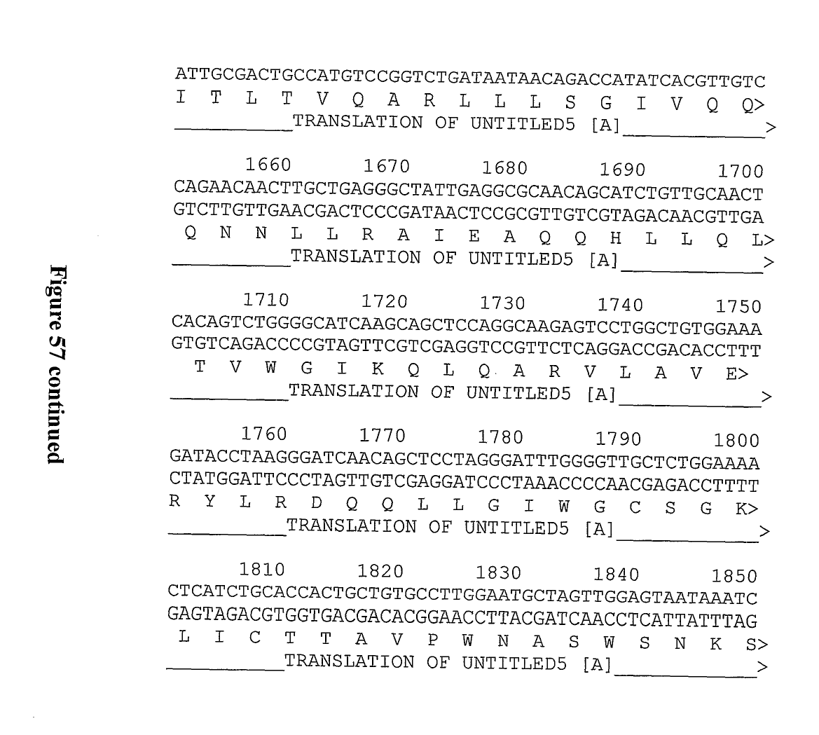

All constructs contain the signal peptide and ectodomain of HIV gp140, which is fused either directly to the N-terminus of the S protein (see FIG. 42A, C); directly to the N-terminus of transmembrane domain 1 of the S protein (FIG. 42B, D), or includes the native transmembrane domain 1 (therefore gp160 rather than gp140) which is fused to the N-terminus of the first cytosolic loop of S, thus replacing the S TM1 (FIG. 42E, F). Wild-type gp140/gp160 contains a furin cleavage site that results in proteolytic processing to give gp120 and gp41 fragments, or in this case gp120 and gp41-S fragments. Mutants which abolish this furin cleavage (gp140unc or gp160unc) are shown in FIGS. 42A, B and E; wild-type cleavage sites (gp140c or gp160c) are shown in FIGS. 42C, D and F. The nucleotide and amino acid sequences of Construct A are shown in FIG. 53. The nucleotide sequence is shown in SEQ ID NO: 26 and the amino acid sequence is shown in SEQ ID NO: 27. The nucleotide sequence of Construct A is also shown in FIG. 43 and SEQ ID NO: 18. In FIG. 43, the nucleotides encoding the S part of the gene at the 5′ end of the gene are boxed. The short furin cleavage site is also boxed (accggt, representing a mutant sequence that encodes a protein that is not cleaved by furin).

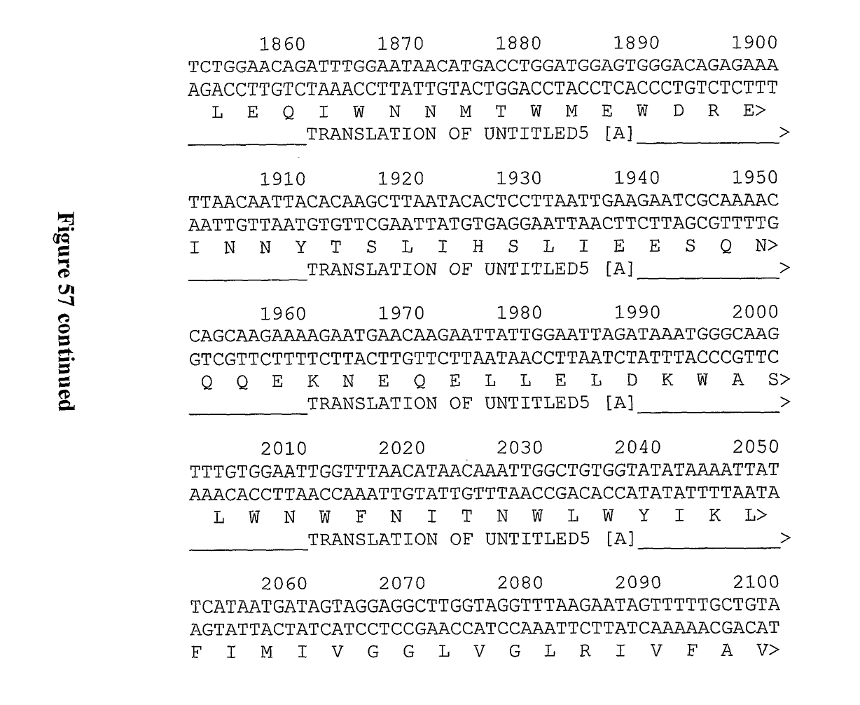

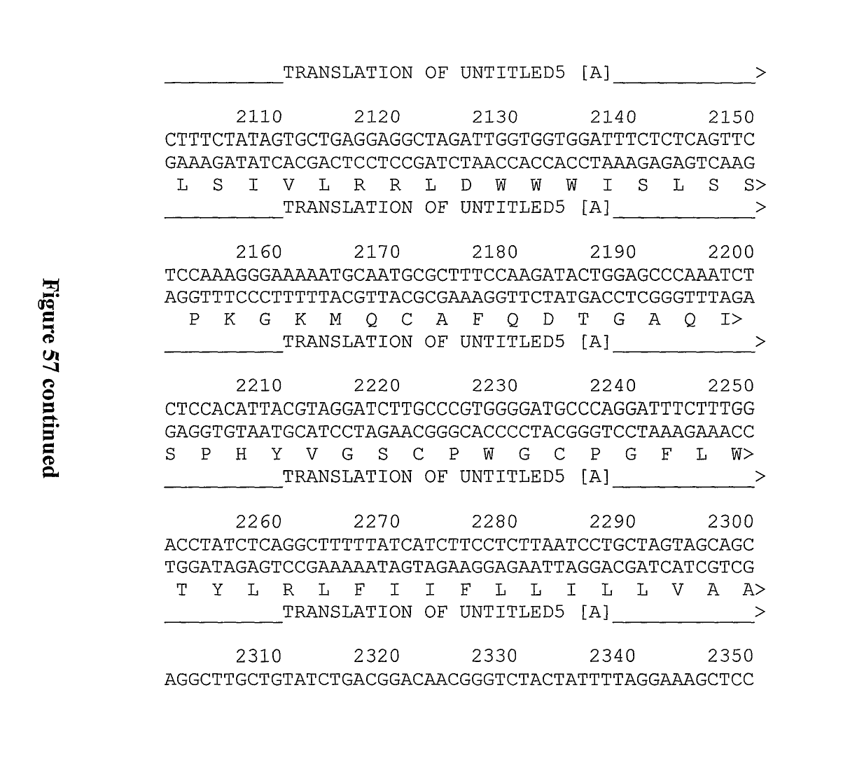

The nucleotide and amino acid sequences of Construct B are shown in FIG. 54. The nucleotide sequence is shown in SEQ ID NO: 28 and the amino acid sequence is shown in SEQ ID NO: 29.

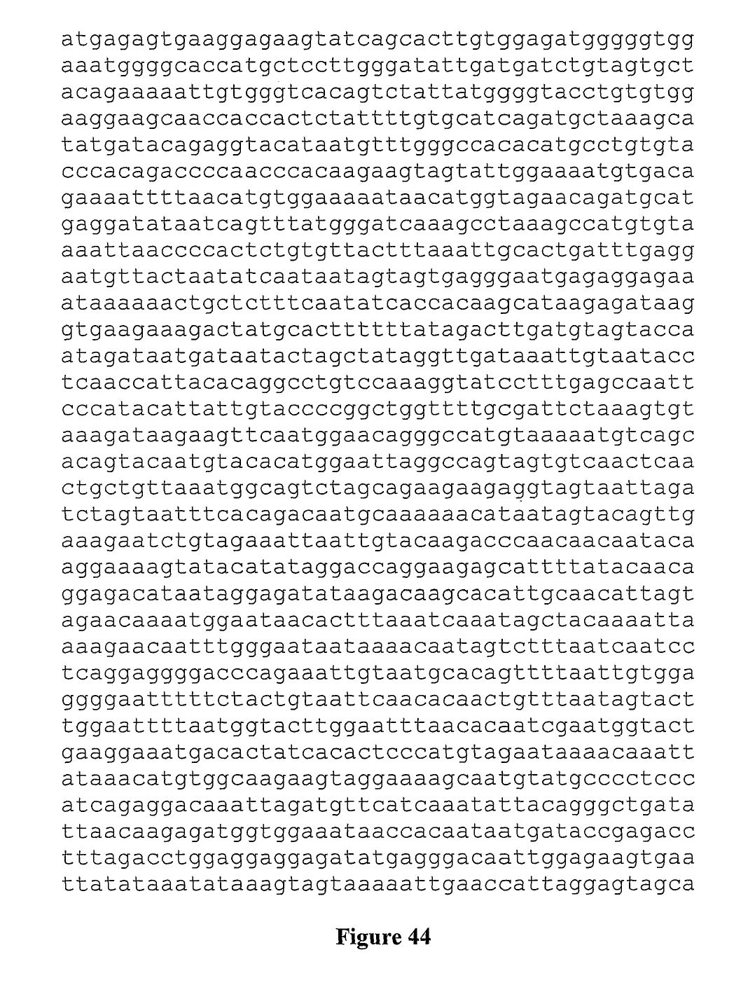

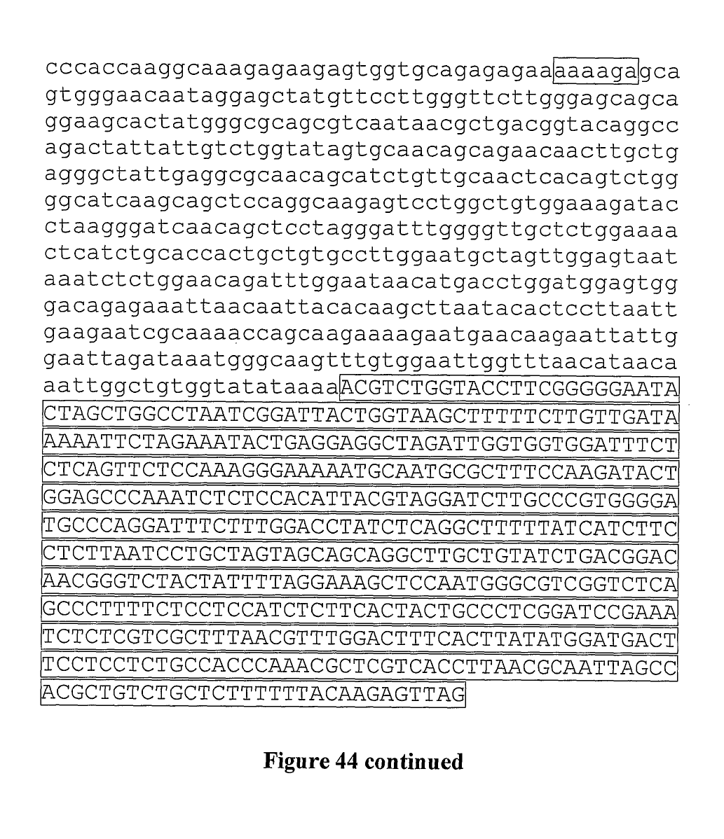

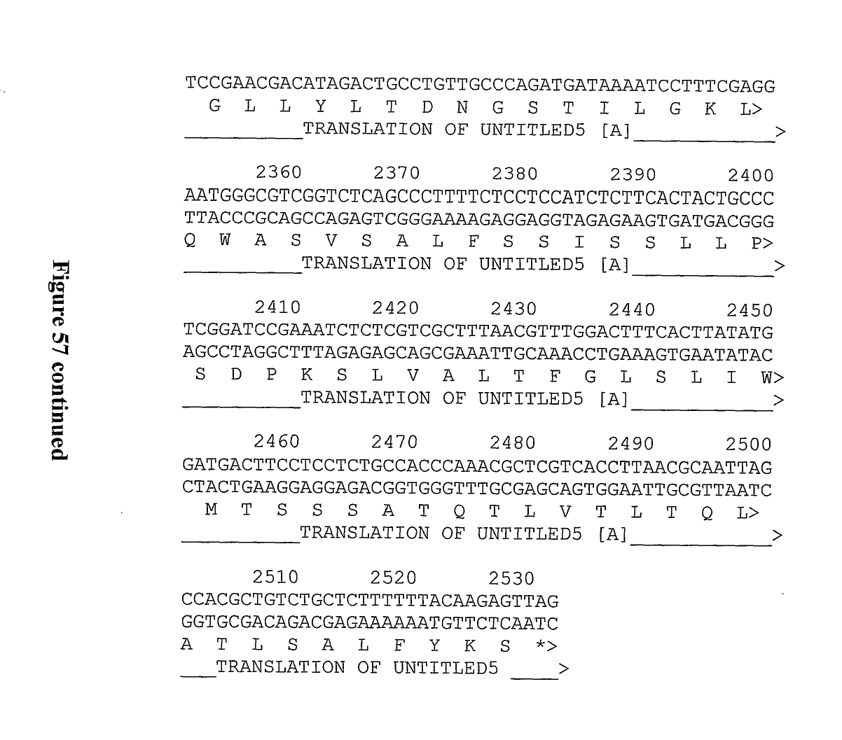

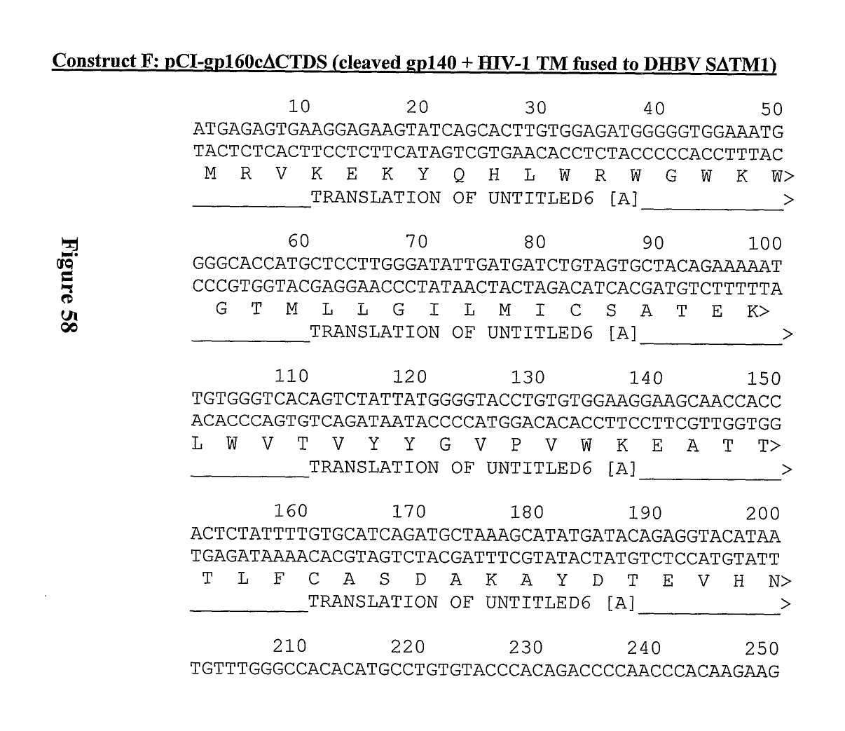

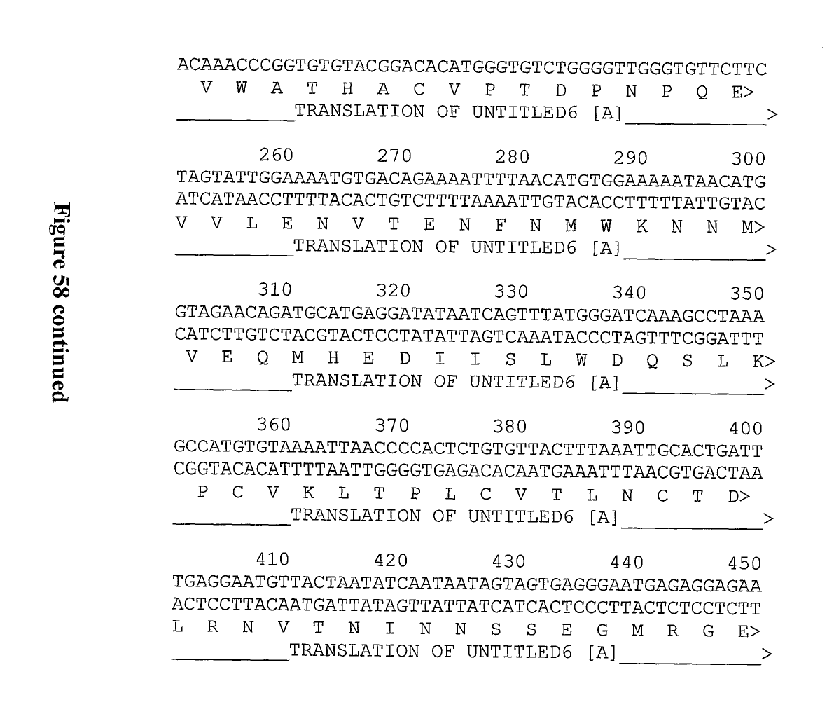

The nucleotide and amino acid sequences of Construct C are shown in FIG. 55. The nucleotide sequence is shown in SEQ ID NO: 30 and the amino acid sequence is shown in SEQ ID NO: 31. The nucleotide sequence of Construct C is also shown in FIG. 44 and SEQ ID NO: 19. In FIG. 44, the nucleotides encoding the S part of the gene at the 5′ end of the gene are boxed. The short furin cleavage site is also boxed (aaaaga, representing the wild-type sequence that encodes a protein that is cleaved by furin).

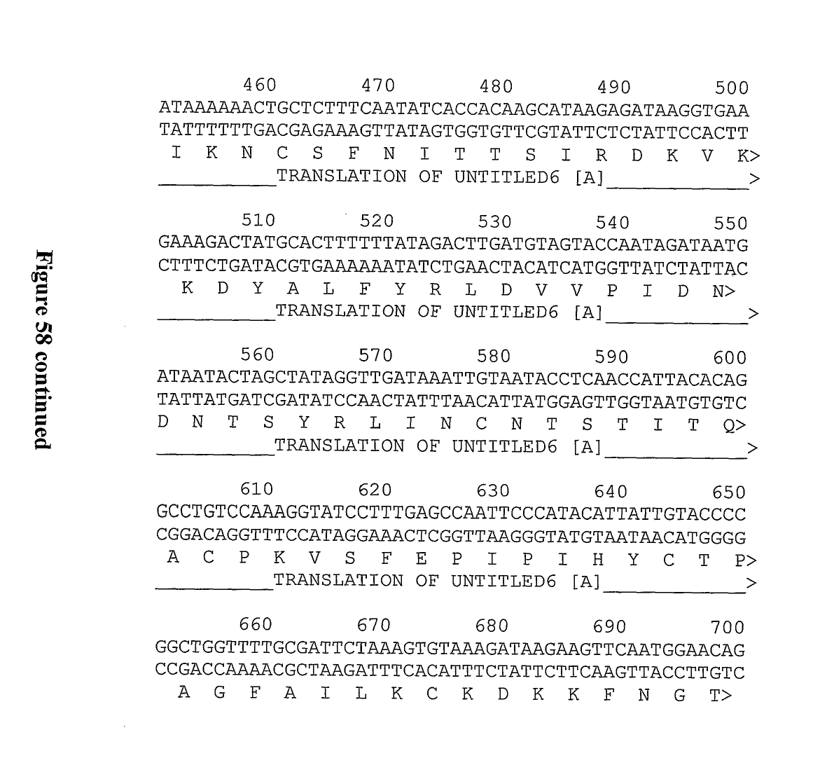

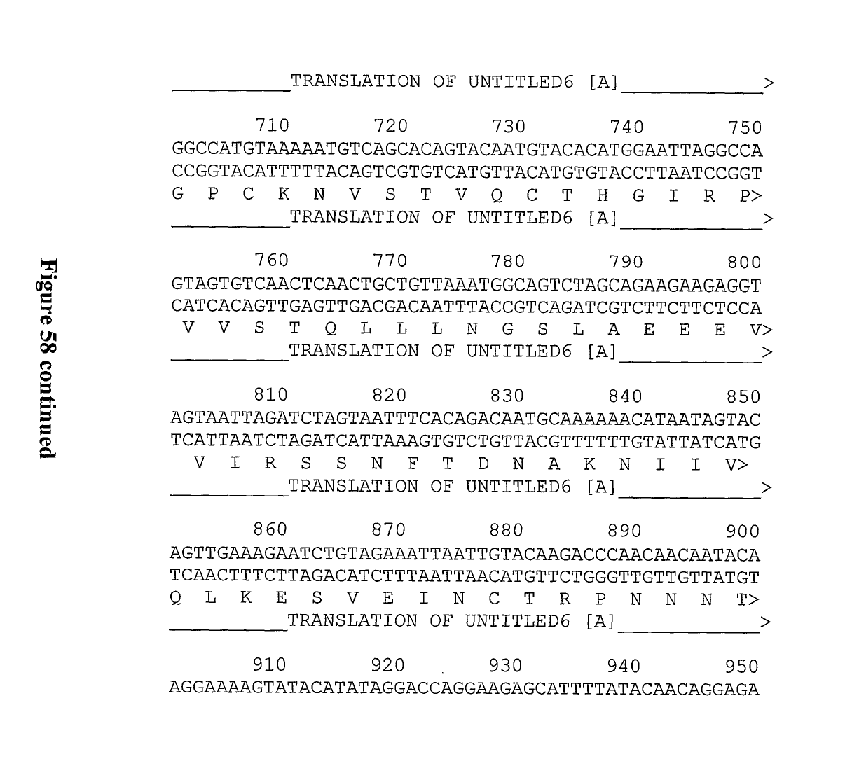

The nucleotide and amino acid sequences of Construct D are shown in FIG. 56. The nucleotide sequence is shown in SEQ ID NO: 32 and the amino acid sequence is shown in SEQ ID NO: 33.

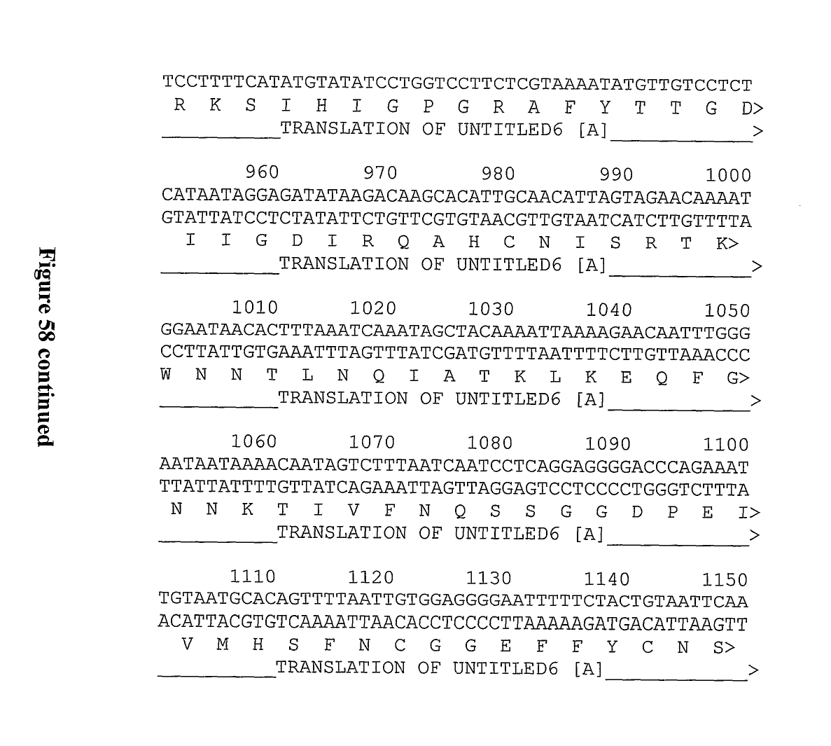

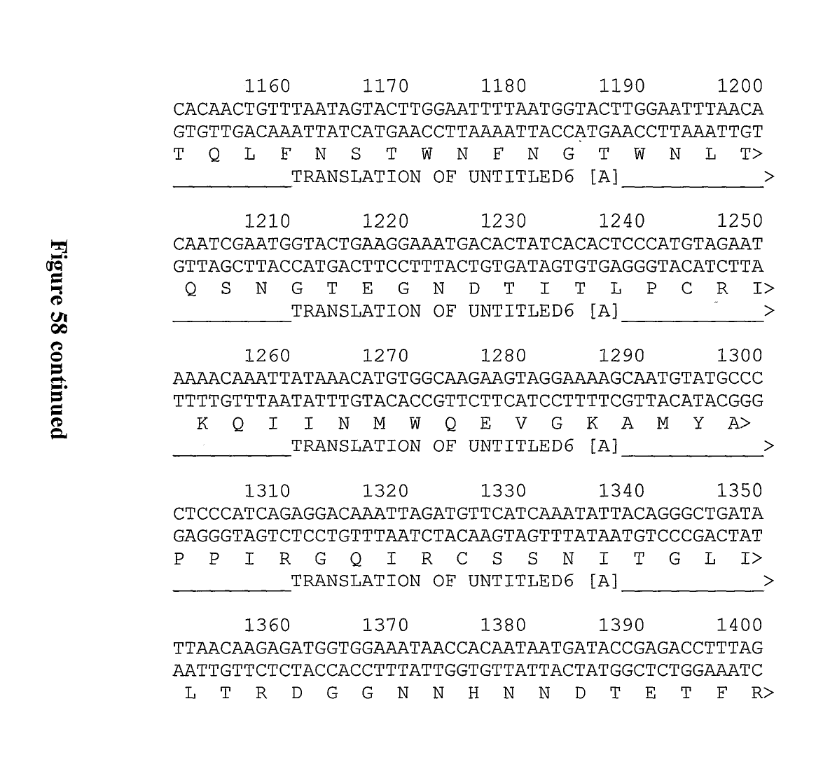

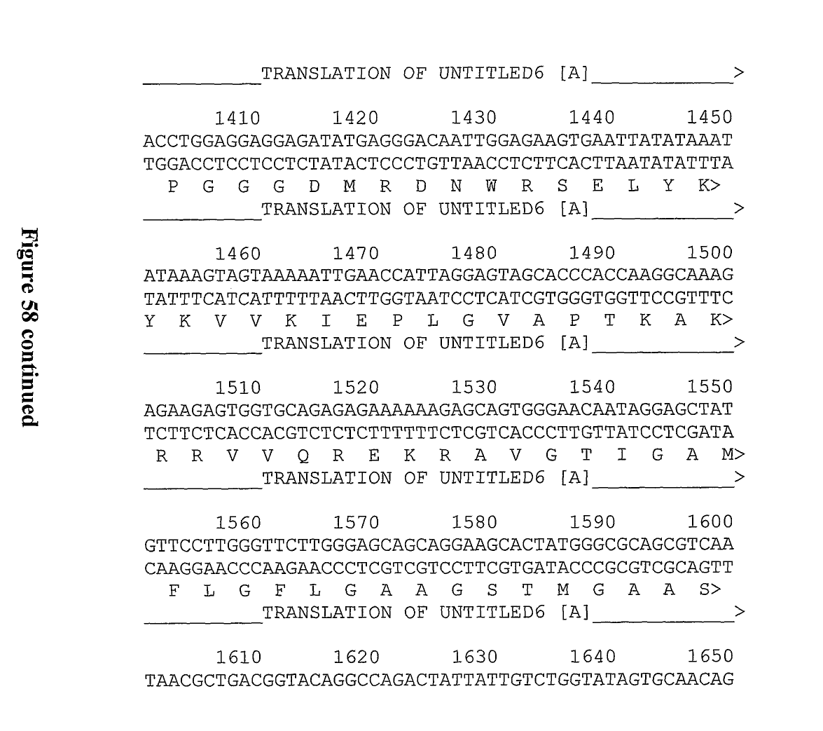

The nucleotide and amino acid sequences of Construct E are shown in FIG. 57. The nucleotide sequence is shown in SEQ ID NO: 34 and the amino acid sequence is shown in SEQ ID NO: 35.

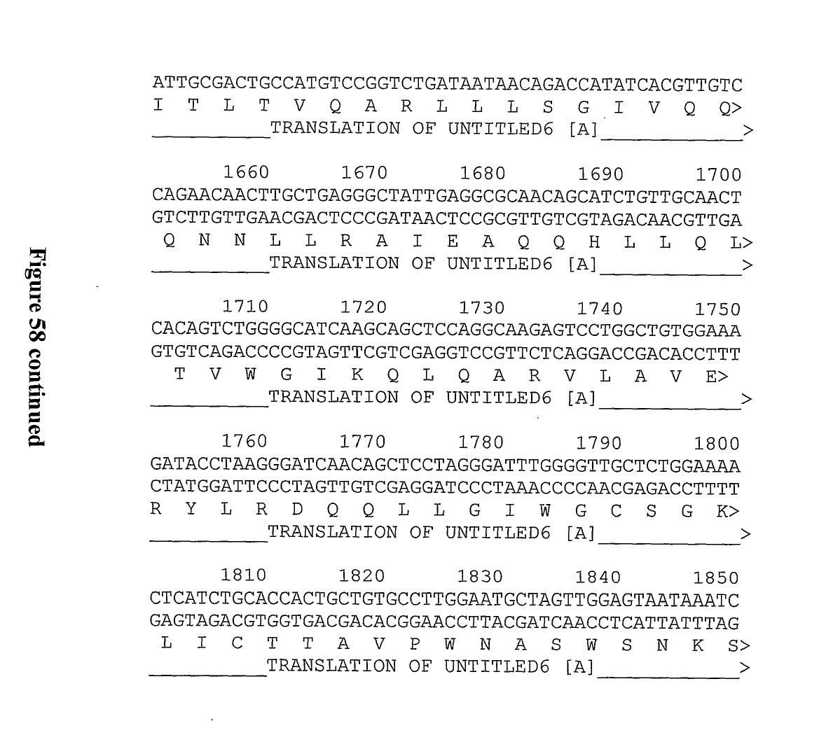

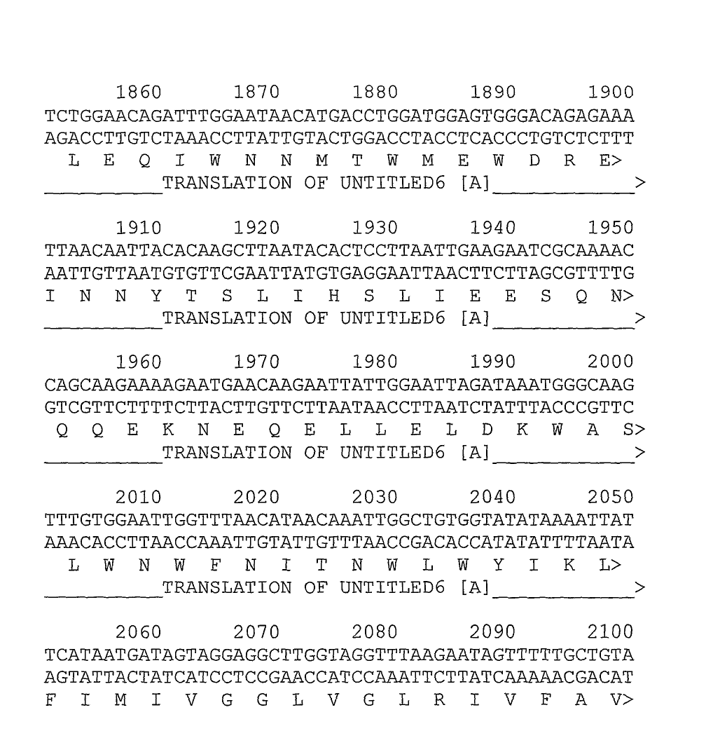

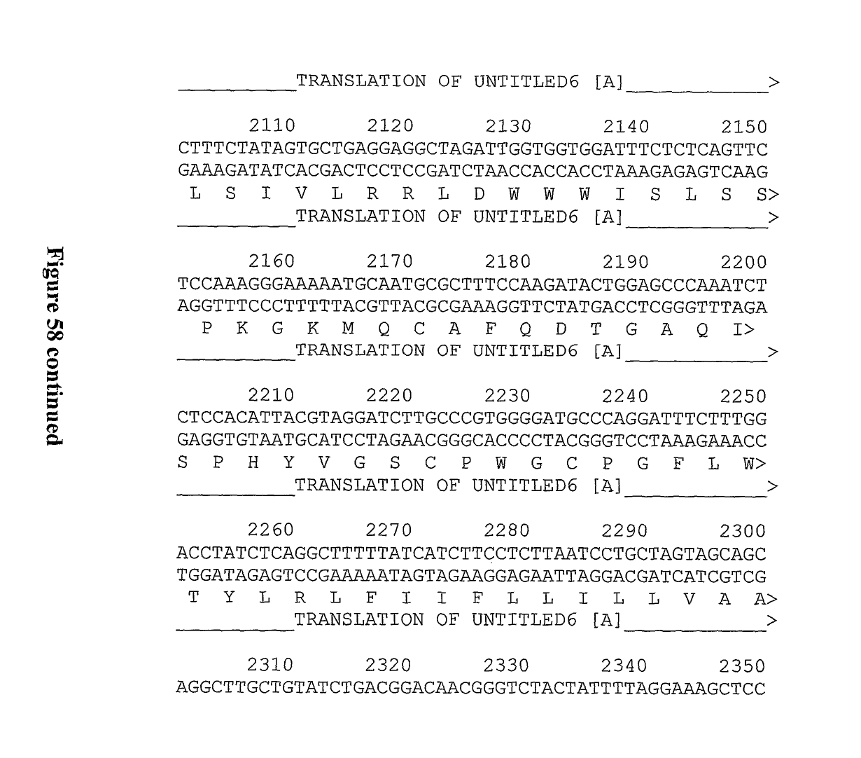

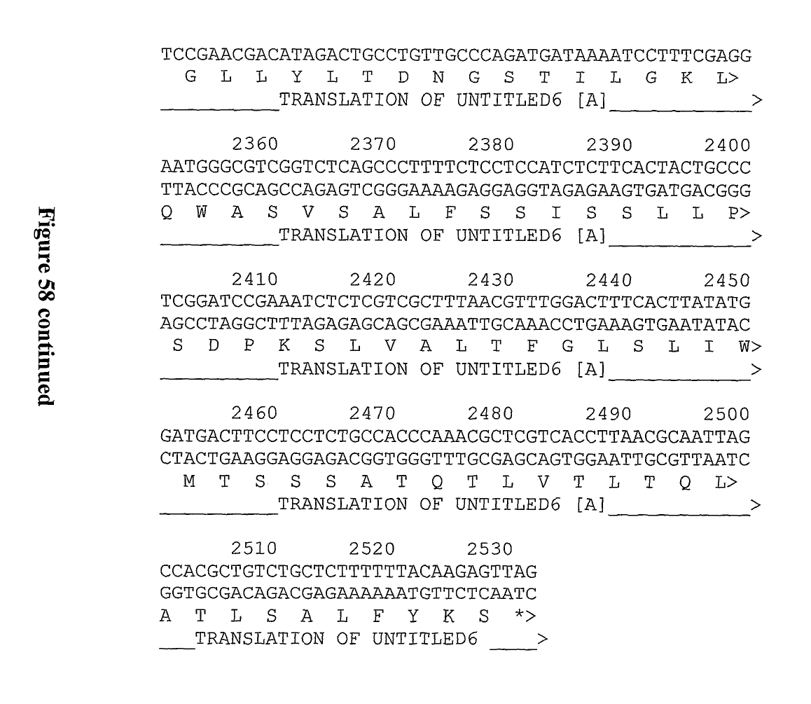

The nucleotide and amino acid sequences of Construct F are shown in FIG. 58. The nucleotide sequence is shown in SEQ ID NO: 36 and the amino acid sequence is shown in SEQ ID NO: 37.

### Example 41

### Expression of HIV gp140-S

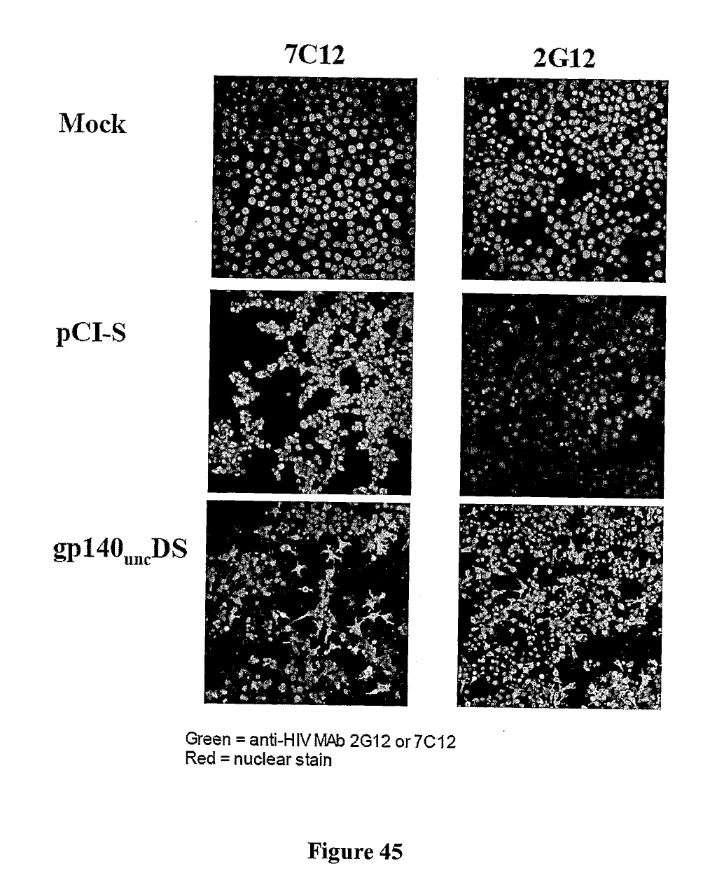

Expression of HIV gp140-S was detected by indirect immunofluorescence with HIV envelope-specific monoclonal antibody 2G12 (see FIG. 45).

### Example 42

### Assembly of HIV gp140-S and S into VLPs Detected by Western Immunoblotting

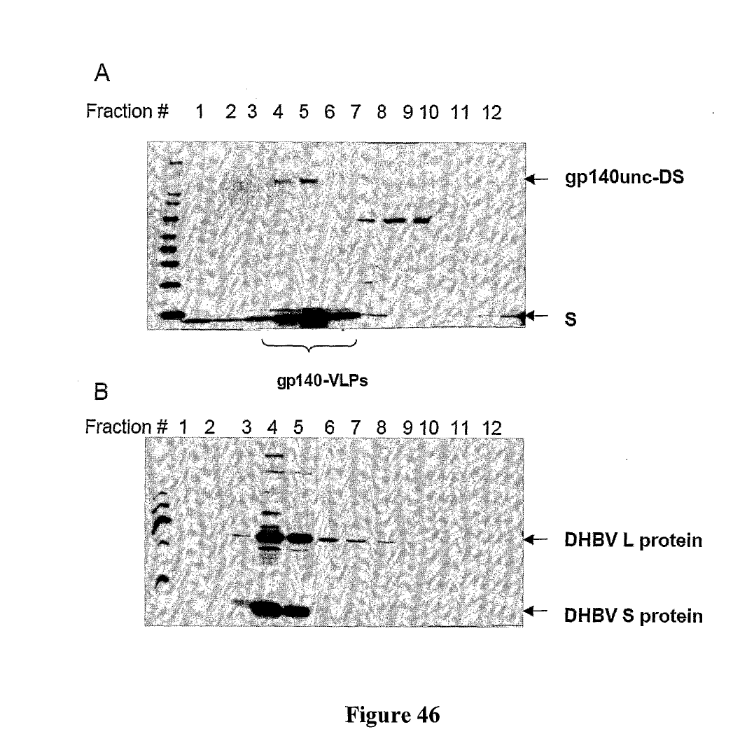

Assembly of HIV gp140-S and S into VLPs was detected by Western immunoblotting with a combination of HIV envelope-specific patient serum and monoclonal antibody 7C12 (FIG. 46A), and cosedimentation with wild-type DHBV VLPs (containing DHBV L protein and S protein) detected by Western immunoblotting with monoclonal antibody 7C12 alone (FIG. 46B).

### Example 43

### Assembly of HIV gp140-S and S into VLPs Detected by ELISA

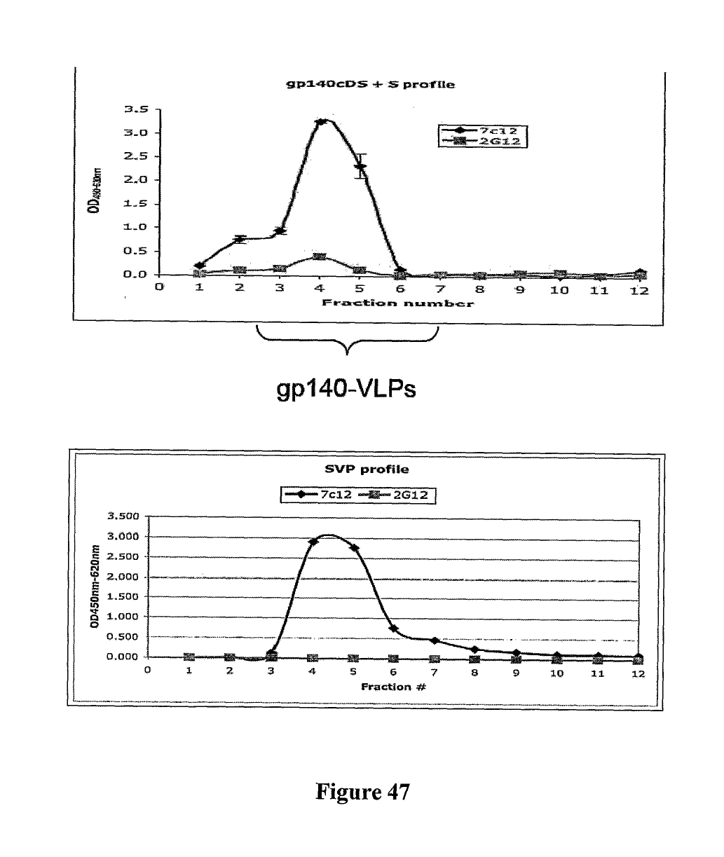

Assembly of HIV gp140-S and S into VLPs was detected by ELISA with a combination of HIV envelope-specific monoclonal antibody 2G12 and monoclonal antibody 7C12 (FIG. 47A), and cosedimentation with wild-type DHBV VLPs (containing DHBV L protein and S protein) detected by ELISA with monoclonal antibody 7C12 and showing no reactivity with 2G12 (FIG. 47B).

### Example 44

### Assembly of VLPs Comprising Constructs A to F Detected by ELISA

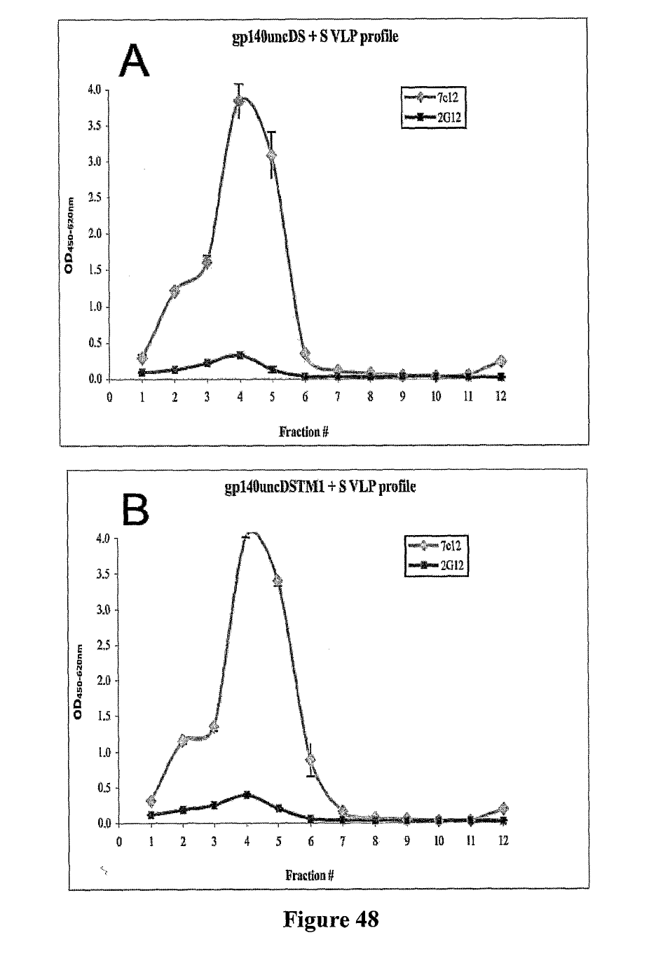

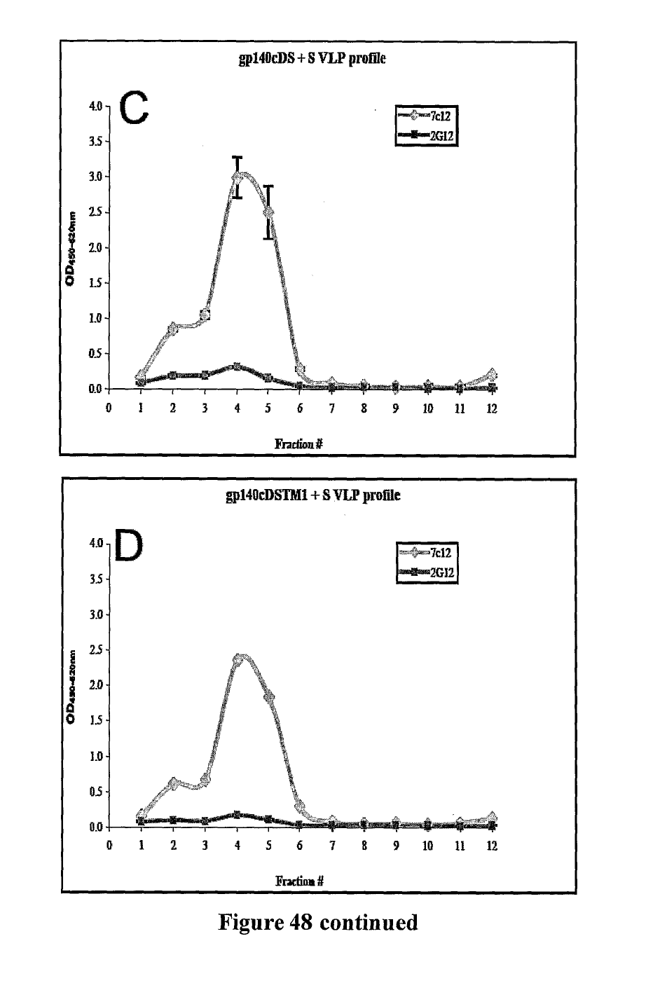

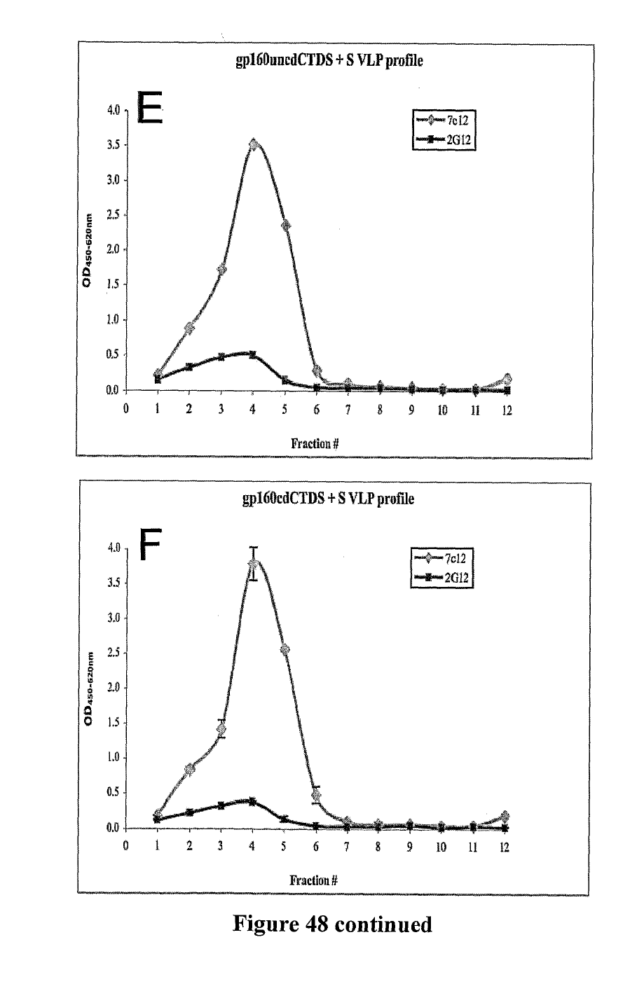

Assembly of various forms of HIV gp140-S or gp160-S together with S into VLPs, detected by ELISA with a combination of HIV envelope-specific monoclonal antibody 2G12 and monoclonal antibody 7C12. FIGS. 48 A to F correspond to constructs A to F in FIG. 42. Both cleaved and uncleaved forms, gp140 and gp160 forms, and TMD or no TMD forms of HIV envelope are able to assemble into VLPs.

### Example 45

### Endogenous Cleavage of gp140 into gp120 and gp41-DS wherein gp120 Remains Associated with the VLP via Non-Peptide Linkage

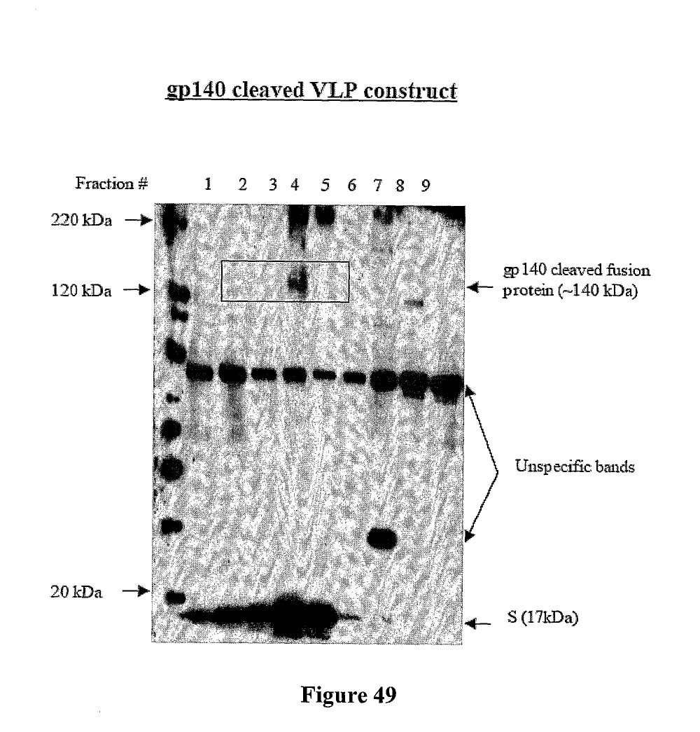

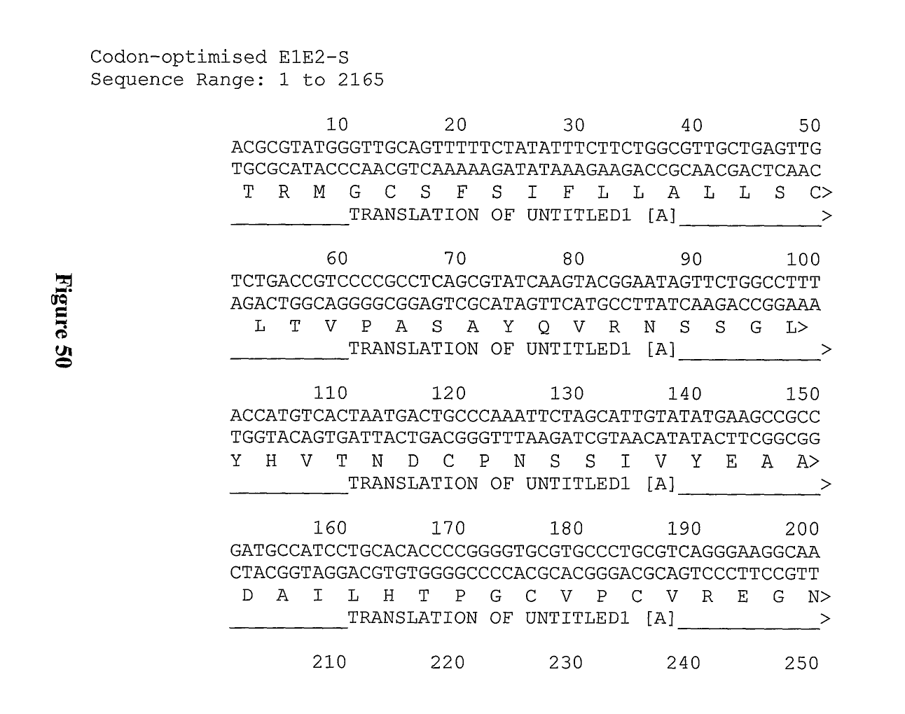

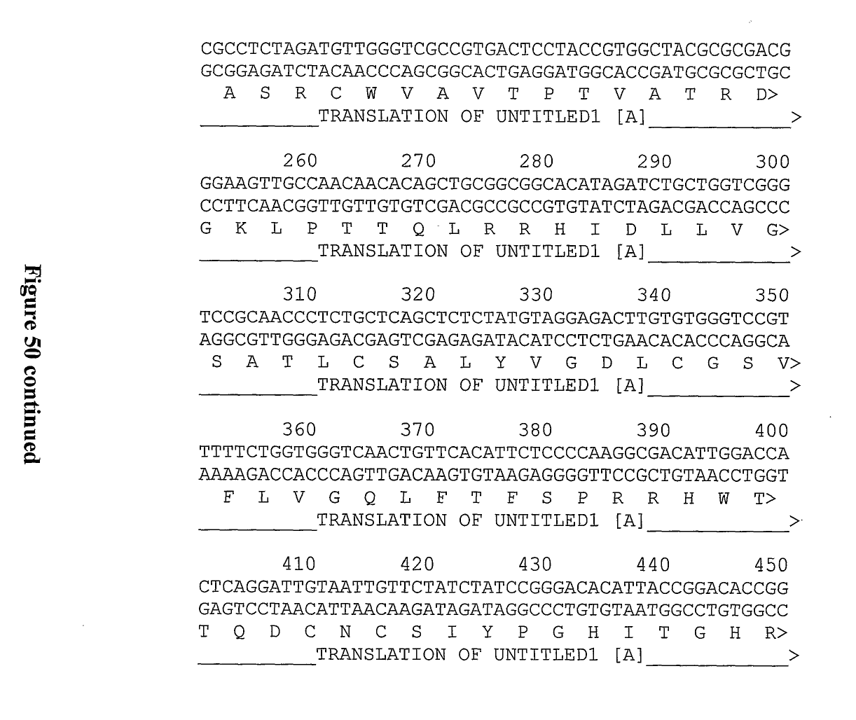

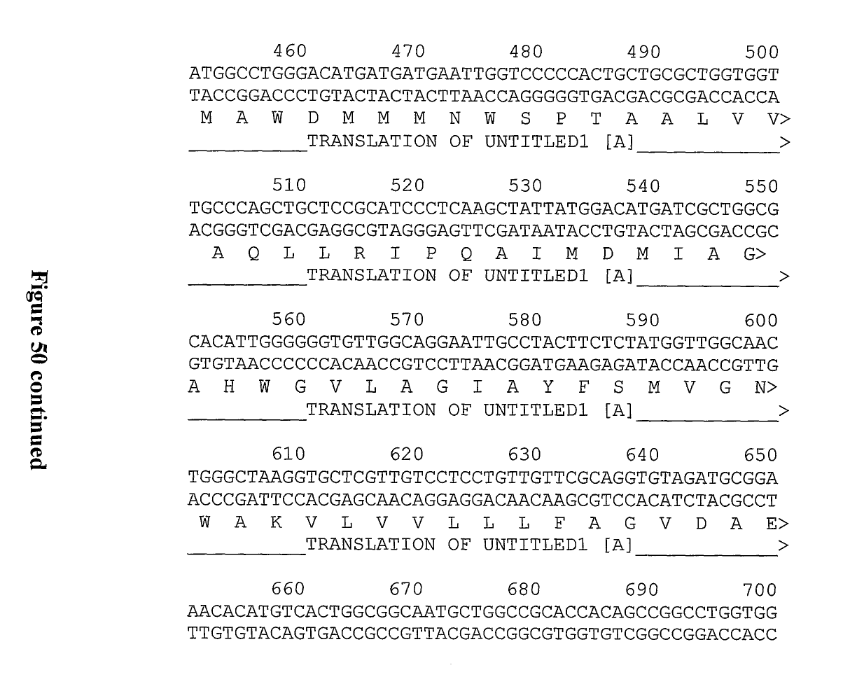

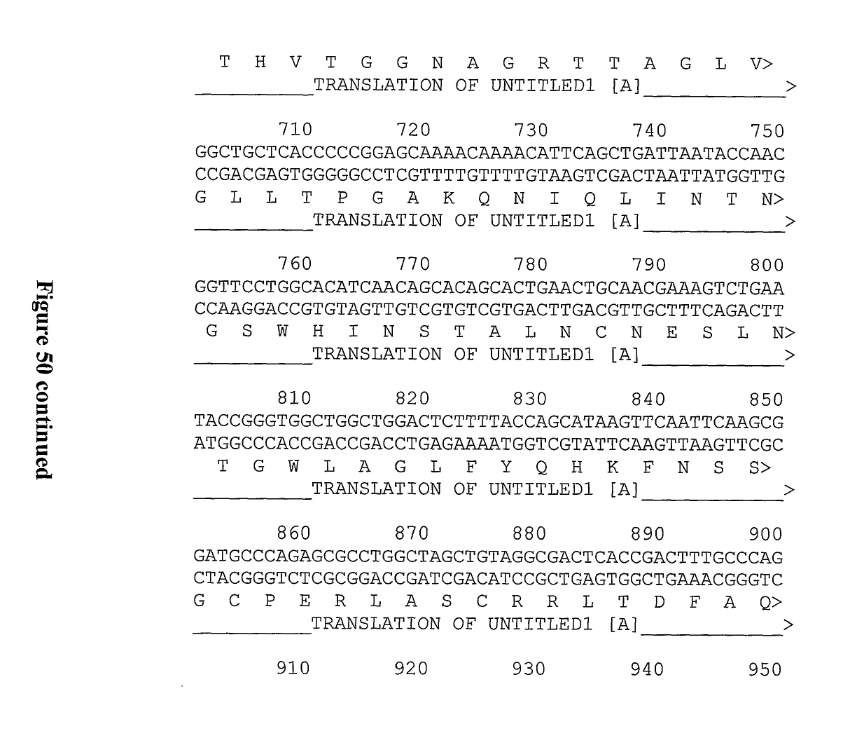

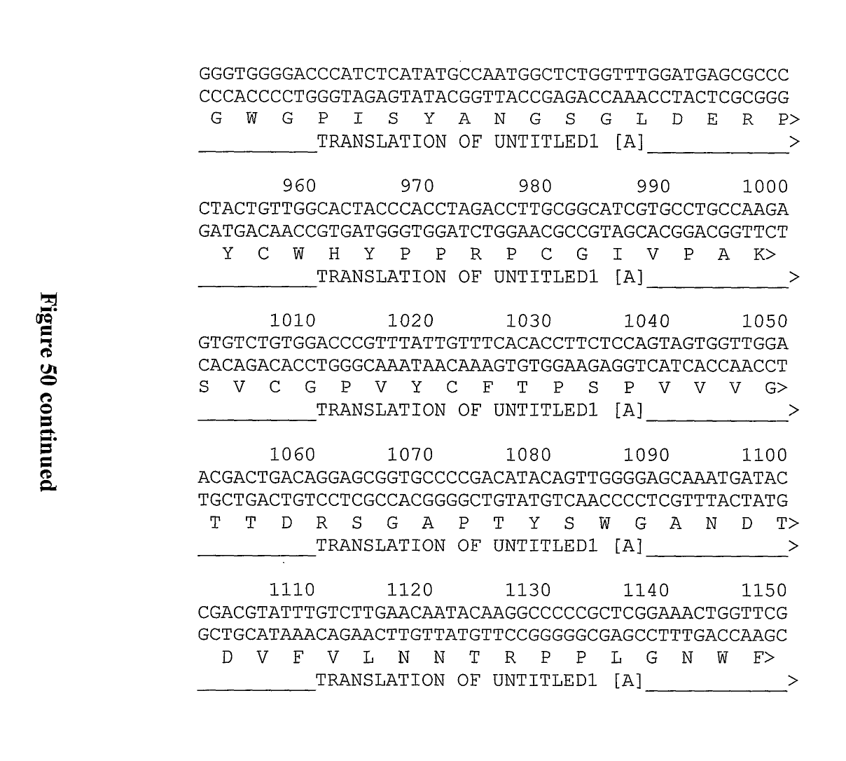

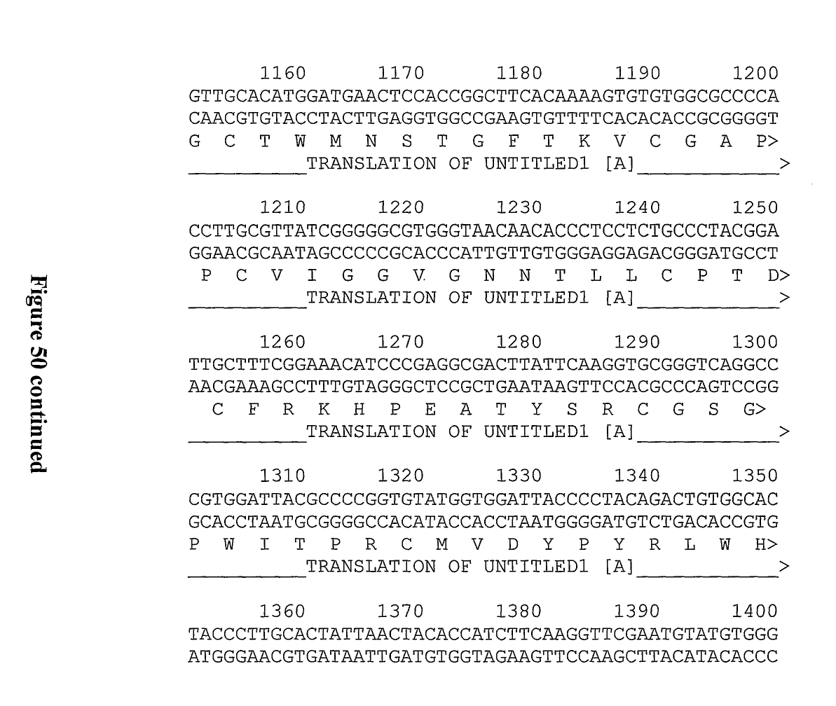

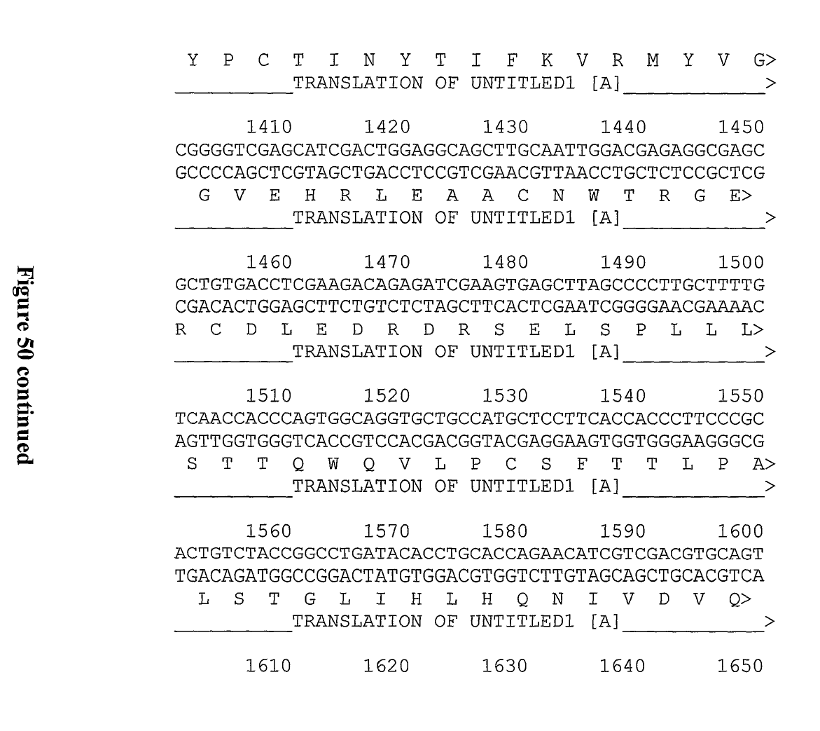

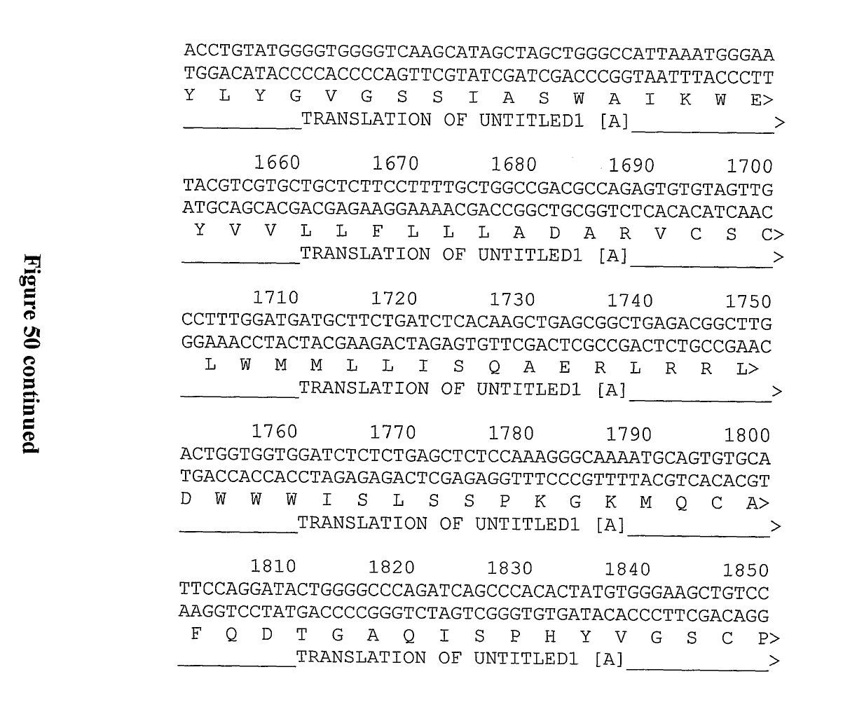

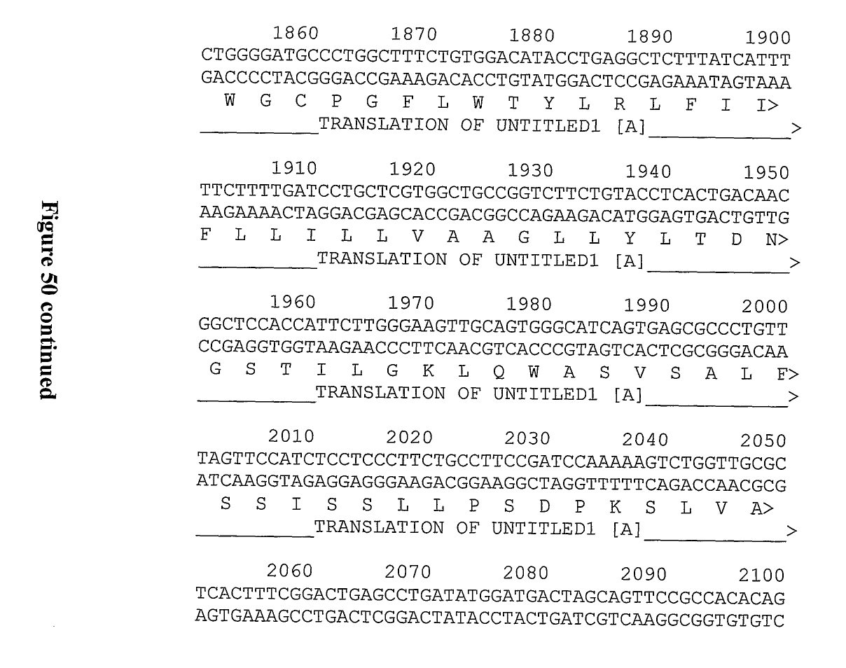

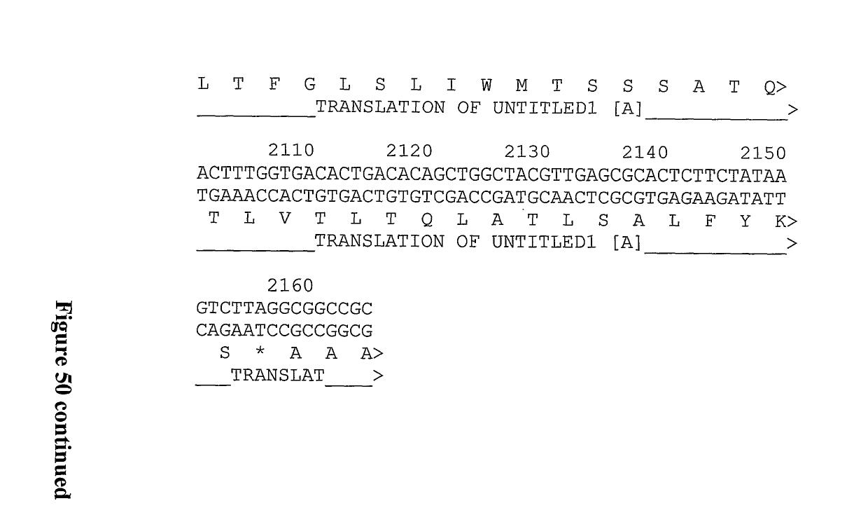

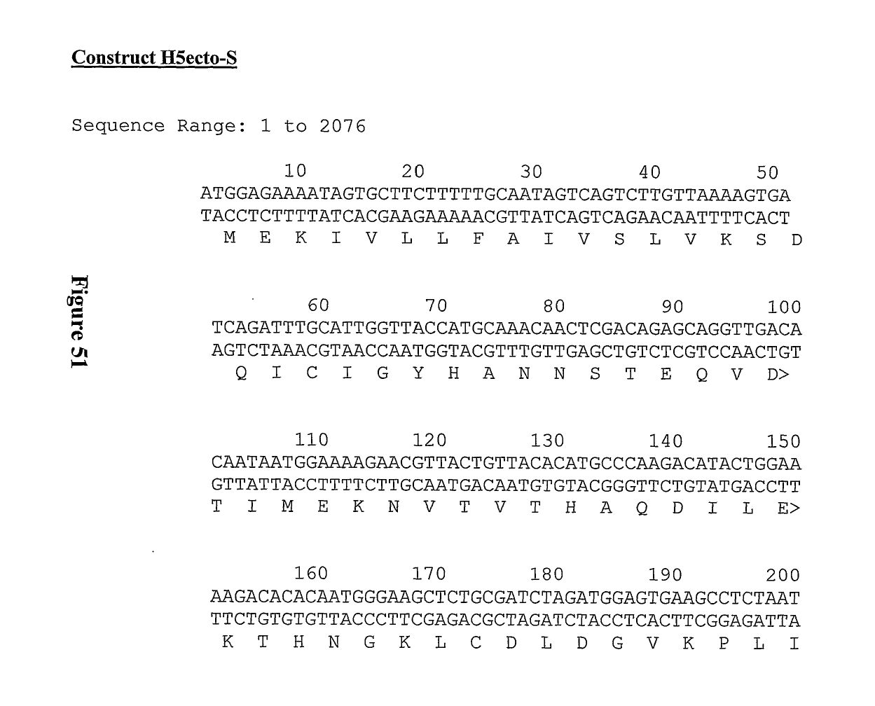

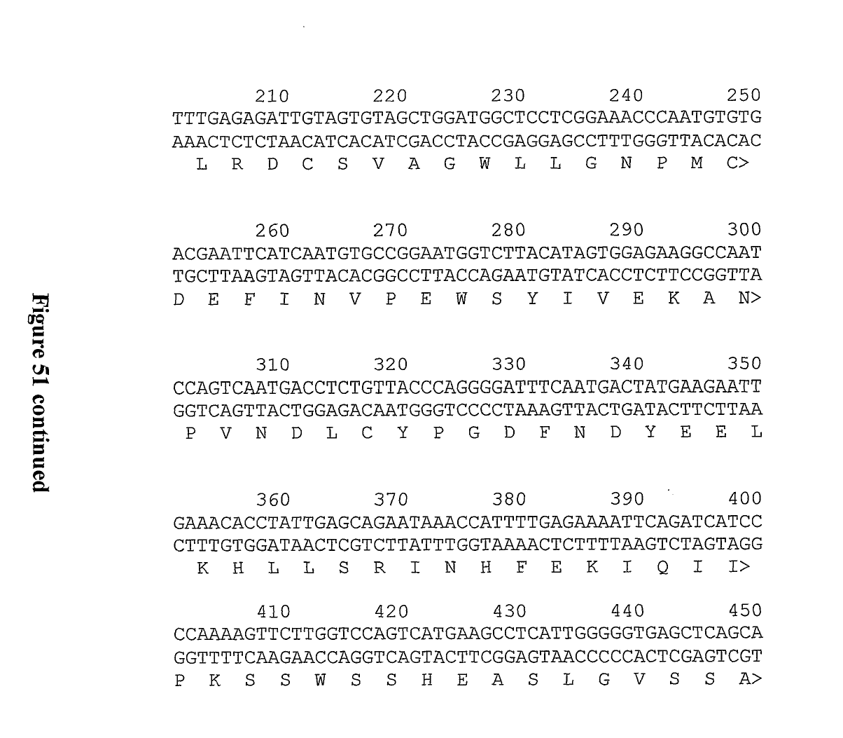

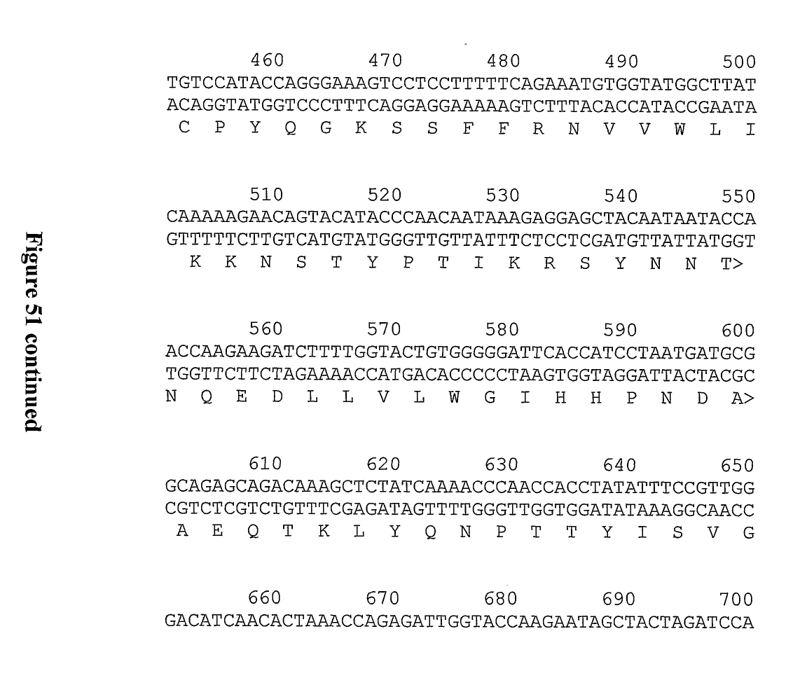

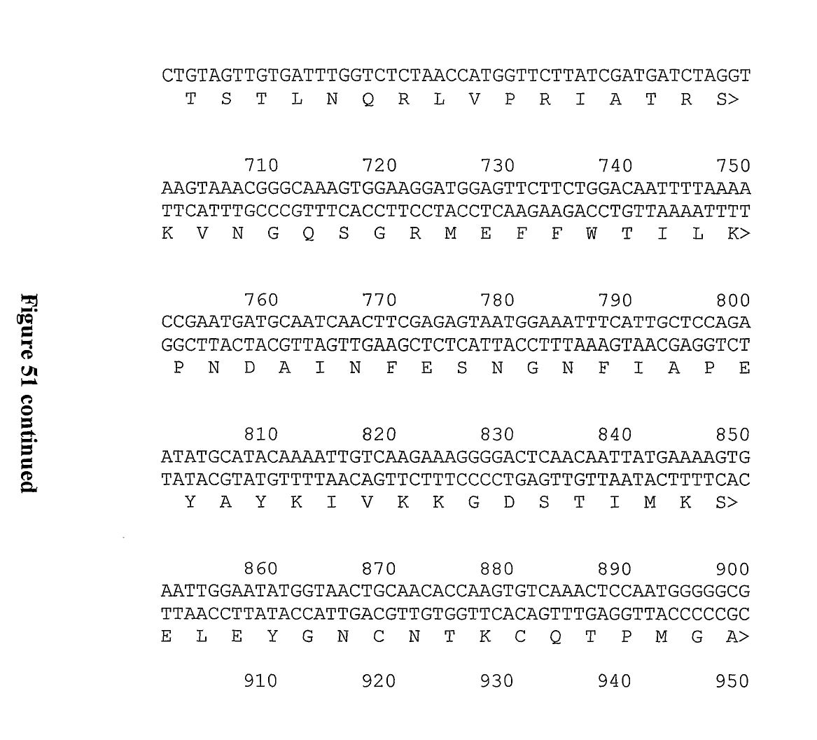

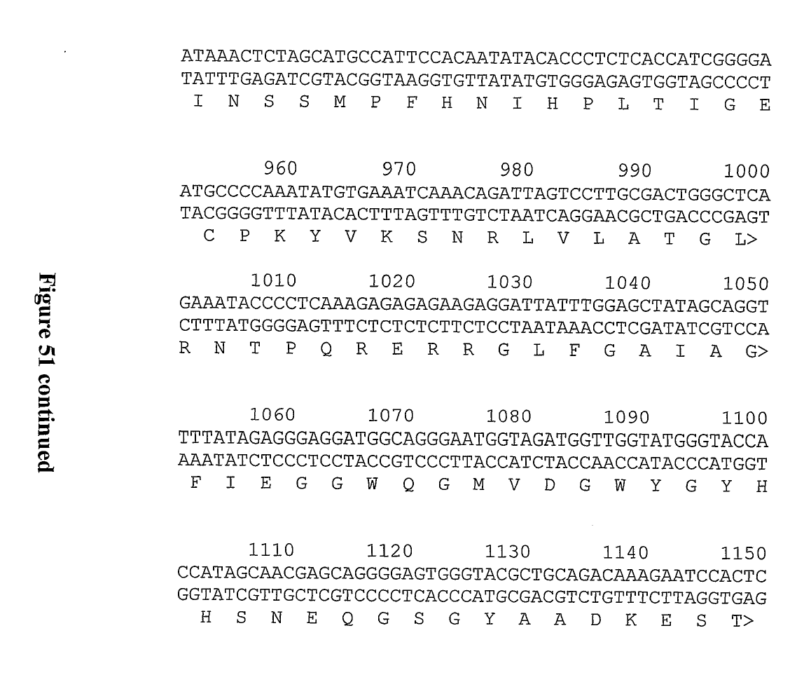

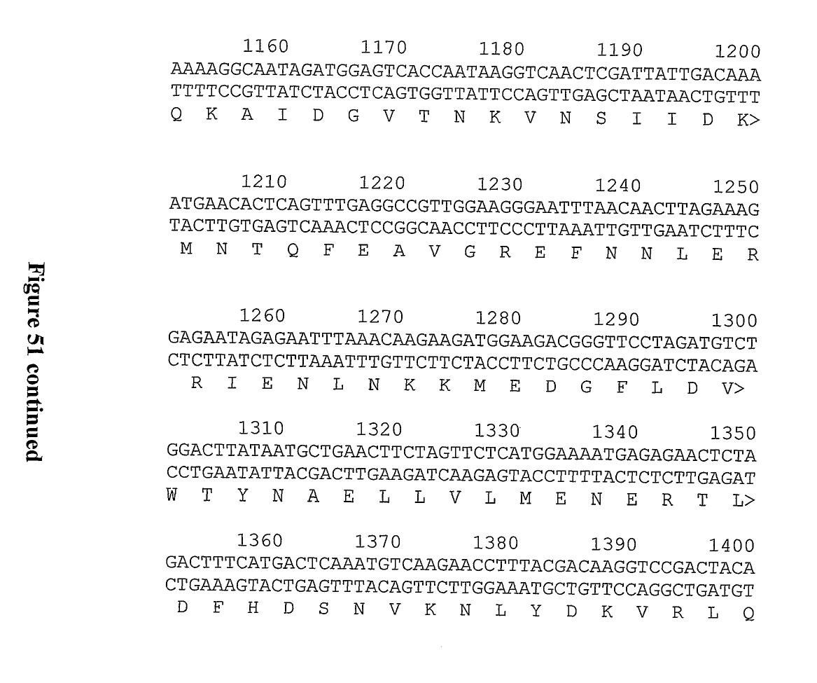

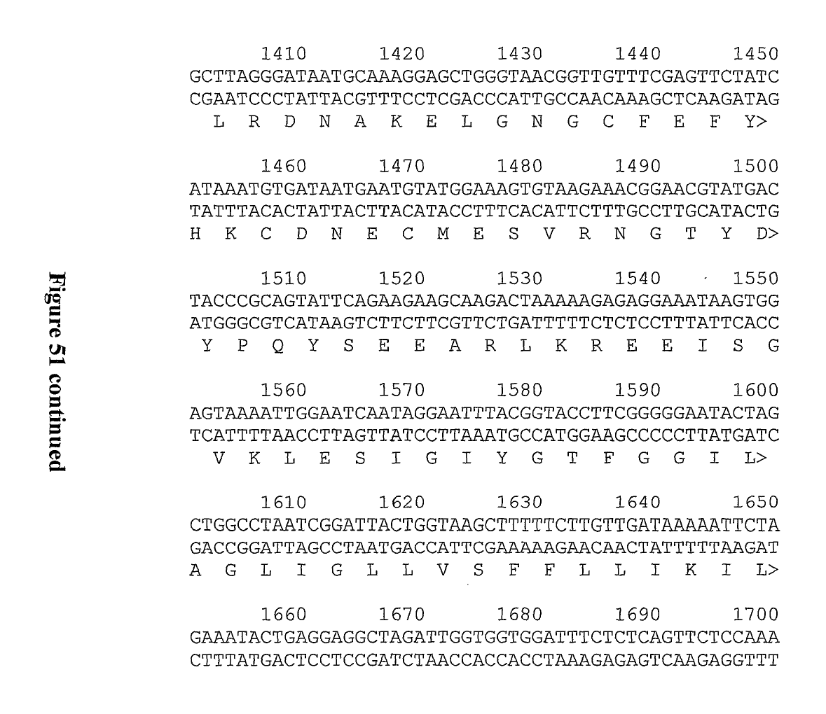

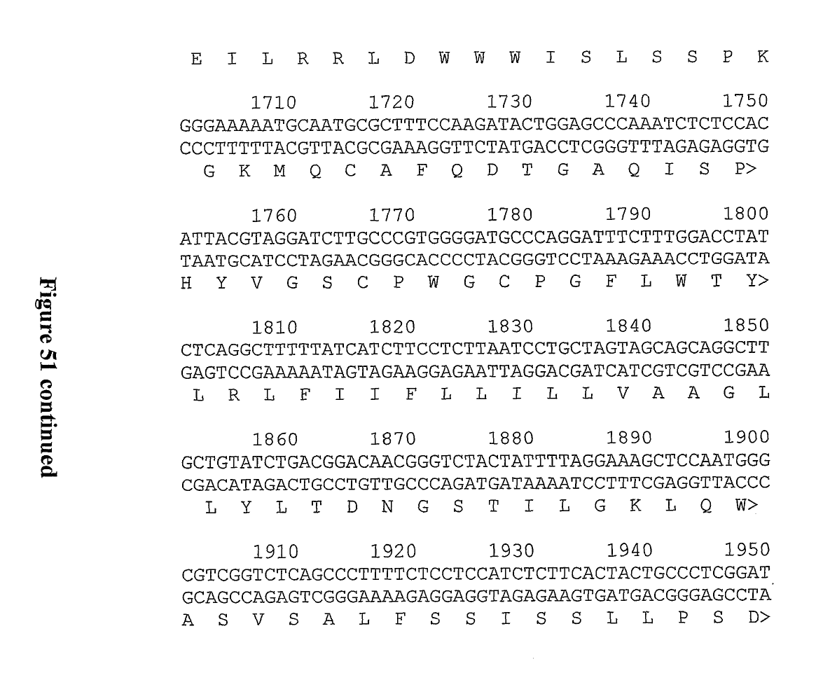

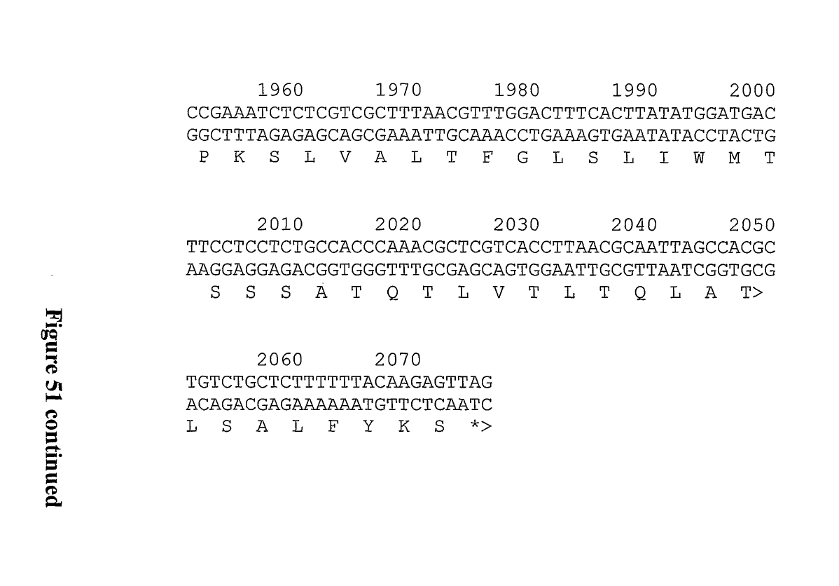

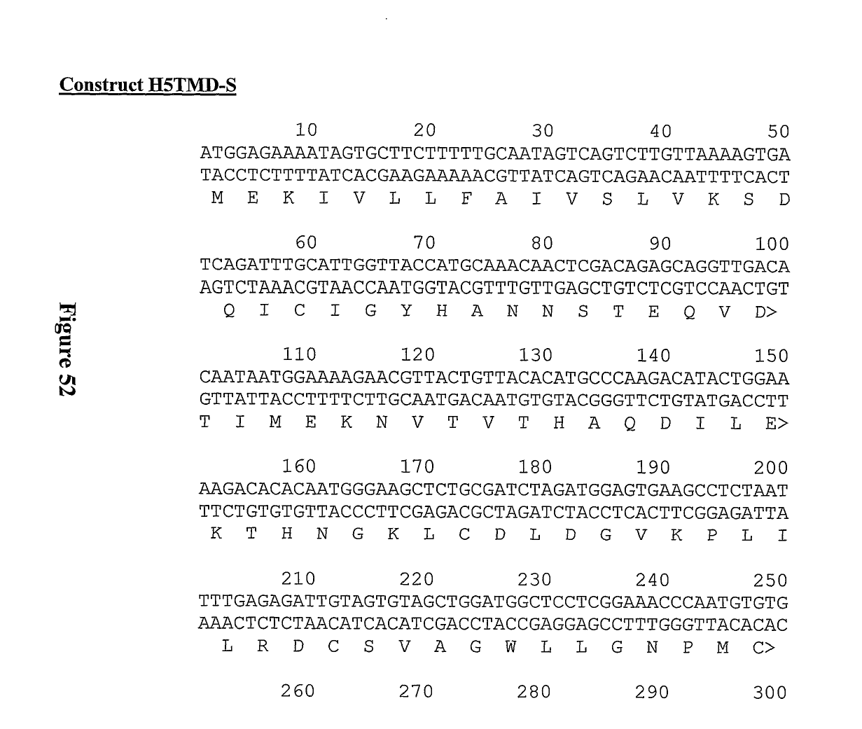

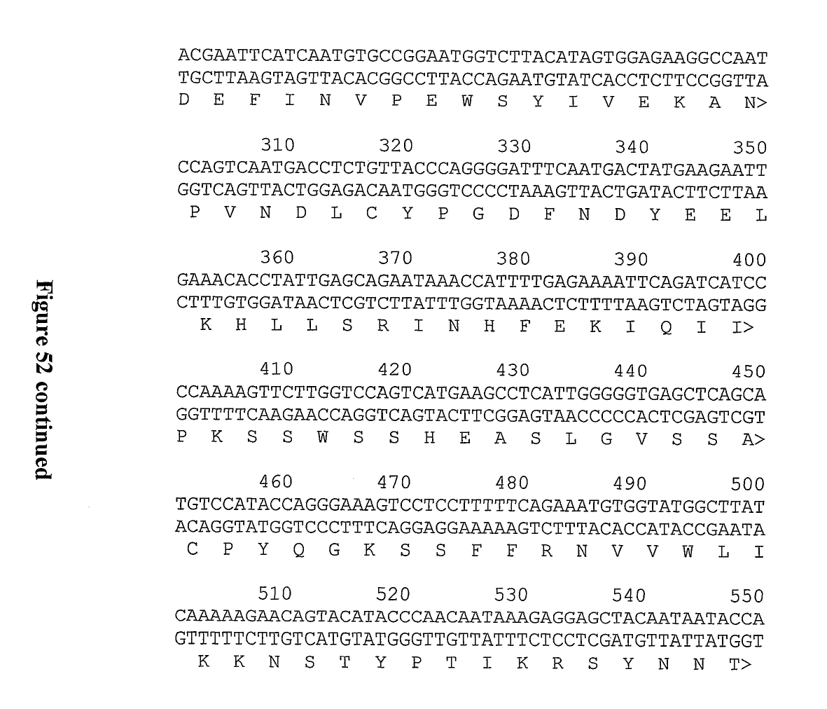

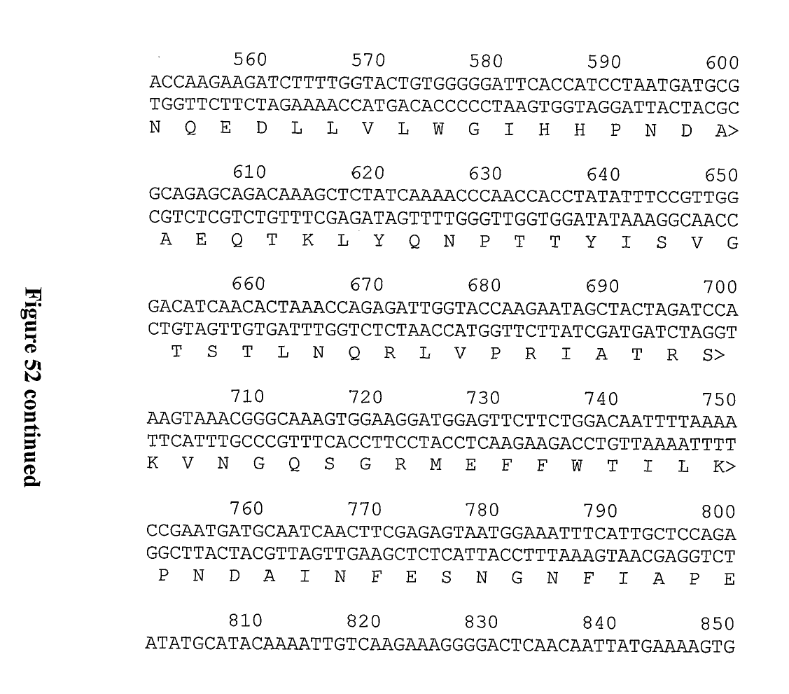

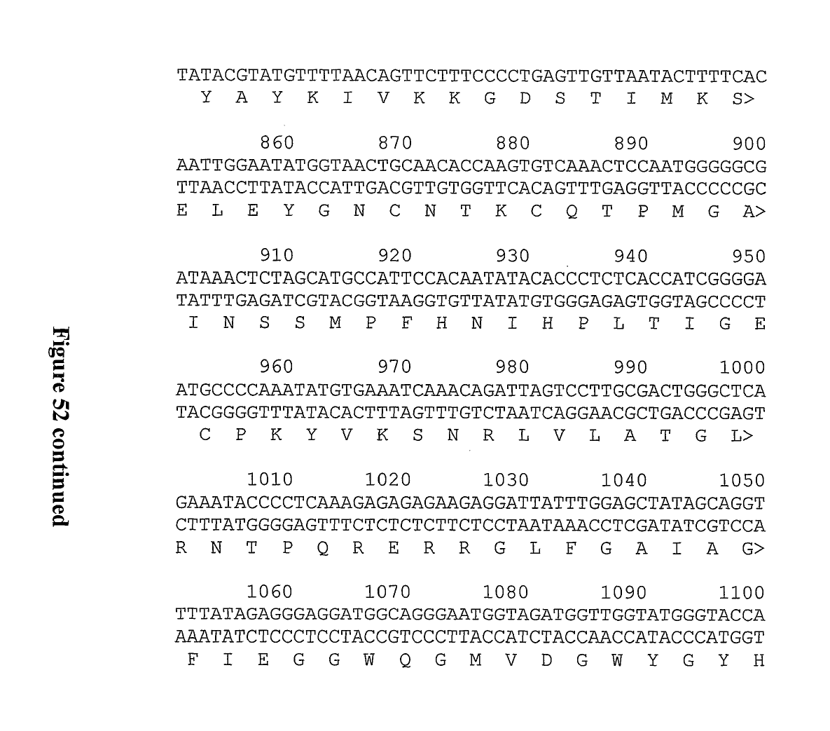

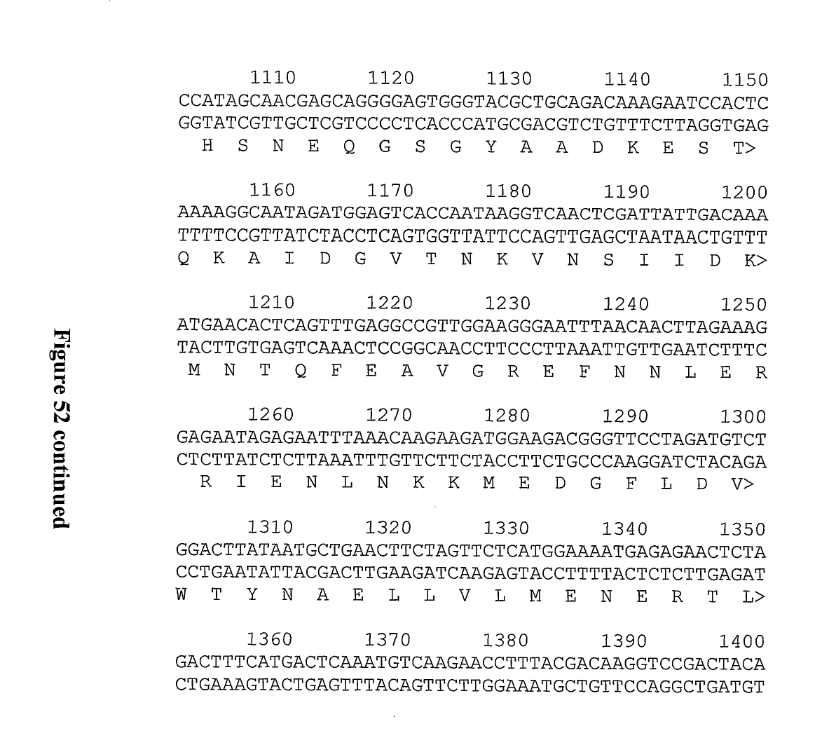

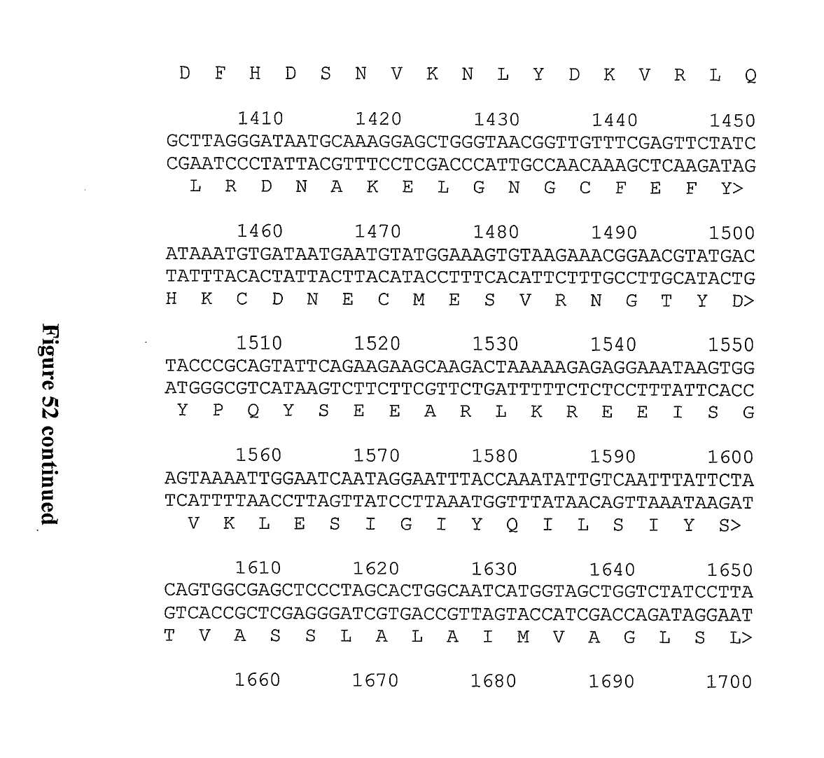

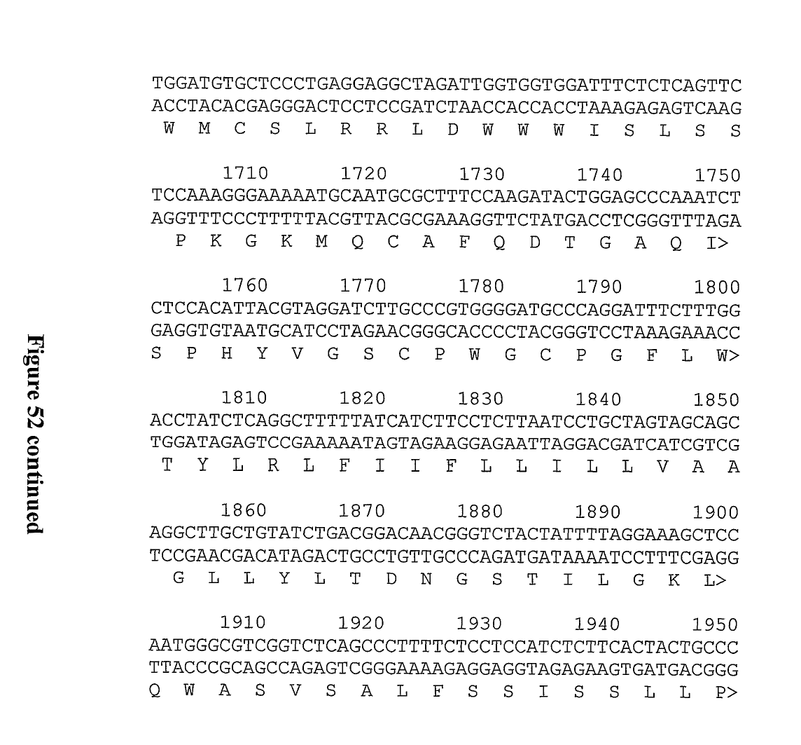

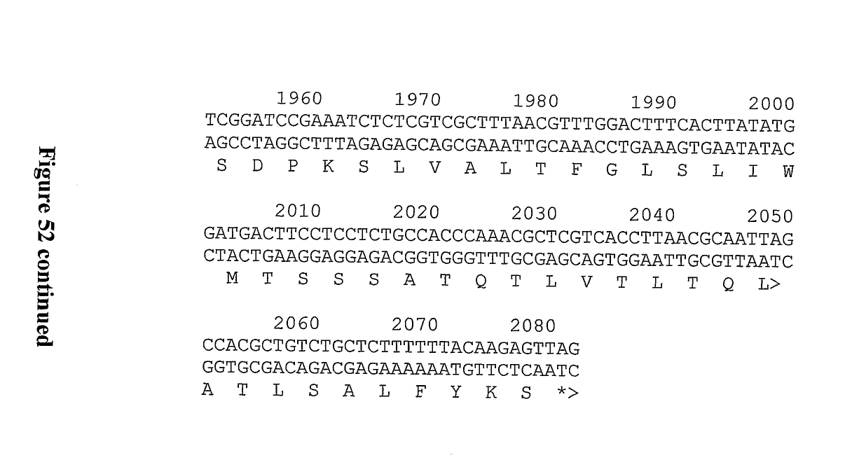

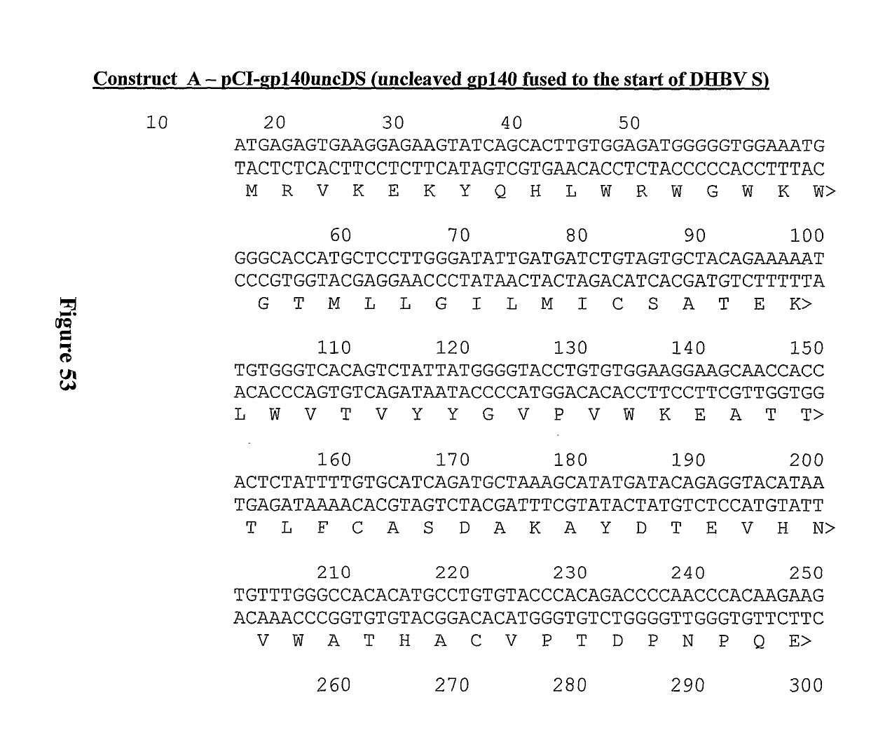

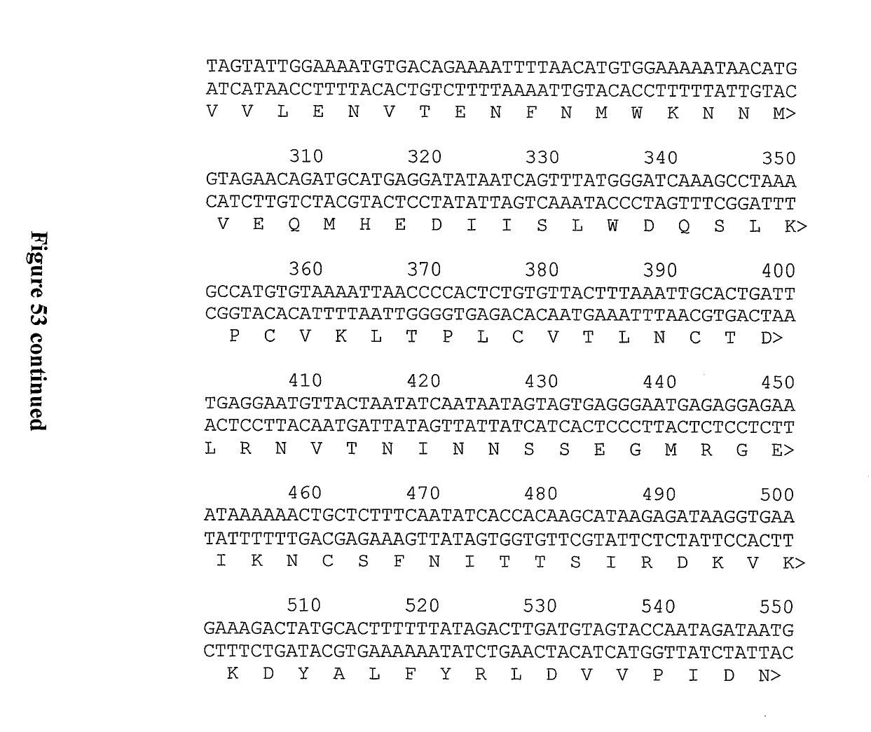

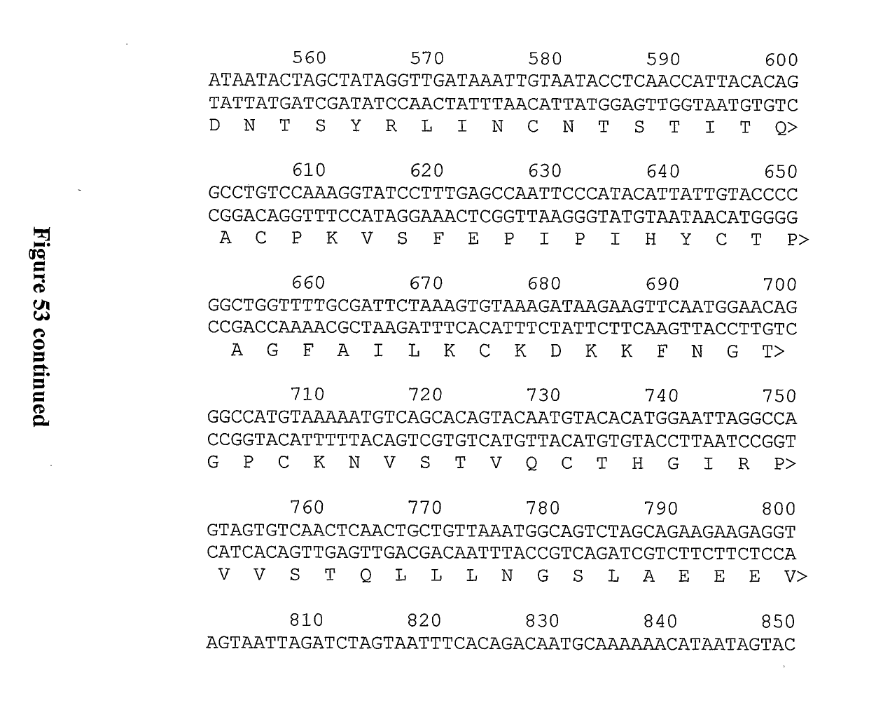

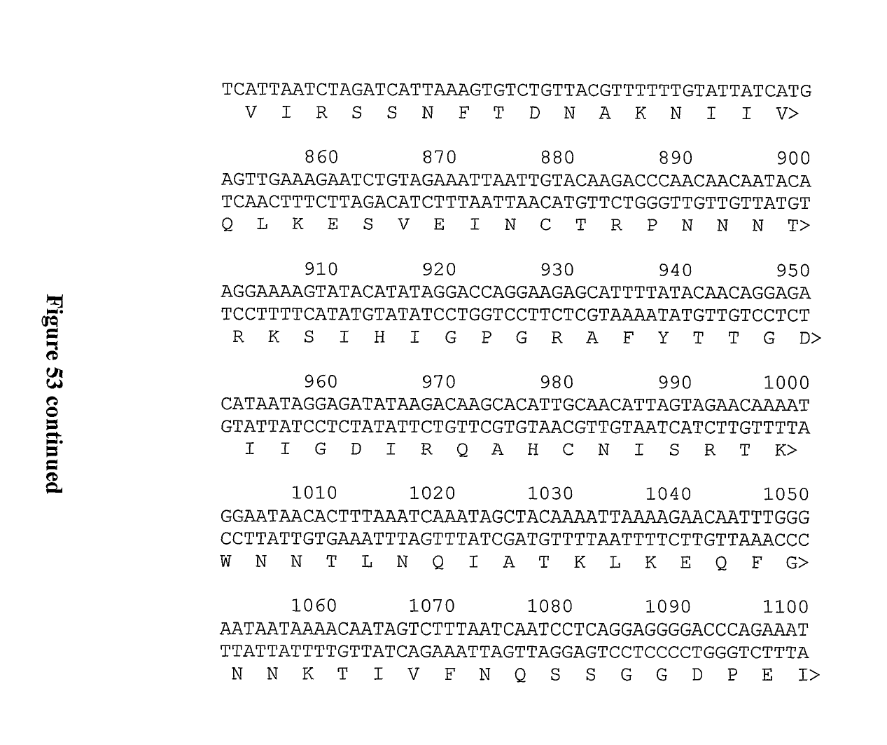

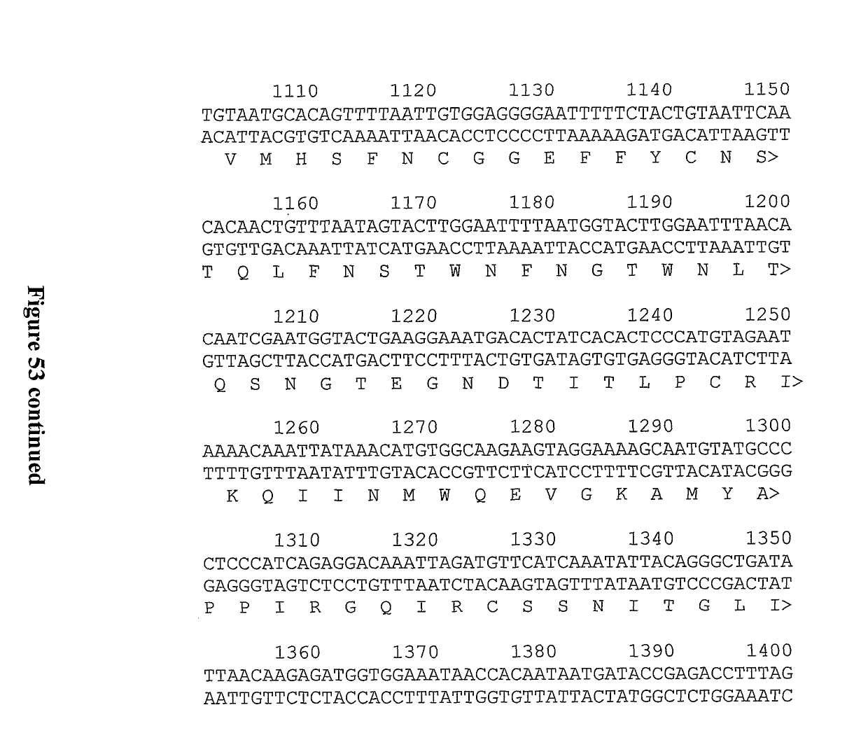

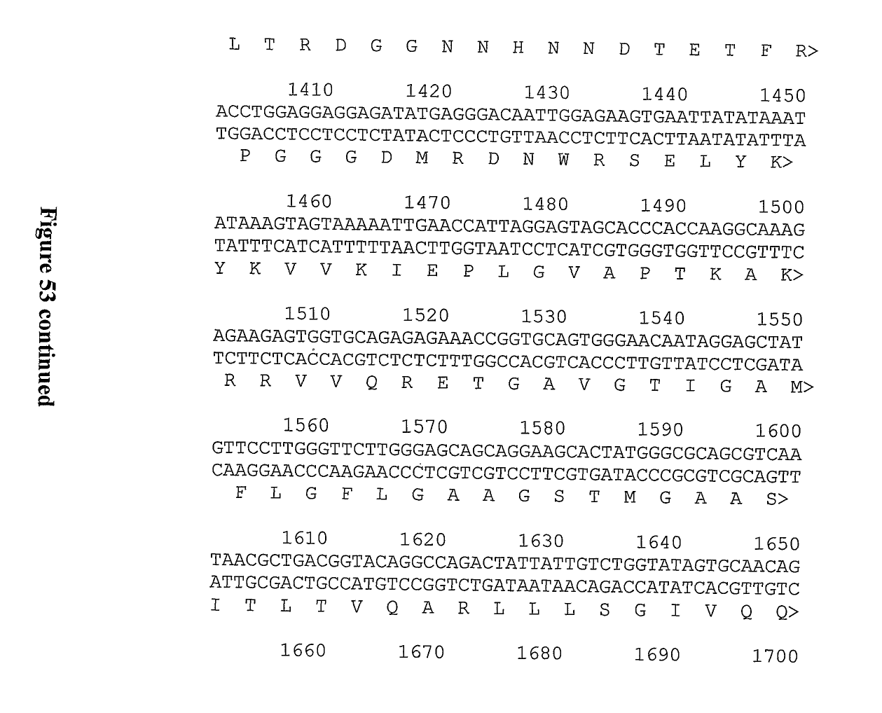

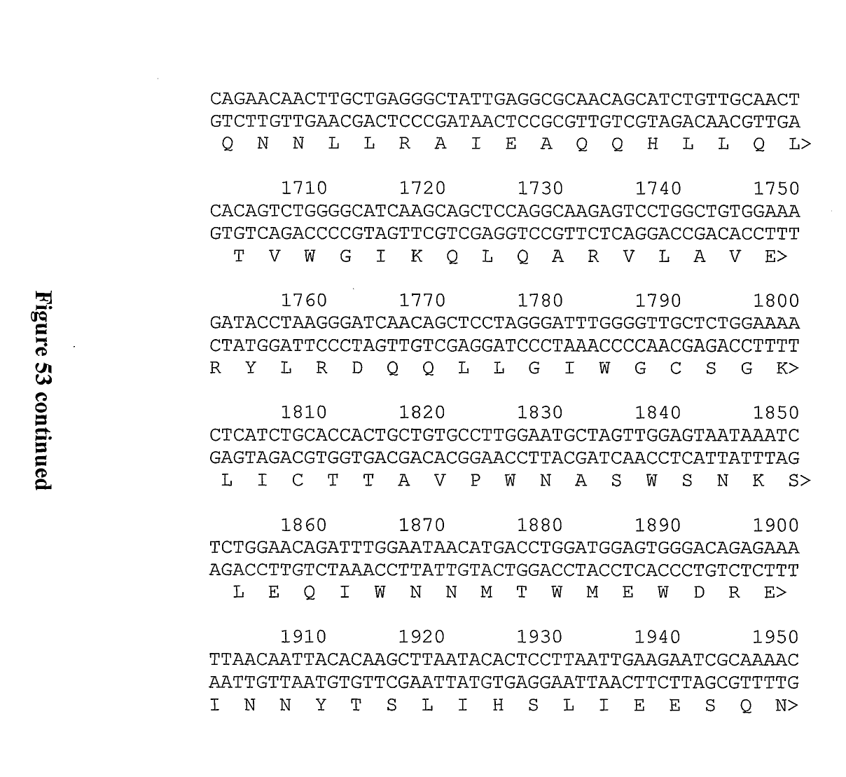

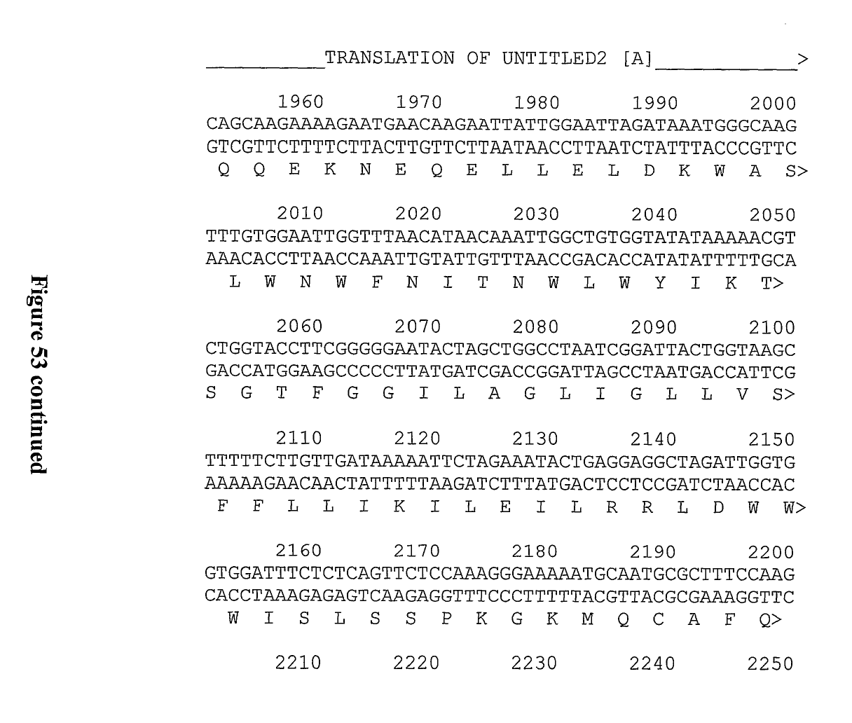

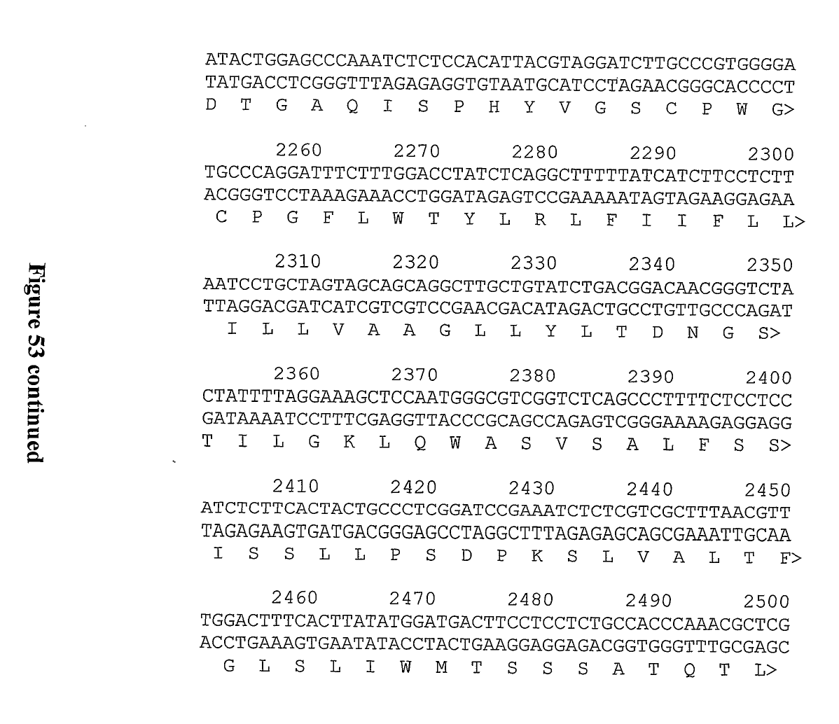

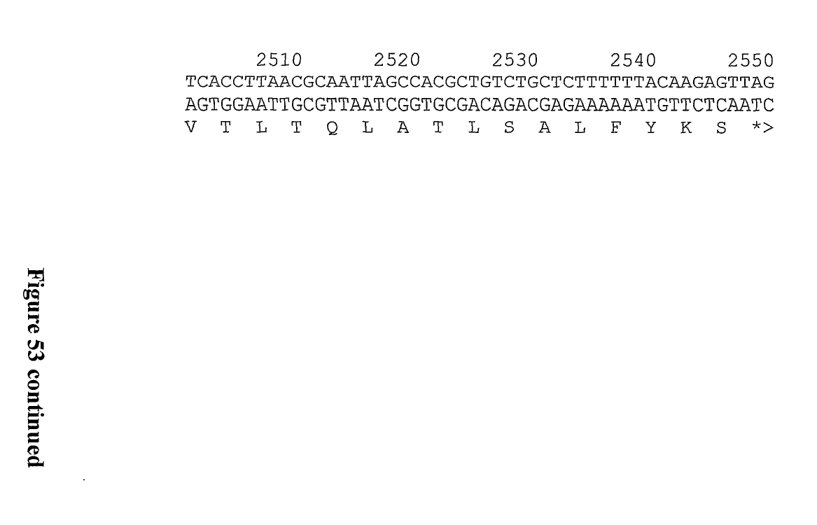

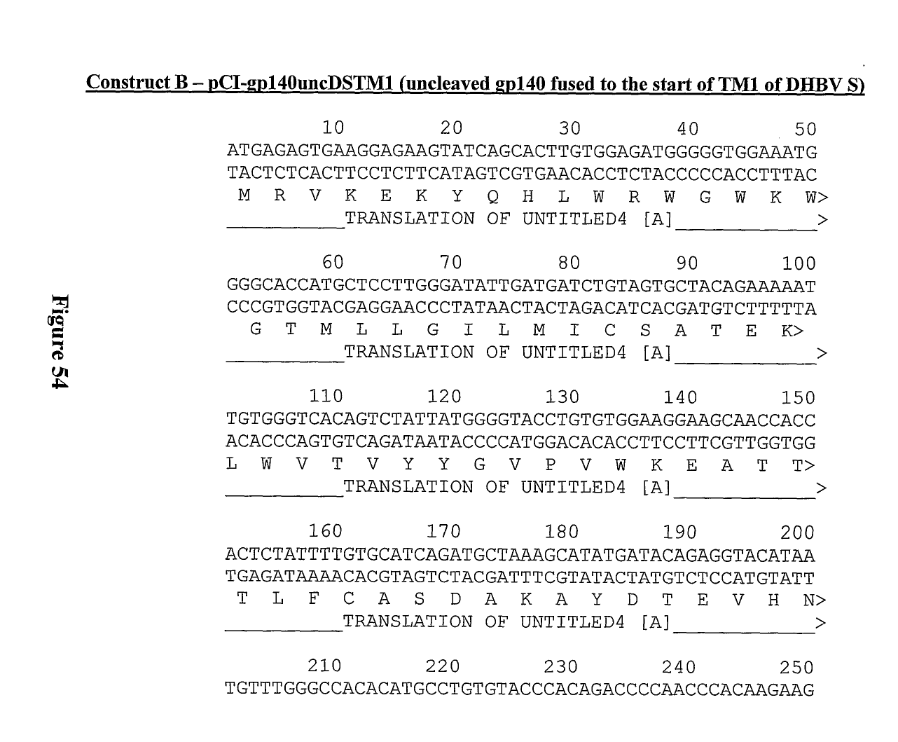

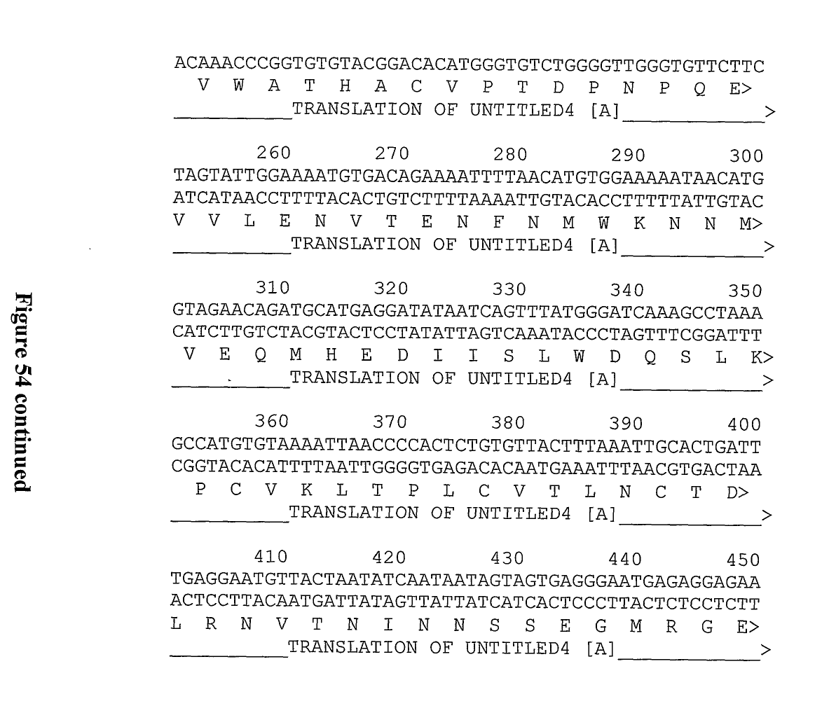

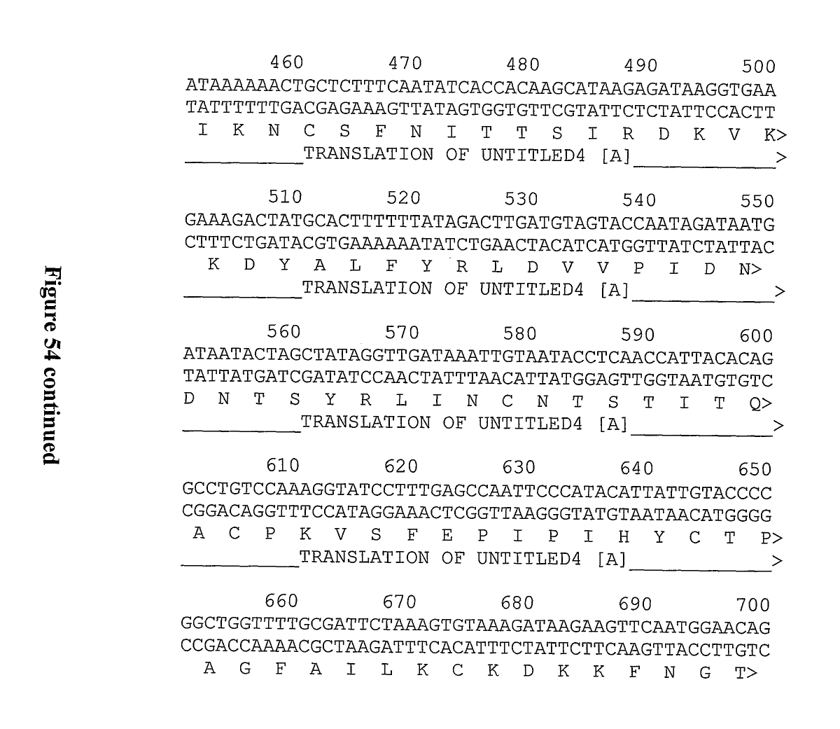

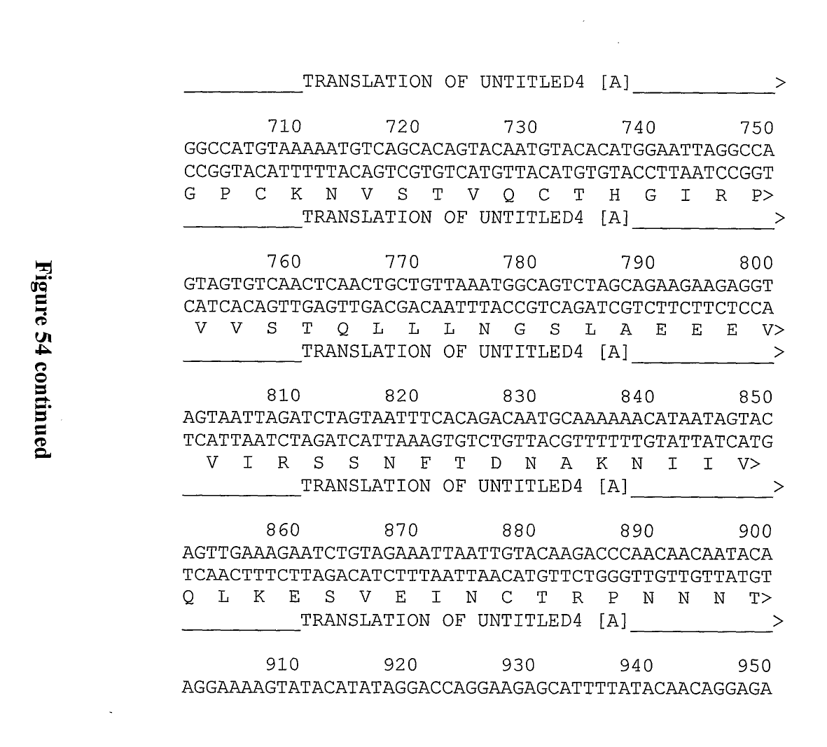

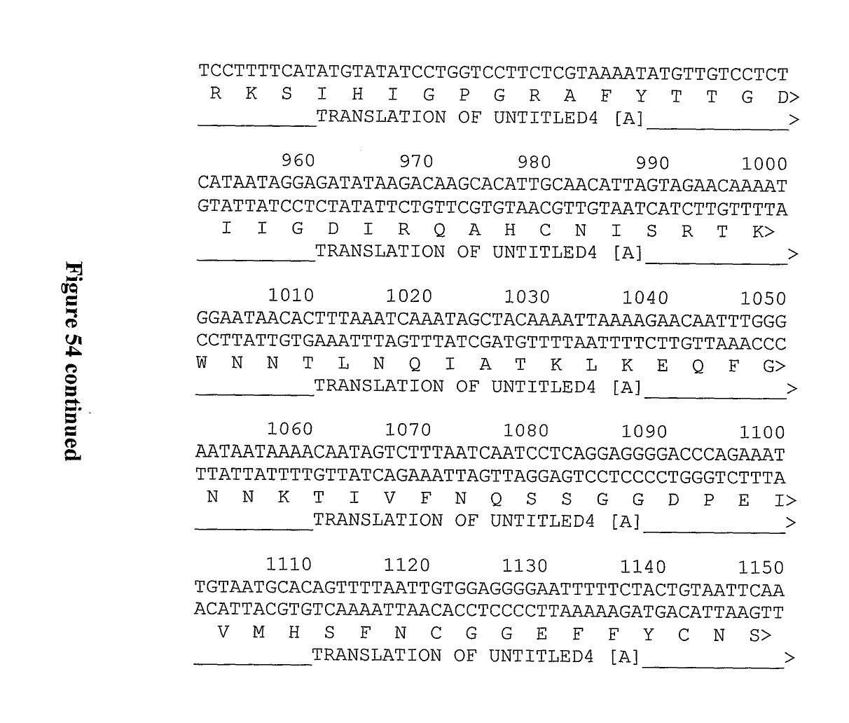

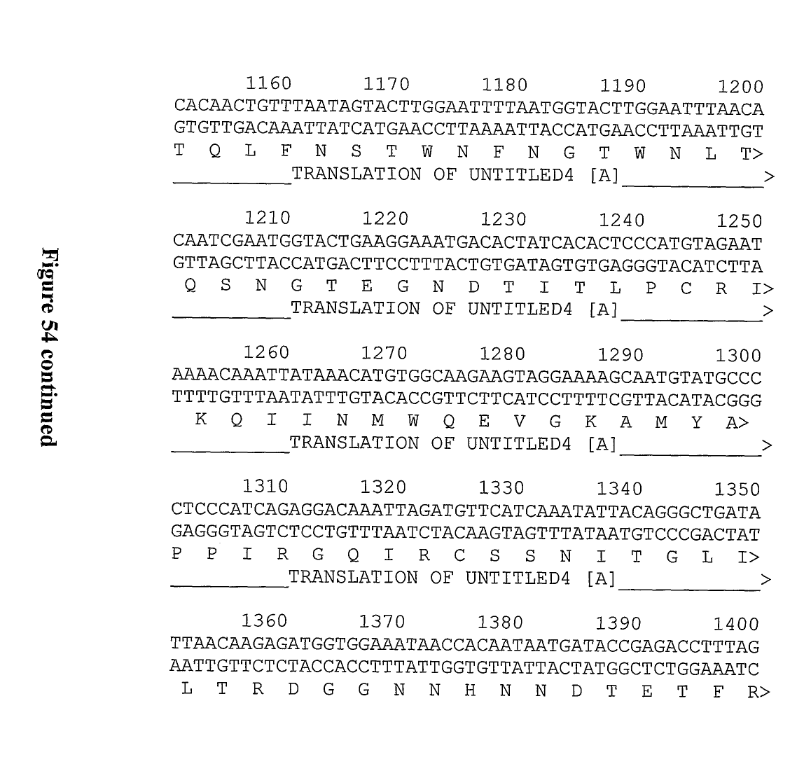

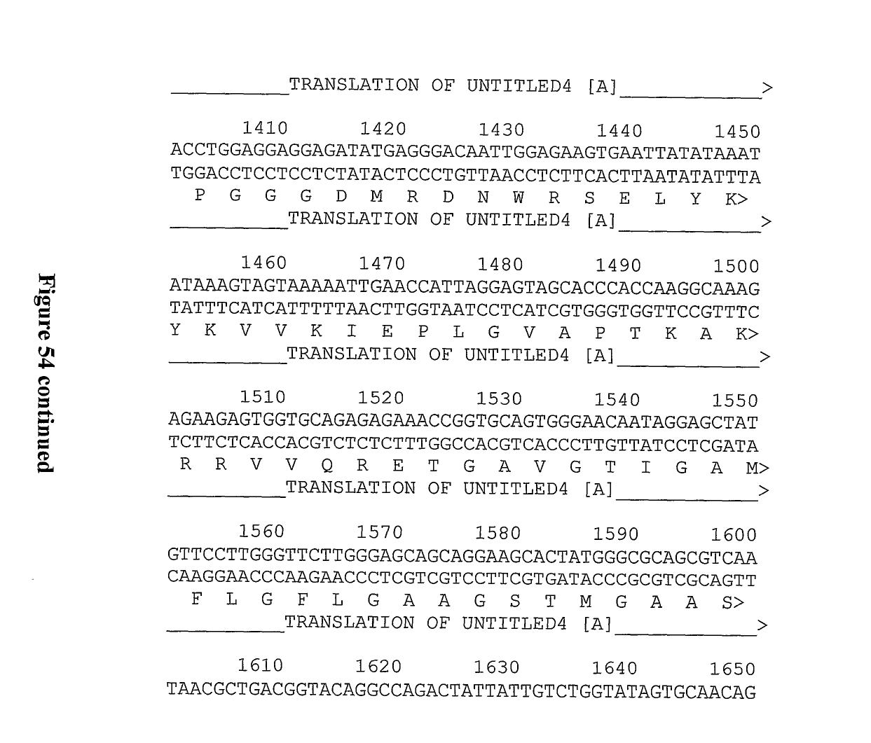

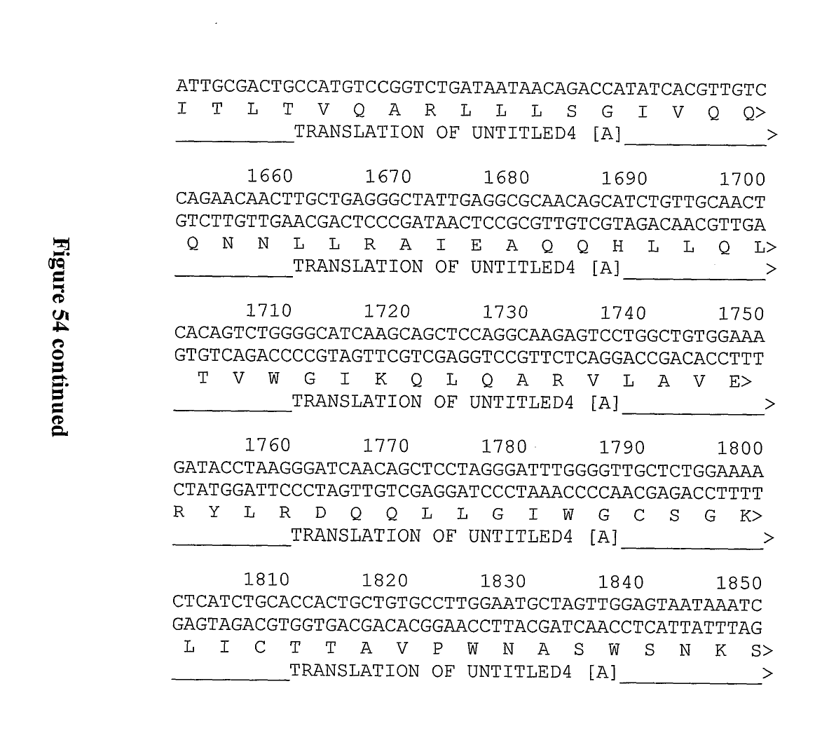

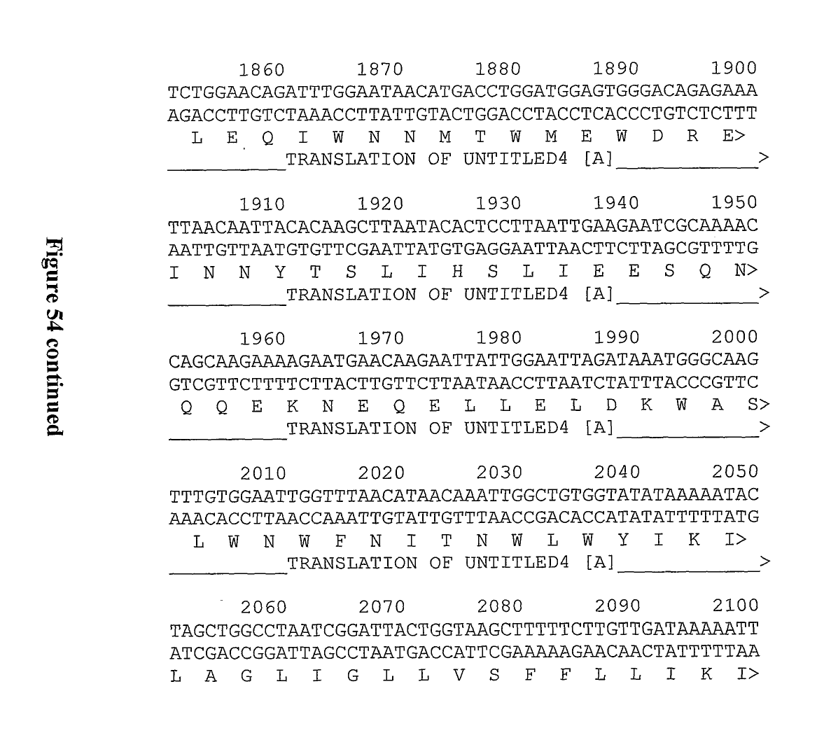

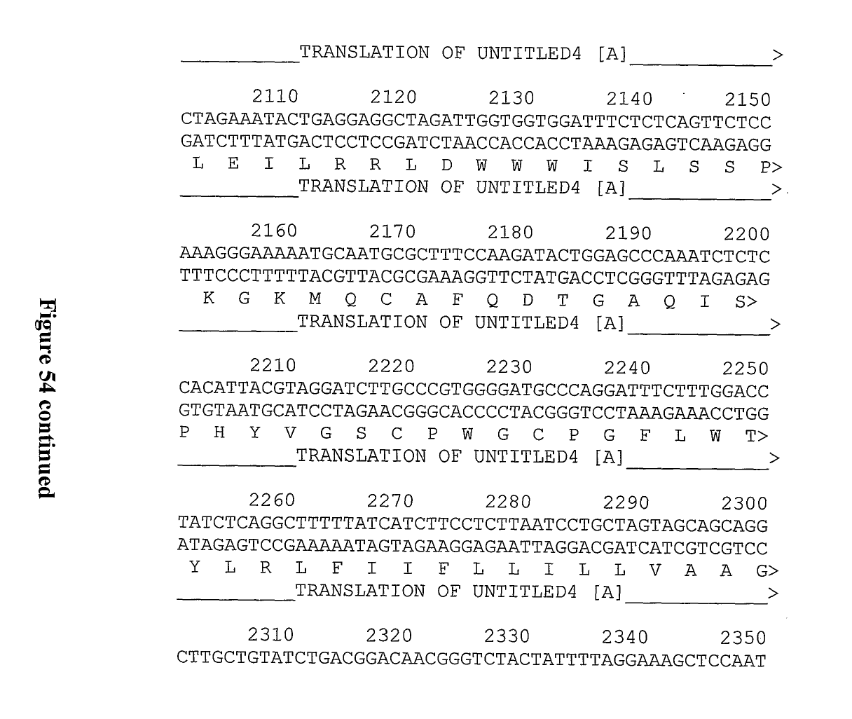

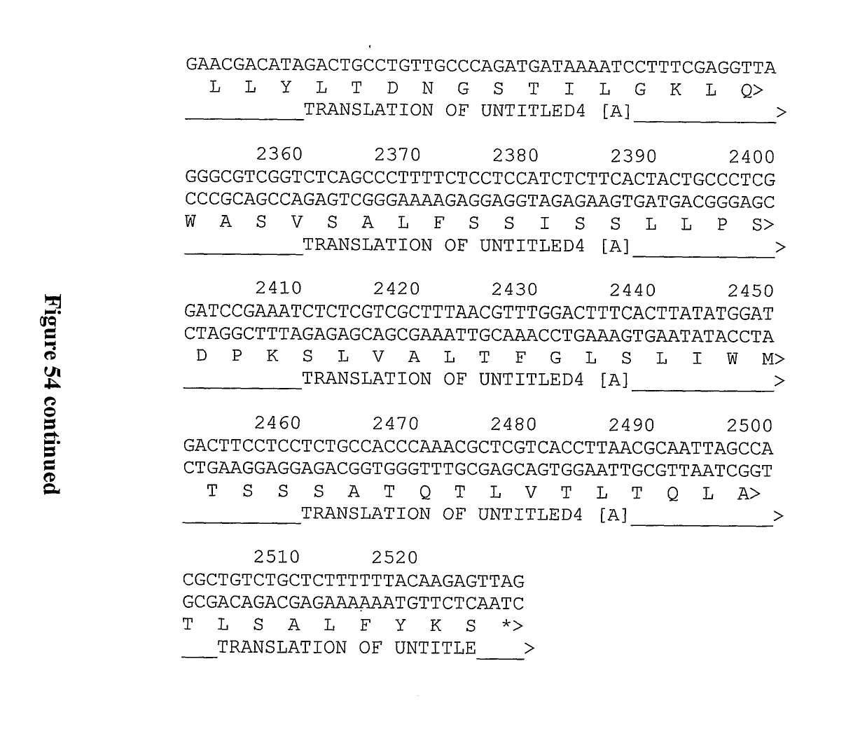

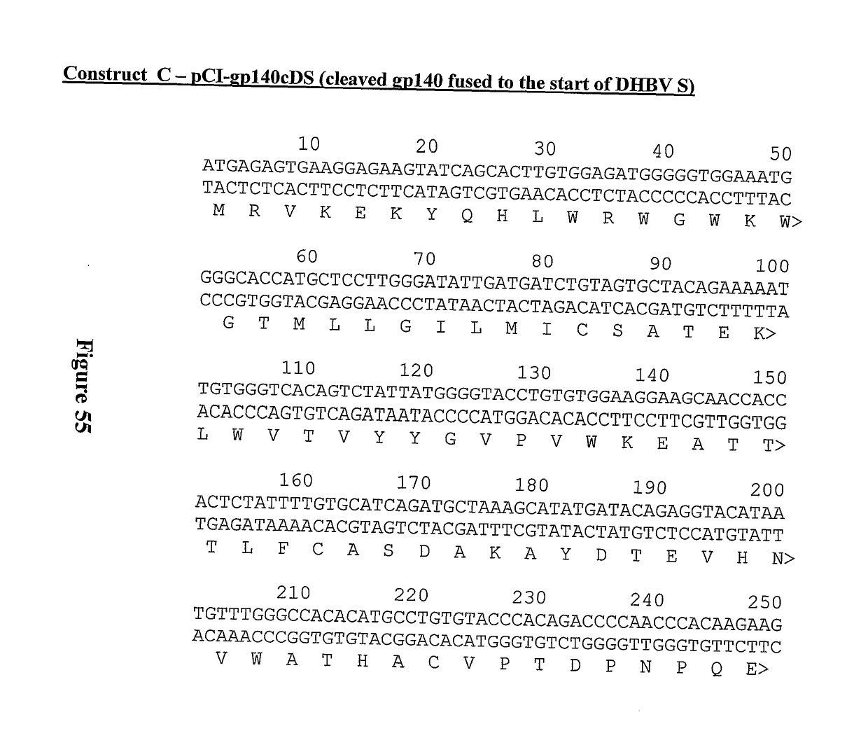

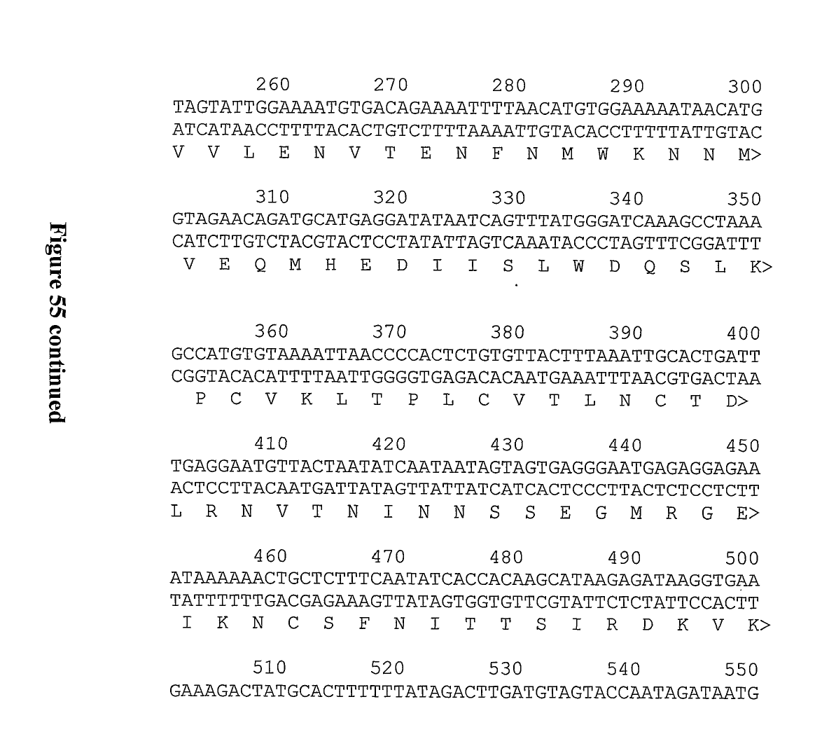

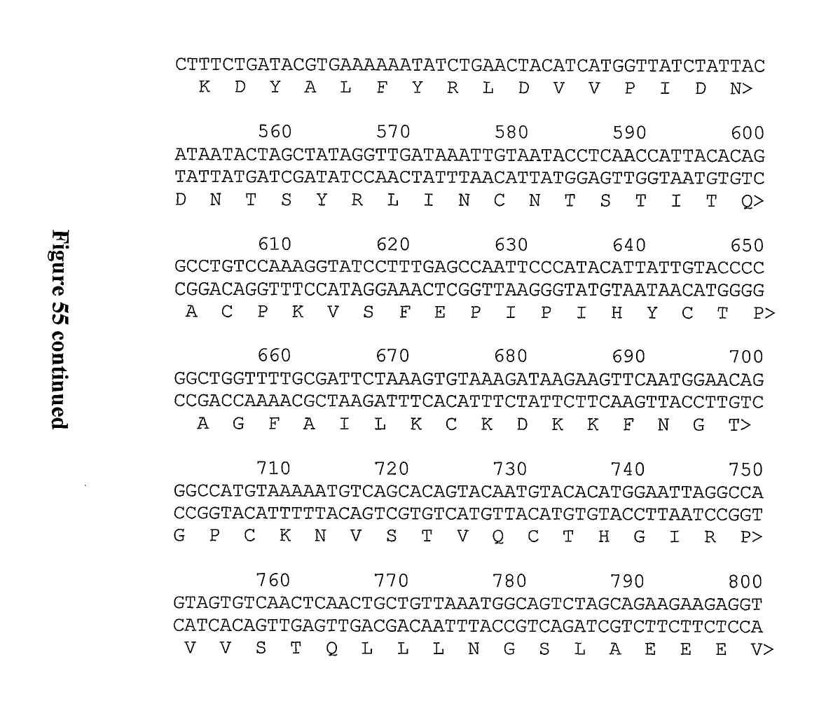

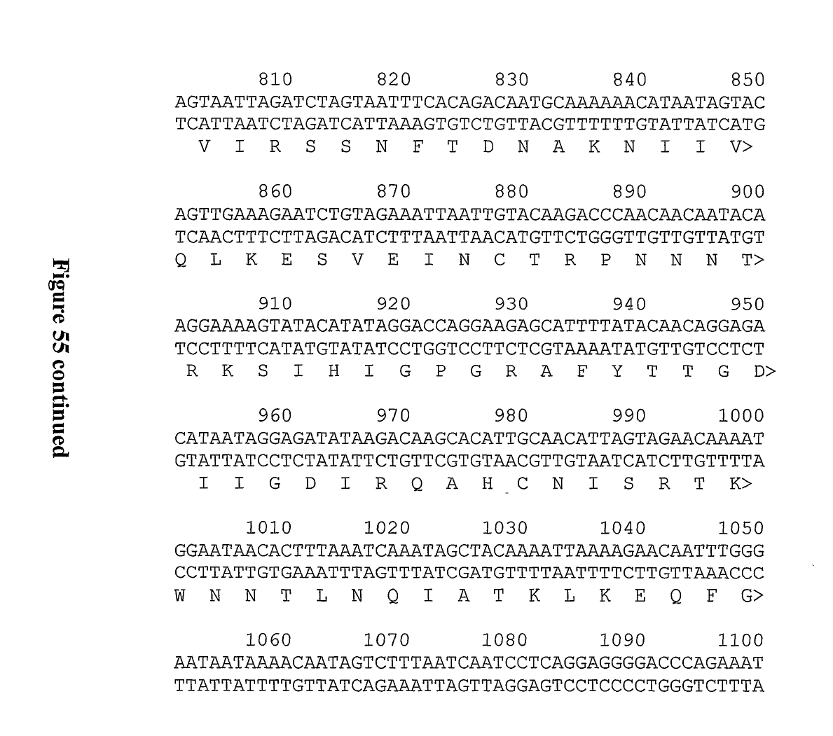

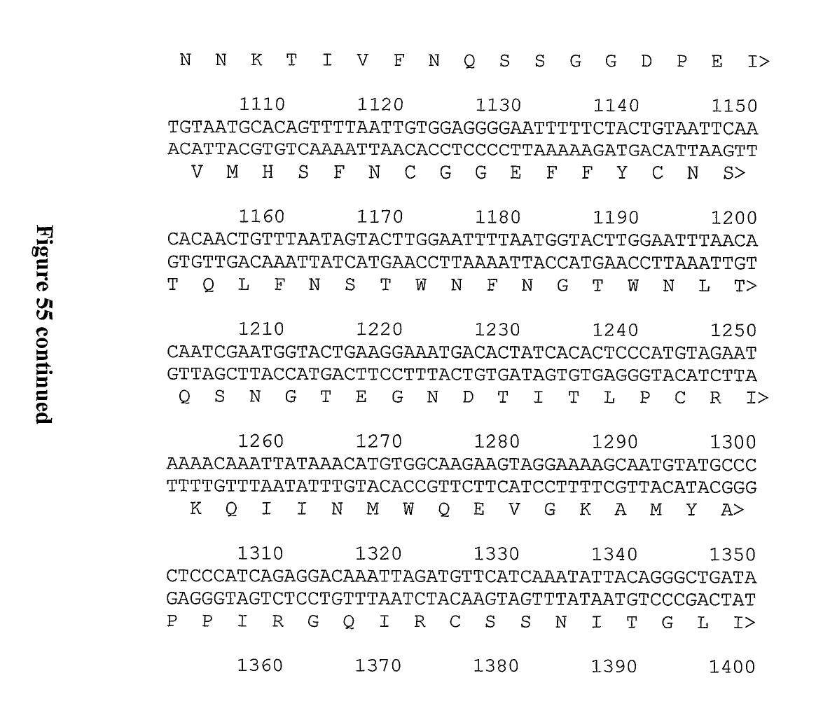

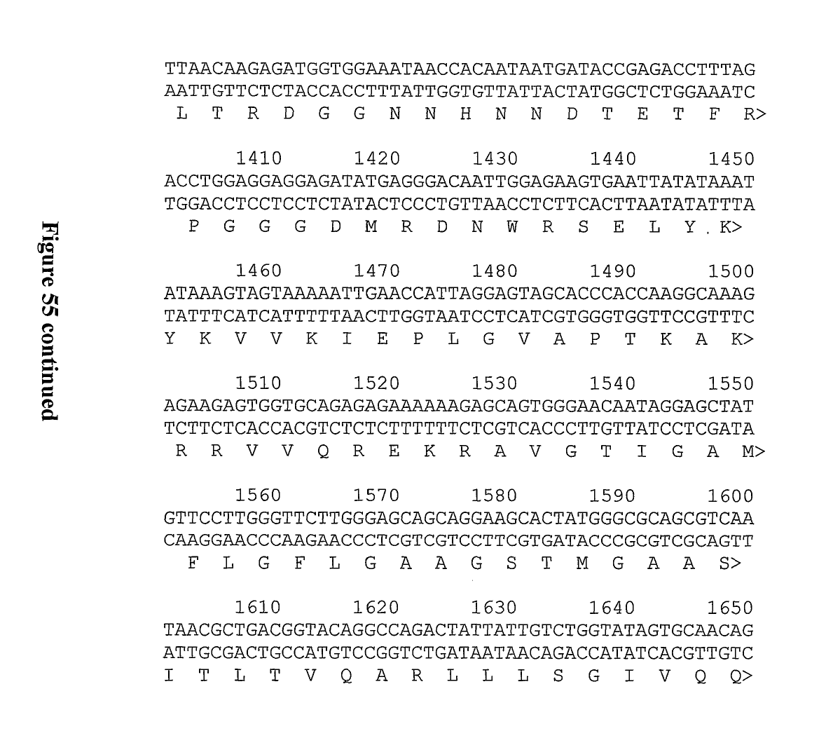

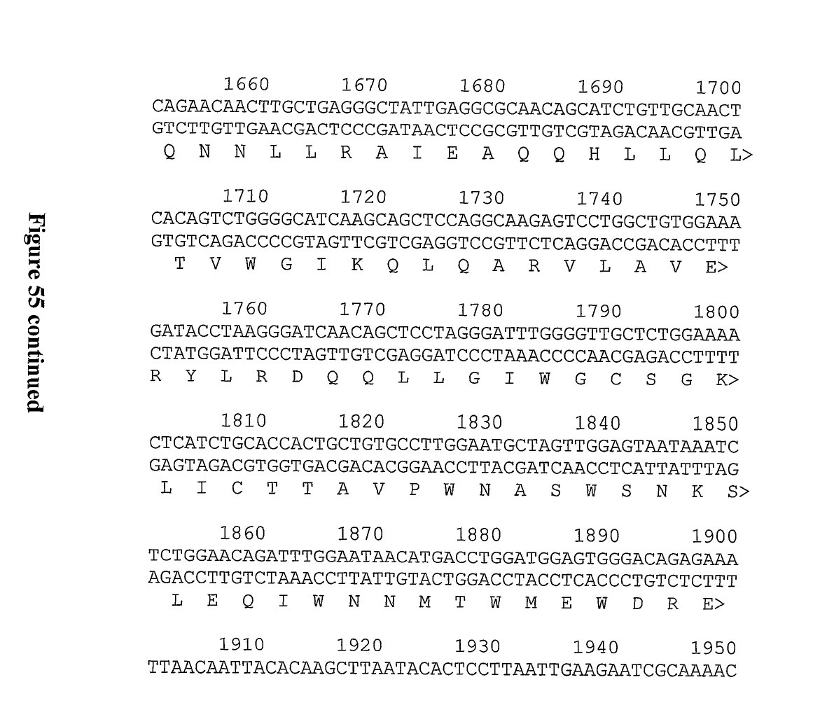

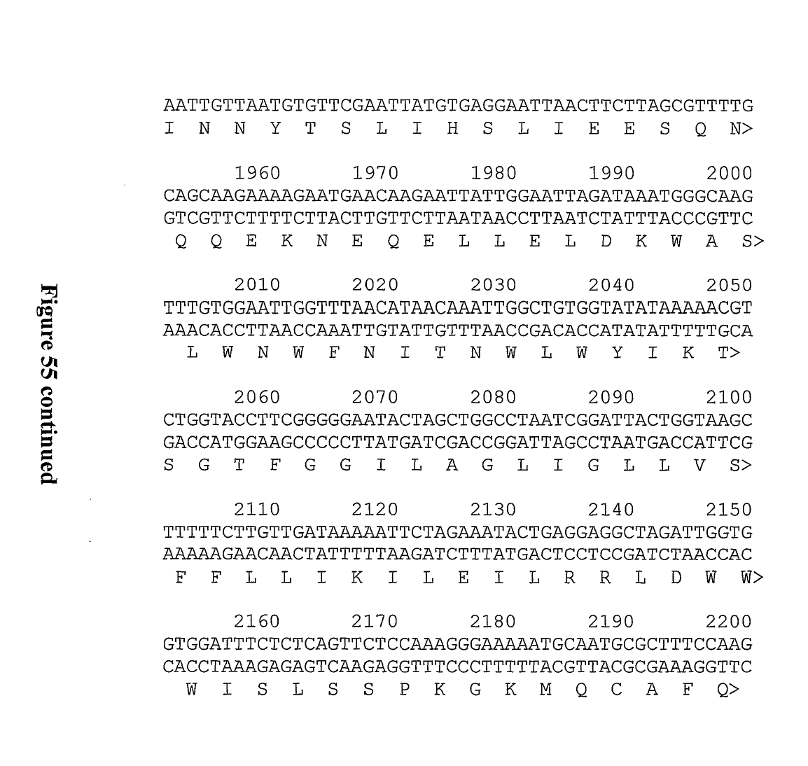

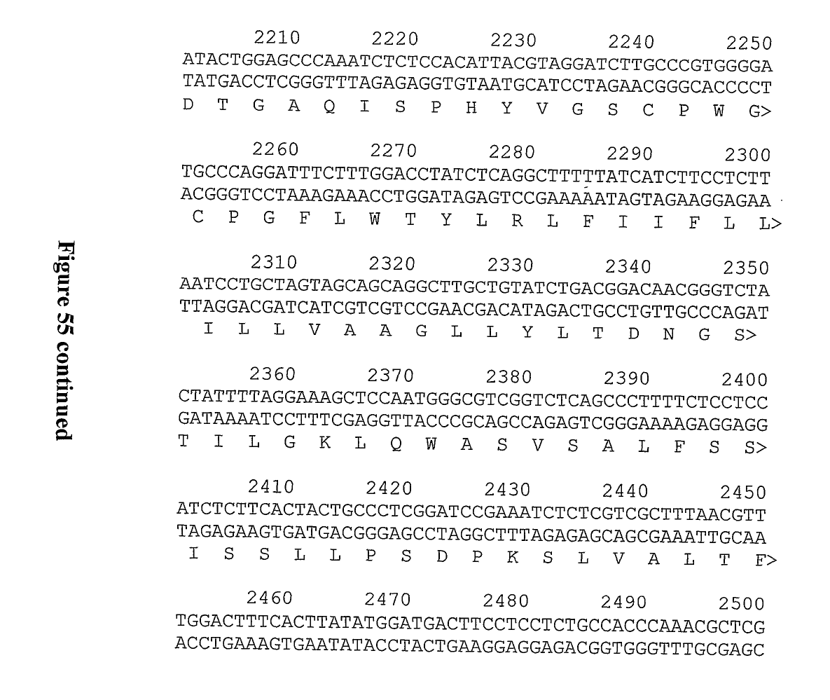

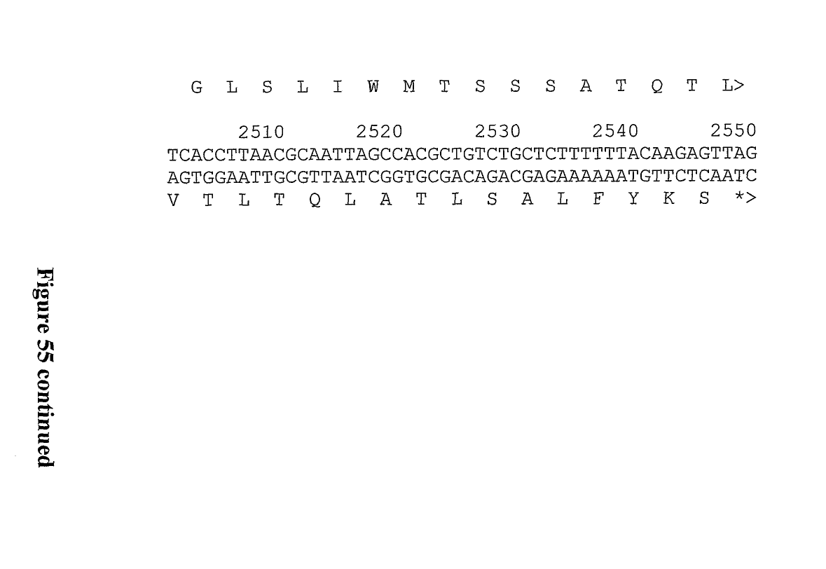

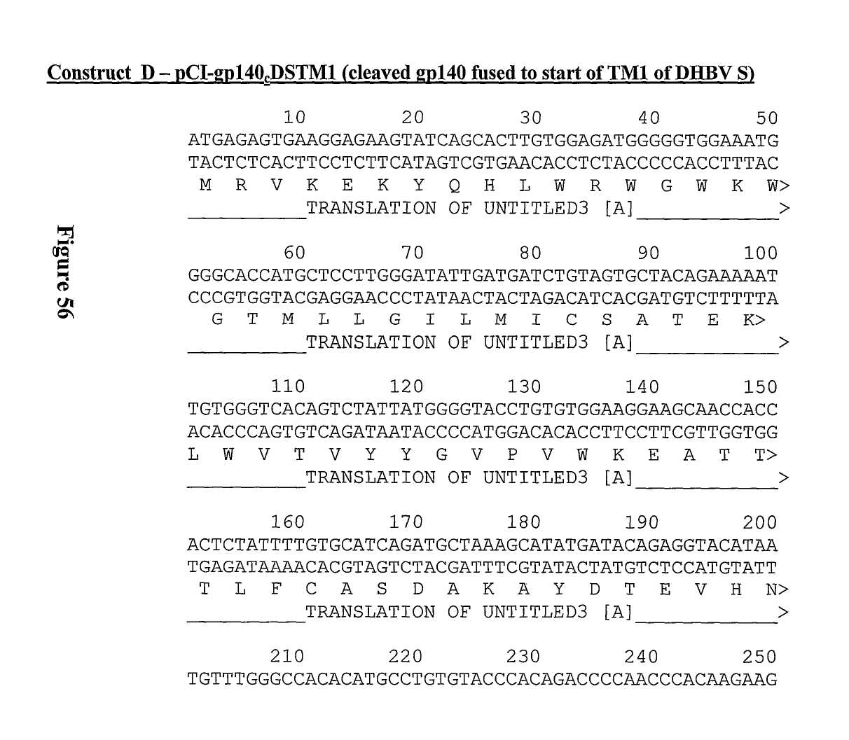

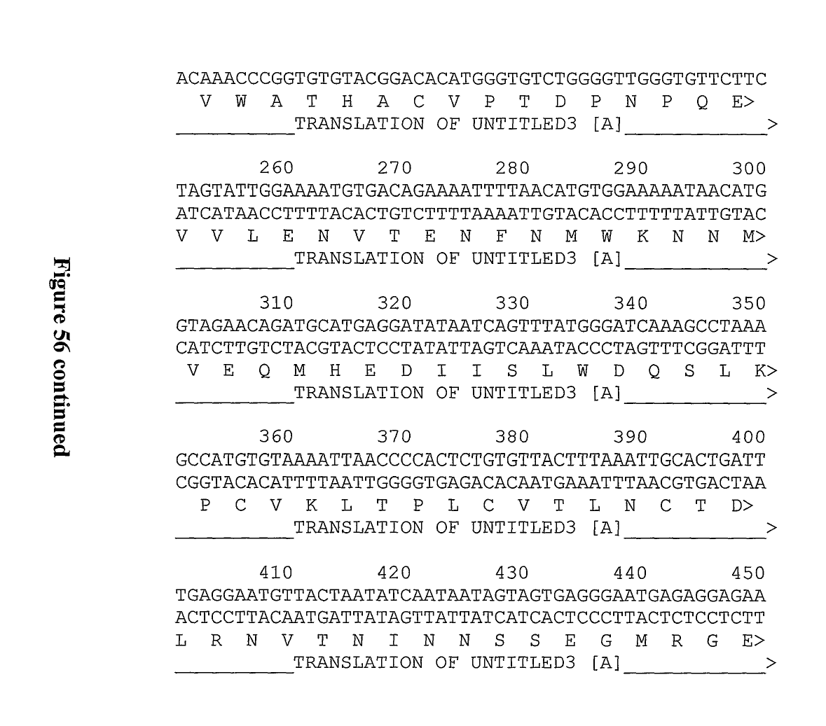

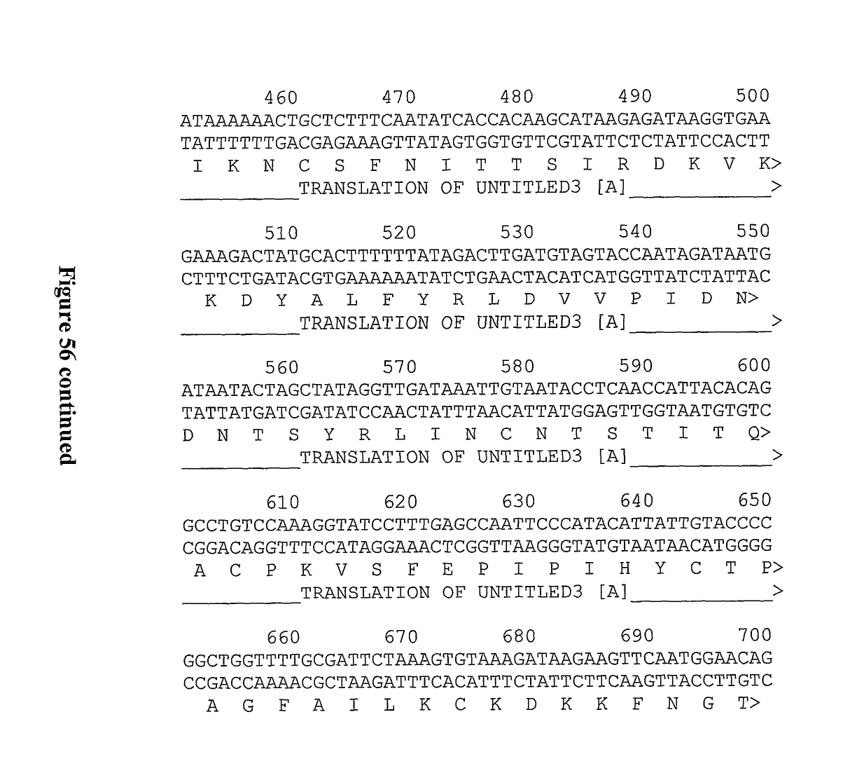

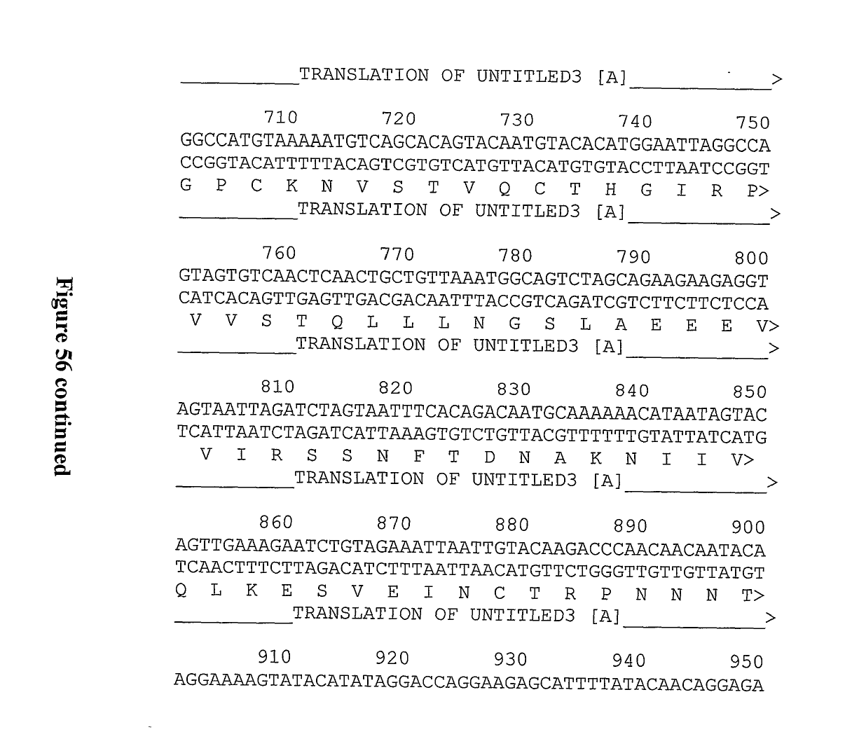

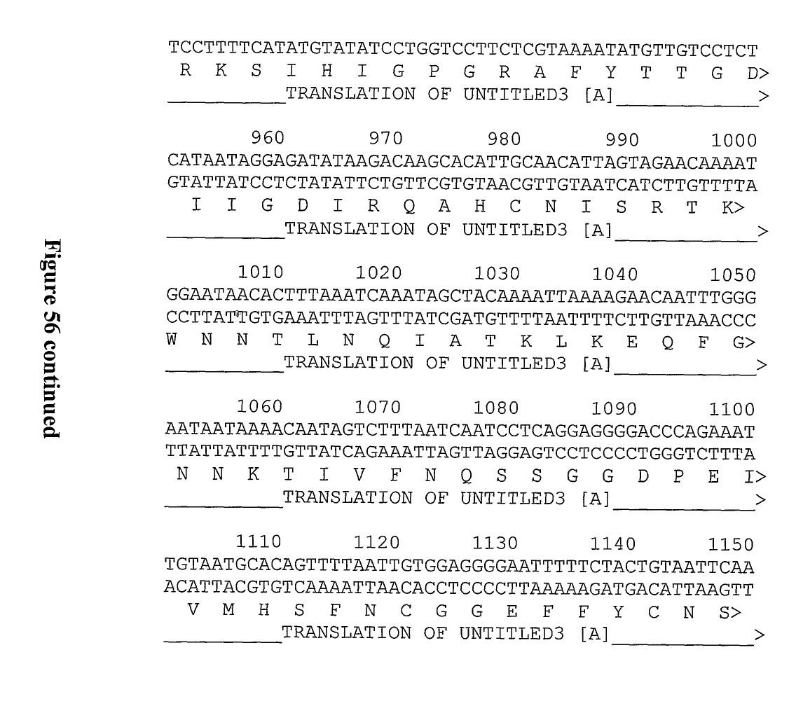

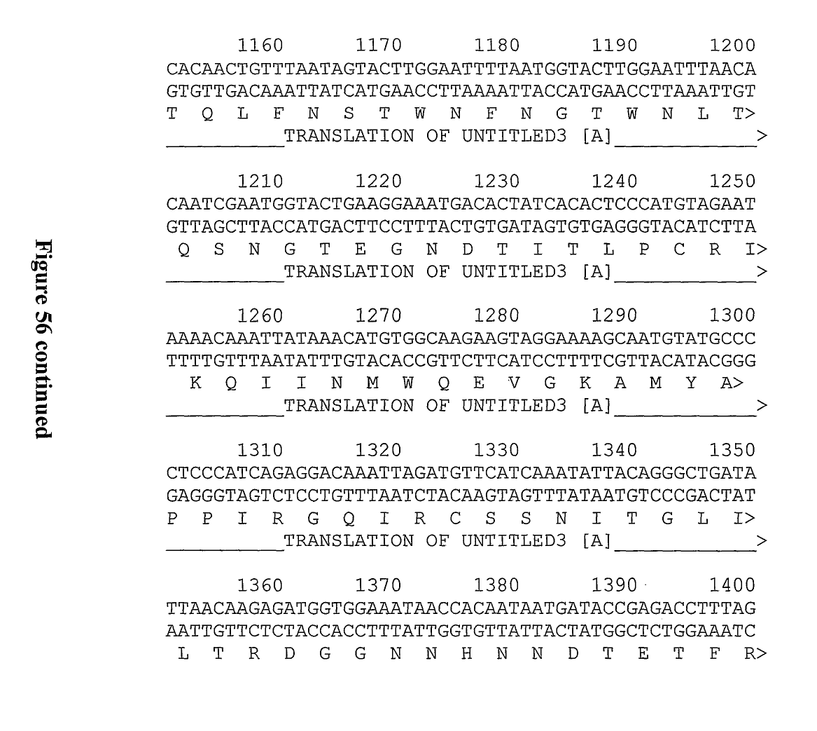

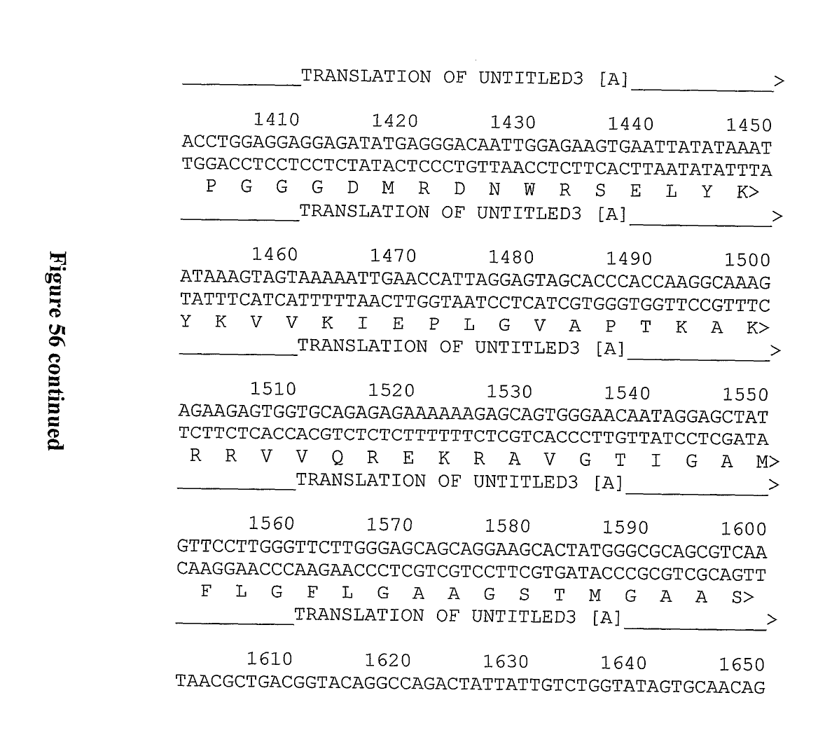

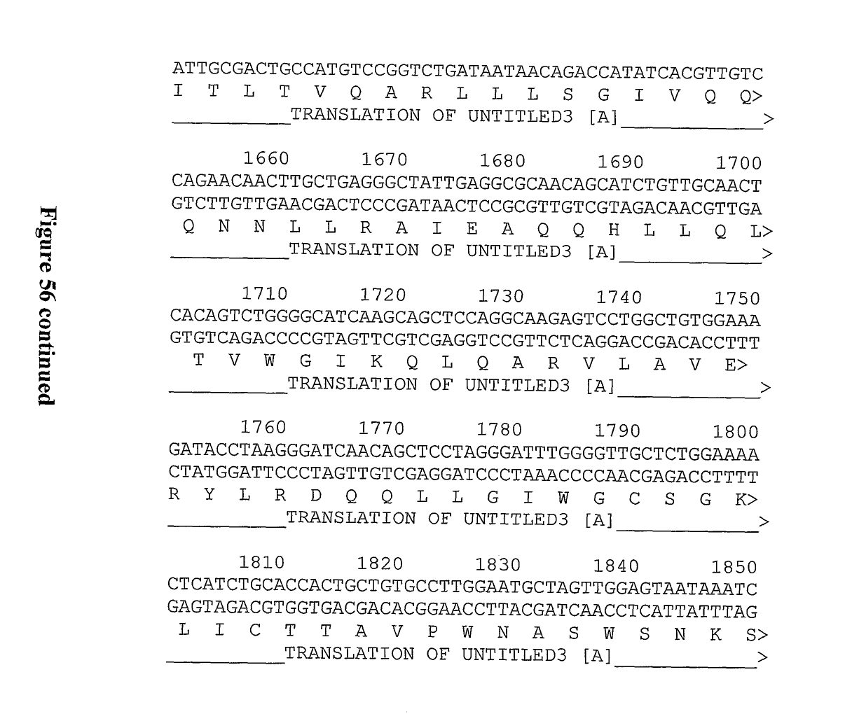

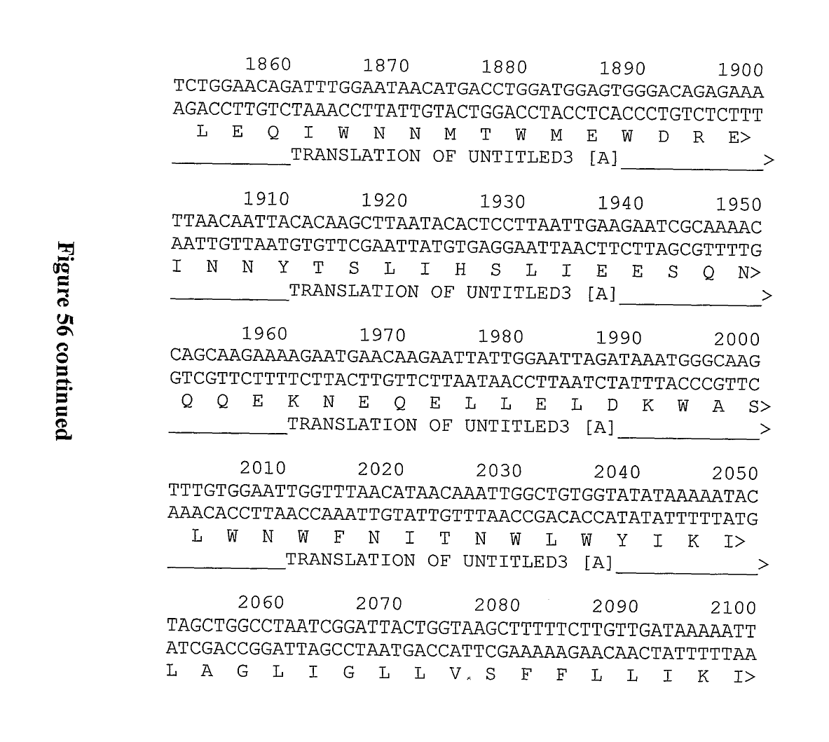

As shown in FIG. 49, gp140cDS construct (Construct F in FIG. 42) in which the furin cleavage site is wild-type, is cleaved by furin protease during synthesis and assembly, but the gp120 part of the HIV envelope protein remains associated with the VLP by virtue of its non-peptide linkage with the 41-DS part of the protein, which is assembled into the VLPs. Gp120 sedimented in association with the VLPs and was detected by Western immunoblotting with patient anti-HIV serum, and is outlined with a box for clarity.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 1
Summary of sequence identifiers

| SEQUENCE ID | |
|---|---|
| NO: | DESCRIPTION |
| SEQ ID NO: 1 | Primers for generating L-fusion proteins |
| SEQ ID NO: 2 | Primers for generating L-fusion proteins |
| SEQ ID NO: 3 | Primers for generating L-fusion proteins |
| SEQ ID NO: 4 | Primers for generating L-fusion proteins |
| SEQ ID NO: 5 | Full genomic nucleotide sequence of DHBV |
| SEQ ID NO: 6 | Nucleotide sequence encoding L polypeptide of DHBV |
| SEQ ID NO: 7 | Amino acid sequence of L polypeptide of DHBV |
| SEQ ID NO: 8 | Nucleotide sequence encoding S domain of L |
| | polypeptide of DHBV |
| SEQ ID NO: 9 | Amino acid sequence of S domain of L |
| | polypeptide of DHBV |
| SEQ ID NO: 10 | Nucleotide sequence encoding preS domain of L |
| | polypeptide of DHBV |
| SEQ ID NO: 11 | Amino acid sequence of pre S domain of L |
| | polypeptide of DHBV |
| SEQ ID NO: 12 | Nucleotide sequence encoding S polypeptide of |
| | DHBV |
| SEQ ID NO: 13 | Amino acid sequence of S polypeptide of DHBV |
| SEQ ID NO: 14 | Nucleotide sequence of Hepatitis C virus strain H77 as |
| | shown in AF011751-3 |
| SEQ ID NO: 15 | Amino acid sequence of Hepatitis C virus strain H77 |
| | as shown in AF011751-3 |
| SEQ ID NO: 16 | Nucleotide sequence encoding fusion protein H5 HA- |
| | H5ecto-S |
| SEQ ID NO: 17 | Nucleotide sequence encoding fusion protein H5HA- |
| | H5TMD-S |
| SEQ ID NO: 18 | Nucleotide sequence encoding fusion protein pCl- |
| | gp140uncDS |
| SEQ ID NO: 19 | Nucleotide sequence encoding fusion protein pCl- |
| | gp140cDS |
| SEQ ID NO: 20 | Nucleotide sequence of codon-optimised HCV E1E2-S |
| SEQ ID NO: 21 | Amino acid sequence of codon-optimised HCV |
| | E1E2-S |
| SEQ ID NO: 22 | Nucleotide sequence of influenza A HA H5ecto-S |
| SEQ ID NO: 23 | Amino acid sequence encoded by SEQ ID NO: 22 |
| SEQ ID NO: 24 | Nucleotide sequence of influenza A HA H5TMD-S |
| SEQ ID NO: 25 | Amino acid sequence encoded by SEQ ID NO: 24 |
| SEQ ID NO: 26 | Nucleotide sequence of fusion protein pCI- |
| | gp140uncDS |
| SEQ ID NO: 27 | Amino acid sequence encoded by SEQ ID NO: 26 |
| SEQ ID NO: 28 | Nucleotide sequence of fusion protein pCI- |
| | gp140uncDSTM1 |
| SEQ ID NO: 29 | Amino acid sequence encoded by SEQ ID NO: 28 |
| SEQ ID NO: 30 | Nucleotide sequence of fusion protein pCI-gp140cDS |
| SEQ ID NO: 31 | Amino acid sequence encoded by SEQ ID NO: 30 |
| SEQ ID NO: 32 | Nucleotide sequence of fusion protein pCI-gp140- |
| | _{c}DSTM1 |
| SEQ ID NO: 33 | Amino acid sequence encoded by SEQ ID NO: 32 |
| SEQ ID NO: 34 | Nucleotide sequence of fusion protein pCI- |
| | gp160uncΔCTDS |
| SEQ ID NO: 35 | Amino acid sequence encoded by SEQ ID NO: 34 |
| SEQ ID NO: 36 | Nucleotide sequence of fusion protein pCI- |
| | gp160cΔCTDS |
| SEQ ID NO: 37 | Amino acid sequence encoded by SEQ ID NO: 36 |

TABLE 2
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenylalanine, Tryptophan |
| Aromatic | Tryptophan, Tyrosine, Phenylalanine |
| Residues that influence | Glycine and Proline |
| chain orientation | |

TABLE 3
Exemplary and Preferred Amino Acid Substitutions

| | | |
|---|---|---|
| Original | | PREFERRED |
| Residue | EXEMPLARY SUBSTITUTIONS | SUBSTITUTIONS |

| | | |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, | Leu |
| | Norleu | |
| Leu | Norleu, Ile, Val, Met, | Ile |
| | Ala, Phe | |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, | Leu |
| | Norleu | |

### BIBLIOGRAPHY

Altschul et al, Nucleic Acid Research, 25:3389-3402, 1997.
Ausubel et al, Eds short protocols in Molecular Biology, John Wiley and Sons, 5^{th} Edition,
Bandyopadhyay et al., Mol. Cell. Biol., 4:749-754, 1984.
Berglund et al., Biotechnology, 11:916-920, 1993.
Berkner et al., BioTechniques, 6:616-629, 1988.
Berkner, Curr. Top. Microbiol. Immunol., 158:39-66, 1992.
Bisht et al., J. Biotechnology, 99:97-110, 2002.
Bonner et al, Cold Spring Harb. Symp. Quant. Biol., 38:308-10, 1974.
Breakefield et al., Mol. Neurobiol., 1:339-371, 1987.
Bruss J. Virol., 65:3813-3820, 1991.
Bruss et al., EMBO J., 13:2273-2279, 1994.
Buchschacher et al., J. Virol., 66:2731-2739, 1982.
Delpeyroux et al., J. Mol. Biol., 195:343-350, 1987.
Drummer et al., FEBS Lett., 546:385, 2003.
Dubuisson et al., J. of Virol. 68(16):6147-6160, 1994.
Falkowska et al., J. Virol., 81(15):8072-8079, 2007.
Fink et al., Hum. Gene Ther., 3:11-19, 1992.
Fink et al., Ann. Rev. Neurosci., 19:265-287, 1996.
Frazer, Virus Research, 89:271-274, 2002.
Freese et al., Biochem. Pharmacal., 40:2189-2199, 1990.
Gazina et al, Virology, 242:266, 1998.
Gorziglia et al., J. Virol., 66:4407-4412, 1992.
Grgacic et al, J. Gen. Virol., 79:2743, 1998.
Grgacic et al, J. Virol., 74:5116, 2000.
Grgacic, J. Gen. Virol., 83:1635, 2002.
Helle et al., J. Virol., 81(15):8101-8111, 2007.
Helseth et al., J. Virol., 64:2416-2420, 1990.
Ho et al, Gene, 77:51, 1989.
Johnson et al., J. Viral., 66:2952-2965, 1992.
Klingmuller et al., J. Viral., 67:7414-7422, 1993.
Krueger et al., Biol. Chem., 380:275-276, 1998.
Losman et al., AIDS Res. Hum. Retroviruses., 17(11):1067-1076, 2001.
Madzak et al., J. Gen. Viral. 73:1533-1536, 1992.
Mann et al., J. Viral., 54:401-407, 1985.
Margolskee, Curt Top., Microbiol. Immunol., 158:67-95, 1992.
Miller et al., Mol. Cell. Biol., 5:431-437, 1985.
Miller et al., J. Viral., 62:4337-4345, 1988.
Miller, Curr. Top. Microbiol. Immunol. 158:1-24, 1992.
Moss, Curr. Top. Microbiol. Immunol., 158:25-38, 1992.
Moss, Proc. Natl. Acad. Sci. USA, 93:11341-11348, 1996.
Muzyczka, Curt Top. Microbiol. Immunol., 158:97-129, 1992.
Naldini et al., Science, 272:263-267, 1996.
Nassal, Curt. Top. Microbial. Immunol., 214:297-337, 1996.
Netter et al., J. Viral., 75:2130-2141, 2001.
Obi et al., Gene, 89:279-282, 1990.
Op de Beek et al., J. Gen. Virology, 82:2589-2595, 2001.
Page et al., J. Viral., 64:5270-5276, 1990.
Petropoulos et al., J. Viol., 66:3391-3397, 1992.
Penin et al., Structural Biology of Hepatitis C Virus Hepatology, 39:5-19, 2004.
Prange et al., J. Gen. Viral., 76:2131-2140, 1995a.
Prange et al., EMBO J., 14:247, 1995b.
Pugh et al, J. Viral., 69:4814, 1995.
Pye et al., Vaccine, 15:1017-1023, 1997.
Quantin et al., Proc. Natl. Acad. Sci. USA, 89:2581-2584, 1992.
Rosenfeld et al., Cell, 68:143-155, 1992.
Russell et al., Nature Genetics 18:323-328, 1998.
Sambrook et al. “Molecular Cloning: A Laboratory Manual” Cold Spring Harbor Laboratory, 3rd Edition, 2001.
Schneider et al., Nature Genetics, 18:180-183, 1998.
Shimada et al., J. Clin. Invest., 88:1043-1047, 1991.
Sorge et al., Mol. Cell. Biol., 4:1730-1737, 1984.
Sprengel et al, J. Med. Virol., 15:323, 1985.
Stratford-Perricaudet et al., Hum. Gene Ther., 1:241-256, 1990.
Swaymeye et al., J. Virol., 71:9434, 1997.
Triyatni et al, J. Gen. Virol, 82:373-378, 2001.
Wilkinson et al., Nucleic Acids Res., 20:2233-2239, 1992.

1. A nucleic acid construct encoding a chimeric fusion protein wherein the nucleic acid comprises i) a contiguous sequence of nucleotides encoding a polyprotein of two or more virus envelope polypeptides and ii) a sequence of nucleotides encoding a virus-like particle-associating portion of an L polypeptide of an avian hepadnavirus.

2. The nucleic acid of claim 1 wherein the chimeric fusion protein comprises a polyprotein of two or more virus envelope polypeptides and comprises a particle-associating portion of L polypeptide, and wherein each of said the polypeptides is operably connected to a transmembrane domain and/or a protein binding domain.

3. The nucleic acid of claim 1 wherein the polyprotein is a precursor of two or more virus envelope polypeptides each comprising a transmembrane domain and/or a protein binding domain.

4. The nucleic acid of claim 2 wherein the transmembrane domain is derived from the viral envelope polyprotein or from an avian hepadnavirus L or S polypeptide.

5. The nucleic acid of claim 2 wherein transmembrane domain or protein binding domain mediates binding of at least one of said viral envelope protein polypeptides to the VLP via non-peptide bonds.

6. The nucleic acid of claim 1 wherein the protein binding domain contains residues for the formation of a disulphide bond between said envelope polypeptides or between an envelope polypeptide and L or S polypeptide.

7. The nucleic acid construct of claim 1 wherein the virus envelope polypeptide is a Flavivirus, Coronavirus, Herpesvirus, Hepadnavirus, Retrovirus, Orthomyxovirus or Paramyxovirus envelope polypeptide or a functional variant thereof.

8. The nucleic acid of claim 7 wherein the virus envelope protein is a Flaviviridae (eg hepatitis C virus), Orthomyxoviridae (eg influenza), Paramyxovirus (eg measles virus) or Retroviridae (eg human immunodeficiency virus (HIV)) virus envelope polypeptide or a functional variant thereof.

9. The nucleic acid of claim 1 wherein the particle-associating portion of L polypeptide comprises all or part of the S domain of L polypeptide of avian hepadnavirus, S domain of L minus the TM1 domain, L polypeptide absent the pre-S domain or absent the TM1 region of the S domain, and sequences of the L polypeptide downstream of the TM1, or at least TM2 including the 5′ cysteine loop between TM1 and TM2 and downstream sequences of L polypeptide.

10. The nucleic acid of claim 1 wherein the sequence of nucleotides encoding a particle-associating portion of L polypeptide is selected from SEQ ID NO: 8, nucleotides 1581 to 2076 of SEQ ID NO: 16, nucleotides 1663 to 2082 of SEQ ID NO: 17, nucleotides 2047 to 2550 of SEQ ID NO: 18, or a functional variant of one of these having at least 90% sequence identity thereto or a functional variant of one of these which hybridises to its complement under at least medium stringency hybridisation conditions.

11. The nucleic acid of claim 1 wherein the polyprotein is E1 E1 of hepatitis C virus.

12. The nucleic acid of claim 11 comprising the nucleotide sequence as set forth in SEQ ID NO: 20 or a functional variant thereof having at least 95% sequence identity thereto or a sequence that hybridises to SEQ ID NO:20 or to a complementary sequence thereof under at least medium stringency hybridisation conditions.

13. The nucleic acid of claim 1 wherein the polyprotein is hemagglutinin (HA) of influenza A virus.

14. The nucleic acid construct of claim 13 comprising the nucleotide sequence as set forth in SEQ ID NO: 22 or 24 or a functional variant thereof having at least 95% sequence identity thereto or a sequence that hybridises to SEQ ID NO: 22 or 24 or a complementary sequence of either of these under at least medium stringency hybridisation conditions.

15. The nucleic acid of claim 1 wherein the polyprotein is gp160 or gp140 of HIV.

16. (canceled)

17. (canceled)

18. (canceled)

19. (canceled)

20. The nucleic acid of claim 15 comprising the nucleotide sequence as set forth in SEQ ID NO: 18, 19, 26, 28, 30, 32, 34, or 36 or a functional variant thereof having at least 95% sequence identity thereto or a sequence that hybridises to a complementary sequence thereof under at least medium stringency hybridisation conditions.

21. The nucleic acid of claim 11 wherein the fusion protein comprises a sequence of amino acids as set forth in SEQ ID NO: 21 or a functional portion thereof or a functional variant thereof having at least 95% sequence identity.

22. The nucleic acid of claim 13 wherein the fusion protein comprises a sequence of amino acids as set forth in SEQ ID NO: 23 or 25 or a functional portion thereof or a functional variant thereof having at least 95% sequence identity.

23. The nucleic acid of claim 15 wherein the fusion protein comprises a sequence of amino acids as set forth in SEQ ID NO: 27, 29, 31, 33, 35, or 37 or a functional portion thereof or a functional variant thereof having at least 95% sequence identity.

24. The nucleic acid of claim 1 wherein the avian hepadnavirus is a duck hepatitis B virus (DHBV).

25. The nucleic acid of claim 1 further comprising a sequence of nucleotides encoding an S polypeptide of an avian hepadnavirus.

26. An expression vector comprising the nucleic acid of claim 1 operably connected to an expression control sequence.

27. (canceled)

28. A cultured cell comprising the vector of claim 26.

29. (canceled)

30. (canceled)

31. The cell of claim 28 wherein the cell is further comprises an expression vector comprising a sequence of nucleotides encoding a polypeptide having the function of an S polypeptide of avian hepadnavirus.

32. (canceled)

33. A method of producing a protein, the method comprising culturing the cell of claim 28 for a time and under conditions permitting expression under the control of the expression control sequence, and optionally purifying the polypeptide from the cell or medium of the cell.

34. A method of producing a virus-like particle, the method comprising culturing the cell of claim 31 for a time and under conditions permitting expression under the control of the expression control sequence and formation of a virus-like particle, and optionally purifying the virus-like particle from the cell or medium of the cell.

35. A virus-like particle produced by the method of claim 34.

36. A chimeric virus-like particle comprising S polypeptide of avian hepadnavirus or a functional variant thereof and i) a chimeric fusion protein comprising a viral envelope polypeptide produced from a polyprotein, covalently attached to a particle-associating portion of L polypeptide of avian hepadnavirus and ii) a second or further viral envelope polypeptide also produced from said polyprotein, associated with the virus-like particle by a non-peptide bond.

37. The virus-like particle of claim 36 wherein the chimeric fusion protein comprises a polyprotein of two or more virus envelope polypeptides and comprises a particle-associating portion of L polypeptide, and wherein each of said polyprotein polypeptides is operably connected to a transmembrane domain and/or a protein binding domain.

38. The virus-like particle of claim 36 wherein the polyprotein is a precursor of two or more virus envelope polypeptides each comprising a transmembrane domain and/or a protein binding domain.

39. The virus-like particle of claim 37 wherein the transmembrane domain is derived from the viral envelope polyprotein or from an avian hepadnavirus L or S polypeptide.

40. The virus-like particle of claim 37 wherein transmembrane domain or protein binding domain mediates binding of at least one viral envelope protein to the VLP via non-peptide bonds.

41. The virus-like particle of claim 36 wherein the protein binding domain contains residues for the formation of a disulphide bond between said envelope polypeptides or between an envelope polypeptide and L or S polypeptide.

42. The virus-like particle of claim 36 wherein the virus envelope polypeptide is a Flavivirus, Coronavirus, Herpesvirus, Hepadnavirus, Retrovirus, Orthomyxovirus or Paramyxovirus envelope polypeptide or a functional variant thereof.

43. The virus-like particle of claim 42 wherein the virus envelope protein is a Flaviviridae (eg hepatitis C virus), Orthomyxoviridae (eg influenza), Paramyxovirus (eg measles virus) or Retroviridae (eg human immunodeficiency virus (HIV)) virus envelope polypeptide or a functional variant thereof.

44. The virus-like particle of claim 36 wherein the particle-associating portion of L polypeptide comprises all or part of the S domain of L polypeptide of avian hepadnavirus, the S domain of L minus the TM1 domain, the L polypeptide absent the pre-S domain or absent the TM1 region of the S domain, or the sequences of the L polypeptide downstream of the TM1, or at least TM2, including the 5′ cysteine loop between TM1 and TM2, and downstream sequences of L polypeptide.

45. The virus-like particle of claim 36 wherein particle-associating portion of L polypeptide is encoded by a sequence of nucleotides selected from SEQ ID NO: 8, nucleotides 1581 to 2076 of SEQ ID NO: 16, nucleotides 1663 to 2082 of SEQ ID NO: 17 or nucleotides 2047 to 2550 of SEQ ID NO: 18, or a functional variant of one of these having at least 95% sequence identity thereto or a functional variant of one of these which hybridises to its complement under at least medium stringency hybridisation conditions.

46. The virus-like particle of claim 36 wherein the polyprotein is E1E1 of hepatitis C virus.

47. The virus-like particle of claim 46 wherein the chimeric fusion protein is encoded by the nucleotide sequence as set forth in SEQ ID NO: 20 or a functional variant thereof having at least 95% sequence identity thereto or a sequence that hybridises to SEQ ID NO:20 or to a complementary sequence thereof under at least medium stringency hybridisation conditions.

48. The virus-like particle of claim 36 wherein the polyprotein is hemagglutinin (HA) of influenza A virus.

49. The virus-like particle of claim 48 wherein the chimeric fusion protein is encoded by the nucleotide sequence set forth in SEQ ID NO: 22 or 24 or a functional variant thereof having at least 95% sequence identity thereto or a sequence that hybridises to SEQ ID NO: 22 or 24 or a complementary sequence of either of these under at least medium stringency hybridisation conditions.

50. The chimeric virus-like particle of claim 36 wherein the polyprotein is gp160 or gp140 of HIV.

51. (canceled)

52. (canceled)

53. (canceled)

54. (canceled)

55. The virus-like particle of claim 50 wherein the chimeric fusion protein is encoded by the nucleotide sequence as set forth in SEQ ID NO: 18, 19, 26, 28, 30, 32, 34, or 36 or a functional variant thereof having at least 95% sequence identity thereto or a sequence that hybridises to a complementary sequence thereof under at least medium stringency hybridisation conditions.

56. The virus-like particle of claim 47 wherein the fusion protein comprises a sequence of amino acids as set forth in SEQ ID NO: 21 or a functional portion thereof or a functional variant thereof having at least 95% sequence identity.

57. The virus-like particle of claim 48 wherein the fusion protein comprises a sequence of amino acids as set forth in SEQ ID NO: 23 or 25 or a functional portion thereof or a functional variant thereof having at least 95% sequence identity.

58. The virus-like particle of claim 50 wherein the fusion protein comprises a sequence of amino acids as set forth in SEQ ID NO: 27, 29, 31, 33, 35, or 37 or a functional portion thereof or a functional variant thereof having at least 95% sequence identity.

59. The virus-like particle of claim 36 wherein the avian hepadnavirus is duck hepatitis B virus (DHBV).

60. (canceled)

61. (canceled)

62. A nucleic acid construct encoding a chimeric fusion protein wherein the nucleic acid comprises i) a contiguous sequence of nucleotides encoding a polyprotein of two or more polypeptides of interest and ii) a sequence of nucleotides encoding a virus-like particle-associating portion of an L polypeptide of an avian hepadnavirus.

63. The nucleic acid of claim 62 wherein the chimeric fusion protein comprises a polyprotein of two or more polypeptides of interest and comprises a particle-associating portion of L polypeptide, and wherein each of said polypeptides is operably connected to a transmembrane domain and/or a protein binding domain.

64. The nucleic acid of claim 62 wherein the polyprotein is a precursor of two or more polypeptides of interest each comprising a transmembrane domain and/or a protein binding domain.

65. The nucleic acid of claim 63 wherein the transmembrane domain is derived from the polyprotein or from an avian hepadnavirus L or S polypeptide.

66. The nucleic acid of claim 65 claim 63 wherein the transmembrane domain or protein binding domain mediates binding of at least one polyprotein derived polypeptide to the VLP via non-peptide bonds.

67. The nucleic acid of claim 62 claim 63 wherein the protein binding domain contains residues for the formation of a disulphide bond between said envelope polypeptides or between an envelope polypeptide and L or S polypeptide.

68. The nucleic acid of claim 62 wherein the polyprotein is Plasmodium MSP2 polypeptide.

69. A chimeric virus-like particle comprising S polypeptide of avian hepadnavirus or a functional variant thereof and i) a chimeric fusion protein comprising a polypeptide of interest produced from a polyprotein, covalently attached to a particle-associating portion of L polypeptide of avian hepadnavirus and ii) a second or further polypeptide of interest also produced from said polyprotein, associated with the virus-like particle by a non-peptide bond.

70. The virus-like particle of claim 69 wherein the chimeric fusion protein comprises a polyprotein of two or more polypeptides of interest and comprises a particle-associating portion of L polypeptide, and wherein each of said polypeptides is operably connected to a transmembrane domain and/or a protein binding domain.

71. The virus-like particle of claim 69 wherein the polyprotein is a precursor of two or more polypeptides each comprising a transmembrane domain and/or a protein binding domain.

72. The virus-like particle of claim 70 wherein the transmembrane domain is derived from the viral envelope polyprotein or from an avian hepadnavirus L or S polypeptide.

73. The virus-like particle of claim 70 wherein transmembrane domain or protein binding domain mediates binding of at least one polyprotein derived polypeptide to the VLP via non-peptide bonds.

74. The virus-like particle of claim 70 wherein the protein binding domain contains residues for the formation of a disulphide bond between said envelope polypeptides or between an envelope polypeptide and L or S polypeptide.

75. The particle of claim 69 wherein the polyprotein is Plasmodium MSP2 polypeptide.
